(19) United States
(12) Patent Application Publication
(10) Pub. No.: US 2003/0207394 A1
(43) Pub. Date: Nov. 6, 2003

(54) Novel proteins and nucleic acids encoding same

(76) Inventors: John P. Alsobrook II, Madison, CT (US); Ferenc L. Boldog, North Haven, CT (US); Catherine E. Burgess, Wethersfield, CT (US); Stacie J. Casman, North Haven, CT (US); William M. Grosse, Branford, CT (US); Vladimir Y. Gusev, Madison, CT (US); Weizhen Ji, Branford, CT (US); Denise M. Lepley, Branford, CT (US); Xiaohong Liu, Branford, CT (US); Amanda J. Mezick, Hamden, CT (US); Muralidhara Padigaru, Branford, CT (US); Meera Patturajan, Branford, CT (US); Luca Rastelli, Guilford, CT (US); Lei Shen, Hamden, CT (US); Suresh G. Shenoy, Branford, CT (US); Richard A. Shimkets, Guilford, CT (US); Steven K. Spaderna, Berlin, CT (US); Kimberly A. Spytek, New Haven, CT (US); Edward S. Szekeres JR., Wallingford, CT (US); Raymond J. Taupier JR., East Haven, CT (US); Velizar T. Tchernev, Branford, CT (US); Bryan D. Zerhusen, Branford, CT (US); Edward Z. Voss, Wallingford, CT (US)

Correspondence Address: Ivor R. Elrifi, Esq., Mintz, Levin, Cohn, Ferris,, Glovsky and Popeo, P.C., One Financial Center, Boston, MA, 02111, US

(21) Appl. No.: 10/190,115

(22) Filed: Jul. 3, 2002

Related U.S. Application Data
(63) Continuation of application No. 09/898,994, filed on Jul. 3, 2001.
(60) Provisional application No. 60/303,168, filed on Jul. 5, 2001.
(60) Provisional application No. 60/368,996, filed on Apr. 1, 2002.
(60) Provisional application No. 60/386,816, filed on Jun. 7, 2002.
(60) Provisional application No. 60/215,854, filed on Jul. 3, 2000.
(60) Provisional application No. 60/215,856, filed on Jul. 3, 2000.
(60) Provisional application No. 60/215,902, filed on Jul. 3, 2000.
(60) Provisional application No. 60/216,585, filed on Jul. 7, 2000.
(60) Provisional application No. 60/216,586, filed on Jul. 7, 2000.
(60) Provisional application No. 60/216,722, filed on Jul. 7, 2000.
(60) Provisional application No. 60/218,622, filed on Jul. 17, 2000.
(60) Provisional application No. 60/218,992, filed on Jul. 17, 2000.
(60) Provisional application No. 60/221,285, filed on Jul. 27, 2000.
(60) Provisional application No. 60/268,734, filed on Feb. 14, 2001.
(60) Provisional application No. 60/274,260, filed on Mar. 8, 2001.
(60) Provisional application No. 60/279,856, filed on Mar. 29, 2001.

(51) Int. Cl. C12P 21/02; C12N 5/06; C07K 14/47; C07H 21/04

(52) U.S. Cl. 435/69.1; 435/320.1; 435/325; 530/350; 536/23.5

(57) ABSTRACT

Disclosed herein are nucleic acid sequences that encode novel polypeptides. Also disclosed are polypeptides encoded by these nucleic acid sequences, and antibodies, which immunospecifically-bind to the polypeptide, as well as derivatives, variants, mutants or fragments of the aforementioned polypeptide, polynucleotide, or antibody. The invention further discloses therapeutic, diagnostic and research methods for diagnosis, treatment, and prevention of disorders involving any one of these novel human nucleic acids and proteins.

### RELATED APPLICATIONS

This application claims priority from U.S. Ser. No. 60/303,168, filed Jul. 5, 2001; U.S. Ser. No. 60/368,996, filed Apr. 1, 2002; U.S. Ser. No. 60/386,816, filed Jun. 7, 2002, Express Mail Label No. EV054302049US, filed Jun. 17, 2002 which is a continuation of Ser. No. 09/898,994, filed Jul. 3, 2001, which claims priority to U.S. Ser. No. 60/215,854, filed Jul. 3, 2000; U.S. Ser. No. 60/215,856, filed Jul. 3, 2000; U.S. Ser. No. 60/215,902, filed Jul. 3, 2000; U.S. Ser. No. 60/216,585, filed Jul. 7, 2000; U.S. Ser. No. 60/216,586, filed on Jul. 7, 2001; U.S. Ser. No. 60/216,722, filed Jul. 7, 2000; U.S. Ser. No. 60/218,622, filed Jul. 17, 2000; U.S. Ser. No. 60/218,992, filed on Jul. 17, 2000; U.S. Ser. No. 60/221,285, filed Jul. 27, 2000; U.S. Ser. No. 60/268,734, filed Feb. 14, 2001; U.S. Ser. No. 60/274,260, filed Mar. 8, 2001; and U.S. Ser. No. 60/279,856, filed Mar. 29, 2001; each of which is incorporated by reference in its entirety.

### BACKGROUND OF THE INVENTION

The invention generally relates to nucleic acids and polypeptides. More particularly, the invention relates to nucleic acids encoding novel molecule (MOL) polypeptides, as well as vectors, host cells, antibodies, and recombinant methods for producing these nucleic acids and polypeptides.

### SUMMARY OF THE INVENTION

The invention is based in part upon the discovery of nucleic acid sequences encoding novel polypeptides. The novel nucleic acids and polypeptides are referred to herein as MOLX, or MOL1, MOL2, MOL3, MOL4, MOL5, MOL6, MOL7, and MOL8 nucleic acids and polypeptides. These nucleic acids and polypeptides, as well as derivatives, homologs, analogs and fragments thereof, will hereinafter be collectively designated as “MOLX” nucleic acid or polypeptide sequences.

In one aspect, the invention provides an isolated MOLX nucleic acid molecule encoding a MOLX polypeptide that includes a nucleic acid sequence that has identity to the nucleic acids disclosed in SEQ ID NOS:1, 3, 5, 7, 9, 11, 13, 15, 17, 19, 21, 23, 101, 103, 105, 107, 109, 112, 115, 117, 119, 121, and 124. In some embodiments, the MOLX nucleic acid molecule will hybridize under stringent conditions to a nucleic acid sequence complementary to a nucleic acid molecule that includes a protein-coding sequence of a MOLX nucleic acid sequence. The invention also includes an isolated nucleic acid that encodes a MOLX polypeptide, or a fragment, homolog, analog or derivative thereof. For example, the nucleic acid can encode a polypeptide at least 80% identical to a polypeptide comprising the amino acid sequences of SEQ ID NOS:2, 4, 6, 8, 10, 12, 14, 16, 18, 20, 22, 24, 102, 104, 106, 108, 111, 114, 116, 118, 120, 123, and 125. The nucleic acid can be, for example, a genomic DNA fragment or a cDNA molecule that includes the nucleic acid sequence of any of SEQ ID NOS:1, 3, 5, 7, 9, 11, 13, 15, 17, 19, 21, 23, 101, 103, 105, 107, 109, 112, 115, 117, 119, 121, and 124.

Also included in the invention is an oligonucleotide, e.g, an oligonucleotide which includes at least 6 contiguous nucleotides of a MOLX nucleic acid (e.g, SEQ ID NOS:1, 3, 5, 7, 9, 11, 13, 15, 17, 19, 21, 23, 101, 103, 105, 107, 109, 112, 115, 117, 119, 121, and 124) or a complement of said oligonucleotide.

Also included in the invention are substantially purified MOLX polypeptides (SEQ ID NOS:2, 4, 6, 8, 10, 12, 14, 16, 18, 20, 22, 24, 102, 104, 106, 108, 111, 114, 116, 118, 120, 123, and 125). In certain embodiments, the MOLX polypeptides include an amino acid sequence that is substantially identical to the amino acid sequence of a human MOLX polypeptide.

The invention also features antibodies that immunoselectively bind to MOLX polypeptides, or fragments, homologs, analogs or derivatives thereof.

In another aspect, the invention includes pharmaceutical compositions that include therapeutically- or prophylactically-effective amounts of a therapeutic and a pharmaceutically-acceptable carrier. The therapeutic can be, e g., a MOLX nucleic acid, a MOLX polypeptide, or an antibody specific for a MOLX polypeptide. In a further aspect, the invention includes, in one or more containers, a therapeutically- or prophylactically-effective amount of this pharmaceutical composition.

In a further aspect, the invention includes a method of producing a polypeptide by culturing a cell that includes a MOLX nucleic acid, under conditions allowing for expression of the MOLX polypeptide encoded by the DNA. If desired, the MOLX polypeptide can then be recovered.

In another aspect, the invention includes a method of detecting the presence of a MOLX polypeptide in a sample. In the method, a sample is contacted with a compound that selectively binds to the polypeptide under conditions allowing for formation of a complex between the polypeptide and the compound. The complex is detected, if present, thereby identifying the MOLX polypeptide within the sample.

The invention also includes methods to identify specific cell or tissue types based on their expression of a MOLX.

Also included in the invention is a method of detecting the presence of a MOLX nucleic acid molecule in a sample by contacting the sample with a MOLX nucleic acid probe or primer, and detecting whether the nucleic acid probe or primer bound to a MOLX nucleic acid molecule in the sample.

In a further aspect, the invention provides a method for modulating the activity of a MOLX polypeptide by contacting a cell sample that includes the MOLX polypeptide with a compound that binds to the MOLX polypeptide in an amount sufficient to modulate the activity of said polypeptide. The compound can be, e.g, a small molecule, such as a nucleic acid, peptide, polypeptide, peptidomimetic, carbohydrate, lipid or other organic (carbon-containing) or inorganic molecule, as further described herein.

Also within the scope of the invention is the use of a therapeutic in the manufacture of a medicament for treating or preventing disorders or syndromes including, e.g., endometriosis, fertility disorders, hypercoagulation, idiopathic thrombocytopenic purpura, immunodeficiencies, systemic lupus erythematosus, asthma, emphysema, scleroderma, allergy, ARDS, Von Hippel-Lindau (VHL) syndrome, Alzheimer's disease, stroke, tuberous sclerosis, hypercalceimia, Parkinson's disease, Huntington's disease, cerebral palsy, epilepsy, multiple sclerosis, ulcers, ataxia-telangiectasia, leukodystrophies, behavioral disorders, addiction, anxiety, pain, neuroprotection, osteoporosis, arthritis, ankylosing spondylitis, scoliosis, diabetes, autoimmune disease, myasthenia gravis, muscular dystrophy, renal artery stenosis, interstitial nephritis, glomerulonephritis, polycystic kidney disease, systemic lupus erythematosus, renal tubular acidosis, IgA nephropathy, Lesch-Nyhan syndrome, developmental disorders, growth disorders, and/or wounds, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, obesity, transplantation, hemophilia, hypercoagulation, idiopathic thrombocytopenic purpura, immunodeficiencies, graft versus host disease (GVHD), lymphaedema, adrenoleukodystrophy, congenital adrenal hyperplasia, neuronal developmental, organizational, mediated and interactive disorders and disease; endocrine dysfunctions, growth and reproductive disorders, injury repair, cancer including but not limited to lung or breast cancer, endocrine disorders, inflammatory disorders, gastro-intestinal disorders and disorders of the respiratory system, Rheumatoid arthritis (RA), CNS disorders, Down syndrome, Schizophrenia, nutritional deficiencies, primary open-angle glaucoma (POAG), and bone disorders, hematopoietic disorders, or other disorders. The therapeutic can be, e.g., a MOLX nucleic acid, a MOLX polypeptide, or a MOLX-specific antibody, or biologically-active derivatives or fragments thereof.

For example, the compositions of the present invention will have efficacy for treatment of patients suffering from: Cancer including endometriosis, fertility disorders, hypercoagulation, idiopathic thrombocytopenic purpura, immunodeficiencies, systemic lupus erythematosus, asthma, emphysema, scleroderma, allergy, ARDS, Von Hippel-Lindau (VHL) syndrome, Alzheimer's disease, stroke, tuberous sclerosis, hypercalceimia, Parkinson's disease, Huntington's disease, cerebral palsy, epilepsy, multiple sclerosis, ulcers, ataxia-telangiectasia, leukodystrophies, behavioral disorders, addiction, anxiety, pain, neuroprotection, osteoporosis, arthritis, ankylosing spondylitis, scoliosis, diabetes, autoimmune disease, myasthenia gravis, muscular dystrophy, renal artery stenosis, interstitial nephritis, glomerulonephritis, polycystic kidney disease, systemic lupus erythematosus, renal tubular acidosis, IgA nephropathy, Lesch-Nyhan syndrome, developmental disorders, growth disorders, and/or wounds, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, obesity, transplantation, hemophilia, hypercoagulation, idiopathic thrombocytopenic purpura, immunodeficiencies, graft versus host disease (GVHD), lymphaedema, adrenoleukodystrophy, congenital adrenal hyperplasia, neuronal developmental, organizational, mediated and interactive disorders and disease; endocrine dysfunctions, growth and reproductive disorders, injury repair, cancer including but not limited to lung or breast cancer, endocrine disorders, inflammatory disorders, gastro-intestinal disorders and disorders of the respiratory system, Rheumatoid arthritis (RA), CNS disorders, Down syndrome, Schizophrenia, nutritional deficiencies, primary open-angle glaucoma (POAG), and bone disorders, hematopoietic disorders and/or other pathologies and disorders of the like.

The polypeptides can be used as immunogens to produce antibodies specific for the invention and as vaccines. They can also be used to screen for potential agonist and antagonist compounds. For example, a cDNA encoding MOLX may be useful in gene therapy, and MOLX may be useful when administered to a subject in need thereof. By way of nonlimiting example, the compositions of the present invention will have efficacy for treatment of patients suffering from endometriosis, fertility disorders, hypercoagulation, idiopathic thrombocytopenic purpura, immunodeficiencies, systemic lupus erythematosus, asthma, emphysema, scleroderma, allergy, ARDS, Von Hippel-Lindau (VHL) syndrome, Alzheimer's disease, stroke, tuberous sclerosis, hypercalceimia, Parkinson's disease, Huntington's disease, cerebral palsy, epilepsy, multiple sclerosis, ulcers, ataxia-telangiectasia, leukodystrophies, behavioral disorders, addiction, anxiety, pain, neuroprotection, osteoporosis, arthritis, ankylosing spondylitis, scoliosis, diabetes, autoimmune disease, myasthenia gravis, muscular dystrophy, renal artery stenosis, interstitial nephritis, glomerulonephritis, polycystic kidney disease, systemic lupus erythematosus, renal tubular acidosis, IgA nephropathy, Lesch-Nyhan syndrome, developmental disorders, growth disorders, and/or wounds, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, obesity, transplantation, hemophilia, hypercoagulation, idiopathic thrombocytopenic purpura, immunodeficiencies, graft versus host disease (GVHD), lymphaedema, adrenoleukodystrophy, congenital adrenal hyperplasia, neuronal developmental, organizational, mediated and interactive disorders and disease; endocrine dysfunctions, growth and reproductive disorders, injury repair, cancer including but not limited to lung or breast cancer, endocrine disorders, inflammatory disorders, gastro-intestinal disorders and disorders of the respiratory system, Rheumatoid arthritis (RA), CNS disorders, Down syndrome, Schizophrenia, nutritional deficiencies, primary open-angle glaucoma (POAG), and bone disorders, hematopoietic disorders and/or other pathologies and disorders.

The invention further includes a method for screening for a modulator of disorders or syndromes including, e.g., endometriosis, fertility disorders, hypercoagulation, idiopathic thrombocytopenic purpura, immunodeficiencies, systemic lupus erythematosus, asthma, emphysema, scleroderma, allergy, ARDS, Von Hippel-Lindau (VHL) syndrome, Alzheimer's disease, stroke, tuberous sclerosis, hypercalceimia, Parkinson's disease, Huntington's disease, cerebral palsy, epilepsy, multiple sclerosis, ulcers, ataxia-telangiectasia, leukodystrophies, behavioral disorders, addiction, anxiety, pain, neuroprotection, osteoporosis, arthritis, ankylosing spondylitis, scoliosis, diabetes, autoimmune disease, myasthenia gravis, muscular dystrophy, renal artery stenosis, interstitial nephritis, glomerulonephritis, polycystic kidney disease, systemic lupus erythematosus, renal tubular acidosis, IgA nephropathy, Lesch-Nyhan syndrome, developmental disorders, growth disorders, and/or wounds, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, obesity, transplantation, hemophilia, hypercoagulation, idiopathic thrombocytopenic purpura, immunodeficiencies, graft versus host disease (GVHD), lymphaedema, adrenoleukodystrophy, congenital adrenal hyperplasia, neuronal developmental, organizational, mediated and interactive disorders and disease; endocrine dysfunctions, growth and reproductive disorders, injury repair, cancer including but not limited to lung or breast cancer, endocrine disorders, inflammatory disorders, gastro-intestinal disorders and disorders of the respiratory system, Rheumatoid arthritis (RA), CNS disorders, Down syndrome, Schizophrenia, nutritional deficiencies, primary open-angle glaucoma (POAG), and bone disorders, hematopoietic disorders or other disorders related to cell signal processing and metabolic pathway modulation. The method includes contacting a test compound with a MOLX polypeptide and determining if the test compound binds to said MOLX polypeptide. Binding of the test compound to the MOLX polypeptide indicates the test compound is a modulator of activity, or of latency or predisposition to the aforementioned disorders or syndromes.

Also within the scope of the invention is a method for screening for a modulator of activity, or of latency or predisposition to an disorders or syndromes including, e g., endometriosis, fertility disorders, hypercoagulation, idiopathic thrombocytopenic purpura, immunodeficiencies, systemic lupus erythematosus, asthma, emphysema, scleroderma, allergy, ARDS, Von Hippel-Lindau (VHL) syndrome, Alzheimer's disease, stroke, tuberous sclerosis, hypercalceimia, Parkinson's disease, Huntington's disease, cerebral palsy, epilepsy, multiple sclerosis, ulcers, ataxia-telangiectasia, leukodystrophies, behavioral disorders, addiction, anxiety, pain, neuroprotection, osteoporosis, arthritis, munankylosing spondylitis, scoliosis, diabetes, autoimmune disease, myasthenia gravis, muscular dystrophy, renal artery stenosis, interstitial nephritis, glomerulonephritis, polycystic kidney disease, systemic lupus erythematosus, renal tubular acidosis, IgA nephropathy, Lesch-Nyhan syndrome, developmental disorders, growth disorders, and/or wounds, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, obesity, transplantation, hemophilia, hypercoagulation, idiopathic thrombocytopenic purpura, immunodeficiencies, graft versus host disease (GVHD), lymphaedema, adrenoleukodystrophy, congenital adrenal hyperplasia, neuronal developmental, organizational, mediated and interactive disorders and disease; endocrine dysfunctions, growth and reproductive disorders, injury repair, cancer including but not limited to lung or breast cancer, endocrine disorders, inflammatory disorders, gastro-intestinal disorders and disorders of the respiratory system, Rheumatoid arthritis (RA), CNS disorders, Down syndrome, Schizophrenia, nutritional deficiencies, primary open-angle glaucoma (POAG), and bone disorders, hematopoietic disorders or other disorders related to cell signal processing and metabolic pathway modulation by administering a test compound to a test animal at increased risk for the aforementioned disorders or syndromes. The test animal expresses a recombinant polypeptide encoded by a MOLX nucleic acid. Expression or activity of MOLX polypeptide is then measured in the test animal, as is expression or activity of the protein in a control animal which recombinantly-expresses MOLX polypeptide and is not at increased risk for the disorder or syndrome. Next, the expression of MOLX polypeptide in both the test animal and the control animal is compared. A change in the activity of MOLX polypeptide in the test animal relative to the control animal indicates the test compound is a modulator of latency of the disorder or syndrome.

In yet another aspect, the invention includes a method for determining the presence of or predisposition to a disease associated with altered levels of a MOLX polypeptide, a MOLX nucleic acid, or both, in a subject (e.g., a human subject). The method includes measuring the amount of the MOLX polypeptide in a test sample from the subject and comparing the amount of the polypeptide in the test sample to the amount of the MOLX polypeptide present in a control sample. An alteration in the level of the MOLX polypeptide in the test sample as compared to the control sample indicates the presence of or predisposition to a disease in the subject. Preferably, the predisposition includes, e.g., endornetriosis, fertility disorders, hypercoagulation, idiopathic thrombocytopenic purpura, immunodeficiencies, systemic lupus erythematosus, asthma, emphysema, scleroderma, allergy, ARDS, Von Hippel-Lindau (VHL) syndrome, Alzheimer's disease, stroke, tuberous sclerosis, hypercalceimia, Parkinson's disease, Huntington's disease, cerebral palsy, epilepsy, multiple sclerosis, ulcers, ataxia-telangiectasia, leukodystrophies, behavioral disorders, addiction, anxiety, pain, neuroprotection, osteoporosis, arthritis, ankylosing spondylitis, scoliosis, diabetes, autoimmune disease, myasthenia gravis, muscular dystrophy, renal artery stenosis, interstitial nephritis, glomerulonephritis, polycystic kidney disease, systemic lupus erythematosus, renal tubular acidosis, IgA nephropathy, Lesch-Nyhan syndrome, developmental disorders, growth disorders, and/or wounds, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, obesity, transplantation, hemophilia, hypercoagulation, idiopathic thrombocytopenic purpura, immunodeficiencies, graft versus host disease (GVHD), lymphaedema, adrenoleukodystrophy, congenital adrenal hyperplasia, neuronal developmental, organizational, mediated and interactive disorders and disease; endocrine dysfunctions, growth and reproductive disorders, injury repair, cancer including but not limited to lung or breast cancer, endocrine disorders, inflammatory disorders, gastro-intestinal disorders and disorders of the respiratory system, Rheumatoid arthritis (RA), CNS disorders, Down syndrome, Schizophrenia, nutritional deficiencies, primary open-angle glaucoma (POAG), and bone disorders, hematopoietic disorders. Also, the expression levels of the new polypeptides of the invention can be used in a method to screen for various cancers as well as to determine the stage of cancers.

In a further aspect, the invention includes a method of treating or preventing a pathological condition associated with a disorder in a mammal by administering to the subject a MOLX polypeptide, a MOLX nucleic acid, or a MOLX-specific antibody to a subject (e.g., a human subject), in an amount sufficient to alleviate or prevent the pathological condition. In preferred embodiments, the disorder, includes, e g., endometriosis, fertility disorders, hypercoagulation, idiopathic thrombocytopenic purpura, immunodeficiencies, systemic lupus erythematosus, asthma, emphysema, scleroderma, allergy, ARDS, Von Hippel-Lindau (VHL) syndrome, Alzheimer's disease, stroke, tuberous sclerosis, hypercalceimia, Parkinson's disease, Huntington's disease, cerebral palsy, epilepsy, multiple sclerosis, ulcers, ataxia-telangiectasia, leukodystrophies, behavioral disorders, addiction, anxiety, pain, neuroprotection, osteoporosis, arthritis, ankylosing spondylitis, scoliosis, diabetes, autoimmune disease, myasthenia gravis, muscular dystrophy, renal artery stenosis, interstitial nephritis, glomerulonephritis, polycystic kidney disease, systemic lupus erythematosus, renal tubular acidosis, IgA nephropathy, Lesch-Nyhan syndrome, developmental disorders, growth disorders, and/or wounds, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, obesity, transplantation, hemophilia, hypercoagulation, idiopathic thrombocytopenic purpura, immunodeficiencies, graft versus host disease (GVHD), lymphaedema, adrenoleukodystrophy, congenital adrenal hyperplasia, neuronal developmental, organizational, mediated and interactive disorders and disease; endocrine dysfunctions, growth and reproductive disorders, injury repair, cancer including but not limited to lung or breast cancer, endocrine disorders, inflammatory disorders, gastro-intestinal disorders and disorders of the respiratory system, Rheumatoid arthritis (RA), CNS disorders, Down syndrome, Schizophrenia, nutritional deficiencies, primary open-angle glaucoma (POAG), and bone disorders, hematopoietic disorders, and/or other diseases or disorders.

In yet another aspect, the invention can be used in a method to identity the cellular receptors and downstream effectors of the invention by any one of a number of techniques commonly employed in the art. Thesc include but are not limited to the two-hybrid system, affinity purification, co-precipitation with antibodies or other specific-interacting molecules.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. All publications, patent applications, patents, and other references mentioned herein are incorporated by reference in their entirety. In the case of conflict, the present specification, including definitions, will control. In addition, the materials, methods, and examples are illustrative only and not intended to be limiting.

Other features and advantages of the invention will be apparent from the following detailed description and claims.

### DETAILED DESCRIPTION OF THE INVENTION

The invention is based, in part, upon the discovery of novel nucleic acid sequences that encode novel polypeptides. The novel nucleic acids and their encoded polypeptides are referred to individually as MOL1, MOL2, MOL3, MOL4, MOL5, MOL6, MOL7, and MOL8. The nucleic acids, and their encoded polypeptides, are collectively designated herein as “MOLX”.

The novel MOLX nucleic acids of the invention include the nucleic acids whose sequences are provided in Tables 1A, 1D, 2A, 3A, 3D, 4A, 5A, 5C, 5E, 6A, 7A, and 8A. inclusive (“Tables 1A-8A”), or a fragment, derivative, analog or homolog thereof. The novel MOLX proteins of the invention include the protein fragments whose sequences are provided in Tables 1B, 1E, 2B, 3B, 3E, 4B, 5B, 5D, 5F, 6B, 7B, and 8B inclusive (“Tables 1B-8B”). The individual MOLX nucleic acids and proteins are described below. Within the scope of this invention is a method of using these nucleic acids and peptides in the treatment or prevention of a disorder related to cell signaling or metabolic pathway modulation.

### MOL1

### MOL1a

A disclosed Notch-like nucleic acid of 7410 nucleotides, MOL1a, alternatively referred to as SC29674552_EXT, is shown in Table 1A. The disclosed MOL1a open reading frame (“ORF”) begins at the ATG initiation codon at nucleotides 1-3 and terminates at a TGA codon at nucleotides 7408-7410. In Table 1A, the start and stop codons are depicted with bold letters.

TABLE 1A

MOL1a nucleotide sequence.

(SEQ ID NO:1)

ATGCCCGCCCTGCGCCCCGCTCTGCTGTGCGCCCTCCTGGCCCTCTCGCTGTGCTGCGCGACCCCCGCGCATGCATT
GCACTGTCGAGATGGCTATCAACCCTGTGTAAATGAAGGAATGTGTGTTACCTACCACAATGGCACAGGATACTGCA
AATGTCCAGAAGGCTTCTTCGGGGAATATTGTCAACATCGAGACCCCTGTGAGAAGAACCGCTGCCAGAATCGTGGC
ACTTGTGTGGCCCAGGCCATGCTGGGGAAAGCCACGTCCCCATGTCCCTCAGGGTTTACAGCAGAGGACTGCCAGTA
CTCGACATCTCATCCATGCTTTGTGTCTCGACCTTGCCTGAATGGCCCCACATGCCATATGCTCAGCCGCGATACCT
ATGAGTGCACCTGTCAAGTCGGGTTTACAGGTAACGAGTGCCAATGGACGGATCCCTGCCTGTCTCATCCCTGTCCA
AATGCAAGTACCTGTACCACTGTGGCCAACCAGTTCTCCTGCAAATCCCTCACAGGCTTCACAGGGCACAAATGTGA
GACTCATGTCAATGAGTGTGACATTCCAGGACACTGCCAGCATGGTGCCACCTCCCTCAACCTGCCTGGTTCCTACC
AGTGCCAGTGCCCTCAGGCCTTCACAGCCCACTACTGTGACAGCCTGTATGTGCCCTCTGCACCCTCACCTTGTGTC
AATGGAGGCACCTGTCCCCAGACTGGTCACTTCACTTTTGACTGCCATTTACCAGGTTTTCAAGGGAGCACCTGTGA
GAGGAATATTGATGACTCCCCTAACCACACGTGTCAGAATGGACGGGTTTGTGTGGATGGGGTCAACACTTACAACT
GCCGCTGTCCCCCACAATGGACAGGACAGTTCTGCACACAGCATGTGGATGAATGCCTCCTGCAGCCCAATGCCTGT
CAAAACTGGGGCACCTCTGCCAACCGCAATGGAGGCTATCCCTGTGTATGTCTCAACGCCTCCACTGGAGATGACTG
CAGTGACAACATTGATGATTGTGCTTTCGCCGCCTGTACTCCAGGCTCCACCTGCATCGACCGTGTCGCCTCCTTCT
CTTGCATGTCCCCAGAGCGGAAGGCAGGTCTCCTGTGTCATCTGGATGATGCATGCATCAGCAATCCTTGCCACAAG
GGGGCACTGTGTGACACCAACCCCCTAAATGGGCAATATATTTGCACCTCCCCACAAGGCTACAAAGGGGCTGACTG
CACAGAAGATGTCCATGAATGTCCCATGGCCAATAGCAATCCTTGTGAGCATGCAGGAAAATGTGTGAACACGGATG
GCGCCTTCCACTGTGAGTGTCTGAAGGGTTATGCAGGACCTCGTTGTGAGATGCACATCAATGAGTCCCATTCAGAC
CCCTGCCAGAATGATGCTACCTCTCTGGATAAGATTGGAGGCTTCACATGTCTGTGCATGCCAGGTTTCAAAGGTGT
GCATTGTGAATTAGAAATAAATGAATGTCAGAGCAACCCTTGTGTGAACAATGGGCAGTCTGTGGATAAAGTCAATC
GTTTCCACTGCCTGTGTCCTCCTGGTTTCACTGGCCCAGTTTGCCAGATTGATATTGATGACTGTTCCAGTACTCCG
TGTCTGAATGGGGCAAAGTGTATCGATCACCCGAATGGCTATCAATGCCAGTGTGCCACAGGTTTCACTCGTGTGTT
GTGTGAGGAGAACATTGACAACTGTGACCCCGATCCTTGCCACCATGGTCAGTGTCAGCATGGTATTGATTCCTACA
CCTGCATCTGCAATCCCGCGTACATGCGCGCCATCTGCAGTGACCACATTGATGAATGTTACAGCAGCCCTTGCCTG
AACGATGGTCGCTGCATTGACCTGGTCAATGCCTACCAGTCCAACTGCCAGCCAGGCACGTCAGGTGTTAATTGTGA
AATTAATTTTGATGACTGTGCAAGTAACCCTTGTATCCATCGAATCTCTATCGATCGCATTAATCGCTACACTTGTG
TCTGCTCACCAGGATTCACACGGCAGAGATGTAACATTCACATTGATGAGTGTGCCTCCAATCCCTGTCGCAAGGGT
GCAACATGTATCAACGGTGTCAATGGTTTCCGCTGTATATGCCCCGAGGGACCCCATCACCCCAGCTCCTACTCACA
GGTGAACGAATGCCTGAGCAATCCCTGCATCCATGCAAACTGTACTGCAGGTCTCAGTGGATATAACTGTCTCTGTG
ATGCACGCTGGGTTCGCATCAACTGTGAAGTGGACAAAAATGAATCCCTTTCGAATCCATGCCAGAATGGAGGAACT
TGTGACAATCTGGTGAATGGATACAGGTGTACTTGCAAGAAGGGCTTTAAACGCTATAACTCCCAGGTGAATATTGA
TGAATGTGCCTCAAATCCATGCCTCAACCAACGAACCTGCTTTCATGACATAAGTGGCTACACTTCCCACTGTGTGC
TGCCATACACAGGTAAGAATTCTCACACAGTATTGGCTCCCTGTTCCCCAAACCCTTGTGAGAATGCTGCTGTTTGC
AAAGAGTCACCAAATTTTGAGAGTTATACTTCCTTGTGTGCTCCTGCCTGGCAAGGTCAGCGGTGTACCATTGACAT
TGACGAGTGTATCTCCAAGCCCTGCATGAACCATGGTCTCTGCCATAACACCCACGCCACCTACATCTGTCAATGTC
CACCAGGCTTCAGTGGTATGGACTGTGAGGAGGACATTGATGACTGCCTTGCCAGTCCTTGCCAGAATGGAGGTTCC
TGTATCCATGCACTCAATACTTTCTCCTGCCTCTGCCTTCCGGCTTTCACTGGGGATAAGTGCCAGACAGACATCAA
TGAGTGTCTGAGTGAACCCTCTAACAATGGAGGGACCTGCTCTGACTACGTCAACAGTTACACTTGCAAGTGCCAGG
CAGGATTTGATGCAGTCCATTGTGAGAACAACATCAATCAGTCCACTGAGAGCTCCTGTTTCAATGGTGGCACATCT
GTTGATGGCATTAACTCCTTCTCTTCCTTGTGCCCTGTCGGTTTCACTGCATCCTTCTCCCTCCATCAGATCAATGA
ATGCAGCTCTCATCCATGCCTGAATGATCGAACGTGTGTTGATCGCCTGCGTACCTACCGCTGCACCTGCCCCCTGG
GCTACACTCGGAAAAACTGTCAGACCCTGGTGAATCTCTGCAGTCGGTCTCCATCTAAAAACAAAGGTACTTGCCTT
CAGAAAAAAGCAGAGTCCCAGTGCCTATGTCCATCTGGATGGGCTGGTCCCTATTGTGACGTGCCCAATGTCTCTTC
TGACATACCAGCCTCCACGAGAGGTGTCCTTGTTCAACACTTGTGCCACCACTCACCTGTCTCCATCAATCCTGGCA
ACACGCATTACTGTCACTGCCCCCTGGGCTATACTGGGACCTACTGTGAGGAGCAACTCGATGAGTGTGCCTCCAAC
CCCTGCCAGCACGGGGCAACATGCAGTGACTTCATTGGTGGATACAGATGCGAGTGTGTCCCACGCTATCAGGGTGT
CAACTGTGAGTATGAACTGCATGAGTGCCAGAATCAGCCCTGCCAGAATGGAGCCACCTGTATTGACCTTGTGAACC
ATTTCAAGTGCTCTTGCCCACCAGCCACTCGGGGTATGAAATCATCCTTATCCATTTTCCATTGCCCGGGTCCCCAT
TGCCTTAATGGTGGTCAGTGCATGGATAGGATTGGACCCTACAGTTGTCGCTGCTTGCCTGGCTTTGCTGGGGAGCG
TTGTGAGGGAGACATCAACGAGTGCCTCTCCAACCCCTGCAGCTCTGAGGGCAGCCTCGACTCTATACAGCTCACCA
ATGACTACCTGTCTCTTTGCCGTAGTGCCTTTACTGGTCCGCACTGTGAAACCTTCGTCGATCTCTGTCCCCAGATG
CCCTGCCTGAATGGAGGGACTTGTGCTCTGCCCACTAACATGCCTGATCGTTCATTTGCCGTTGTCCCCCAGGCATT
TTCCCGCGCAAGGTCCCAGAGCAGCTGTGGACAAGTGAAATGTACCAAGGGCGAGCAGTGTATGCACACCGCCTCTG
GACCCCGCTGCTTCTGCCCCAGTCCCCGGGACTGCGAGTCAGGCTGTGCCAGTAGCCCCTGCCAGCACGGGGCCAGC
TGCCACCCTCAGCGCCAGCCTCCTTATTACTCCTGCCAGTGTGCCCCACCATTCTCGGGTAGCCGCTGTGAACTCTA
CACGGCACCCCCCACCACCCCTCCTGCCACCTCTCTCAGCCAGTATTGTGCCGACAAAGCTCGGGATGGCCTCTGTC
ATGAGCCCTCCAACAGCCATGCCTGCCAGTGGCATGGGGGTCACTGTTCTCTCACCATGCAGAACCCCTGGGCCAAC
TGCTCCTCCCCACTTCCCTGCTCGCATTATATCAACAACCACTGTGATGAGCTGTGCAACACGGTCGAGTCCCTGTT
TGACAACTTTGAATGCCACGCGAACACCAAGACATGCAAGTATGACAAATACTGTCCACACCACTTCAAAGACAACC
ACTGTGACCAGCGGTGCAACAGTGAGGAGTGTGGTTGGGATGGCCTGGACTGTGCTCCTGACCAACCTGACAACCTG
GCAGAAGCTACCCTCCTTATTGTGGTATTGATGCCACCTGAACAACTGCTCCAGGATGCTCGCAGCTTCTTGCGGGC
ACTGGGTACCCTGCTCCACACCAACCTCCGCATTAAGCGGGACTCCCAGGGGGAACTCATGGTGTACCCCTATTATG
GTGAGAAGTCAGCTCCTATGAAGAAACAGACGATGACACGCAGATCCCTTCCTGGTGAACAAGAACAGGACCTCCCT
GGGTCTAAACTCTTTCTGGAAATTCACAACCGCCAGTCTGTTCAAGACTCAGACCACTGCTTCAAGAACACGCATGC
AGCAGCAGCTCTCCTGGCCTCTCACGCCATACAGGGGACCCTGTCATACCCTCTTGTGTCTCTCGTCAGTGAGTCCC
TGACTCCAGAACGCACTCAGCTCCTCTATCTCCTTGCTGTTCCTCTTGTCATCATTCTCTTTATTATTCTGCTGGCG
GTAATCATGCCAAAACGAAAGCGTAAGCATGGCTCTCTCTGGCTCCCTCAAGGTTTCACTCTTCCCCGAGATGCAAG
CAATCACAACCGTCGTCAGCCAGTGGGACAGGATGCTGTCGGCCTCAAAAATCTCTCAGTGCAAGTCTCAGAAGCTA
ACCTAATTCGTACTGCAACAAGTGAACACTGGGTCGATGATGAAGGGCCCCAGCCAAAGAAAGTAAAGGCTGAAGAT
GAGGCCTTACTCTCAGAAGAAGATGACCCCATTGATCGACGGCCATGGACACAGCAGCACCTTGAAGCTGCACACAT
CCGTAGGACACCATCGCTGGCTCTCACCCCTCCTCACGCAGAGCAGGAGGTGGATGTGTTAGATGTCAATCTCCGTG
GCCCAGATGCCTCCACCCCATTGATGTTCCCTTCTCTCCGAGGAGGCAGCTCAGATTTCAGTGATCAAGATGAACAT
GCAGAGGACTCTTCTGCTAACATCATCACAGACTTGGTCTACCAGGGTGCCAGCCTCCAGGCCCAGACACACCGGAC
TGGTGAGATGGCCCTGCACCTTGCAGCCCGCTACTCACGCGCTGATGCTGCCAAGCGTCTCCTGGATGCAGGTGCAG
ATGCCAATGCCCAGGACAACATGGGCCGCTGTCCACTCCATGCTGCAGTGGCACCTCATGCCCAAGCTGTCTTCCAG
ATTCTGATTCGCAACCGACTAACTGATCTACATGCCAGCATGAATGATGGTACTACACCCCTGATCCTGGCTGCCCG
CCTGGCTCTGGAGGCAATGGTGGCAGAACTGATCAACTGCCAAGCGGATGTGAATGCAGTGGATGACCATGGAAAAT
CTGCTCTTCACTGGCCAGCTGCTGTCAATAATCTGGAGGCAACTCTTTTGTTGTTGAAAAATCGCGCCAACCCAGAC
ATGCAGGACAACAAGGAAGAGACACCTCTGTTTCTTCCTGCCCCGGACGCCACCTATCAAGCAGCCAAGATCCTGTT
AGACCATTTTGCCAATCGACACATCACAGACCATATGGATCGTCTTCCCCCGCATCTGGCTCGGGATCGCATGCACC
ATGACATTGTGCGCCTTCTGGATCAATACAATGTGACCCCAAGCCCTCCAGGCACCGTGTTGACTTCTGCTCTCTCA
CCTGTCATCTGTGGGCCCAACAGATCTTTCCTCAGCCTGAACCACACCCCAATGGGCPAGAAGTCTAGACGGCCCAG
TGCCAAGAGTACCATGCCTACTAGCCTCCCTAACCTTGCCAAGCAGGCAAAGGATGCCAAGGCTAGTAGGAGGAACA
AGTCTCTGACTCAGAAGGTCCAACTGTCTGACAGTTCAGTAACTTTATCCCCTGTTCATTCCCTACAATCTCCTCAC
ACGTATGTTTCCGACACCACATCCTCTCCAATCATTACATCCCCTGGGATCTTACAGGCCTCACCCAACCCTATGTT
GGCCACTGCCCCCCCTCCTGCCCCAGTCCATGCCCAGCATGCACTATCTTTTTCTAACCTTCATCAAATGCAGCCTT
TGGCACATCGGGCCAGCACTGTGCTTCCCTCAGTGACCCAGTTGCTATCCCACCACCACATTGTGTCTCCAGCCAGT
GGCAGTGCTGGAACCTTCAGTAGCCTCCATCCAGTCCCAGTCCCAGCACATTGGATGAACCGCATGGAGGTGAATGA
GACCCAGTACAATGACATGTTTGGTATGGTCCTGGCTCCAGCTCTAGGCCACCCATCCTGGCATAGCTCCCCACAGA
GGCCACCTGAAGGGAAGCACATAACCACCCCTCGGGAGCCCTTCCCCCCCATTGTCACTTTCCAGCTCATCCCTAAA
GGCAGTATTGCCCAACCAGCGGGGGCTCCCCAGCCTCAGTCCACCTCCCCTCCACCTGTTGCGGGCCCCCTCCCCAC
CATGTACCAGATTCCAGAAATGGCCCGTTTCCCCAGTGTGGCTTTCCCCACTCCCATGATGCCCCAGCAGGACGGGC
AGGTAGCTCAGACCATTCTCCCAGCCTATCATCCTTTCCCAGCCTCTGTGGGCAAGTACCCCACACCCCCTTCACAG
CACAGTTATGCTTCCTCAAATCCTGCTGAGCGAACACCCAGTCACAGTGGTCACCTCCAGGGTCAGCATCCCTACCT
GACACCATCCCCAGAGTCTCCTGACCAGTGGTCAAGTTCATCACCCCACTCTGCTTCTCACTGGTCAGATGTGACCA
CCAGCCCTACCCCTCGCGGAGCTCGAGCACGTCAGCCGGGACCTCGGACACACATGTCTCACCCACCACACAACAAC
TGCAGGTTTATCCGTGA

The disclosed MOL1a nucleotide encodes a protein which has 2469 amino acid residues, referred to as the MOL1a protein. The MOL1a protein was analyzed for signal peptide prediction and cellular localization. SignalP results predict that MOL1a is cleaved between position 25 and 26 (AHA-LQ) of SEQ ID NO:2. Psort and Hydropathy profiles also predict that MOL1a contains a signal peptide and is likely to be localized in the plasma membrane (Certainty=0.4600). A disclosed MOL1a polypeptide sequence is presented in Table 1B using the one-letter amino acid code.

TABLE 1B

Encoded MOL1a protein sequence.

(SEQ ID NO:2)

MPALRPALLWALLALWLCCATPAHALQCRDGYEPCVNEGMCVTYHNGTGYCKCPEGFLGEYCQHRDPCEKNRCQNGG
TCVAQAMLCKATCRCASCFTCEDCQYSTSHPCFVSRPCLNGGTCHMLSRDTYECTCQVGFTGKECQWTDACLSHPCA
NGSTCTTVANQFSCKCLTGFTGQKCETDVNECDIPGHCQHGGTCLNLPGSYQCQCPQGFTGQYCDSLYVPCAPSPCV
NGGTCRQTCDFTFECHLPGEEGSTCERNIDDCPNHRCQNGGVCVDGVNTYNCRCPPQWTCQFCTEDVDECLLQPNAC
QNWGTCANRNGGYGCVCVNGWSCDDCSENIDDCAFCACTPCSTCIDRVASFSCMCPEGKAGLLCHLDDACISNPCHK
GALCDTNPLNGQYICTCPQCYKGADCTEDVDECANANSNPCEHAGKCVNTDGAFHCECLKCYAGPRCEMDTNECHSD
PCQNDATCLDKIGGFTCLCMPGFKGVHCELEINECQSNPCVNNGQCVDKVNRFQCLCFFGFTGFVCQIDIDDCSSTP
CLNGAKCIDHPNGYECQCATGFTGVLCEENIDNCDPDPCHHGQCQDGIDSYTCICNPGYMGAICSDQIDECYSSPCL
NDGRCIDLVNGYQCNCQPGTSGVNCEINFDDCASNPCIHGICMDGINRYSCVCSPGFTGQRCNIDIDECASNRCRKG
ATCINGVNGFRCICPEGPHHPSCYSQVNECLSNFCIHGNCTGGLSCYKCLCDACWVGTNCEVDKNECLSNPCQNGCT
CDNLVNGYRCTCKKGFKGYNCQVNIDECASNPCLNQGTCFDDISGYTCHCVLPYTGKNCQTVLAFCSPNPCENAAVC
KESPNFESYTCLCAPGWQGQRCTIDIDECISKPCMNHGLCHNTQGSYMCECPPGFSGMDCEEDIDDCLASPCQNGGS
CMDGVNTFSCLCLPGFTGDKCQTDMNECLSEPCKNGGTCSDYVNSYTCKCQAGFDGVHCENNINECTESSCFNGCTC
VDGINSFSCLCPVGFTGSFCLHEINECSSHPCLNDGTCVDCLGTYPCSCPLGYTCRNCQTLVNLCSRSPCKNKGTCV
QKKAESQCLCPSGWAGAYCDVPNVSCDIAASRRGVLVEHLCQHSGVCINAGNTHYCQCPLGYTCSYCEEQLDECASN
PCQHGATCSDFIGGYRCECVPCYQGVNCEYEVDECQNQPCQNGGTCIDLVNHFKCSCPPGTWGMKSSLSIFHCPGPH
CLNGGQCMDRICGYSCRCLPGFAGERCECDTNECLSNPCSSECSLDCIQLTNDYLCVCRSAFTGRHCETFVDVCPQM
PCLNGGTCAVASNMPDGSFAVVPQGFSGARCQSSCGQVKCRKCFQCVHTASGPRCFCFSPRDCESGCASSPCQHGGS
CHPQRQPPYYSCQCAPPFSCSRCELYTAPPSTPPATCLSQYCADKARDGVCDEACNSHACQWDCCDCSLTMENPWAN
CSSPLPCWDYTNNQCDELCNTVECLFDNFECQGNSKTCKYDKYCADHFKDNHCDQCCNSEECCWDGLDCAADQPENL
AEGTLVIVVLMPPEQLLQDARSFLRALGTLLHTNLRTKRDSQCELMVYPYYCEKSAAMKKQRMTRRSLPCEQEQEVA
GSKVFLEIDNRQCVQPSDHCFKNTDAAAALLASHATQGTLSYPLVSVVSESLTPERTQLLYLLAVAVVIILPIILLG
VIMAKRKRKHGSLWLREGFTLRRDASNHKRREPVGQDAVCLKNLSVQVSEANLICTCTSEHWVDDECPQPKKVKAED
EALLSEEDDPIDRRPWTQQHLEAADIRRTPSLALTPPQAEQEVDVLDVNVRGPDGCTPLMLASLRGGSSDLSDEDED
AEDSSANIITDLVYQCASLQAQTDPTCEMALHLAARYSRADAAKRLLDAGADANAQDNMGRCPLHAAVAADAQCVFQ
ILIRNRVTDLDARMNDGTTPLILAARLAVECMVAELINCQADVNAVDDHCKSALHWAAAVNNVEATLLLLKNGANRD
MQDNKEETPLPLAAREGSYEAAKILLDHFANRDITDHMDRLPPDVARDRMHHDIVRLLDEYNVTPSPPGTVLTSALS
PVICCPNRSFLSLKHTPMCKKSRRFSARSTMPTSLPNLAKEAKDAKCSRRKKSLSEKVQLSESSVTLSPVDSLESPH
TYVSDTTSSPMITSPCILQASPNPMLATAAPPAPVHAQHALSFSNLHEMQPLAHGASTVLPSVSQLLSHHHIVSPCS
GSAGSLSRLHPVPVPADWMNPMEVNETQYNEMFGMVLAPAVGHPSWHSSPERPPECKHITTPREPLPPIVTFQLIPK
GSIAQPACAPQPQSTCPPAVAGPLPTMYQIPEMARLPSVAFPTAMMPQQDCQVAQTILPAYHPFPASVCKYPTPPSQ
HSYASSNAAERTPSHSCHLQGEHPYLTPSPESPDQWSSSSPHSASDWSDVTTSPTPCGAGGCQRGPCTHMSEPPHNN
MQVYA

A region of the MOL1a nucleic acid sequence has 6436 of 7416 bases (86%) identical to a Rattus norvegicus Notch-like protein mRNA (GENBANK-ID:RATNOTCHX|acc:M93661), with an E-value of 0.0. In all BLAST alignments herein, the “E-value” or “Expect” value is a numeric indication of the probability that the aligned sequences could have achieved their similarity to the BLAST query sequence by chance alone, within the database that was searched. For example, the probability that the subject (“Sbjct”) retrieved from the MOL1a BLAST analysis, e.g., the Rattus norvegicus Notch-like protein mRNA, matched the Query MOL1a sequence purely by chance is 0.0. MOL1a also has 2443 of 2471 amino acid residues (98%) positive with patp:AAY06816 Human Notch2 (humN2) protein sequence—Homo sapiens, 2471 aa.

The Expect value is used as a convenient way to create a significance threshold for reporting results. The default value used for blasting is typically set to 0.0001. In BLAST 2.0, the Expect value is also used instead of the P value (probability) to report the significance of matches. For example, an E value of one assigned to a hit can be interpreted as meaning that in a database of the current size one might expect to see one match with a similar score simply by chance. An E value of zero means that one would not expect to see any matches with a similar score simply by chance. See, e.g., http://www.ncbi.nlm.nih.gov/Education/BLASTinfo/. Occasionally, a string of X's or N's will result from a BLAST search. This is a result of automatic filtering of the query for low-complexity sequence that is performed to prevent artifactual hits. The filter substitutes any low-complexity sequence that it finds with the letter “N” in nucleotide sequence (e.g., “NNNNNNNNNNNNN”) or the letter “X” in protein sequences (e.g., “XXXXXXXXX”). Low-complexity regions can result in high scores that reflect compositional bias rather than significant position-by-position alignment. Wootton and Federhen, Methods Enzymol 266:554-571, 1996.

Utilities for the MOLX nucleic acids and their encoded polypeptides can be inferred based on the homology of the disclosed MOLX nucleic acids and/or polypeptides (including domains of the encoded polypeptides) to previously described sequences.

MOL1a expression in different tissues was examined through TaqMan as described below in Example 1.

MOL1a is expressed in at least the following tissues: kidney, brain, lymph node, muscle, hippocampus, bone marrow, placenta, thyroid, para-thyroid, prostate, testis, epidermis, ovary, coronary artery, liver, lung, spinal cord, stomach, breast, lung, uterus, and colon. It is likely that Notch proteins are expressed in all tissues, so the widespread expression of MOL1a agrees with its homology with Notch.

One or more consensus positions (Cons. Pos.) of the nucleotide sequence of MOL1a have been identified as single nucleotide polymorphisms (SNPs) as shown in Table 1C. A dash (“-”), when shown, means that a base is not present. The sign “>” means “is changed to”. SNPs were identified using the techniques disclosed in Example 3.

TABLE 1C
SNPs for MOL1a

| | | AA | |
|---|---|---|---|
| Consensus | Base | change | |
| Position | Change | Position | Residue Change |
| 4288 | G > A | 1429 | A > T |
| 5858 | T > C | 1952 | L > P |
| 5833 | A > G | 1944 | T > A |
| 5366 | C > T | 1788 | T > I |

MOL1b

MOL1a was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated MOL1b, or alternatively Accession Number CG56250-02. This differs from the previously identified sequence in lacking 996 internal amino acids in addition to a few minor changes.

A disclosed Notch-like nucleic acid of 6728 nucleotides, MOL1b, is shown in Table 1C. The disclosed MOL1b open reading frame (“ORF”) begins at the ATG initiation codon at nucleotides 13-15, and terminates at a TGA codon at nucleotides 4431-4434. In Table 1D the start and stop codons are in bold letters, and the untranslated regions are underlined.

TABLE 1D

MOL1b nucleotide sequence.

(SEQ ID NO:3)

TCATCTGGAATTATGCCCGCCCTGCGCCCCGCTCTGCTGTGGGCGCTGCTGGCGCTCTGGCTGTGCTGCGCGGCCCC
CGCGCATCCATTGCAGTGTCCAGATCGCTATGAACCCTGTGTAAATGAAGGAATGTGTGTTACCTACCACAATGGCA
CAGGATACTGCAAATGTCCACAAGGCTTCTTGGGGGAATATTGTCAACATCGAGACCCCTGTGAGAAGAACCGCTGC
CAGAATGCTGCGACTTGTGTGCCCCAGGCCATGCTCGGGAAAGCCACGTGCCGATGTCCCTCAGGGTTTACAGGAGA
GGACTGCCAGTACTCAACATCTCATCCATGCTTTGTGTCTCGACCCTGCCTGAATCGCGGCACATGCCATATGCTCA
GCCGCGATACCTATGAGTCCACCTGTCAAGTCGGGTTTACAGGTAAGGAGTGCCAATGGACGGATGCCTCCCTGTCT
CATCCCTGTGCAAATCCAAGTACCTGTACCACTCTGCCCAACCAGTTCTCCTCCAAATGCCTCACACGCTTCACAGC
GCAGAAATGTGAGACTCATGTCAATGAGTGTGACATTCCAGGACACTGCCAGCATGGTCCCACCTGCCTCAACCTGC
CTGGTTCCTACCAGTGCCAGTGCCCTCAGGGCTTCACAGGCCAGTACTGTCACACCCTGTATGTGCCCTGTCCACCC
TCACCTTGTGTCAATGGAGGCACCTCTCGGCACACTGGTGACTTCACTTTTGAGTCCAACTCCCTTCCAGGTTTTGA
AGGGAGCACCTGTGAGAGGAATATTGATGACTGCCCTAACCACAGGTGTCAGAATGGACGCCTTTGTGTCGATGGGG
TCAACACTTACAACTGCCGCTGTCCCCCACAATGGACAGGACAGTTCTGCACAGAGGATGTCGATGAATGCCTGCTG
CAGCCCAATGCCTCTCAAAATCGCGGCACCTCTGCCAACCCCAATGGAGGCTATGGCTGTGTATGTCTCAACGGCTG
GAGTGGAGATGACTGCAGTGAGAACATTGATGATTGTCCCTTCGCCTCCTGTACTCCAGCCTCCACCTGCATCGACC
GTGTCGCCTCCTTCTCTTCCATGTGCCCAGAGGCGAAGGCACGTCTCCTGTGTCATCTGCATGATGCATGCATCAGC
AATCCTTCCCACAAGGGGGCACTGTGTGACACCAACCCCCTAAATCCGCAATATATTTGCACCTCCCCACAAGCCTA
CAAAGCGGCTCACTGCACAGAAGATGTCCATGAATGTGCCATGGCCAATAGCAATCCTTGTGACCATGCAGGAAAAT
GTGTGAACACGCATGGCGCCTTCCACTGTGAGTGTCTGAACGGTTATCCAGGACCTCGTTCTGAGATGGACATCAAT
GAGTGCCATTCAGACCCCTGCCAGAATGATGCTACCTGTCTGGATAAGATTCCAGGCTTCACATGTCTGTGCATGCC
AGGTTTCAAAGGTGTGCATTGTGAATTAGAAATAAATGAATGTCAGAGCAACCCTTGTGTGAACAATCCGCAGTGTG
TGGATAAAGTCAATCGTTTCCAGTCCCTGTCTCCTCCTGGTTTCACTCGGCCAGTTTCCCAGATTGATATTGATGAC
TGTTCCAGTACTCCGTGTCTGAATGGGGCAAAGTGTATCGATCACCCGAATGGCTATGAATCCCAGTGTGCCACAGG
TTTCACTCCTGTGTTCTGTCAGCAGAACATTCACAACTGTGACCCCGATCCTTGCCACCATGGTCACTGTCAGGATC
GTATTGATTCCTACACCTGCATCTGCAATCCCGGGTACATGGGCGCCATCTGCAGTGACCACATTGATGAATGTTAC
AGCAGCCCTTCCCTGAACGATGCTCCCTGCATTGACCTCCTCAATGGCTACCAGTCCAACTGCCAGCCAGGCACGTC
AGGGGTTAATTGTGAAATTAATTTTGATGACTGTGCAAGTAACCCTTGTATCCATGGAATCTGTATGGATGGCATTA
ATCGCTACAGTTGTGTCTGCTCACCAGGATTCACAGGGCAGAGATGTAACATTGACATTGATGAGTGTGCCTCCAAT
CCCTGTCGCAAGCCTGCAACATGTATCAACGGTCTGAATGGTTTCCGCTGTATATGCCCCGAGGGACCCCATCACCC
CAGCTGCTACTCACAGGTGAACGAATGCCTGAGCAATCCCTGCATCCATCCAAACTCTACTGGAGGTCTCAGTCGAT
ATAAGTGTCTCTGTGATGCAGCCTCGCTTGGCATCAACTGTGAACTGGACAAAAATGAATGCCTTTCCAATCCATGC
CAGAATGCAGCAACTPCTGACAATCTGGTGAATGGATACAGGTGTACTTGCAAGAAGGGCTTTAAACGCTATAACTG
CCAGGTGAATATTGATGAATGTGCCTCAAATCCATGCCTGAACCAAGGAACCTGCTTTGATGACATAAGTGGCTACA
CTTGCCACTGTGTGCTGCCATACACAGGCAAGAATTGTCAGACAGTATTGGCTCCCTGTTCCCCAAACCCTTCTGAC
AATCCTGCTCTTTCCAAAGAGTCACCAAATTTTCAGACTTATACTTGCTTCTGTCCTCCTGGCTGGCAAGGTCAGCG
GTGTACCATTGACATTGACGAGTGTATCTCCAAGCCCTGCATGAACCATGGTCTCTGCCATAACACCCAGCGCAGCT
ACATGTGTGAATGTCCACCAGGCTTCAGTGGTATGGACTGTGACGAGCACATTGATGACTGCCTTGCCAATCCTTGC
CAGAATGGAGGTTCCTGTATGGATGGAGTCAATACTTTCTCCTGCCTCTGCCTTCCCCGTTTCACTGGGGATAAGTG
CCAGACACACATGAATGAGTGTCTGACTGAACCCTGTAAGAATGGAGGGACCTGCTCTGACTACGTCAACAGTTACA
CTTGCAAGTGCCAGCCAGGATTTGATGGAGTCCATTGTGAGAACAACATCAATCACEGCACTGACAGCTCCTGTTTC
AATGGTGGCACATGTGTTGATGGGATTAACTCCTTCTCTTGCTTGTGCCCTCTGCCTTTCACTGGATCCTTCTGCCT
CCATGAGATCAATGAATGCAGCTCTCATCCATGCCTGAATGAGGGAACGTGTGTTCATGGCCTGGGTACCTACCGCT
GCAGCTCCCCCCTGGGCTACACTGCGAAAAACTGTCAGACCCTGGTGAATCTCTCCAGTCGGTCTCCATCTAAAAAC
AAAGGTACTTGTGTTCAGAAAAAAGCAGAGTCCCACTGCCTATGTCCATCTGGATGGGCTGGTGCCTATTGTGACGT
GCCCAATCTCTCTTGTCACATAGCACCCTCCAGGAGAGGTGTGCTTGTTCAACACTTGTGCCAGCACTCAGGTGTCT
GCATCAATGCTGGCAACACGCATTACTGTCAGTGCCCCCTGGGCTATACTCGGAGCTACTGTGACGAGCAACTCGAT
GAGTGTGCGTCCAACCCCTGCCAGCACGGGGCAACATGCACTGACTTCATTGCTGGATACAGATGCGAGTCTGTCCC
AGGCTATCAGGGTCTCAACTGTGAGTATGAAGTGGATGAGTGCCAGAATCAGCCCTCCCACAATGGACCCACCTGTA
TTGACCTTGTCAACCATTTCAAGTGCTCTTGCCCACCACGCACTCCGGCCCTACTCTGTGAAGAGAACATTGATGAC
TGTGCCCGGGGTCCCCATTGCCTTAATGGTCGTCACTGCATGGATAGGATTGGAGGCTACAGTTGTCGCTGCTTGCC
TGGCTTTGCTGGCGACCCTTCTGAGGGAGACATCAACCAGTGCCTCTCCAACCCCTGCACCTCTGAGCGCAGCCTGC
ACTGTATACACCTCACCAATGACTACCTGTGTGTTTGCCCTAGTCCCTTTACTGGCCGGCACTCTGAAACCTTCCTC
GATGTGTGTCCCCAGATGCCCTGCCTGAATGCAGGGACTTGTGCTGTGGCCAGTAACATGCCTGATGCTTTCATTTC
CCGTTGTCCCCCGGGATTTTCCGGGGCAAGGTACCAGATTCCAGAAATCGCCCGTTTGCCCAGTGTGGCTTTCCCCA
CTGCCATCATGCCCCAGCAGGACGGGCAGGTAGCTCAGACCATTCTCCCAGCCTATCATCCTTTCCCACCCTCTCTG
GGCAACTACCCCACACCCCCTTCACAGCACAGTTATCCTTCCTCAAATCCTGCTGAGCCAACACCCAGTCACAGTGG
TCACCTCCAGGGTGAGCATCCCTACCTGACACCATCCCCAGAGTCTCCTCACCAGTCGTCAAGTTCATCACCCCACT
CTGCTTCTGACTGGTCAGATGTGACCACCAGCCC
ACCCCTCGGGCTGCTCGACGACGTCAGCGGGGACCTGCGACA
CACATGTCTCAGCCACCACACAACAACATGCACGTTTATGCGTGAGAGACTCCACCTCCAGTGTAGACACATAACTG
ACTTTTGTAAATCCTCCTGAGGAACAAATGAAGCTCATCCGGGAGAGAAATGAAGAAATCTCTGGAGCCAGCTTCTA
GAGGTAGGAAAGAGAAGATGTTCTTATTCAGATAATGCAAGAGAAGCAATTCCTCAGTTTCACTGGCTATCTGCAAG
GCTTATTGATTATTCTAATCTAATAAGACAAGTTTGTGGAAATGCAAGATGAATACAAGCCTTGCGTCCATGTTTAC
TCTCTTCTATTTCCAGAATAAGATGGATGCTTATTGAAGCCCACACATTCTTGCAGCTTGGACTGCATTTTAAGCCC
TGCAGCCTTCTGCCATATCCATGAGAAGATTCTACACTACCGTCCTCTTGGGAATTATGCCCTGCCCTTCTGCCTGA
ATTGACCTACGCATCTCCTCCTCCTTGGACATTCTTTTGTCTTCATTTGGTCCTTTTGGTTTTCCACCTCTCCGTCA
TTGTAGCCCTACCAGCATGTTATAGGGCAAGACCTTTCTGCTTTTGATCATTCTGGCCCATGAAAGCAACTTTGGTC
TCCTTTCCCCTCCTGTCTTCCCGGTATCCCTTGGAGTCTCACAAGGTTTACTTTCGEATGGTTCTCAGCACAAACCT
TTCAAGTATGTTGTTTCTTTGGAAAATGGACATACTGTATTGTGTTCTCCTGCATATATCATTCCTGGAGAGAGAAG
GGGAGAAGAATACTTTTCTTCAACAAATTTTGGGGGCAGGAGATCCCTTCAAGAGGCTGCACCTTAATTTTTCTTGT
CTGTGTGCACGTCTTCATATAAACTTTACCAGGAAGAAGGGTGTGACTTTGTTGTTTTTCTGTGTATCGGCCTGGTC
AGTGTAAAGTTTTATCCTTGATAGTCTAGTTACTATGACCCTCCCCACTTTTTTAAAACCAGAAAAAGGTTTGGAAT
GTTGGAATGACCAAGAGACAAGTTAACTCCTCCAAGAGCCAGTTACCCACCCACAGGTCCCCCTACTTCCTGCCAAG
CATTCCATTGACTCCCTGTATGGAACACATTTGTCCCACATCTCAGCATTCTAGGCCTGTTTCACTCACTCACCCAG
CATATGAAACTAGTCTTAACTGTTCACCCTTTCCTTTCATATCCACAGAAGACACTGTCTCAAATGTTGTACCCTTG
CCATTTAGGACTCAACTTTCCTTAGCCCAAGCCACCCAGTGACAGTTCTCTTCCGTTTGTCAGATGATCAGTCTCTA
CTGATTATCTTGCTGCTTAAAGGCCTCCTCACCAATCTTTCTTTCACACCGTCTCGTCCGTGTTACTGGTATACCCA
GTATGTTCTCACTGAAGACATGGACTTTATATGTTCAAGTGCAGGAATTGGAAACTTGCACTTGTTTTCTATCATCC
AAAACAGCCCTATAAGAACGTTGGAAAAGCACGAACTATATAGCAGCCTTTGCTATTTTCTGCTACCATTTCTTTTC
CTCTGAACCGGCCATGACATTCCCTTTCGCAACTAACGTAGAAACTCAACAGAACATTTTCCTTTCCTAGACTCACC
TTTTAGATGATAATGGACAACTATAGACTTGCTCATTGTTCACACTGATTGCCCCTCACCTGAATCCACTCTCTGTA
TTCATGCTCTTGGCAATTTCTTTGACTTTCTTTTAAGGGCAGAAGCATTTTAGTTAATTGTAGATAAAGAATAGTTT
TCTTCCTCTTCTCCTTGGCCCAGTTAATAATTCGTCCATGGCTACACTGCPACTTCCCTCCAGTCCTGTGATGCCCA
TGACACCTGCAAAATAAGTTCTGCCTGGGCATTTTGTAGATATTAACACGTGAATTCCCGACTCTTTTGGTTTGAAT
GACAGTTCTCATTCCTTCTATGGCTGCAACTATGCATCAGTCCTTCCCACTTACCTGATTTGTCTGTCCGTCGCCCC
ATATCCAACCCTGCGTGTCTCTTGGCATAATAGTTTACAAATGGTTTTTTCAGTCCTATCCAAATTTATTGIAACCA
ACAAAAATAATTACTTCTCCCCTGAGATAAGCACATTAAGTTTGTTCATTCTCTGCTTTATTCTCTCCATGTGGCAA
CATTCTGTCAGCCTCTTTCATAGTGTCCAAACATTTTATCATTCTAAATGGTGACTCTCTGCCCTTGGACCCATTTA
TTATTCACAGATGGGGACAACCTATCTCCATGGACCCTCACCATCCTCTGTGCAGCACACACAGTGCAGGGAGCCAG
TGCCGATGGCGATGACTTTCTTCCCCTGG

The protein encoded by the MOL1b nucleic acid sequence has 2469 amino acid residues, and is disclosed in Table 1E. The MOL1b protein was analyzed for signal peptide prediction and cellular localization. SignalP results predict that MOL1b is cleaved between position 25 and 26 (AHA-LQ) of SEQ ID NO:4. Psort and Hydropathy profiles also predict that MOL1b contains a signal peptide and is likely to be localized extracellularly (Certainty=0.7666).

TABLE 1E

Encoded MOL1b protein sequence.

(SEQ ID NO:4)

MPALRPALLWALLALWLCCAAPAHALQCRDGYEPCVNEGMCVTYHNCTGYCKCPEGFLGEYCQHRDPCEKNRCQNGC
TCVAQAMLGKATCRCASGFTGEDCQYSTSHPCFVSRPCLNCCTCHMLSRDTYECTCQVCFTCKECQWTDACLSHPCA
NGSTCTTVANQFSCKCLTGFTCQKCETDVNECDIPGHCQHGCTCLNLPGSYQCQCPQGFTGQYCDSLYVPCAPSPCV
NGGTCRQTGDFTFECNCLPGFEGSTCERNIDDCPNHRCQNGGVCVDGVNTYNCRCPPQWTGQFCTEDVDECLLQPNA
CQNGGTCANRNCGYGCVCVNGWSGDDCSENIDDCAFASCTPCSTCTDRVASFSCMCPEGKAGLLCHLDDACISNPCH
KGALCDTNPLNCQYICTCPQCYKCADCTEDVDECAMANSNPCEHAGKCVNTDGAFHCECLKCYACPRCEMDINECHS
DPCQNDATCLDKIGGFTCLCMPGFKGVHCELEINECQSNPCVNNGQCVDKVNRFQCLCPPGFTGPVCQIDIDDCSST
PCLNGAKCIDHPNCYECQCATGFTGVLCEENIDNCDPDPCHHGQCQDGIDSYTCICNPGYMGAICSDQTDECYSSPC
LNDGRCIDLVNGYQCNCQPGTSGVNCETNFDDCASNFCTHCICMDGINRYSCVCSPCFTCQRCNIDIDECASNPCRK
GATCINGVNGFRCICPEGPHHPSCYSQVNECLSNPCIHGNCTGGLSGYKCLCDAGWVGINCEVDKNECLSNPCQNGG
TCDNLVNGYRCTCRKGFKGYNCQVNIDECASNPCLNQGTCFDDISCYTCHCVLPYTCKNCQTVLAPCSPNPCENAAV
CKESPNFESYTCLCAPGWQGQRCTIDIDECISKPCMNHGLCHNTQGSYMCECFPGFSCMDCEEDIDDCLANPCQNGG
SCMDGVNTFSCLCLPGFTGDKCQTDMNECLSEPCKNCGTCSDYVNSYTCKCQAGFDGVHCENNINECTESSCFNGCT
CVDGINSFSCLCPVCFTCSFCLHEINECSSHPCLNECTCVDCLCTYRCSCPLGYTGKNCQTLVNLCSRSPCKNKGTC
VQKKAESQCLCPSGWAGAYCDVPNVSCDIAASRKGVLVEHLCQHSCVCINAGNTHYCQCPLGYTGSYCEEQLDECAS
NPCQHGATCSDFIGCYRCECVPGYQCVNCEYEVDECQNQPCQNGGTCIDLVNHFKCSCPPGTRCLLCEENIDDCARG
PHCLNGGQCMDRIGGYSCRCLPGFAGERCEGDINECLSNPCSSEGSLDCIQLTNDYLCVCRSAFTGRHCETFVDVCP
QMPCLNGGTCAVASNMPDCEICRCPPCFSGARYQTPEMARLPSVAFPTAMMPQQDGQVAQTILPAYHPFPASVCKYP
TPPSQHSYASSNAAERTPSHSGHLQGEHPYLTPSPESPDQWSSSSPHSASDWSDVTTSPTPGGAGGGQRGPGTHMSE
PPHNNMQVYA

A region of the MOL1b nucleic acid sequence, localized to chromosome 1, has 4041 of 4042 bases (99%) identical to a gb:GENBANK-ID:AF308601|acc:AF308601.1 mRNA from Homo sapiens (Homo sapiens NOTCH 2 (N2) mRNA, complete cds), with an E-value of 0.0.

The amino acid sequence of MOL1b has 1340 of 1343 amino acid residues (99%) identical to, and 1340 of 1343 amino acid residues (99%) similar to, the 2471 amino acid residue ptnr:TREMBLNEW-ACC:AAG37073 protein from Homo sapiens (Human) (NOTCH2 PROTEIN).

MOL1b expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus, Aorta, Ascending Colon, Bone, Cartilage, Cochlea, Colon, Coronary Artery, Epidermis, Foreskin, Liver, Lung, Lymph node, Lymphoid tissue, Muscle, Nasoepithelium, Ovary, Parathyroid Gland, Parotid Salivary glands, Peripheral Blood, Respiratory Bronchiole, Retina, Synovium/Synovial membrane, Thymus, Tonsils, Umbilical Vein, Vein, Whole Organism.

One or more consensus positions (Cons. Pos.) of the nucleotide sequence of MOL1b have been identified as single nucleotide polymorphisms (SNPS) as shown in Table 1F. “Depth” represents the number of clones covering the region of the SNP. The Putative Allele Frequency (PAF) is the fraction of all the clones containing the SNP. A dash (“-”), when shown, means that a base is not present. The sign “>” means “is changed to”.

TABLE 1F
SNPs for MOL1b

| Consensus | | Base | |
|---|---|---|---|
| Position | Depth | Change | PAF |
| 100 | 24 | T > C | 0.083 |
| 204 | 23 | G > A | 0.087 |
| 369 | 23 | G > A | 0.087 |

The amino acid sequence of MOL1a also had high homology to other proteins as shown in table 1G.

TABLE 1G
BLAST results for MOL1a

| Gene Index/ | Protein/ | | | | |
|---|---|---|---|---|---|
| Identifier | Organism | Length (aa) | Identity (%) | Positives (%) | Expect |
| gi\|104252\|pir\|\| | Notch protein - | 2524 | 1323/2515 | 1676/2515 | 0.0 |
| A35844 | African clawed | | (52%) | (66%), | |
| | frog | | | | |
| >gi\|6679096\|ref\| | Notch gene | 2318 | 665/1515 | 860/1515 | 0.0 |
| NP 032742.1\| | homolog 3, | | (43%) | (55%) | |
| | (Drosophila) | | | | |
| | [Mus musculus] | | | | |
| gi\|13242247\|ref\| | Notch gene | 2471 | 2251/2472 | 2340/2472 | 0.0 |
| NP 077334.1\| | homolog 2, | | (91%) | (94%) | |
| | (Drosophila) | | | | |
| | [Rattus | | | | |
| | norvegicus] | | | | |
| gi\|2209059\|dbj\| | Notch 2 | 2447 | 1240/2203 | 1546/2203 | 0.0 |
| BAA20535.1\| | [Takifugu | | (56%) | (69%) | |
| | rubripes] | | | | |
| gi\|6093542\|sp\| | NEUROGENIC | 2531 | 1340/2538 | 1683/2538 | 0.0 |
| Q07008\| | LOCUS NOTCH | | (52%) | (65%) | |
| NTC1 RAT | HOMOLOG | | | | |
| | PROTEIN | | | | |
| | 1 PRECURSOR | | | | |

A ClustalW analysis comparing disclosed proteins of the invention with related OR protein sequences is given in Table 1H, with MOL1a shown on line 1 and MOL1b on line 2.

In the ClustalW alignment of the MOL1a and MOL1b proteins, as well as all other ClustalW analyses herein, the black outlined amino acid residues indicate regions of conserved sequence (i.e, regions that may be required to preserve structural or functional properties), whereas non-highlighted amino acid residues are less conserved and can potentially be mutated to a much broader extent without altering protein structure or function. Residue differences between any MOLX variant sequences herein are written to show the residue in the “a” variant and the residue position with respect to the “a” variant. MOL residues in all following sequence alignments that differ between the individual MOL variants are highlighted with a box and marked with the (o) symbol above the variant residue in all alignments herein.

[image]

[image]

[image]

[image]

[image]

[image]

[image]

[image]

[image]

[image]

When the sequences of the invention are referred to as MOL1, this refers to the sequences disclosed as MOL1a and MOL1b.

The presence of identifiable domains in MOL1, as well as all other MOLX proteins, was determined by searches using software algorithms such as PROSITE, DOMAIN, Blocks, Pfam, ProDomain, and Prints, and then determining the Interpro number by crossing the domain match (or numbers) using the Interpro website (http:www.ebi.ac.uk/interpro). DOMAIN results, e.g., for MOL1 as disclosed in Table 1I, were collected from the Conserved Domain Database (CDD) with Reverse Position Specific BLAST analyses. This BLAST analysis software samples domains found in the Smart and Pfam collections. For Table1I and all successive DOMAIN sequence alignments, fully conserved single residues are indicated by black shading and “strong” semi-conserved residues are indicated by grey shading. The “strong” group of conserved amino acid residues may be any one of the following groups of amino acids: STA, NEQK, NHQK, NDEQ, QHRK, MILV, MILF, HY, FYW.

Tables 1I-1N list the domain description from DOMAIN analysis results against MOL1. The region from amino acid residue 1416 through 1454 (SEQ ID NO:2) most probably (E=1e⁻⁶) contains a domain found in Notch and Lin-12, aligned here in table 1I. Notch and Lin-12 are both involved in organismal development, cell cycle, and apoptosis. The region from amino acid residue 1504 through 1532 (SEQ ID NO:2) most probably (E=2e⁻⁴) also contains a domain found in Notch and Lin-12, aligned here in table 1J. The region from amino acid residue 1875 through 1906 (SEQ ID NO:2) most probably (E=6e⁻⁵) contains an Ank repeat, aligned here in table 1K. Ank repeats are markers for the protein ankyrin which is involved in cell adhesion and contact inhibition. The region from amino acid residue 1974 through 2006 (SEQ ID NO:2) most probably (E=2e⁻⁴) also contains an Ank repeat, aligned here in table 1L. The region from amino acid residue 182 through 215 (SEQ ID NO:2) most probably (E=1e⁻⁴) contains a Calcium binding EGF-like domain, aligned here in table 1M. EGF is a growth factor that modulates the proliferation of many cell types. The region from amino acid residue 872 through 908 (SEQ ID NO:2) most probably (E=9e⁻⁴) also contains a Calcium binding EGF-like domain, aligned here in table 1N. This indicates that the MOL1 sequence has properties similar to those of other proteins known to contain these domains.

[image]

[image]

### Uses of the Compositions of the Invention

The protein similarity information, expression pattern, cellular localization, and map location for the protein and nucleic acid disclosed herein suggest that MOL1 may have important structural and/or physiological functions characteristic of the EGF-like domain containing protein family. Therefore, the nucleic acids and proteins of the invention are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The MOL1 nucleic acids and proteins have applications in the diagnosis and/or treatment of various diseases and disorders. For example, the compositions of MOL1 may have efficacy for the treatment of patients suffering from endometriosis, fertility disorders, cancer, hypercoagulation, idiopathic thrombocytopenic purpura, immunodeficiencies, systemic lupus erythematosus, asthma, emphysema, scleroderma, allergy, ARDS, Von Hippel-Lindau (VHL) syndrome, Alzheimer's disease, stroke, tuberous sclerosis, hypercalceimia, Parkinson's disease, Huntington's disease, cerebral palsy, epilepsy, multiple sclerosis, ulcers, ataxia-telangiectasia, leukodystrophies, behavioral disorders, addiction, anxiety, pain, neuroprotection, osteoporosis, hypercalceimia, arthritis, ankylosing spondylitis, scoliosis, diabetes, autoimmune disease, myasthenia gravis, muscular dystrophy, renal artery stenosis, interstitial nephritis, glomerulonephritis, polycystic kidney disease, systemic lupus erythematosus, renal tubular acidosis, IgA nephropathy, hypercalceimia, Lesch-Nyhan syndrome, developmental disorders, growth disorders, and/or wounds, as well as other diseases, disorders and conditions. The reactivation of the Notch signaling pathway during wound healing has been demonstrated and the similarity between developmental and regenerative processes has been suggested (Exp Cell Res 1999 February 1;246(2):312-8).

These materials are further useful in the generation of antibodies that bind immuno-specifically to the novel MOL1 substances for use in therapeutic or diagnostic methods. These antibodies may be generated according to methods known in the art, using prediction from hydrophobicity charts, as described in the “Anti-MOLX Antibodies” section below. The disclosed MOL1 protein has multiple hydrophilic regions, each of which can be used as an immunogen. In one embodiment, a contemplated MOL1 epitope is from about amino acids 10 to 150. In another embodiment, a MOL1 epitope is from about amino acids 160 to 190. In additional embodiments, MOL1 epitopes are from about amino acids 195 to 350, 400 to 525, 550 to 575, 590 to 600, 610 to 650, 780 to 880, 900 to 1000, 1100 to 1150, 1175 to 1200, 1225 to 1300, and from about amino acids 1380 to 1500. These novel proteins can also be used to develop assay systems for functional analysis.

### MOL2

### MOL2a

MOL2a is a novel insulin-like growth factor binding protein-like protein. The novel nucleic acid of 2631 nucleotides, (SC98428706_EXT, SEQ ID NO:5) encoding a novel insulin-like growth factor binding protein-like protein is shown in Table 2A. The start and stop codons are in bold.

TABLE 2A

MOL2a Nucleotide Sequence

(SEQ ID NO:5)

ATGATTTACATACAAGTAATTTTTCAAGTAATGACCATTGAAAAAATGTTTTCTTTTTATTTTTTAGATTATTTCTC
TTTATTCAGAAGCATACAGTTGTTTGCTGATTGCAAGAAGATGTTTCTGTGGCTGTTTCTGATTTTGTCAGCCCTGA
TTTCTTCCACAAATGCAGATTCTGACATATCCGTGGAAATTTGCAATCTCTGTTCCTGCGTGTCAGTTGAGAATGTG
CTCTATGTCAACTGTGAGAAGGTTTCAGTCTACAGACCAAATCAGCTGAAACCACCTTGGTCTAATTTTTATCACCT
CAATTTCCAAAATAATTTTTTAAATATTCTGTATCCAAATACATTCTTGAATTTTTCACATGCAGTCTCCCTGCATC
TGGGGAATAATAAACTGCAGAACATTGAGGGAGGAGCCTTTCTTGGGCTCAGTGCATTAAAGCAGTTGCACTTGAAC
AACAATGAATTAAAGATTCTCCGAGCTGACACTTTCCTTGGCATAGAGAACTTGGAGTATCTCCAGGCTGACTACAA
TTTAATCAAGTATATTGAACGAGGAGCCTTCAATAAGCTCCACAAACTGAAAGTTCTCATTCTTAATGACAATCTGA
TTTCATTCCTTCCTGATAATATTTTCCGATTCGCATCTTTGACCCATCTGGATATACGAGGGAACAGAATCCAGAAG
CTCCCTTATATCGGGGTTCTGGAACACATTGGCCGTGTCGTTGAATTGCAACTGGAAGATAACCCTTGGAACTGTAG
CTGTGATTTATTGCCCTTAAAAGCTTGGCTGGACAACATGCCATATAACATTTACATAGGAGAAGCTATCTGTGAAA
CTCCCAGTCACTTATATGGAAGCCTTTTAAAAGAAACCAACAAACAAGAGCTATGTCCCATGGGCACCGGCAGTGAT
TTTCACCTGCGCATCCTGCCTCCATCTCAGCTGGAAAATGGCTACACCACTCCCAATCGTCACACTACCCAAACATC
TTTACACAGATTAGTAACTAAACCACCAAAAACAACAAATCCTTCCAAGATCTCTGGAATCGTTGCAGGCAAAGCCC
TCTCCAACCGCAATCTCACTCACATTCTGTCTTACCAAACAAGGGTGCCTCCTCTAACACCTTGCCCGGCACCTTGC
TTCTGCAkAACACACCCTTCAGATTTCGGACTAAGTGTGAACTCCCAAGAGAAAAATATACAGTCTATGTCTGPACT
GATACCGAAACCTTTAAATGCCAAGAAGCTGCACCTCAATGGCAATAGCATCAAGGATGTGGACGTATCAGACTTCA
CTGACTTTGAAGGACTGGATTTGCTTCATCTAGGCAGCAATCAAATTACAGTGATTAAGGGAGACGTATTTCACAAT
CTCACTAATTTACGCACGCTATATCTCPATGGCAATCAAATTGAGAGACTCTATCCTGAAATATTTTCAGGTCTTCA
TAACCTGCAGTATCTGTATTTCGAATACPATTTGATTAAGGAAATCTCAGCAGGCACCTTTGACTCCATGCCAAATT
TGCAGTTACTGTACTTAAACAATAATCTCCTAAACAGCCTGCCTGTTTACATCTTTTCCGGAGCACCCTTAGCTAGA
CTGAACCTGACGAACAACAAATTCATCTACCTGCCTGTCACTGGGGTCCTTGATCAGTTGCAATCTCTTACACACAT
TGACTTGGAGCGCAACCCATGGGACTGTACTTGTGACTTGGTGGCATTAAAGCTGTCGGTGGAGAAGTTGAGCCACG
GGATTGTTGTGAAAGAACTGAAATGTGACACCCCTGTTCAGTTTGCCPACATTGAACTCAAGTCCCTCAAAAATGAA
ATCTTATGTCCCAAACTTTTAAATAAGCCCTCTGCACCATTCACAAGCCCTGCACCTGCCATTACATTCACCACTCC
TTTGCGTCCCATTCGAAGTCCTCCTGGTGCGCCAGTGCCTCTGTCTATTTTAATCTTAAGTATCTTAGTGGTCCTCA
TTTTAACGGTGTTTGTTGCTTTTTGCCTTCTTGTTTTTGTCCTGCGACGCAACAAGAAACCCACAGTGAAGCACGAA
GGCCTGGGGAATCCTGACTGTGGCTCCATGCAGCTGCAGCTAAGGAAGCATGACCACAAAACCAATAAAAAAGATGG
ACTGAGCACAGAAGCTTTCATTCCACAAACTATAGAACAGATGAGCAAGAGCCACACTTGTGGCTTGAAAGAGTCAC
AAACTCGGTTCATGTTTTCAGATCCTCCAGGACAGAAAGTTGTTATGAGAAATGTGCCCGACAAGGAGAAAGATTTA
TTACATGTAGATACCACGAAGAGACTGAGCACAATTGATCACCTGCATGAATTATTCCCTACCACCCATTCCAATGT
GTTTATTCAGAATTTTCTTGAAAGCAAAAAGGACTATAATAGCATAGGTGTCAGTGGCTTTGAGATCCGCTATCCAG
AAAAACAACCAGACAAAAAAAGTAAGAAGTCACTGATAGCTGGCAACCACAGTAAAATTGTTGTGGAACAAACGAAG
AGTGAGTATTTTGAACTGAAGGCGAAACTCCAGAGTTCCCCTCACTACCTACAGGTCCTTGAGGACCAAACAGCTTT
GAACAAGATCTAG

An open reading frame (ORF) for MOL2a was identified from nucleotides1 to 2628. The disclosed MOL2a polypeptide (SEQ ID NO:6) encoded by SEQ ID NO:5 has 876 amino acid residues and is presented using the one-letter code in Table 2B. The SignalP, Psort and Hydropathy profile of MOL2a indicate that this sequence does have a signal peptide localized between amino acids 57 and 58 (TNA-DS) and is likely to be localized to the plasma membrane (0.4600 certainty). Therefore it is likely that MOL2a is available at the appropriate sub-cellular localization and hence accessible for the therapeutic uses described in this application.

TABLE 2B

Encoded MOL2a protein sequence.

(SEQ ID NO:6)

MIYIQVIFQVMTIEKMFSFYFLDYFSLFRSTQLFADCKKMFLWLFLILSALISSTNADSDISVEICNVCSCVSVENV
LYVNCEKVSVYRPNQLKPPWSNFYHLNFQNNFLNILYPNTFLNFSHAVSLHLCNNKLQNIEGGAFLCLSALKQLHLN
NNELKTLPADTFLGIENLEYLQADYNLIKYTERGAFNKLHKLKVLILNDNLISFLPDNIFRFASLTHLDIRGNRIQK
LPYIGVLEHIGRVVELQLEDNPWNCSCDLLPLKAWLENMPYNIYIGEAICETPSDLYGRLLKETNKQELCPMGTGSD
FDVRILPPSQLENGYTTPNGHTTQTSLHRLVTKPPKTTNPSKISGIVAGKALSNRNLSQIVSYQTRVPPLTPCPAPC
FCKTHPSDLCLSVNCQEKNTQSMSELIPKPLNAKKLHVNGNSIKDVDVSDPTDFECLDLLHLGSNQITVIKCDVFHN
LTNLRRLYLNGNQIERLYPETFSCLHNLQYLYLEYNLIKEISAGTFDSMPNLQLLYLNNNLLKSLPVYIFSGAPLAR
LNLRNNKFMYLPVSGVLDQLQSLTQIDLEGNPWDCTCDLVALKLWVEKLSDGIVVKELKCETPVQEANIELKSLKNE
ILCPKLLNKPSAPFTSRAPAITFTTPLGPIRSPPGGRVPLSILILSILVVLILTVEVAFCLLVFVLRRNKKPTVKHE
GLGNFDCCSMQLQLRKHDHKTNKKDGLSTEAFIPOTIEQMSKSHTCGLKESETGFMFSDPPGQKVVMRNVADKEKDL
LHVDTRKRLSTIDELDELFPSRDSNVFIQNFLESKKEYNSIGVSGFEIRYPEKQPDKKSKKSLIGGNHSKIVVEQRK
SEYFELKAKLQSSPDYLQVLEEQTALNKI

The MOL2a nucleic acid sequence, localized on the q26.3-28 region of the X chromosome, has 532 of 854 bases (62%) identical to a Homo sapiens Insulin-like growth factor binding protein-like protein mRNA (GENBANK-ID:AB020655|acc:AB020655).

The full amino acid sequence of the protein of the invention was found to have 318 of 672 amino acid residues (47%) identical to, and 445 of 672 residues (66%) similar to, the 977 amino acid residue Insulin-like growth factor binding protein-like protein from Homo sapiens (SPTREMBL-ACC:O94933).

MOL2a expression in different tissues was examined through TaqMan as described below in Example 1.

### MOL2b

MOL2b is a novel insulin-like growth factor binding protein-like protein. The novel nucleic acid of 1800 nucleotides, (191999007, SEQ ID NO:101) encoding a novel insulin-like growth factor binding protein-like protein is shown in Table 2C. The start and stop codons are in bold. Since the start and stop codons are not traditional initiation and termination codons, MOL2b could be a partial reading frame that could extend in the 5′ and/or 3′ directions.

TABLE 2C

MOL2b Nucleotide Sequence

(SEQ ID NO:101)

GGATCCGATTCTGACATATCCGTGGAAATTTGCAATGTGTGTTCCTGCGTGTCACTTGAGAATGTCCTCTATGTCAA
CTGTGAGAAGGTTTCAGTCTACAGACCAAATCAGCTGAAACCACCTTGGTCTAATTTTTATCACCTCAATTTCCAAA
ATAATTTTTTAAATATTCTGTATCCAAATACATTCTTGAATTTTTCACATGCAGTCTCCCTGCATCTGGGGAATAAT
AAACTGCAGAACATTGAGGGAGGAGCCTTTCTTGGGCTCAGTGCATTAAAGCAGTTGCACTTGAACAACAATGAATT
AAAGATTCTCCGAGCTGACACTTTCCCTGGCATAGAGAACTTGGAGTATCTCCAGGCTGACTACAATTTAATCAAGT
ATATTGAACGAGGACCCTTCAATAAGCTCCACAAACTGAAAGTTCTCATTCTTAATGACAATCTGATTTCATTCCTT
CCTGATAATATTTTCCGATTCGCATCTTTGACCCATCTGGATATACGACGGAACAGAATCCAGAAGCTCCCTTATAT
CGGGGTTCTGGAACACATTGGCCGTGTCGTTGAATTGCAACTGGAAGATAACCCTTGGAACTGTAGCTGTGATTTAT
TGCCCTTAAAAGCTTGGCTGGAGAACATGCCATATAACATTTACATAGGAGAAGCTATCTGTGAAACTCCCAGTGAC
TTATATGCAACGCTTTTAAAAGAAACCAACAAACAAGACCTATGTCCCATGGGCACCGGCAGTGATTTTGACGTCCG
CATCCTGCCTCCATCTCAGCTGGAAAATGCCTACACCACTCCCAATCGTCACACTACCCAAACATCTTTACACAGAT
TAGTAACTAAACCACCAAAAACAACAAATCCTTCCAAGATCTCTGGAATCGTTGCAGGCAAAGCCCTCTCCAACCGC
AATCTCAGTCAGATTGTGTCTTACCAAACAAGGGTGCCTCCTCTAACACCTTGCCCGGCACCTTGCTTCTGCAAAAC
ACACCCTTCAGATTTGGGACTAAGTCTGAACTGCCAAGAGAAAAATATACAGTCTATGTCTGAACTGATACCGPAAC
CTTTAAATGCGAAGAAGCTGCACGTCAATGGCAATAGCATCAAGGATGTGGACGTATCAGACTTCACTGACTTTGAA
GGACTGGATTTGCTTCATTTAGGCAGCAATCAAATTACAGTGATTAAGGGAGACGTATTTCACAATCTCACTAATTT
ACGCAGGCTATATCTCAATGGCAATCAAATTGAGAGACTCTATCCTGAAATATTTTCAGGTCTTCATAACCTGCAGT
ATCTGTATTTGGAATACAATTTGATTAAGGAAATCTCAGCAGGCACCTTTGACTCCATGCCAAATTTGCAGTTACTG
TACTTAAACAATAATCTCCTAAACAGCCTGCCTGTTTACATCTTTTCCGGAGCACCCTTAGCTAGACTGAACCTGAG
GAACAACAAATTCATGTACCTGCCTCTCAGTGGGGTCCTTCATCAGTTGCAATCTCTTACACAGATTGACTTGGAGC
GCAACCCATGCGACTGTACTTGTGACTTGGTGGCATTAAAGCTGTGGGTGGGGAAGTTCAGCCACCGGATTGTTCTG
AAAGAACTGAAATGTGAGACGCCTGTTCAGTTTGCCAACATTGAACTGAAGTCCCTCAAAAATGAAATCTTATGTCC
CAAACTTTTAAATAAGCCGTCTGCACCATTCACAAGCCCTGCACCTACCATTACATTCACCACTCCTTTGGGTCCCA
TTCGAAGTCCTCCTCGTGGCCCACTCGAG

An open reading frame (ORF) for MOL2b was identified from nucleotides 1 to 1800. The disclosed MOL2b polypeptide (SEQ ID NO:102) encoded by SEQ ID NO:101 has 600 amino acid residues and is presented using the one-letter code in Table 2D.

TABLE 2D

Encoded MOL2b protein sequence.

(SEQ ID NO:102)

GSDSDISVEICNVCSCVSVENVLYVNCEKVSVYRPNQLKPPWSNFYHLNFQNNFLNILYPNTFLNFSHAVSLHLGNN
KLQNIEGCAFLGLSALKQLHLNNNELKILRADTFPGTENLEYLQADYNLIKYIERGAFNKLHKLKVLTLNDNLISFL
PDNIFRFASLTHLDTRGNRTQKLPYICVLEHIGRVVELQLEDNPWNCSCDLLPLKAWLENMPYNIYICEAICETPSD
LYGRLLKETNKQELCPMGTGSDFDVRILPPSQLENGYTTPNGHTTQTSLHRLVTKPPKTTNPSKISGIVAGKALSNR
NLSQIVSYQTRVPPLTPCPAPCFCKTHPSDLGLSVNCQEKNIQSMSELIPKPLNAKKLHVNGNSIKDVDVSDFTDFE
GLDLLHLGSNQITVIKGDVFHNLTNLRRLYLNCNQTERLYPEIFSCLHNLQYLYLEYNLIKETSAGTFDSMPNLQLL
YLNNNLLKSLPVYIFSGAPLARLNLRNNKFMYLPVSGVLDQLQSLTQIDLEGNPWDCTCDLVALKLWVGKLSDGIVV
KELKCETPVQFANIELKSLKNEILCPKLLNKPSAPFTSPAPTITFTTPLGPIRSPPGCPLE

### MOL2c

MOL2c is a novel insulin-like growth factor binding protein-like protein. The novel nucleic acid of 1800 nucleotides, (192586956, SEQ ID NO:103) encoding a novel insulin-like growth factor binding protein-like protein is shown in Table 2E. The start and stop codons are in bold. Since the start and stop codons are not traditional initiation and termination codons, MOL2c could be a partial reading frame that could extend in the 5′ and/or 3′ directions.

TABLE 2E

MOL2c Nucleotide Sequence

(SEQ ID NO:103)

GGATCCGATTCTGACATATCGGTGGAAATTTGCAATGTGTGTTCCTGCGTGTCAGTTGAGAATGTGCTCTATGTCAA
CTGTGAGAAGGTTTCAGTCTACAGACCAAATCAGCTGAAACCACCTTGGTCTAATTTTTATCACCTCAATTTCCAAA
ATAATTTTTTAAATATTCTGTATCCAAATACATTCTTGAATTTTTCACATGCAGTCTCCCTGCATCTGGGGAATAAT
AAACTGCAGAACATTGAGGGAGGAGCCTTTCTTGGGCTCAGTACATTAAAGCAGTTGCACTTGAACAACAATGAATT
AAAGATTCTCCCAGCTGACACTTTCCTTGGCATAGAGAACTTCGACTATCTCCAGGCTGACTACAATTTAATCAAGT
ATATTGAACGACGAGCCTTCAATAAGCTCCACAAACTGAAAGTTCTCATTCTTAATGACAATCTGATTTCATTCCTT
CCTGATAATATTTTCCGATTCGCATCTTTGACCCATCTGGATATACGAGGGAACAGAATCCAGAAGCTCCCTTATAT
CGGGGTTCTGGAACACATTGGTCGTGTCGTTGAATTGCAACTGGAAGATAACCCTTGGAACTGTAGCTGTGATTTAT
TGCCCTTAAAAGCTTGGCTGGAGAACATGCCATATAACATTTACATAGGAGAAGCTATCTGTGAAACTCCCAGTGAC
TTATATGGAAGGCTTTTAAAAGAAACCAACAAACAAGAGCTATGTCCCATGCCCACCGGCAGTCATTTTGACGTGCG
CATCCTGCCTCCATCTCAGCTCCAAAATGCCTACACCACTCCCAATGGTCACACTACCCAAACATCTTTACACAGAT
TAGTAACTAAACCACCAAAAACAACAAATCCTTCCAAGATCTCTGGAATCGTTGCAGGTAAAGCCCTCTCCAACCGC
AATCTCAGTCAGATTGTGTCTTACCAAACAAGGGTGCCTCCTCTAACACCTTGCCCGGCACCTTGCTTCTGCAAAAC
ACACCCTTCAGATTTGGGACTAAGTGTGAACTGCCAAGAGAAAAATATACAGTCTATGTCTGAACTGATACCGAAAC
CTTTAAATGCGAAGAAGCTGCACGTCAATGGCAATAGCATCAAGGATGTGGACGTATCAGACTTCACTGACTTTGAA
GGACTGGATTTGCTTCATTTAGGCAGCAATCAAATTACAGTGATTAAGGGAGACGTATTTCACAATCTCACTAATTT
ACGCAGGCTATATCTCAATGGCAATCAAATTGAGAGACTCTATCCTGAAATATTTTCAGGTCTTCATAACCTGCAGT
ATCTCTATTTGGAATACAATTTGATTAAGGAAATCTCAGCAGGCACCTTTGACTCCATCCCAAATTTGCAGTTACTG
TACTTAAGCAATAATCTCCTAAACAGCCTGCCTGTTTACATCTTTTCCGCAGCACCCTTAGCTACACTGAACCTGAG
GAACAACAAATTCATGTACCTGCCTGTCAGTGGGGTCCTTGATCAGTTGCAATCTCTTACACAGATTGACTTGGAGC
GCAGCCCATGGGACTATACTTGTGACTTGGTGGCATTAAAGCTGTGGGTGGAGAAGTTGAGCGACGCCATTGTTCTG
AAAGAACTGAAATGTGAGACGCCTGTTCAGTTTACCAACATTGAACTGAAGTCCCTCAAAAATGAAATCTTATGTCC
CAAACTTTTAAATAAGCCGTCTGCACCATTCACAACCCCTCCACCTGCCATTACATTCACCACTCCTTTGGGTCCCA
TTCCAAGTCCTCCTGCTGGGCCACTCGAC

An open reading frame (ORF) for MOL2c was identified from nucleotides 1 to 1800. The disclosed MOL2c polypeptide (SEQ ID NO:104) encoded by SEQ ID NO:103 has 600 amino acid residues and is presented using the one-letter code in Table 2F.

TABLE 2F

Encoded MOL2c protein sequence.

(SEQ ID NO:104)

GSDSDISVEICNVCSCVSVENVLYVNCEKVSVYRPNQLKPPWSNFYHLNFQNNFLNTLYPNTFLNFSHAVSLHLGNN
KLQNIEGCAFLGLSTLKQLHLNNNELKILRADTFLGIENLEYLQADYNLIKYIERGAFNKLHKLKVLILNDNLISFL
PDNIFRFASLTHLDIRGNRIQKLPYIGVLEHIGRVVELQLEDNPWNCSCDLLPLKAWLENMPYNIYIGEAICETPSD
LYGRLLKETNKQELCPMGTGSDFDVRILPPSQLENGYTTPNGHTTQTSLHRLVTKPPKTTNPSKISGIVAGKALSNR
NLSQIVSYQTRVPPLTPCPAPCFCKTHPSDLGLSVNCQEKNIQSMSELIPKPLNAKKLHVNGNSIKDVDVSDFTDFE
GLDLLHLGSNQTTVTKGDVFHNLTNLRRLYLNGNQIERLYPEIFSGLHNLQYLYLEYNLIKEISAGTFDSMFNLQLL
YLSNNLLKSLPVYIFSGAPLARLNLRNNKFMYLFVSGVLDQLQSLTQIDLEGSPWDYTCDLVALKLWVEKLSDGIVV
KELKCETPVQFTNIELKSLKNEILCPKLLNKPSAPFTSPAPAITFTTPLGPIRSPPGGPLE

Table 2G shows a ClustalW alignment of the MOL2 variants.

[image]

[image]

Other BLAST results including the sequences used for ClustalW analysis are presented in Table 2H

TABLE 2H
BLAST results for MOL2a

| Gene Index/ | Protein/ | Length | Identity | Positives | |
|---|---|---|---|---|---|
| Identifier | Organism | (aa) | (%) | (%) | Expect |
| gi\|6691962\|emb\| | bG256O22.1 | 853 | 853/853 | 853/853 | 0.0 |
| CAB65788.1\| (AL080239) | (similar to | | (100%) | (100%) | |
| | IGFALS | | | | |
| | (insulin-like | | | | |
| | growth factor | | | | |
| | binding | | | | |
| | protein, acid | | | | |
| | labile | | | | |
| | subunit)) | | | | |
| | [Homo | | | | |
| | sapiens] | | | | |
| gi\|14424224\|sp\| | HYPOTHETICAL | 966 | 347/699 | 470/699 | 1e−173 |
| O94991\|Y918_HUMAN | PROTEIN | | (49%) | (66%) | |
| | KIAA0918 | | | | |
| | [Homo | | | | |
| | Sapiens] | | | | |
| gi\|11877257\|emb\| | bG115M3.1 | 845 | 372/868 | 516/868 | 1e−172 |
| CAC18888.1\| (AL109653) | (novel | | (42%) | (58%) | |
| | protein) | | | | |
| | [Homo | | | | |
| | sapiens] | | | | |
| gi\|12733935\|ref\| | KIAA0848 | 977 | 313/675 | 438/675 | e−150 |
| XP_011654.1\| | protein [Homo | | (46%) | (64%) | |
| | sapiens] | | | | |
| gi\|7662336\|ref\| | KIAA0848 | 977 | 313/675 | 438/675 | e−150 |
| NP_055741.1\| | protein [Homo | | (46%) | (64%) | |
| | sapiens] | | | | |

This information is presented graphically in the multiple sequence alignment given in Table 2I (with MOL2a being shown on line 1) as a ClustalW analysis comparing MOL2a with related protein sequences.

[image]

[image]

[image]

[image]

Table 2J lists the domain description from DOMAIN analysis results against MOL2a. The region from amino acid residue 252 through 302 (SEQ ID NO:6) most probably (E=1e−⁶) contains a “leucine rich repeat C-terminal” domain, aligned here in Table 2J This indicates that the MOL2a sequence has properties similar to those of other proteins known to contain this domain.

[image]

### Chromosomal information

The Insulin-like growth factor binding protein-like protein disclosed in this invention maps to chromosome Xq26.3-28.

### Tissue expression

### MOL2 is expressed in at least the following tissues: adrenal gland, lymphatic tissues, and heart. Other tissues known to express insulin-like growth factor binding proteins are likely.

### Uses of the Compositions of the Invention

The expression pattern, map location and protein similarity information for MOL2 suggest that this a Insulin-like growth factor binding protein-like protein may function as a member of the Insulin-like growth factor binding protein-like protein family. Therefore, the MOL2 nucleic acids and proteins are useful in potential therapeutic applications implicated, for example but not limited to, in various pathologies/disorders as described below and/or other pathologies/disorders. Potential therapeutic uses for MOL2 are, for example but not limited to, the following: (i) Protein therapeutic, (ii) small molecule drug target, (iii) antibody target (therapeutic, diagnostic, drug targeting/cytotoxic antibody), (iv) diagnostic and/or prognostic marker, (v) gene therapy (gene delivery/gene ablation), (vi) research tools, and (vii) tissue regeneration in vitro and in vivo (regeneration for all these tissues and cell types composing these tissues and cell types derived from these tissues).

The MOL2 nucleic acids and proteins are useful in potential therapeutic applications implicated in various diseases and disorders described below and/or other pathologies and disorders. For example, but not limited to, a cDNA encoding the a Insulin-like growth factor binding protein-like protein may be useful in gene therapy, and the a Insulin-like growth factor binding protein-like protein may be useful when administered to a subject in need thereof. By way of nonlimiting example, the compositions of the present invention will have efficacy for treatment of patients suffering from cancer, diabetes, cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, hemophilia, hypercoagulation, idiopathic thrombocytopenic purpura, autoimmune disease, allergies, immunodeficiencies, graft versus host disease (GVHD), lymphaedema, adrenoleukodystrophy, and/or congenital adrenal hyperplasia. MOL2, or fragments thereof, may further be useful in diagnostic applications, wherein the presence or amount of the nucleic acid or the protein are to be assessed.

These materials are further useful in the generation of antibodies that bind immuno-specifically to the novel MOL2 substances for use in therapeutic or diagnostic methods. These antibodies may be generated according to methods known in the art, using prediction from hydrophobicity charts, as described in the “Anti-MOLX Antibodies” section below. The disclosed MOL2 protein has multiple hydrophilic regions, each of which can be used as an immunogen. In one embodiment, a contemplated MOL2 epitope is from about amino acids 75 to 120. In another embodiment, a MOL2 epitope is from about amino acids 180 to 200. In additional embodiments, MOL2 epitopes are from about amino acids 280 to 380, 400 to 450, 475 to 500, and from about amino acids 680 to 850. These novel proteins can also be used to develop assay systems for functional analysis.

### MOL3

### MOL3a

An additional protein of the invention, referred to herein as MOL3a, is a human Semaphorin B-like protein. The novel nucleic acid of 2271 nucleotides (SC85516573_EXT, SEQ ID NO:7) encoding a novel olfactory receptor-like protein is shown in Table 3A. An open reading friame (ORF) was identified beginning with an ATG initiation codon at nucleotides 1-3 and ending with a TAA codon at nucleotides 2269-2271. The nucleotide sequence is presented in Table 3A with the start and stop codons are in bold letters.

TABLE 3A
MOL3a Nucleotide Sequence

| | |
|---|---|
| ATGGCCCTCCCAGCCCTGGGCCTGGACCCCTGGAGCCTCCTGGGCCTTTTCCTCTTCCAACTGCTTC | (SEQ ID NO:7) |
| AGCTGCTGCTGCCGACGACGACCGCGGGGGGAGGCGGGCAGGGGCCCATGCCCAGGGTCAGATACTA | |
| TGCAGGGGATGAACGTAGGGCACTTAGCTTCTTCCACCAGAAGGGCCTCCAGGATTTTGACACTCTG | |
| CTCCTGAGTGGTGATGGAAATACTCTCTACGTGGGGGCTCGAGAAGCCATTCTGGCCTTGGATATCC | |
| AGGATCCAGGGGTCCCCAGGCTAAAGAACATGATACCGTGGCCAGCCAGTGACAGAAAAAAGAGTGA | |
| ATGTGCCTTTAAGAAGAAGAGCAATGAGACACAGTGTTTCAACTTCATCCGTGTCCTGGTTTCTTAC | |
| AATGTCACCCATCTCTACACCTGCGGCACCTTCGCCTTCAGCCCTGCTTGTACCTTCATTGAACTTC | |
| AAGATTCCTACCTGTTGCCCATCTCGGAGGACAAGGTCATGGAGGGAAAAGGCCAAAGCCCCTTTGA | |
| CCCCGCTCACAAGCATACGGCTGTCTTGGTGGATGGGATGCTCTATTCTGGTACTATGAACAACTTC | |
| CTGGGCAGTGAGCCCATCCTGATGCGCACACTGGGATCCCAGCCTGTCCTCAAGACCGACAACTTCC | |
| TCCGCTGGCTGCATCATGACGCCTCCTTTGTGGCAGCCATCCCTTCGACCCAGGTCGTCTACTTCTT | |
| CTTCGAGGAGACAGCCAGCGAGTTTGACTTCTTTGAGAGGCTCCACACATCGCGGGTGGCTAGAGTC | |
| TGCAAGAATGACGTGGGCGGCGAAAAGCTGCTGCAGAAGAAGTGGACCACCTTCCTGAAGGCCCAGC | |
| TGCTCTGCACCCAGCCGGGGCAGCTGCCCTTCAACGTCATCCGCCACGCGGTCCTGCTCCCCGCCGA | |
| TTCTCCCACAGCTCCCCACATCTACGCAGTCTTCACCTCCCAGTGGCAGGTTGGCGGGACCAGGAGC | |
| TCTGCGGTTTGTGCCTTCTCTCTCTTGGACATTGAACGTGTCTTTAAGGGGAAATACAAAGAGTTGA | |
| ACAAAGAAACTTCACGCTGGACTACTTATAGGGGCCCTGAGACCAACCCCCGGCCAGGCAGTTGCTC | |
| AGTGGGCCCCTCCTCTGATAAGGCCCTGACCTTCATGAAGGACCATTTCCTGATGGATGAGCAAGTG | |
| GTGGGGACGCCCCTGCTGGTGAAATCTGGCGTGGAGTATACACGGCTTGCAGTGGAGACAGCCCAGG | |
| GCCTTGATGGGCACAGCCATCTTGTCATGTACCTGGGAACCAGTACAGGGTCGCTCCACAAGGCTGT | |
| GGTAAGTGGGGACAGCAGTGCTCATCTGGTGGAAGAGATTCAGCTGTTCCCTGACCCTGAACCTGTT | |
| CGCAACCTGCAGCTGGCCCCCACCCAGGGTGCAGTGTTTGTAGGCTTCTCAGGAGGTGTCTGGAGGG | |
| TGCCCCGAGCCAACTGTAGTGTCTATGAGAGCTGTGTGGACTGTGTCCTTGCCCGGGACCCCCACTG | |
| TGCCTGGGACCCTGAGTCCCGACTCTGCTCTCTTAGGAACTCCTGGAAGCAGGACATGGAGCGGGGG | |
| AACCCAGAGTGGGCATGTGCCAGTGGCCCCATGAGCAGGAGCCTTCGGCCTCAGAGCCGCCCGCAAA | |
| TCGTTAAAGAAGTCCTGGCTGTCCCCAACTCCATCCTGGAGCTCCCCTGCCCCCACCTGTCAGCCTT | |
| GGCCTCTTATTATTGGAGTCATGGCCCAGCAGCAGTCCCAGAAGCCTCTTCCACTGTCTACAATGGC | |
| TCCCTCTTGCTGATAGTGCAGGATGGAGTTGGGGGTCTCTACCAGTGCTGGGCAACTGAGAATGGCT | |
| TTTCATACCCTGTGATCTCCTACTGGGTGGACAGCCAGGACCAGACCCTGGCCCTGGATCCTGAACT | |
| GGCAGGCATCCCCCGGGAGCATGTGAAGGTCCCGTTGACCAGGGTCAGTGGTGGGGCCGCCCTGGCT | |
| GCCCAGCAGTCCTACTGGCCCCACTTTGTCACTGTCACTGTCCTCTTTGCCTTAGTGCTTTCAGGAG | |
| CCCTCATCATCCTCGTGGCCTCCCCATTGAGAGCACTCCGGGCTCGGGGCAAGGTTCAGGGCTGTGA | |
| GACCCTGCGCCCTGGGGAGAAGGCCCCGTTAAGCAGAGAGCAACACCTCCAGTCTCCCAAGGAATGC | |
| AGGACCTCTGCCAGTGATGTGGACGCTGACAACAACTGCCTAGGCACTGAGGTAGCTTAA | |

The disclosed MOL3a polypeptide (SEQ ID NO:8) encoded by SEQ ID NO:7 has 756 amino acid residues, and is presented using the one-letter code in Table 3B. The MOL3a protein was analyzed for signal peptide prediction and cellular localization. SignalP results predict that MOL3a is cleaved between position 31 and 32 (TTA-GG) of SEQ ID NO:8. Psort and Hydropathy profiles also predict that MOL3a is likely to be localized at the plasma membrane (certainty of 0.7300).

TABLE 3B
Encoded MOL3a protein sequence.

(SEQ ID NO:8)

MALPALGLDPWSLLGLFLFQLLQLLLPTTTAGGGGQGPMPRVRYYAGDERRALSFFHQKGLQDFDTLLLSGDGNT
LYVGAREAILALDIQDPGVPRLKNMIPWPASDRKKSECAFKKKSNETQCFNFIRVLVSYNVTHLYTCGTFAFSPA
CTFIELQDSYLLPISEDKVMEGKGQSPFDPAHKHTAVLVDGMLYSGTMNNFLGSEPILMRTLGSQPVLKTDNFLR
WLHHDASFVAAIPSTQVVYFFFEETASEFDFFERLHTSRVARVCKNDVGGEKLLQKKWTTFLKAQLLCTQPGQLP
FNVIRHAVLLPADSPTAPHIYAVFTSQWQVGGTRSSAVCAFSLLDIERVFKGKYKELNKETSRWTTYRGPETNPR
PGSCSVGPSSDKALTFMKDHFLMDEQVVGTPLLVKSGVEYTRLAVETAQGLDGHSHLVMYLGTSTGSLHKAVVSG
DSSAHLVEEIQLFPDPEPVRNLQLAPTQGAVFVGFSGGVWRVPRANCSVYESCVDCVLARDPHCAWDPESRLCSL
RNSWKQDMERGNPEWACASGPMSRSLRPQSRPQIVKEVLAVPNSILELPCPHLSALASYYWSHGPAAVPEASSTV
YNGSLLLIVQDGVGGLYQCWATENGFSYPVISYWVDSQDQTLALDPELAGIPREHVKVPLTRVSGGAALAAQQSY
WPHFVTVTVLFALVLSGALIILVASPLRALRARGKVQGCETLRPGEKAPLSREQHLQSPKECRTSASDVDADNNC
LGTEVA

The MOL3a nucleic acid sequence has 1398/1672 (83%) identical to a mouse Semaphorin B mRNA (GENBANK-ID: X85991).

The full amino acid sequence of MOL3a was found to have 628 of 760 (82%) identical to, and 674 of 760 residues (88%) homologous with, the 760 amino acid residue Semaphorin B protein from mouse (ptnr: SWISSNEW-ACC:Q62178).

MOL3a expression in different tissues was examined through TaqMan as described below in Example 1.

MOL3a also has high homology to the proteins disclosed in the BLASTP searches of the proprietary PATP database shown in Table 3C.

TABLE 3C
BLAST results for MOL3a

| Gene Index/ | Protein/ | | | Positives | |
|---|---|---|---|---|---|
| Identifier | Organism | Length (aa) | Identity (%) | (%) | Expect |
| patp: AAB24084 | Human PRO1317 | 761 | 751/761 | 753/761 | 0.0 |
| | protein | | (98%) | (98%) | |
| patp: AAY99418 | Human PRO1317 | 761 | 751/761 | 753/761 | 0.0 |
| | (UNQ783) | | (98%) | (98%) | |
| patp: AAB66043 | Human TANGO | 761 | 751/761 | 753/761 | 0.0 |
| | 265 | | (98%) | (98%) | |
| patp: AAB66167 | Unidentified | 761 | 751/761 | 753/761 | 0.0 |
| | | | (98%) | (98%) | |
| patp: AAB37984 | Human secreted | 762 | 743/761 | 745/761 | 0.0 |
| | protein encoded | | (97%) | (97%) | |
| | by gene 1 clone | | | | |
| | HTDAA93 | | | | |
| patp: AAB66045 | Human | 730 | 720/730 | 722/730 | 0.0 |
| | TANGO 265 | | (98%) | (98%) | |
| | mature protein | | | | |
| patp: AAB66046 | Human | 652 | 642/652 | 644/652 | 0.0 |
| | TANGO 265 | | (98%) | (98%) | |
| | extracellular | | | | |
| | domain | | | | |

### Tissue Localization

MOL3a is expressed in at least the following tissues: Pituitary Gland, Thalamus

### Chromosomal Localization

MOL3a maps to chromosome 1.

### MOL3b

In the present invention, the target sequence identified previously, MOL3a, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated MOL3b, or alternatively Accession Number CG53027-02. This is a spliced variant of the previously identified sequence (Accession Number SC85516573_EXT) at amino acids 293-329.

A protein of the invention, referred to herein as MOL3b, is a human Semaphorin B-like protein. The novel nucleic acid of 2281 nucleotides (CG53027-02, SEQ ID NO:9) encoding a Semaphorin B-like protein is shown in Table 3D. An open reading frame (ORF) was identified beginning with a non-initiating codon for the mature protein at nucleotides 2-4 and ending with non-stop codon at nucleotides 2264-2266. The open reading frame may be extendable in both the 5′ and 3′ directions because of the lack of traditional start and stop codons. The nucleotide sequence is presented in Table 3D with the start and stop codons in bold letters and the 5′ and 3′ untranslated regions underlined.

TABLE 3D
MOL3b Nucleotide Sequence

(SEQ ID NO:9)

GCCTGTGCCTAGAGTTTAAGCTACCTCAGTGCCTAGGCAGTTGTTGTCAGCGTCCACATCACTGGCAGAGGTCCT
GCATTCCTTGGGAGACTGGAGGTGTTGCTCTCTGCTTAACGGGGCCTTCTCCCCAGGGCGCAGGGTCTCACAGCC
CTGAACCTTGCCCCGAGCCCGGAGTGCTCTCAATGGGGAGGCCACGAGGATGATGAGGGCTCCTGAAAGCACTAA
GGCAAAGAGGACAGTGACAGTGACAAAGTGGGGCCAGTAGGACTGCTGGGCAGCCAGGGCGGCCCCACCACTGAC
CCTGGTCAACGGGACCTTCACATGCTCCCGGGGGATGCCTGCCAGTTCAGGATCCAGGGCCAGGGTCTGGTCCTG
GCTGTCCACCCAGTAGGAGATCACAGGGTATGAAAAGCCATTCTCAGTTGCCCAGCACTGGTAGAGACCCCCAAC
TCCATCCTGCACTATCAGCAAGAGGGAGCCATTGTAGACAGTGGAAGAGGCTTCTGGGACTGCTGCTGGGCCATG
ACTCCAATAATAAGAGGCCAAGGCTGACAGGTGGGGGCAGGGGAGCTCCAGGATGGAGTTGGGGACAGCCAGGAC
TTCTTTAATGATTTGCGGGCGGCTCTGAGGCCGAAGGCTCCTGCTCATGGGGCCACTGGCACATGCCCACTCTGG
GTTCCCCCGCTCCATGTCCTGCTTCCAGGAGTTCAGGTTGGGGGCAGACAGGAGGCAACAGGTTCGGGACTCAGG
GTCCCAGGCACAGTGGGGGTCCCGGGCAAGGACACAGTCCACACAGCTCTCATAGACACTACAGTTGGCTCGGGG
CACCCTCCAGACACCTCCTGAGAAGCCTACAAACACTGCACCCTGGGTGGGGGCCAGCTGCAGGTTGCGAACAGG
TTCAGGGTCAGGGAACAGCTGAATCTCTTCCACCAGATGAGCACTGCTGTCCCCACTTACCACAGCCTTGTGGAG
CGACCCTGTGGTGGTTCCCAGGTACATGACAAGATGGCTGTGCCCATCAAGGCCCTGGGCTGTCTCCACTGCAAG
CCGTGTATACTCCACGCCAGATTTCACCAGCAGGGGCGTCCCCACCACTTGCTCATCCATCAGGAAATGGTCCTT
CATGAAGGTCAGGGCCTTATCAGAGGAGGGGCCCACTGAGCAACTGCCTGGCCGGGGGTTGGTCTCAGGGCCCCT
ATAAGTAGTCCAGCGTGAAGTTTCTTTGTTCAACTCTTTGTATTTCCCCTTAAAGACACGTTCAATGTCCAAGAG
AGAGAAGGCACAAACCGCAGAGCTCCTGGTCCCGCCAACCTGCCACTGGGAGGTGAAGACTGCGTAGATGTGGGG
AGCTGTGGGAGAATCGGCGGGGAGCAGGACCGCGTGGCGGATGACGTTGAAGGGCAGCTGCCCCGGCTGGGTGCA
GAGCAGCTGGGCCTTCAGGAAGGTGGTCCACTTCTTCTGCAGCAGCTTTTCGCCGCCCACGTCATTCTTGCAGAC
TCTAGCCACCCGCGATGTGTGGAGCCTCTCAAAGAAGTCAAACTCGCTGGCTGTCTCCTCGAAGAAGAAGTAGAC
GACCTGGGTCGAAGGGATGGCTGCCACAAAGGAGGCGTCATGATGCAGCCAGCGGAGGAAGTTGTCGGTCTTGAG
GACAGGCTGGGATCCCAGTGTGCGCATCAGGATGGGCTCACTGCCCAGGAAGTTGTTCATAGTACCAGAATAGAG
CATCCCATCCACCAAGACAGCCGTATGCTTGTGAGCGGGGTCAAAGGGGCTTTGGCCTTTTCCCTCCATGACCTT
GTCCTCCGAGATGGGCAACAGGTAGGAATCTTGAAGTTCAATGAAGGTACAAGCAGGGCTGAAGGCGAAGGTGCC
GCAGGTGTAGAGATGGGTGACATTGTAAGAAACCAGGACACGGATGAAGTTGAAACACTGTGTCTCATTGCTCTT
CTTCTTAAAGGCACATTCACTCTTTTTTCTGTCACTGGCTGGCCACGGTATCATGTTCTTTAGCCTGGGGACCCC
TGGATCCTGGATATCCAAGGCCAGAATGGCTTCTCGAGCCCCCACGTAGAGAGTATTTCCATCACCACTCAGGAG
CAGAGTGTCAAAATCCTGGAGGCCCTTCTGGTGGAAGAAGCTAAGTGCCCTACGTTCATCCCCTGCATAGTATCT
GACCCTGGGCATGGGCCCCTGCCCGCCTCCCCCCGCGGTCGTCGTCGGCAGCAGCAGCTGAAGCAGTTGGAAGAG
GAAAAGGCCCAGGAGGCTCCAGGGGTCCAGG

The disclosed MOL3b polypeptide (SEQ ID NO:10) encoded by SEQ ID NO:9 has 754 amino acid residues, and is presented using the one-letter code in Table 3E. The MOL3b protein was analyzed for signal peptide prediction and cellular localization. SignalP results predict that MOL3b is cleaved between position 24 and 25 (TTA-GG) of SEQ ID NO:10. Psort and Hydropathy profiles also predict that MOL3b is likely to be localized at the plasma membrane (certainty of 0.7300).

TABLE 3E
Encoded MOL3b protein sequence

(SEQ ID NO:10)

LDPWSLLGLFLFQLLQLLLPTTTAGGGGQGPMPRVRYYAGDERRALSFFHQKGLQDFDTLLLSGDGNTLYVGARE
AILALDIQDPGVPRLKNMIPWPASDRKKSECAFKKKSNETQCFNFIRVLVSYNVTHLYTCGTFAFSPACTFIELQ
DSYLLPISEDKVMEGKGQSPFDPAHKHTAVLVDGMLYSGTMNNFLGSEPILMRTLGSQPVLKTDNFLRWLHHDAS
FVAAIPSTQVVYFFFEETASEFDFFERLHTSRVARVCKNDVGGEKLLQKKWTTFLKAQLLCTQPGQLPFNVIRHA
VLLPADSPTAPHIYAVFTSQWQVGGTRSSAVCAFSLLDIERVFKGKYKELNKETSRWTTYRGPETNPRPGSCSVG
PSSDKALTFMKDHFLMDEQVVGTPLLVKSGVEYTRLAVETAQGLDGHSHLVMYLGTTTGSLHKAVVSGDSSAHLV
EEIQLFPDPEPVRNLQLAPTQGAVFVGFSGGVWRVPRANCSVYESCVDCVLARDPHCAWDPESRTCCLLSAPNLN
SWKQDMERGNPEWACASGPMSRSLRPQSRPQIIKEVLAVPNSILELPCPHLSALASYYWSHGPAAVPEASSTVYN
GSLLLIVQDGVGGLYQCWATENGFSYPVISYWVDSQDQTLALDPELAGIPREHVKVPLTRVSGGAALAAQQSYWP
HFVTVTVLFALVLSGALIILVASPLRALRARGKVQGCETLRPGEKAPLSREQHLQSPKECRTSASDVDADNNCLG
TEVA

The MOL3b nucleic acid sequence has 1910 of 2279 bases (83%) identical to a gb:GENBANK-ID:MMRNASEMB|acc:X85991.1 mRNA from Mus musculus (M. musculus mRNA for semaphorin B).

The full amino acid sequence of the protein of the invention was found to have 722 of 755 amino acid residues (95%) identical to, and 723 of 755 amino acid residues (95%) similar to, the 762 amino acid residue ptnr:TREMBLNEW-ACC:BAB20087 protein from Homo sapiens (Human) (SEMB).

The presence of identifiable domains in the protein disclosed herein was determined by searches versus domain databases such as Pfam, PROSITE, ProDom, Blocks or Prints and then identified by the Interpro domain accession number. Significant domains are summarized in Table 3F.

TABLE 3F
Domain search for MOL3b
HMMER is freely distributed under the GNU General Public
License (GPL).
HMM file: pfamHMMs
Sequence file:/
data4/genetools/kspytek35060Cg53027_01ProteinFasta.txt
Query: CG53027_01
Scores for sequence family classification (score includes
all domains):

| Model | Description | Score | E-value | N |
|---|---|---|---|---|
| Sema | Sema domain | 618.4 | 4.2e−182 | 1 |
| Plexin_repeat | Plexin repeat | 22.0 | 0.013 | 1 |
| integrin_B | Integrins, beta chain | 6.5 | 0.063 | 1 |

Parsed for domains:

| | | | | hmm- | | | |
|---|---|---|---|---|---|---|---|
| Model | Domain | seq-f | seq-t | f | hmm-t | score | E-value |
| Sema | 1/1 | 57 | 471 . . . | 1 | 490 [ ] | 618.4 | 4.2e−182 |
| integrin_{—} | 1/1 | 495 | 509 . . . | 1 | 14 [ . | 6.5 | 0.063 |
| B | | | | | | | |
| Plexin_{—} | 1/1 | 489 | 555 . . . | 1 | 67 [ ] | 22.0 | 0.013 |
| repeat | | | | | | | |

### Tissue Localization

MOL3b is expressed in at least the following tissues: thalamus and Pituitary Gland. Expression information was derived from the tissue sources of the sequences that were included in the derivation of the sequence of MOL3b.

### Chromosomal Localization

MOL3b maps to chromosome 1. This assignment was made using mapping information associated with genomic clones, public genes and ESTs sharing sequence identity with the disclosed sequence and CuraGen Corporation's Electronic Northern bioinformatic tool.

The disclosed MOL3a protein (SEQ ID NO:8) also has good identity with a number of other proteins, as shown in Table 3G.

TABLE 3G
BLAST results for MOL3a

| Gene Index/ | Protein/ | Length | Identity | Positives | |
|---|---|---|---|---|---|
| Identifier | Organism | (aa) | (%) | (%) | Expect |
| gi\|12248382\|dbj\| | SEMB [Homo | 762 | 719/762 | 722/762 | 0.0 |
| BAB20087.1\| | sapiens] | | (94%) | (94%) | |
| (AB029394) | | | | | |
| gi\|7305469\|ref\| | sema domain, | 760 | 626/760 | 671/760 | 0.0 |
| NP_038686.1\| | immunoglobulin | | (82%) | (87%) | |
| | domain (Ig), | | | | |
| | transmembrane | | | | |
| | domain (TM) | | | | |
| | and short | | | | |
| | cytoplasmic | | | | |
| | domain, | | | | |
| | (semaphorin) 4A | | | | |
| | [Mus musculus] | | | | |
| gi\|11641291\|ref\| | hypothetical | 328 | 318/328 | 320/328 | 0.0 |
| NP_071762.1\| | protein | | (96%) | (96%) | |
| | FLJ12287 | | | | |
| | similar to | | | | |
| | semaphorins | | | | |
| | [Homo sapiens] | | | | |
| gi\|12698035\|dbj\| | KIAA1745 | 893 | 296/760 | 422/760 | e−123 |
| BAB21836.1\| | protein [Homo | | (38%) | (54%) | |
| (AB051532) | sapiens] | | | | |
| gi\|8134698\|sp\| | SEMAPHORIN 4B | 782 | 268/684 | 382/684 | e−123 |
| Q62179\|SM4B_MOUSE | (SEMAPHORIN C) | | (39%) | (55%) | |
| | (SEMA C) | | | | |

This information is presented graphically in the multiple sequence alignment given in Table 3H (with MOL3a being shown on line 1 and MOL3b on line 2) as a ClustalW analysis comparing MOL3 with related protein sequences.

[image]

[image]

[image]

[image]

Table 3I lists the domain description from DOMAIN analysis results against MOL3. The region from amino acid residue 64 through 478 (SEQ ID NO:8) most probably (E=1e⁻¹²¹) contains a PSI, domain found in Plexins, Semaphorins and Integrins, aligned here in Table 3I. Semaphorins are involved in growth cone guidance as well as other developmental processes. Plexins and integrins are involved in developmental processes. The MOL1 sequence likely has properties similar to those of other proteins known to contain this domain

[image]

The protein similarity information, expression pattern, cellular localization, and map location for the protein and nucleic acid for MOL3 suggest that this Semaphorin B-like protein may have important structural and/or physiological functions characteristic of the Semaphorin B family. This family is involved in developmental processes including growth cone guidance. MOL3 likely plays a similar role in those developmental processes. Therefore, the MOL3 nucleic acids and proteins are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The MOL3 nucleic acids and proteins have applications in the diagnosis and/or treatment of various diseases and disorders. For example, the compositions of the present invention will have efficacy for the treatment of patients suffering from: neuronal developmental, organizational, mediated and interactive disorders and disease; endocrine dysfunctions, diabetes, obesity, growth and reproductive disorders, injury repair as well as other diseases, disorders and conditions.

These materials are further useful in the generation of antibodies that bind immuno-specifically to the novel MOL3 substances for use in therapeutic or diagnostic methods. These antibodies may be generated according to methods known in the art, using prediction from hydrophobicity charts, as described in the “Anti-MOLX Antibodies” section below. The disclosed MOL3 protein has multiple hydrophilic regions, each of which can be used as an immunogen. In one embodiment, a contemplated MOL3 epitope is from about amino acids 30 to 100. In another embodiment, a MOL3 epitope is from about amino acids 110 to 150. In additional embodiments, MOL3 epitopes are from about amino acids 160 to 200, 210 to 230, 250 to 300, 350 to 400, 450 to 475, 500 to 575, 620 to 630, and from about amino acids 700 to 750. These novel proteins can also be used to develop assay systems for functional analysis.

### MOL4

### MOL4a

The disclosed novel semaphorin-like protein, MOL4a (also referred to herein as SC_{—}111750277_A), is encoded by a nucleic acid, 6408 nucleotides long (SEQ ID NO:11). An open reading frame was identified beginning with an ATG initiation codon at nucleotides 1400-1402 and ending with a TGA codon at nucleotides 5456-5458. Putative untranslated regions upstream from the initiation codon and downstream from the termination codon are underlined in Table 4A, and the start and stop codons are in bold letters.

TABLE 4A
MOL4a Nucleotide Sequence.

(SEQ ID NO:11)

CCTGGGACTCTGGGAGAATGGTCCAGAGCTCATTGTCCTTGATAAAATGATAGATTTGGACTCAATATCCCA
TGCTGCCTCTTCCAACTTGATTTTTACCCCAGACTGGGCTACCAGACTGGTATGCCCACACATGCCCGTTTCCTT
TCTTTTCTTCTCTGCATCTCTGCCTTTGTGTCCAGAGCGTGTTTTCCCTTTGCAAGTTTCTCTCCATTCTGCACA
TTATGAGTTTCAGCATTTCTGTTGCCCTAGAAAGTCTATCTTTGAGATCTTGCACTGTTTCTCTTTTTACAGTGT
CTCATAAACTCCCTTCTTGGATTCAGAACCACCCTTTCTTTCCCATTATCCTGTCAAACTGCTTCTTGCCATGGT
CCAGGGGTAGGAGGATGGCAGGCAGGAGGTGCTTCTCTGGGGCTCTTAGTGTCTCAATTCTTCTGCTTTATCTGG
GTTTTCCTTTACCCAGAATTTTATTATGTAAAATGCTTCACTCAGACTTTGTTCTAATTATCCAATTTTTGGCAT
ACTCTAGAAAGTCTTTTGATATTTTCCTTCCTCCAACTTATCTATTTTTATTTCATAGTTCTCTTTGGTTATCTC
TTAGAATCACACTTTCCTGGTTTTAATTTTTCAAATCCTTTGTCTTTCTCACTCGTTCTTAGGTCACCTTTTTTT
ACATTTTCAAATATATTTTTTGTTCAGCAGAGGGCTCCCTTCCCATCCCTCTTGCAGCCCGGGCAGCTAGGATTT
GAAGCTTGCCCCTTGAATCTTTCTCTCCCGCCTTCTAGCCATCAGAAACACTAGATCACTTAAACTTGTAAACAA
TTCGGCCTCGCTCCTTGTGATTGCGCTAAACCTTCCGTCCTCAGCTGAGAACGCTCCACCACCTCCCCGGATCGC
TCATCTCTTGGCTGCCCTCCCACTGTTCCTGATGTTATTTTACTCCCCGTATCCCCTACTCGTTCTTCACAATTC
TGTAGGGTGCGTATTACTAACCCCAGTTTACAGCTGAGGAAACTGAGGCTTGGAGAGGTTCGCTCGGTATCGTAC
AGTTTGCAAGGTTAACCCTAATCCGGCCAGTTCTGGCTTTCCAGCCCAGCCCAGCAGCCTAGCCTCCCTCTCTGC
CGCTGCAGGTTATAACGGCTCTCCCCCGTTTTACACGAGGTCCCTTCCCCTTCAAATCCACAGGCAGGAAGATCG
TTCCGAACTGACGGGGCTGGGGAATGTGGGAGTCCGGAGTGGGGTTTGGGGGAGCTTCCTCAGGCCCTGAGTGTT
GGGGTGGGCAGGCCGCGCCGATGGCCCTCGGGGATGTCACATTCGAGATGGGGTGACCGAGAACGGCAAGGCGGG
ATGTGGCAAACGGCGGCAAGTGCTCGGAGTCCTAGGTCTTGCCGCCGGAATGCCGGCCGGGGGAAGGGGCTTCGGC
CCACCGGGCTGGTCACCACACTCGGCAGGCCCGGGGCGGGAGTCGGCCGAGCAGCCGCGGGATGCAGGGCGCCCC
CTCGCGCTCCTCCGCGCGCCTCGAGGCTGGCGGGTGCAGCGCCCGCCGCGGCAGGTCTGCTCCAGCCCCCTCCTC
TTTTTCGCTCCCGCTCCCCTCCTTCTCTCCCTTTGCTTGCAACTCCTCCCCCACCGCCCCCTCCCTCCTTCTGCT
CCCGCGGTCTCCTCCTCCCTGCTCTCTCCGAGCGCCGGGTCGGGAGCTAGTTGGAGCGCGGGGGTTGGTGCCAGA
GCCCAGCTCCGCCGAGCCGGGCGGGTCGGCAGCGCATCCAGCGGCTGCTGGGAGCCCGAGCGCAGCGGGCGCGGG
CCCGGGTGGGGACTGCACCGGAGCGCTGAGAGCTGGAGGCCGTTCCTGCGCGGCCGCCCCATTCCCAGACCGGCC
GCCAGCCCATCTGGTTAGCTCCCGCCGCTCCGCGCCGCCCGGGAGTCGGGAGCCGCGGGGAACCGGGCACCTGCA
CCCGCCTCTGGGAGTGAGTGGTTCCAGCTGGTGCCTGGCCTGTGTCTCTTGGATGCCCTGTGGCTTCAGTCCGTC
TCCTGTTGCCCACCACCTCGTCCCTGGGCCGCCTGATACCCCAGCCCAACAGCTAAGGTGTGGATGGACAGTAGG
GGGCTGGCTTCTCTCACTGGTCAGGGGTCTTCTCCCCTGTCTGCCTCCCGGAGCTAGGACTGCAGAGGGGCCTAT
CATGGTGCTTGCAGGCCCCCTGGCTGTCTCGCTGTTGCTGCCCAGCCTCACACTGCTGGTGTCCCACCTCTCCAG
CTCCCAGGATGTCTCCAGTGAGCCCAGCAGTGAGCAGCAGCTGTGCGCCCTTAGCAAGCACCCCACCGTGGCCTT
TGAAGACCTGCAGCCGTGGGTCTCTAACTTCACCTACCCTGGAGCCCGGGATTTCTCCCAGCTGGCTTTGGACCC
CTCCGGGAACCAGCTCATCGTGGGAGCCAGGAACTACCTCTTCAGACTCAGCCTTGCCAATGTCTCTCTTCTTCA
GGCCACAGAGTGGGCCTCCAGTGAGGACACGCGCCGCTCCTGCCAAAGCAAAGGGAAGACTGAGGAGGAGTGTCA
GAACTACGTGCGAGTCCTGATCGTCGCCGGCCGGAAGGTGTTCATGTGTGGAACCAATGCCTTTTCCCCCATGTG
CACCAGCAGACAGGTGGGGAACCTCAGCCGGACTACTGAGAAGATCAATGGTGTGGCCCGCTGCCCCTATGACCC
ACGCCACAACTCCACAGCTGTCATCTCCTCCCAGGGGGAGCTCTATGCAGCCACGGTCATCGACTTCTCAGGTCG
GGACCCTGCCATCTACCGCAGCCTGGGCAGTGGGCCACCGCTTCGCACTGCCCAATATAACTCCAAGTGGCTTAA
TGAGCCAAACTTCGTGGCAGCCTATGATATTGGGCTGTTTGCATACTTCTTCCTGCGGGAGAACGCAGTGGAGCA
CGACTGTGGACGCACCGTGTACTCTCGCGTGGCCCGCGTGTGCAAGAATGACGTGGGGGGCCGATTCCTGCTGGA
GGACACATGGACCACATTCATGAAGGCCCGGCTCAACTGCTCCCGCCCGGGCGAGGTCCCCTTCTACTATAACGA
GCTGCAGAGTGCCTTCCACTTGCCAGAGCAGGACCTCATCTATGGAGTTTTCACAACCAACGTAAACAGCATCGC
GGCTTCTGCTGTCTGCGCCTTCAACCTCAGTGCTATCTCCCAGGCTTTCAATGGCCCATTTCGCTACCAGGAGAA
CCCCAGGGCTGCCTGGCTCCCCATAGCCAACCCCATCCCCAATTTCCAGTGTGGCACCCTGCCTGAGACCGGTCC
CAACGAGAACCTGACGGAGCGCAGCCTGCAGGACGCGCAGCGCCTCTTCCTGATGAGCGAGGCCGTGCAGCCGGT
GACACCCGAGCCCTGTGTCACCCAGGACAGCGTGCGCTTCTCACACCTCGTGGTGGACCTGGTGCAGGCTAAAGA
CACGCTCTACCATGTACTCTACATTGGCACCGAGTCGGGCACCATCCTGAAGGCGCTGTCCACGGCGAGCCGCAG
CCTCCACGGCTGCTACCTGGAGGAGCTGCACGTGCTGCCCCCCGGGCGCCGCGAGCCCCTGCGCAGCCTGCGCAT
CCTGCACAGCGCCCGCGCGCTCTTCGTGGGGCTGAGAGACGGCGTCCTGCGGGTCCCACTGGAGAGGTGCGCCGC
CTACCGCAGCCAGGGGGCATGCCTGGGGGCCCGGGACCCGTACTGTGGCTGGGACGGGAAGCAGCAACGTTGCAG
CACACTCGAGGACAGCTCCAACATGAGCCTCTGGACCCAGAACATCACCGCCTGTCCTGTGCGGAATGTGACACG
GGATGGGGGCTTCGGCCCATGGTCACCATGGCAACCATGTGAGCACTTGGATGGGGACAACTCAGGCTCTTGCCT
GTGTCGAGCTCGATCCTGTGATTCCCCTCGACCCCGCTGTGGGGGCCTTGACTGCCTGGGGCCAGCCATCCACAT
CGCCAACTGCTCCAGGAATGGGGCGTGGACCCCGTGGTCATCGTGGGCGCTGTGCAGCACGTCCTGTGGCATCGG
CTTCCAGGTCCGCCAGCGAAGTTGCAGCAACCCTGCTCCCCGCCACGGGGGCCGCATCTGCGTGGGCAAGAGCCG
CGAGGAACGGTTCTGTAATGAGAACACGCCTTGCCCGGTGCCCATCTTCTGGGCTTCCTGGGGCTCCTGGAGCAA
GTGCAGCAGCAACTGTGGAGGGGGCATGCAGTCGCGGCGTCGGGCCTGCGAGAACGGCAACTCCTGCCTGGGCTG
CGGCGTGGAGTTCAAGACGTGCAACCCCGAGGGCTGCCCCGAAGTGCGGCGCAACACCCCCTGGACGCCGTGGCT
GCCCGTGAACGTGACGCAGGGCGGGGCACGGCAGGAGCAGCGGTTCCGCTTCACCTGCCGCGCGCCCCTTGCAGA
CCCGCACGGCCTGCAGTTCGGCAGGAGAAGGACCGAGACGAGGACCTGTCCCGCGGACGGCTCCGGCTCCTGCGA
CACCGACGCCCTGGTGGAGGACCTCCTGCGCAGCGGGAGCACCTCCCCGCACACGGTGAGCGGGGGCTGGGCCGC
CTGGGGCCCGTGGTCGTCCTGCTCCCGGGACTGCGAGCTGGGCTTCCGCGTCCGCAAGAGAACGTGCACTAACCC
GGAGCCCCGCAACGGGGGCCTGCCCTGCGTGGGCGATGCTGCCGAGTACCAGGACTGCAACCCCCAGGCTTGCCC
AGTTCGGGGTGCTTGGTCCTGCTGGACCTCATGGTCTCCATGCTCAGCTTCCTGTGGTGGGGGTCACTATCAACG
CACCCGTTCCTGCACCAGCCCCGCACCCTCCCCAGGTGAGGACATCTGTCTCGGGCTGCACACGGAGGAGGCACT
ATGTGCCACACAGGCCTGCCCAGAAGGCTGGTCGCCCTGGTCTGAGTGGAGTAAGTGCACTGACGACGGAGCCCA
GAGCCGAAGCCGGCACTGTGAGGAGCTCCTCCCAGGGTCCAGCGCCTGTGCTGGAAACAGCAGCCAGAGCCGCCC
CTGCCCCTACAGCGAGATTCCCGTCATCCTGCCAGCCTCCAGCATGGAGGAGGCCACCGGCTGTGCAGGGTTCAA
TCTCATCCACTTGGTGGCCACGGGCATCTCCTGCTTCTTGGGCTCTGGGCTCCTGACCCTAGCAGTGTACCTGTC
TTGCCAGCACTGCCAGCGTCAGTCCCAGGAGTCCACACTGGTCCATCCTGCCACCCCCAACCATTTGCACTACAA
GGGCGGAGGCACCCCGAAGAATGAAAAGTACACACCCATGGAATTCAAGACCCTGAACAAGAATAACTTGATCCC
TGATGACAGAGCCAACTTCTACCCATTGCAGCAGACCAATGTGTACACGACTACTTACTACCCAAGCCCCCTGAA
CAAACACAGCTTCCGGCCCGAGGCCTCACCTGGACAACGGTGCTTCCCCAACAGCTGATACCGCCGTCCTGGGGA
CTTGGGCTTCTTGCCTTCATAAGGCACAGAGCAGATGGAGATGGGACAGTGGAGCCAGTTTGGTTTTCTCCCTCT
GCACTAGGCCAAGAACTTGCTGCCTTGCCTGTGGGGGGTCCCATCCGGCTTCAGAGAGCTCTGGCTGGCATTGAC
CATGGGGGAAAGGGCTGGTTTCAGGCTGACATATGGCCGCAGGTCCAGTTCAGCCCAGGTCTCTCATGGTTATCT
TCCAACCCACTGTCACGCTGACACTATGCTGCCATGCCTGGGCTGTGGACCTACTGGGCATTTGAGGAACTGGAG
AATGGAGATGGCAAGAGGGCAGGCTTTTAAGTTTGGGTTGGAGACAACTTCCTGTGGCCCCCACAAGCTGAGTCT
GGCCTTCTCCAGCTGGCCCCAAAAAAGGCCTTTGCTACATCCTGATTATCTCTGAAAGTAATCAATCAAGTGGCT
CCAGTAGCTCTGGATTTTCTGCCAGGGCTGGGCCATTGTGGTGCTGCCCCAGTATGACATGGGACCAAGGCCAGC
GCAGGTTATCCACCTCTGCCTGGAAGTCTATACTCTACCCAGGGCATCCCTCTGGTCAGAGGCAGTGAGTACTGG
GAACTGGAGGCTGACCTGTGCTTAGAAGTCCTTTAATCTGGGCTGGTACAGGCCTCAGCCTTGCCCTCAATGCAC
GAAAGGTGGCCCAGGAGAGAGGATCAATGCCACAGGAGGCAGAAGTCTGGCCTCTGTGCCTCTATGGAGACTATC
TTCCAGTTGCTGCTCAACAGAGTTGTTGGCTGAGACCTGCTTGGGAGTCTCTGCTGGCCCTTCATCTGTTCAGGA
ACACACACACACACACACTCACACACGCACACACAATCACAATTTGCTACAGCAACAAAAAAGACATTGGGCTGT
GGCATTATTAATTAAAGATGATATCCAGTCTCC

The 1352 amino acid MOL4a polypeptide (SEQ ID NO:12) encoded by SEQ ID NO:11 is presented using the one-letter amino acid code in Table 4B. The Psort profile for MOL4a predicts that this sequence has no signal peptide and is likely to be localized in the plasma membrane with a certainty of 0.7900. MOL4a has a molecular weight of 145674.1 Daltons.

TABLE 4B
MOL4a protein sequence

(SEQ ID NO:12)

MPAGEGASAHRAGHHTRQARGGSRPSSRGMQGAPSRSSARLEAGGCSARRGRSAPAPSSFSLPLPSFSPFACNSSP
TAPSLLLLPRSPPPCSLRAPGRELVGARGLVPEPSSAEPGGSAAHPAAAGSPSAAGAGPGGDCTGALRAGGRSCAA
APFPDRPPAHLVSSRRSAPPGSREPRGTGHLHPPLGVSGSSWCLACVSWMPCGFSPSPVAHHLVPGPPDTPAQQLR
CGWTVGGWLLSLVRGLLPCLPPGARTAEGPIMVLAGPLAVSLLLPSLTLLVSHLSSSQDVSSEPSSEQQLCALSKH
PTVAFEDLQPWVSNFTYPGARDFSQLALDPSGNQLIVGARNYLFRLSLANVSLLQATEWASSEDTRRSCQSKGKTE
EECQNYVRVLIVAGRKVFMCGTNAFSPMCTSRQVGNLSRTTEKINGVARCPYDPRHNSTAVISSQGELYAATVIDF
SGRDPAIYRSLGSGPPLRTAQYNSKWLNEPNFVAAYDIGLFAYFFLRENAVEHDCGRTVYSRVARVCKNDVGGRFL
LEDTWTTFMKARLNCSRPGEVPFYYNELQSAFHLPEQDLIYGVFTTNVNSIAASAVCAFNLSAISQAFNGPFRYQE
NPRAAWLPIANPIPNFQCGTLPETGPNENLTERSLQDAQRLFLMSEAVQPVTPEPCVTQDSVRFSHLVVDLVQAKD
TLYHVLYIGTESGTILKALSTASRSLHGCYLEELHVLPPGRREPLRSLRILHSARALFVGLRDGVLRVPLERCAAY
RSQGACLGARDPYCGWDGKQQRCSTLEDSSNMSLWTQNITACPVRNVTRDGGFGPWSPWQPCEHLDGDNSGSCLCR
ARSCDSPRPRCGGLDCLGPAIHIANCSRNGAWTPWSSWALCSTSCGIGFQVRQRSCSNPAPRHGGRICVGKSREER
FCNENTPCPVPIFWASWGSWSKCSSNCGGGMQSRRRACENGNSCLGCGVEFKTCNPEGCPEVRRNTPWTPWLPVNV
TQGGARQEQRFRFTCRAPLADPHGLQFGRRRTETRTCPADGSGSCDTDALVEDLLRSGSTSPHTVSGGWAAWGPWS
SCSRDCELGFRVRKRTCTNPEPRNGGLPCVGDAAEYQDCNPQACPVRGAWSCWTSWSPCSASCGGGHYQRTRSCTS
PAPSPGEDICLGLHTEEALCATQACPEGWSPWSEWSKCTDDGAQSRSRHCEELLPGSSACAGNSSQSRPCPYSEIP
VILPASSMEEATGCAGFNLIHLVATGISCFLGSGLLTLAVYLSCQHCQRQSQESTLVHPATPNHLHYKGGGTPKNE
KYTPMEFKTLNKNNLIPDDRANFYPLQQTNVYTTTYYPSPLNKHSFRPEASPGQRCFPNS

The disclosed nucleic acid MOL4a sequence has 3226 of 3664 bases (88%) identical to a Mus musculus semaphorin mRNA (GENBANK-ID: ACC: X97818).

The full amino acid sequence of the disclosed MOL4a polypeptide has 1021 of 1093 amino acid residues (93%) identical to, and 1053 of 1093 residues (96%) positive with, the 1093 amino acid residue semaphorin 5B precursor protein from Mus musculus (ptnr:SPTREMBL-ACC:Q60519), and 971 of 973 amino acid residues (99%) identical to, and 972 of 973 residues (99%) positive with patp:AAY94990 Human secreted protein vb21_{—}1, having 999 aa. The C-terminal 1202 amino acid residues of MOL4a are 100% identical to human KIAA1445 PROTEIN (TREMBLNEW-ACC:BAA95969).

MOL4a expression in different tissues was examined through TaqMan as described below in Example 1.

A SNP for MOL4a and the corresponding amino acid change it would cause is shown in Table 4C. The SNP was identified using the techniques disclosed in Example 3.

TABLE 4C
SNP for MOL4a

| | | AA | |
|---|---|---|---|
| Consensus | Base | Change | |
| Position | Change | Position | Residue Change |
| 2046 | C > T | 682 | A > V |

In a search of CuraGen's proprietary human expressed sequence assembly database, assemblies 111750277 (589 nucleotides) and 87739769 (896 nucleotides) were identified as having >95% homology to this predicted semaphorin sequence (FIG. 3A2). This database is composed of the expressed sequences (as derived from isolated mRNA) from more than 96 different tissues. The mRNA is converted to cDNA and then sequenced. These expressed DNA sequences are then pooled in a database and those exhibiting a defined level of homology are combined into a single assembly with a common consensus sequence. The consensus sequence is representative of all member components. Since the nucleic acid of the described invention has >95% sequence identity with the CuraGen assembly, the nucleic acid of the invention likely represents an expressed semaphorin sequence.

The DNA assembly 111750277 has 3 components and was found by CuraGen to be expressed in the following tissues: Lymph node and Lung. The DNA assembly 87739769 has 7 components and was found by CuraGen to be expressed in the following tissues: Brain, Uterus, and Lung.

### MOL4b

The disclosed novel semaphorin-like protein, MOL4b (also referred to herein as CG106951-02), is encoded by a nucleic acid, 4233 nucleotides long (SEQ ID NO:105). An open reading frame was identified beginning with an ATG initiation codon at nucleotides 2-4 and ending with a TGA codon at nucleotides 3281-3283. Putative untranslated regions upstream from the initiation codon and downstream from the termination codon are underlined in Table 4D, and the start and stop codons are in bold letters.

TABLE 4D
MOL4b Nucleotide Sequence.

(SEQ ID NO:106)

CATGGTGCTTGCAGGCCCCCTGGCTGTCTCGCTGTTGCTGCCCAGCCTCACACTGCTGGTGTCCCACCTCTCCAG
CTCCCAGGATGTCTCCAGTGAGCCCAGCAGTGAGCAGCAGCTGTGCGCCCTTAGCAAGCACCCCACCGTGGCCTT
TGAAGACCTGCAGCCGTGGGTCTCTAACTTCACCTACCCTGGAGCCCGGGATTTCTCCCAGCTGGCTTTGGACCC
CTCCGGGAACCAGCTCATCGTGGGAGCCAGGAACTACCTCTTCAGACTCAGCCTTGCCAATGTCTCTCTTCTTCA
GGCCACAGAGTGGGCCTCCAGTGAGGACACGCGCCGCTCCTGCCAAAGCAAAGGGAAGACTGAGGAGGAGTGTCA
GAACTACGTGCGAGTCCTGATCGTCGCCGGCCGGAAGGTGTTCATGTGTGGAACCAATGCCTTTTCCCCCATGTG
CACCAGCAGACAGGTGGGGAACCTCAGCCGGACTACTGAGAAGATCAATGGTGTGGCCCGCTGCCCCTATGACCC
ACGCCACAACTCCACAGCTGTCATCTCCTCCCAGGGGGAGCTCTATGCAGCCACGGTCATCGACTTCTCAGGTCG
GGACCCTGCCATCTACCGCAGCCTGGGCAGTGGGCCACCGCTTCGCACTGCCCAATATAACTCCAAGTGGCTTAA
TGAGCCAAACTTCGTGGCAGCCTATGATATTGGGCTGTTTGCATACTTCTTCCTGCGGGAGAACGCAGTGGAGCA
CGACTGTGGACGCACCGTGTACTCTCGCGTGGCCCGCGTGTGCAAGAATGACGTGGGGGGCCGATTCCTGCTGGA
GGACACATGGACCACATTCATGAAGGCCCGGCTCAACTGCTCCCGCCCGGGCGAGGTCCCCTTCTACTATAACGA
GCTGCAGAGTGCCTTCCACTTGCCAGAGCAGGACCTCATCTATGGAGTTTTCACAACCAACGTAAACAGCATCGC
GGCTTCTGCTGTCTGCGCCTTCAACCTCAGTGCTATCTCCCAGGCTTTCAATGGCCCATTTCGCTACCAGGAGAA
CCCCAGGGCTGCCTGGCTCCCCATAGCCAACCCCATCCCCAATTTCCAGTGTGGCACCCTGCCTGAGACCGGTCC
CAACGAGAACCTGACGGAGCGCAGCCTGCAGGACGCGCAGCGCCTCTTCCTGATGAGCGAGGCCGTGCAGCCGGT
GACACCCGAGCCCTGTGTCACCCAGGACAGCGTGCGCTTCTCACACCTCGTGGTGGACCTGGTGCAGGCTAAAGA
CACGCTCTACCATGTACTCTACATTGGCACCGAGTCGGGCACCATCCTGAAGGCGCTGTCCACGGCGAGCCGCAG
CCTCCACGGCTGCTACCTGGAGGAGCTGCACGTGCTGCCCCCCGGGCGCCGCGAGCCCCTGCGCAGCCTGCGCAT
CCTGCACAGCGCCCGCGCGCTCTTCGTGGGGCTGAGAGACGGCGTCCTGCGGGTCCCACTGGAGAGGTGCGCCGC
CTACCGCAGCCAGGGGGCATGCCTGGGGGCCCGGGACCCGTACTGTGGCTGGGACGGGAAGCAGCAACGTTGCAG
CACACTCGAGGACAGCTCCAACATGAGCCTCTGGACCCAGAACATCACCGCCTGTCCTGTGCGGAATGTGACACG
GGATGGGGGCTTCGGCCCATGGTCACCATGGCAACCATGTGAGCACTTGGATGGGGACAACTCAGGCTCTTGCCT
GTGTCGAGCTCGATCCTGTGATTCCCCTCGACCCCGCTGTGGGGGCCTTGACTGCCTGGGGCCAGCCATCCACAT
CGCCAACTGCTCCAGGAATGGGGCGTGGACCCCGTGGTCATCGTGGGCGCTGTGCAGCACGTCCTGTGGCATCGG
CTTCCAGGTCCGCCAGCGAAGTTGCAGCAACCCTGCTCCCCGCCACGGGGGCCGCATCTGCGTGGGCAAGAGCCG
GGAGGAACGGTTCTGTAATGAGAACACGCCTTGCCCGGTGCCCATCTTCTGGGCTTCCTGGGGCTCCTGGAGCAA
GTGCAGCAGCAACTGTGGAGGGGGCATGCAGTCGCGGCGTCGGGCCTGCGAGAACGGCAACTCCTGCCTGGGCTG
CGGCGTGGAGTTCAAGACGTGCAACCCCGAGGGCTGCCCCGAAGTGCGGCGCAACACCCCCTGGACGCCGTGGCT
GCCCGTGAACGTGACGCAGGGCGGGGCACGGCAGGAGCAGCGGTTCCGCTTCACCTGCCGCGCGCCCCTTGCAGA
CCCGCACGGCCTGCAGTTCGGCAGGAGAAGGACCGAGACGAGGACCTGTCCCGCGGACGGCTCCGGCTCCTGCGA
CACCGACGCCCTGGTGGAGGACCTCCTGCGCAGCGGGAGCACCTCCCCGCACACGGTGAGCGGGGGCTGGGCCGC
CTGGGGCCCGTGGTCGTCCTGCTCCCGGGACTGCGAGCTGGGCTTCCGCGTCCGCAAGAGAACGTGCACTAACCC
GGAGCCCCGCAACGGGGGCCTGCCCTGCGTGGGCGATGCTGCCGAGTACCAGGACTGCAACCCCCAGGCTTGCCC
AGTTCGGGGTGCTTGGTCCTGCTGGACCTCATGGTCTCCATGCTCAGCTTCCTGTGGTGGGGGTCACTATCAACG
CACCCGTTCCTGCACCAGCCCCGCACCCTCCCCAGGTGAGGACATCTGTCTCGGGCTGCACACGGAGGAGGCACT
ATGTGCCACACAGGCCTGCCCAGAAGGCTGGTCGCCCTGGTCTGAGTGGAGTAAGTGCACTGACGACGGAGCCCA
GAGCCGAAGCCGGCACTGTGAGGAGCTCCTCCCAGGGTCCAGCGCCTGTGCTGGAAACAGCAGCCAGAGCCGCCC
CTGCCCCTACAGCGAGATTCCCGTCATCCTGCCAGCCTCCAGCATGGAGGAGGCCACCGGCTGTGCAGGGTTCAA
TCTCATCCACTTGGTGGCCACGGGCATCTCCTGCTTCTTGGGCTCTGGGCTCCTGACCCTAGCAGTGTACCTGTC
TTGCCAGCACTGCCAGCGTCAGTCCCAGGAGTCCACACTGGTCCATCCTGCCACCCCCAACCATTTGCACTACAA
GGGCGGAGGCACCCCGAAGAATGAAAAGTACACACCCATGGAATTCAAGACCCTGAACAAGAATAACTTGATCCC
TGATGACAGAGCCAACTTCTACCCATTGCAGCAGACCAATGTGTACACGACTACTTACTACCCAAGCCCCCTGAA
CAAACACAGCTTCCGGCCCGAGGCCTCACCTGGACAACGGTGCTTCCCCAACAGCTGATACCGCCGTCCTGGGGA
CTTGGGCTTCTTGCCTTCATAAGGCACAGAGCAGATGGAGATGGGACAGTGGAGCCAGTTTGGTTTTCTCCCTCT
GCACTAGGCCAAGAACTTGCTGCCTTGCCTGTGGGGGGTCCCATCCGGCTTCAGAGAGCTCTGGCTGGCATTGAC
CATGGGGGAAAGGGCTGGTTTCAGGCTGACATATGGCCGCAGGTCCAGTTCAGCCCAGGTCTCTCATGGTTATCT
TCCAACCCACTGTCACGCTGACACTATGCTGCCATGCCTGGGCTGTGGACCTACTGGGCATTTGAGGAACTGGAG
AATGGAGATGGCAAGAGGGCAGGCTTTTAAGTTTGGGTTGGAGACAACTTCCTGTGGCCCCCACAAGCTGAGTCT
GGCCTTCTCCAGCTGGCCCCAAAAAAGGCCTTTGCTACATCCTGATTATCTCTGAAAGTAATCAATCAAGTGGCT
CCAGTAGCTCTGGATTTTCTGCCAGGGCTGGGCCATTGTGGTGCTGCCCCAGTATGACATGGGACCAAGGCCAGC
GCAGGTTATCCACCTCTGCCTGGAAGTCTATACTCTACCCAGGGCATCCCTCTGGTCAGAGGCAGTGAGTACTGG
GAACTGGAGGCTGACCTGTGCTTAGAAGTCCTTTAATCTGGGCTGGTACAGGCCTCAGCCTTGCCCTCAATGCAC
GAAAGGTGGCCCAGGAGAGAGGATCAATGCCACAGGAGGCAGAAGTCTGGCCTCTGTGCCTCTATGGAGACTATC
TTCCAGTTGCTGCTCAACAGAGTTGTTGGCTGAGACCTGCTTGGGAGTCTCTGCTGGCCCTTCATCTGTTCAGGA
ACACACACACACACACACTCACACACGCACACACAATCACAATTTGCTACAGCAACAAAAAAGACATTGGGCTGT
GGCATTATTAATTAAAGATGATATCCAGTCTCC

The 1093 amino acid MOL4b polypeptide (SEQ ID NO:106) encoded by SEQ ID NO:105 is presented using the one-letter amino acid code in Table 4E. The Psort profile for MOL4b predicts that this sequence has no signal peptide and is likely to be a Type II (Ncyt Cexo) membrane protein with a certainty of 0.7900.

TABLE 4E
MOL4b protein sequence

(SEQ ID NO:106)

MVLAGPLAVSLLLPSLTLLVSHLSSSQDVSSEPSSEQQLCALSKHPTVAFEDLQPWVSNFTYPGARDFSQLALDPS
GNQLIVGARNYLFRLSLANVSLLQATEWASSEDTRRSCQSKGKTEEECQNYVRVLIVAGRKVFMCGTNAFSPMCTS
RQVGNLSRTTEKINGVARCPYDPRHNSTAVISSQGELYAATVIDFSGRDPAIYRSLGSGPPLRTAQYNSKWLNEPN
FVAAYDIGLFAYFFLRENAVEHDCGRTVYSRVARVCKNDVGGRFLLEDTWTTFMKARLNCSRPGEVPFYYNELQSA
FHLPEQDLIYGVFTTNVNSIAASAVCAFNLSAISQAFNGPFRYQENPRAAWLPIANPIPNFQCGTLPETGPNENLT
ERSLQDAQRLFLMSEAVQPVTPEPCVTQDSVRFSHLVVDLVQAKDTLYHVLYIGTESGTILKALSTASRSLHGCYL
EELHVLPPGRREPLRSLRILHSARALFVGLRDGVLRVPLERCAAYRSQGACLGARDPYCGWDGKQQRCSTLEDSSN
MSLWTQNITACPVRNVTRDGGFGPWSPWQPCEHLDGDNSGSCLCRARSCDSPRPRCGGLDCLGPAIHIANCSRNGA
WTPWSSWALCSTSCGIGFQVRQRSCSNPAPRHGGRICVGKSREERFCNENTPCPVPIFWASWGSWSKCSSNCGGGM
QSRRRACENGNSCLGCGVEFKTCNPEGCPEVRRNTPWTPWLPVNVTQGGARQEQRFRFTCRAPLADPHGLQFGRRR
TETRTCPADGSGSCDTDALVEDLLRSGSTSPHTVSGGWAAWGPWSSCSRDCELGFRVRKRTCTNPEPRNGGLPCVG
DAAEYQDCNPQACPVRGAWSCWTSWSPCSASCGGGHYQRTRSCTSPAPSPGEDICLGLHTEEALCATQACPEGWSP
WSEWSKCTDDGAQSRSRHCEELLPGSSACAGNSSQSRPCPYSEIPVILPASSMEEATGCAGFNLIHLVATGISCFL
GSGLLTLAVYLSCQHCQRQSQESTLVHPATPNHLHYKGGGTPKNEKYTPMEFKTLNKNNLIPDDRANFYPLQQTNV
YTTTYYPSPLNKHSFRPEASPGQRCFPNS

The disclosed nucleic acid MOL4b sequence has 3864 of 3873 (99%) identical to an alpha gene treating neurodegenerative disorders, autoimmune diseases and cancer (WO200011015-A1). The disclosed MOOL4b nucleic acid is also 100% identical to Kiaa 1445.

The full amino acid sequencee of the disclosed MOL4b polypeptide has 972 of 973 amino acid residues (99%) identical to the alpha gene treating neurodegenerative disorders, autoimmune diseases and cancer (WO200011015-A1). The disclosed MOL4b polypeptide is also 100% identical to Kiaa 1445. The disclosed MOL4b polypeptide is also 93% identical to mouse semaphorin.

Analysis of the MOL4b sequence against the Pfam database showed that the sequence contains a Sema domain, a Thrombospondin type 1 domain, and a Plexin repeat

### MOL4c

The disclosed novel semaphorin-like protein, MOL4c (also referred to herein as CG106951-04), is encoded by a nucleic acid, 3631 nucleotides long (SEQ ID NO:107). An open reading frame was identified beginning with an ATG initiation codon at nucleotides 154-156 and ending with a TGA codon at nucleotides 3544-3546. Putative untranslated regions upstream from the initiation codon and downstream from the termination codon are underlined in Table 4F, and the start and stop codons are in bold letters.

TABLE 4F
MOL4c Nucleotide Sequence.

(SEQ ID NO:107)

GCGGCCGCCCCATTCCCAGACCGGCCGCCAGCCCATCTGGTTAGCTCCCGCCGCTCCGCGCCGCCCGGGAGTCGG
GAGCCGCGGGGAACCGGGCACCTGCACCCGCCTCTGGGAGTGAGTGGTTCCAGCTGGTGCCTGGCCTGTGTCTCT
TGGATGCCCTGTGGCTTCAGTCCGTCTCCTGTTGCCCACCACCTCGTCCCTGGGCCGCCTGATACCCCAGCCCAA
CAGCTAAGGTGTGGATGGACAGTAGGGGGCTGGCTTCTCTCACTGGTCAGGGGTCTTCTCCCCTGTCTGCCTCCC
GGAGCTAGGACTGCAGAGGGGCCTATCATGGTGCTTGCAGGCCCCCTGGCTGTCTCGCTGTTGCTGCCCAGCCTC
ACACTGCTGGTGTCCCACCTCTCCAGCTCCCAGGATGTCTCCAGTGAGCCCAGCAGTGAGCAGCAGCTGTGCGCC
CTTAGCAAGCACCCCACCGTGGCCTTTGAAGACCTGCAGCCGTGGGTCTCTAACTTCACCTACCCTGGAGCCCGG
GATTTCTCCCAGCTGGCTTTGGACCCCTCCGGGAACCAGCTCATCGTGGGAGCCAGGAACTACCTCTTCAGACTC
AGCCTTGCCAATGTCTCTCTTCTTCAGGCCACAGAGTGGGCCTCCAGTGAGGACACGCGCCGCTCCTGCCAAAGC
AAAGGGAAGACTGAGGAGGAGTGTCAGAACTACGTGCGAGTCCTGATCGTCGCCGGCCGGAAGGTGTTCATGTGT
GGAACCAATGCCTTTTCCCCCATGTGCACCAGCAGACAGGTGGGGAACCTCAGCCGGACTACTGAGAAGATCAAT
GGTGTGGCCCGCTGCCCCTATGACCCACGCCACAACTCCACAGCTGTCATCTCCTCCCAGGGGGAGCTCTATGCA
GCCACGGTCATCGACTTCTCAGGTCGGGACCCTGCCATCTACCGCAGCCTGGGCAGTGGGCCACCGCTTCGCACT
GCCCAATATAACTCCAAGTGGCTTAATGAGCCAAACTTCGTGGCAGCCTATGATATTGGGCTGTTTGCATACTTC
TTCCTGCGGGAGAACGCAGTGGAGCACGACTGTGGACGCACCGTGTACTCTCGCGTGGCCCGCGTGTGCAAGAAT
GACGTGGGGGGCCGATTCCTGCTGGAGGACACATGGACCACATTCATGAAGGCCCGGCTCAACTGCTCCCGCCCG
GGCGAGGTCCCCTTCTACTATAACGAGCTGCAGAGTGCCTTCCACTTGCCAGAGCAGGACCTCATCTATGGAGTT
TTCACAACCAACGTAAACAGCATCGCGGCTTCTGCTGTCTGCGCCTTCAACCTCAGTGCTATCTCCCAGGCTTTC
AATGGCCCATTTCGCTACCAGGAGAACCCCAGGGCTGCCTGGCTCCCCATAGCCAACCCCATCCCCAATTTCCAG
TGTGGCACCCTGCCTGAGACCGGTCCCAACGAGAACCTGACGGAGCGCAGCCTGCAGGACGCGCAGCGCCTCTTC
CTGATGAGCGAGGCCGTGCAGCCGGTGACACCCGAGCCCTGTGTCACCCAGGACAGCGTGCGCTTCTCACACCTC
GTGGTGGACCTGGTGCAGGCTAAAGACACGCTCTACCATGTACTCTACATTGGCACCGAGTCGGGCACCATCCTG
AAGGCGCTGTCCACGGCGAGCCGCAGCCTCCACGGCTGCTACCTGGAGGAGCTGCACGTGCTGCCCCCCGGGCGC
CGCGAGCCCCTGCGCAGCCTGCGCATCCTGCACAGCGCCCGCGCGCTCTTCGTGGGGCTGAGAGACGGCGTCCTG
CGGGTCCCACTGGAGAGGTGCGCCGCCTACCGCAGCCAGGGGGCATGCCTGGGGGCCCGGGACCCGTACTGTGGC
TGGGACGGGAAGCAGCAACGTTGCAGCACACTCGAGGACAGCTCCAACATGAGCCTCTGGACCCAGAACATCACC
GCCTGTCCTGTGCGGAATGTGACACGGGATGGGGGCTTCGGCCCATGGTCACCATGGCAACCATGTGAGCACTTG
GATGGGGACAACTCAGGCTCTTGCCTGTGTCGAGCTCGATCCTGTGATTCCCCTCGACCCCGCTGTGGGGGCCTT
GACTGCCTGGGGCCAGCCATCCACATCGCCAACTGCTCCAGGAATGGGGCGTGGACCCCGTGGTCATCGTGGGCG
CTGTGCAGCACGTCCTGTGGCATCGGCTTCCAGGTCCGCCAGCGAAGTTGCAGCAACCCTGCTCCCCGCCACGGG
GGCCGCATCTGCGTGGGCAAGAGCCGGGAGGAACGGTTCTGTAATGAGAACACGCCTTGCCCGGTGCCCATCTTC
TGGGCTTCCTGGGGCTCCTGGAGCAAGTGCAGCAGCAACTGTGGAGGGGGCATGCAGTCGCGGCGTCGGGCCTGC
GAGAACGGCAACTCCTGCCTGGGCTGCGGCGTGGAGTTCAAGACGTGCAACCCCGAGGGCTGCCCCGAAGTGCGG
CGCAACACCCCCTGGACGCCGTGGCTGCCCGTGAACGTGACGCAGGGCGGGGCACGGCAGGAGCAGCGGTTCCGC
TTCACCTGCCGCGCGCCCCTTGCAGACCCGCACGGCCTGCAGTTCGGCAGGAGAAGGACCGAGACGAGGACCTGT
CCCGCGGACGGCTCCGGCTCCTGCGACACCGACGCCCTGGTGGAGGACCTCCTGCGCAGCGGGAGCACCTCCCCG
CACACGGTGAGCGGGGGCTGGGCCGCCTGGGGCCCGTGGTCGTCCTGCTCCCGGGACTGCGAGCTGGGCTTCCGC
GTCCGCAAGAGAACGTGCACTAACCCGGAGCCCCGCAACGGGGGCCTGCCCTGCGTGGGCGATGCTGCCGAGTAC
CAGGACTGCAACCCCCAGGCTTGCCCAGTTCGGGGTGCTTGGTCCTGCTGGACCTCATGGTCTCCATGCTCAGCT
TCCTGTGGTGGGGGTCACTATCAACGCACCCGTTCCTGCACCAGCCCCGCACCCTCCCCAGAAGGCTGGTCGCCC
TGGTCTGAGTGGAGTAAGTGCACTGACGACGGAGCCCAGAGCCGAAGCCGGCACTGTGAGGAGCTCCTCCCAGGG
TCCAGCGCCTGTGCTGGAAACAGCAGCCAGAGCCGCCCCTGCCCCTACAGCGAGATTCCCGTCATCCTGCCAGCC
TCCAGCATGGAGGAGGCCACCGGCTGTGCAGGGTTCAATCTCATCCACTTGGTGGCCACGGGCATCTCCTGCTTC
TTGGGCTCTGGGCTCCTGACCCTAGCAGTGTACCTGTCTTGCCAGCACTGCCAGCGTCAGTCCCAGGAGTCCACA
CTGGTCCATCCTGCCACCCCCAACCATTTGCACTACAAGGGCGGAGGCACCCCGAAGAATGAAAAGTACACACCC
ATGGAATTCAAGACCCTGAACAAGAATAACTTGATCCCTGATGACAGAGCCAACTTCTACCCATTGCAGCAGACC
AATGTGTACACGACTACTTACTACCCAAGCCCCCTGAACAAACACAGCTTCCGGCCCGAGGCCTCACCTGGACAA
CGGTGCTTCCCCAACAGCTGATACCGCCGTCCTGGGGACTTGGGCTTCTTGCCTTCATAAGGCACAGAGCAGATG
GAGATGGGACAGTGGAGCCAGTTTGGTTTCT

The nucleic acid MOL4c of the invention, localized to human chromosome 3, was found, using a BLASTN search to have 3117 of 3221 (99%) nucleotides identical to the 4559 nucleotide mRNA for KIAA1445 protein from Homo sapiens (GENBANK-ID:AB040878|acc:AB040878) (E=0.0). It also has 678 of 678 (100%) nucleotides identical to the 819 nucleotide sequence for NT2RM2 Homo sapiens cDNA clone NT2RM2001930 5′, mRNA sequence (GENBANK-ID:AU124266|acc:AU124266.1 AU124266) (E=5.2e⁻¹⁴⁷).

The 1130 amino acid MOL4c polypeptide (SEQ ID NO:108) encoded by SEQ ID NO:107 is presented using the one-letter amino acid code in Table 4G. The Psort profile for MOL4c predicts that this sequence has a signal peptide between amino acids 42 and 43 (VRG-LL). It is also likely to be localized to the plasma membrane with a certainty of 0.7900. In other embodiments, MOL4c could also be localized to the microbody (peroxisome) with a certainty of 0.3000, to the Golgi body with a certainty of 0.3000, or to the endoplasmic reticulum (membrane) with a certainty of 0.2000.

TABLE 4G
MOL4c protein sequence

(SEQ ID NO:108)

MPCGFSPSPVAHHLVPGPPDTPAQQLRCGWTVGGWLLSLVRGLLPCLPPGARTAEGPIMVLAGPLAVSLLLPSLTL
LVSHLSSSQDVSSEPSSEQQLCALSKHPTVAFEDLQPWVSNFTYPGARDFSQLALDPSGNQLIVGARNYLFRLSLA
NVSLLQATEWASSEDTRRSCQSKGKTEEECQNYVRVLIVAGRKVFMCGTNAFSPMCTSRQVGNLSRTTEKINGVAR
CPYDPRHNSTAVISSQGELYAATVIDFSGRDPAIYRSLGSGPPLRTAQYNSKWLNEPNFVAAYDIGLFAYFFLREN
AVEHDCGRTVYSRVARVCKNDVGGRFLLEDTWTTFMKARLNCSRPGEVPFYYNELQSAFHLPEQDLIYGVFTTNVN
SIAASAVCAFNLSAISQAFNGPFRYQENPRAAWLPIANPIPNFQCGTLPETGPNENLTERSLQDAQRLFLMSEAVQ
PVTPEPCVTQDSVRFSHLVVDLVQAKDTLYHVLYIGTESGTILKALSTASRSLHGCYLEELHVLPPGRREPLRSLR
ILHSARALFVGLRDGVLRVPLERCAAYRSQGACLGARDPYCGWDGKQQRCSTLEDSSNMSLWTQNITACPVRNVTR
DGGFGPWSPWQPCEHLDGDNSGSCLCRARSCDSPRPRCGGLDCLGPAIHIANCSRNGAWTPWSSWALCSTSCGIGF
QVRQRSCSNPAPRHGGRICVGKSREERFCNENTPCPVPIFWASWGSWSKCSSNCGGGMQSRRRACENGNSCLGCGV
EFKTCNPEGCPEVRRNTPWTPWLPVNVTQGGARQEQRFRFTCRAPLADPHGLQFGRRRTETRTCPADGSGSCDTDA
LVEDLLRSGSTSPHTVSGGWAAWGPWSSCSRDCELGFRVRKRTCTNPEPRNGGLPCVGDAAEYQDCNPQACPVRGA
WSCWTSWSPCSASCGGGHYQRTRSCTSPAPSPEGWSPWSEWSKCTDDGAQSRSRHCEELLPGSSACAGNSSQSRPC
PYSEIPVILPASSMEEATGCAGFNLIHLVATGISCFLGSGLLTLAVYLSCQHCQRQSQESTLVHPATPNHLHYKGG
GTPKNEKYTPMEFKTLNKNNLIPDDRANFYPLQQTNVYTTTYYPSPLNKHSFRPEASPGQRCFPNS

The disclosed amino acid MOL4c sequence has 964 of 1010 amino acids (95%) identical to and 971 of 1010 amino acids (96%) positive with the 1202 namino acid sequence for KIAA1445 Protein from Homo sapiens (Human) (SPTREMBL-ACC:Q9P283) (E=0.0).

MOL4c is expressed in at least NT2 teratocarcinoma cell line. Expression information was derived from the tissue sources of the sequences that were included in the derivation of the sequence of MOL4c.

The presence of identifiable domains in the protein disclosed herein was determined by searches versus domain databases sulch as Pfam, PROSITE, ProDom, Blocks or Prints and then identified by the Interpro domain accession number. Significant domains are summarized in Table 4H.

TABLE 4H
Domain Searh for MOL4c
Scores for sequence family classification (score includes
all domains):

| Model | Description | Score | E-value | N |
|---|---|---|---|---|
| Sema(InterPro) | Sema domain | 682.4 | 2.3e−201 | 1 |
| tsp_1(InterPro) | Thrombospondin type | 119.4 | 6.5e−32 | 6 |
| | 1 domain | | | |
| Plexin_repeat(InterPro) | Plexin repeat | 61.0 | 2.5e−14 | 1 |

Note: Please compare the score with TC (trusted cutoff) and NC
(noise cutoff) scores
in each model to evaluate its significance. More on Pfam Scores

Parsed for domains:

| Model | Domain | seq-f | seq-t | hmm-f | hmm-t | score | E-value |
|---|---|---|---|---|---|---|---|
| Sema | 1/1 | 126 | 537 . . . | 1 | 490 [ ] | 682.4 | 2.3e−201 |
| Plexin_{—} | 1/1 | 555 | 602 . . . | 1 | 67 [ ] | 61.0 | 2.5e−14 |
| repeat | | | | | | | |
| tsp_1 | 1/6 | 613 | 661 . . . | 1 | 54 [ ] | −3.9 | 1.9 |
| tsp_1 | 2/6 | 668 | 719 . . . | 1 | 54 [ ] | 55.3 | 1.4e−12 |
| tsp_1 | 3/6 | 726 | 770 . . . | 1 | 54 [ ] | 33.4 | 5.1e−06 |
| tsp_1 | 4/6 | 857 | 907 . . . | 1 | 54 [ ] | 48.7 | 1.3e−10 |
| tsp_1 | 5/6 | 914 | 946 . . . | 1 | 54 [ ] | 2.6 | 0.35 |
| tsp_1 | 6/6 | 948 | 988 . . . | 1 | 54 [ ] | 20.9 | 0.0027 |

The Sema domain occurs in semaphorins, which are a large family of secreted and transmembrane proteins, some of which function as repellent signals during axon guidance. Sema domains also occur in a hepatocyte growth factor receptor, in SEX protein and in viral proteins.

Plexin repeats have been found in plexins, semaphorins and integrins. Plexin is involved in the development of neural and epithelial tissues; semaphorins induce the collapse and paralysis of neuronal growth cones; and integrins may mediate adhesive or migratory functions of epithelial cells.

Thrombospondin type 1 domain repeat was first found in the thrombospondin protein where it is repeated 3 times. Now a number of proteins involved in the complement pathway (properdin, C6, C7, C8A, C8B, C9) as well as extracellular matrix protein like mindin, F-spondin, SCO-spondin and even the circumsporozoite surface protein 2 and TRAP proteins of Plasmodium contain one or more instance of this repeat. It has been involved in cell-cell interraction, inhibition of angiogenesis, apoptosis.

### MOL4d

The disclosed novel semaphorin-like protein, MOL4d (also referred to herein as 209829549), is encoded by a nucleic acid, 1203 nucleotides long (SEQ ID NO:109). An open reading frame was identified beginning with an GGA initiation codon at nucleotides 1-3 and ending after a GAC codon at nucleotides 1201-1203. In Table 4I, the start codon is in bold letters. Because the start and stop codons are not traditional initiation and termination codons, MOL4d could be a partial reading frame that extends further in the 5′ and/or 3′ directions.

TABLE 4I
MOL4d Nucleotide Sequence.

(SEQ ID NO:109)

GGATCCGGCCCATCGTCACCATGGCAACCATGTGAGCACTTGGATGCGGACAACTCAGCCTCTTCCCTCTGTCGA
GCTCGATCCTGTGATTCCCCTCCACCCCGCTGTCGGGGCCTTGACTGCCTGGCGCCAGCCATCCACATCGCCAAC
TGCTCCACGAATCGGGCGTGGACCCCGTGGTCATCGTGGGCGCTGTGCAGCACGTCCTGTGGCATCGGCTTCCAG
GTCCGCCAGCGAAGTTGCAGCAACCCTGCTCCCCGCCACCGGGGCCGCATCTGCCTGGCCAAGAGCCGCCAGGAA
CGCTTCTGTAATGAGAACACGCCTTGCCCGGTGCCCATCTTCTGCGCTTCCTGGGGCTCCTGGACCAAGTGCAGC
AGCAACTGTGGACGGCGCATGCGGTCGCGGCGTCGGGCCTGCGAGAACGGCAACTCCTGCCTCGGCTGCGGCGTG
GAGTTCAACACGTGCAACCCCGAGGGCTGCCCCGAACTGCCGCGCAACACCCCCTCCACGCCCTGCCTGCCCCTG
AACGTGACGCAGCGCGGCGCACCGCAGGAGCAGCGGTTCCGCTTCACCTGCCGCGCGCCCCTTGCAGACCCGCAC
GGCCTGCAGTTCGGCAGGAGAACGACCGAGACGAGCACCTGTCCCGCGGACCGCTCCGGCTCCTGCCACACCGAC
GCCCTGGTGGACGACCTCCTGCGCAGCGGGAGCACCTCCCCGCACACCGTGACCGGGGGCTGGCCCGCCTGGGGC
CCGTGGTCGTCCTGCTCCCGGGACTGCGAGCTGCCCTTCCGCGTCCGCAAGAGAACCTGCACTAACCCGGAGCCC
CGCAACCCGGGCCTGCCCTGCGTGGGCGATGCTGCCGAGTACCAGGACTGCAACCCCCAGGCTTCCCCAGTTCGG
GGTCCTTGGTCCTGCTGGACCTCATGGTCTCCATGCTCAGCTTCCTGTGCTGGGCGTCACTATCAACGCACCCGT
TCCTGCACCAGCCCCGCACCCTCCCCAGGTGAGGACATCTGTCTCGGGCTGCACACGGAGGAGGCACTATGTGCC
ACACAGGCCTGCCCAGAAGCCTCGTCGCCCTGGTCTCAGTGGAGTAAGTGCACTGACGACGGAGCCCACAGCCCA
AGCCGGCACTGTGACGAaCTCCTCCCAGGGTCCACCGCCTGTGCTCGAAACAGCACCCAGAGCCGCCCCTGCGTC
GAC

The reverse complement for MOL4d is shown in Table 4J.

TABLE 4J
MOL4d Nucleotide Sequence reverse complement.

(SEQ ID NO:110)

GTCGACGCACGCGCGCCTCTGGCTGCTGTTTCCAGCACAGGCGCTGCACCCTCGGAGGAGCTCCTCACAGTGCCG
GCTTCGGCTCTGGGCTCCGTCCTCAGTCCACTTACTCCACTCACACCATGGCGACCAGCCTTCTGGGCAGGCCTG
TGTGGCACATAGTGCCTCCTCCGTCTCCAGCCCCACACAGATGTCCTCACCTGGGGAGGGTCCCCGGCTGGTGCA
GGAACGGCTCCGTTCATAGTGACCCCCACCACAGGAAGCTGAGCATGGAGACCATGAGGTCCACCAGGACCAAGC
ACCCCGAACTCCGCAAGCCTGGGGGTTGCAGTCCTGGTACTCGCCACCATCGCCCACGCAGGGCAGCCCCCCGTT
GCGGGGCTCCGGGTTAGTGCACGTTCTCTTGCCGACGCGGAAGCCCAGCTCGCAGTCCCCGGAGCAGGACGACCA
CGGGCCCCAGGCGGCCCAGCCCCCCCTCACCGTCTGCGGGGAGGTGCTCCCCCTGCGCAGGAGGTCCTCCACCAG
GGCGTCGGTGTCGCAGGAGCCCCACCCGTCCCCCGGACAGGTCCTCGTCTCGCTCCTTCTCCTGCCGAACTGCAG
GCCGTGCCCCTCTGCAAGGGGCGCGCGGCAGGTGAAGCGCAACCGCTGCTCCTGCCGTGCCCCGCCCTGCGTCAC
GTTCACGGGCAGCCACGCCGTCCAGGGCGTGTTGCCCCGCACTTCGGGGCAGCCCTCGGGGTTGCACGTCTTGAA
CTCCACGCCGCAGCCCAGGCACCAGTTGCCGTTCTCGCAGGCCCCACGCCGCGACCGCATGCCcCCTCCACAGTT
GCTGCTGCACTTGCTCCAGGAGCCCCAGGAAGCCCAGAAGATGGGCACCGGGCAAGGCGTGTTCTCATTACAGAA
CCGTTCCTCCCGGCTCTTGCCCACGCAGATCCGCCCCCCGTGCCCGGGACCACGCTTGCTGCAACTTCGCTGGCG
GACCTGGAAGCCCATGCCACAGGACCTGCTGCACAGCGCCCACGATGACCACGGGGTCCACGCCCCATTCCTGCA
GCAGTTGGCCATCTCGATGGCTGGCCCCAGCCAGTCAACGCCCCCACAGCGGGGTCGAGGGCAATCACAGGATCG
AGCTCGACACAGGCAAGAGCCTCACTTGTCCCCATCCAAGTGCTCACATGGTTGCCATGGTGACCATCGGCCGGA
TCC

The 401 amino acid MOL4d polypeptide (SEQ ID NO:111) encoded by SEQ ID NO:109 is presented using the one-letter amino acid code in Table 4K.

TABLE 4K
MOL4d protein sequence

(SEQ ID NO:111)

GSGPWSPWQPCEHLDGDNSGSCLCRARSCDSPRPRCGGLDCLGPAIHIAN
CSRNGAWTPWSSWALCSTSCGIGFQVRQRSCSNPAPRHGGRICVGKSREE
RFCNENTPCPVPIFWASWGSWSKCSSNCGGGMRSRRRACENGNSCLGCGV
EFKTCNPEGCPEVRRNTPWTPWLPVNVTQGGARQEQRFRFTCRAPLADPH
GLQFGRRRTETRTCPADGSGSCDTDALVEDLLRSGSTSPHTVSGGWAAWG
PWSSCSRDCELGFRVRKRTCTNPEPRNGGLPCVGDAAEYQDCNPQACPVR
GAWSCWTSWSPCSASCGGGHYQRTRSCTSPAPSPGEDICLGLHTEEALCA
TQACPEGWSPWSEWSKCTDDGAQSRSRHCEELLPGSSACAGNSSQSRPCV
D

### MOL4e

The disclosed novel semaphorin-like protein, MOL4e (also referred to herein as 209829553), is encoded by a nucleic acid, 1203 nucleotides long (SEQ ID NO:112). An open reading frame was identified beginning with an GGA initiation codon at nucleotides 1-3 and ending after a GAC codon at nucleotides 1201-1203. In Table 4 L, the start codon is in bold letters. Because the start and stop codons are not traditional initiation and termination codons, MOL4e could be a partial reading frame that extends further in the 5′ and/or 3′ directions.

TABLE 4L
MOL4e Nucleotide Sequence.

(SEQ ID NO:112)

GGATCCGGCCCATGGTCACCATGGCAACCATGTGACCACTTGGATGGGGACAACTCACGCTCTTCCCTGTGTCGA
GCTCGATCCTGTGATTCCCCTCGACCCCGCTGTGGGGGCCTTGACTCCCTCGGGCCAGCCATCCACATCCCCAAC
TGCTCCAGGAATGCCCCGTCCACCCCGTGGTCATCGTGGGCGCTGTCCAGCACCTCCTCTCGCATCGGCTTCCAG
GTCCGCCAGCGAAGTTGCAGCAACCCTGCTCCCCGCCACGGGCGCCCCATCTGCGTGGGCAAGAGCCGGGAGGAA
CGGTTCTGTAATCAGAACACGCCTTGCCCGGTGCCCATCTTCTGCGCTTCCTGGCGCTCCTGCAGCAAGTCCACC
AGCAACTCTGCACGGGGCATGCAGTCGCGGCGTCGGGCCTGCGAGAACGGCAACTCCTGCCTGGGCTGCGGCGTG
GAGTTCAAGACGTGCAACCCCGAGCGCTCCCCCGAAGTCCGGCGCAACACCCCCTGGACGCCGTGCCTGCCCGTG
AACGTGACGCAGGGCGGGGCACGGCACCAGCAGCGGTTCCCCTTCACCTGCCGCGCCCCCCTTGCAGACCCGCAC
GGCCTGCAGTTCGGCAGCACAAGCACCGAGACCAGGACCTGTCCCGCGGACGGCTCCGCCTCCTCCGACACCCAC
GCCCTGGTGGAGGACCTCCTGCGCAGCGGGAGCACCTCCCCGCACACGGTGAGCGCGGCCTGGCCCGCCTGGGGC
CCGTGGTCGTCCTGCTCCCGGGACTGCGAGCTGGGCTTCCGCGTCCGCAAGAGAACGTGCACTAACCCGGAGTCC
CGCAACCGGCCCCTCCCCTGCCTCGCCGATGCTGCCGAGTACCAGGACTGCAACCCCCAGGCTTGCCCACTTCGG
GGTGCTTGGTCCTCCTCGACCTCATGGTCTCCATGCTCAGCTTCCTGTGGTGCGGGTCACTATCAACGCACCCCT
TCCTGCACCAGCCCCCCACCCTCCCCAGCTGAGCACATCTCTCTCCGGCTGCACACCGAGGAGCCACTATGTCCC
ACACAGGCCTCCCCAGAAGGCTGGTCGCCCTGCTCTGAGTCCACTAAGTGCACTGACGACGCAGCCCAGAGCCGA
AGCCGGCACTGTCAGGAGCTCCTCCCAGGGTCCAGCGCCTCTGCTGGAAACAGCAGCCAGAGCCGCCCCTGCGTC
GAC

The reverse complement for MOL4e is shown in Table 4M.

TABLE 4M
MOL4e Nucleotide Sequence reverse complement.

(SEQ ID NO:113)

GTCGACGCAGGGGCGGCTCTGGCTGCTGTTTCCACCACAGGCGCTGGACCCTGGGAGGAGCTCCTCACAGTGCCG
GCTTCGGCTCTGGGCTCCGTCGTCAGTGCACTTACTCCACTCAGACCAGGGCGACCAGCCTTCTGGGCAGGCCTG
TGTGGCACATAGTGCCTCCTCCGTGTGCAGCCCGAGACAGATGTCCTCACCTGGGGAGGGTGCGGGGCTGGTGCA
GGAACGGCTGCGTTGATAGTCACCCCCACCACAGGAAGCTGAGCATGGAGACCATGAGGTCCAGCAGCACCAAGC
ACCCCGAACTGGGCAACCCTCGCGGTTCCACTCCTGCTACTCGGCAGCATCGCCCACCCAGGGCAGCCCCCCGTT
GCGGGACTCCGGGTTAGTGCACGTTCTCTTGCCGACGCGGAAGCCCAGCTCGCAGTCCCGGCACCAGGACGACCA
CGGGCCCCAGGCGGCCCAGCCCCCCCTCACCCTCTGCGCGGAGCTGCTCCCGCTGCGCAGGAGGTCCTCCACCAG
GGCGTCCGTGTCGCAGGACCCGGACCCGTCCCCGCGACAGGTCCTCGTCTCCGTCCTTCTCCTGCCGAACTCCAC
GCCGTGCGCCTCTGCAACGGGCGCGCGGCAGGTGAAGCGGAACCCCTGCTCCTGCCGTGCCCCGCCCTGCGTCAC
GTTCACGGGCAGCCACGGCGTCCAGGCCCTCTTCCCCCGCACTTCGGCCCAGCCCTCCGGGTTGCACGTCTTGAA
CTCCACGCCGCAGCCCAGGCAGGAGTTGCCGTTCTCGCAGGCCCGACGCCCCCACTGCATGCCCCCTCCACAGTT
GCTGCTGCACTTCCTCCAGGAGCCCCAGGAAGCCCAGAAGATGGGCACCGGGCAAGGCGTGTTCTCATTACAGAA
CCGTTCCTCCCGCCTCTTGCCCACGCAGATGCGGCCCCCGTGGCGGCGAGCAGGGTTGCTGCAACTTCGCTGGCC
GACCTGGAAGCCGATGCCACACGACCTGCTGCACACCCCCCACGATCACCACGGGGTCCACGCCCCATTCCTGGA
GCAGTTGGCGATGTCCATCGCTGGCCCCACGCACTCAAGGCCCCCACAGCGGGGTCGAGCGGAATCACAGGATCG
AGCTCCACACAGGCAAGAGCCTGAGTTGTCCCCATCCAAGTGCTCACATCGTTGCCATCGTCACCATGCGCCGGA
TCC

The 401 amino acid MOL4e polypeptide (SEQ ID NO:114) encoded by SEQ ID NO:112 is presented using the one-letter amino acid code in Table 4N.

TABLE 4N
MOL4e protein sequence

(SEQ ID NO:114)

GSGPWSPWQPCEHLDGDNSGSCLCRARSCDSPRPRCGGLDCLGPAIHIAN
CSRNGAWTPWSSWALCSTSCGICFQVRQRSCSNPAPRHGGRICVGKSREE
RFCNENTPCPVPIFWASWGSWSKCSSNCGGGMQSRRRACENGNSCLGCGV
EFKTCNFEGCREVRRNTPWTRWLPVNVTQGGARQEQRFRFTCRAPLADPH
GLQFCRRRTETRTCPADGSGSCDTDALVEDLLRSGSTSPHTVSGGWAAWG
PWSSCSRDCELGFRVRKPTCTNPESRNGGLPCVGDAAEYQDCNFQACRVR
GAWSCWTSWSPCSASCGCCHYQRTRSCTSPAPSPGEDICLGLHTEEALCA
TQACPEGWSPWSEWSKCTDDGAQSRSRHCEELLPGSSACAGNSSQSRPCV
D

### MOL4f

The disclosed novel semaphorin-like protein, MOL4f (also referred to herein as 209829642), is encoded by a nucleic acid, 1203 nucleotides long (SEQ ID NO:115). An open reading frame was identified beginning with an GGA initiation codon at nucleotides 1-3 and ending after a GAC codon at nucleotides 1201-1203. In Table 4O, the start codon is in bold letters. Because the start and stop codons are not traditional initiation and termination codons, MOL4f could be a partial reading frame that extends further in the 5′ and/or 3′ directions.

TABLE 4O
MOL4f Nucleotide Sequence.

(SEQ ID NO:115)

GGATCCGGCCCATGGTCACCATGGCAACCATGTGAGCACTTGGATGGGGACAACTCACGCTCTTGCCTGTGTCGA
GCTCGATCCTGTGATTCCCCTCCACCCCCCTGTGCCCCCCTTGACTGCCTCCCGCCAGCCATCCACATCGCCAAC
TGCTCCACGAATGGGCCGTGGACCCCCTGGTCATCCTGCGCGCTGTGCAGCACCTCCTGTGGCATCGGCTTCCAG
GTCCGCCAGCGAAGTTGCAGCAACCCTGCTCCCCGCCACGGGGCCCCCATCTGCCTGGGCAAGAGCCGGGAGGAA
CGGTTCTGTAATCACAACACGCCTTGCCCGGTCCCCATCTTCTGGGCTTCCTGGGGCTCCTGGAGCAAGTCCAGC
AGCAACTGTGGAGGGGGCATGCAGTCGCGCCGTCGGGCCTCCGAGAACGGCAACTCCTGCCTGGGCTGCCGCGTG
GAGTTCAACACGTGCAACCCCGAGGGCTGCCCCCAAGTGCGGCGCAACACCCCCTCGACGCCGTGGCTGCCCGTG
AACGTGACCCAGGGCGGGGCACCGCAGGAGCACCGCTTCCGCTTCACCTGCCGCCCCCCCCTTGCACACCCGCAC
GGCCTCCACTTCGCCAGGAGAACGACCGAGACCAGGACCTGTCCCGCGGACGGCTCCCGCTCCTGCGACACCGAC
GCCCTGGTGGAGGACCTCCTCCGCACCGGCACCACCTCCCCCCACACCGTCACCGGGGGCTGGGCCGCCTCGGGC
CCGTGGTCGTCCTGCTCCCGGGACTGCCAGCTGGGCTTCCGCGTCCGCAAGAGAACGTCCACTAACCCCGAGCCC
CGCAACGGGGGCCTGCCCTCCGTGGGCCATGCTGCCGAGTACCACGACTGCAACCCCCAGGCTTCCCCAGTTCGG
GGTGCTTGGTCCTGCTGGACCTCATGGTCTCCATGCTCAGCTTCCTGTCCTGGGGGTCACTATCAACCCACCCGT
TCCTGCACCACCCCCGCACCCTCCCCACCTCACGACATCTGTCTCGGGCTGCACACGGAGGAGGCACTATGTGCC
ACACAGGCCTGCCCAGAACGCTCGTCGCCCTCGTCTGAGTGGAGTAAGTGCACTCACGACGGACCCCAGAGCCGA
AGCCGCCACTGTCACGACCTCCTCCCAGGCTCCAGCGCCTGTGCTGCAAACAGCACCCACAGCCCCCCCTGCGTC
GAC

The 401 amino acid MOL4f polypeptide (SEQ ID NO:116) encoded by SEQ ID NO:115 is presented using the one-letter amino acid code in Table 4P.

TABLE 4P
MOL4f protein sequence

(SEQ ID NO:116)

CSGPWSPWQRCEHLDGDNSGSCLCRARSCDSPRPRCGGLDCLGPAIHIAN
CSRNCAWTPWSSWALCSTSCGICFQVRQRSCSNPAPRHGGRICVCKSREE
RFCNENTPCPVPIFWASWGSWSKCSSNCGCGMQSRRPACENGNSCLGCGV
EFKTCNPEGCPEVRRNTPWTPWLPVNVTQGGARQEQRFRFTCPAPLADPH
GLQPGRRRTETRTCPADGSGSCDTDALVEDLLRSCSTSPHTVSCGWAAWG
PWSSCSRDCELGFRVRKRTCTNPEPRNCGLPCVGDAAEYQDCNPQACPVR
GAWSCWTSWSPCSASCCGGHYQRTRSCTSRAPSPGEDICLGLHTEEALCA
TQACPEGWSPWSEWSKCTDDGAQSRSRHCEELLFGSSACAGNSSQSRPCV
D

### MOL4g

The disclosed novel semaphorin-like protein, MOL4g (also referred to herein as 209829670), is encoded by a nucleic acid, 1203 nucleotides long (SEQ ID NO:117). An open reading frame was identified beginning with an GGA initiation codon at nucleotides 1-3 and ending after a GAC codon at nucleotides 1201-1203). in Table 4Q, the start codon is in bold letters. Because the start and stop codons are not traditional initiation and termination codons, MOL4g could be a partial reading frame that extends further in the 5′ and/or 3′ directions.

TABLE 4Q
MOL4g Nucleotide Sequence.

(SEQ ID NO:117)

GGATCCGCCCCATGGTCACCATGGCAACCATGTCAGCACTTGGATGGGGACAACTCAGGCTCTTGCCTGTCTCCA
GCTCGATCCTCTGATTCCCCTCGACCCCGCTGTCGGGGCCTTGACTGCCTGGCCCCAACCATCCACATCGCCAAC
TGCTCCAGGAATGGGGCGTGGACCCCGTGGTCATCGTGGGCGCTGTGCAGCACGTCCTGTGGCATCGGCTTCCAG
GTCCGCCAGCGAAGTTGCAGCAACCCTGCTCCCCGCCACGGGGGCCGCATCTGCGTGGGCAAGAGCCGGGAGGAA
CGGTTCTGTAATGAGAACACGCCTTCCCCCGTGCCCATCTTCTGGCCTTCCTCGGGCTCCTCGAGCAAGTGCGGC
AGCAACTGTGGAGGGGGCATGCAGTCGCGGCGTCGCGCCTGCGAGAACGGCAACTCCTCCCTGGGCTGCCGCGTC
GAGTTCAAGACGTGCAACCCCGAGGGCTGCCCCGAAGTGCGGCCCAACACCCCCTCGACGCCGTGGCTGCCCGTG
AACGTGACGCAGGGCCGGCCACCGCAGGACCACCGGTTCCGCTTCACCTGCCGCGCGCCCCTTGCAGACCCGCAC
GGCCTGCACTTCCGCAGCAGAACCACCCAGACGACGACCTCTCCCGCGGACGGCTCCGGCTCCTGCGACACCGAC
GCCCTGGTGCAGGTCCTCCTCCGCACCGGGAGCACCTCCCCGCACACGGTGAGCGGGGCCTGGGCCGCCTGGGGC
CCGTGGTCGTCCTGCTCCCCGGACTGCGAGCTGGGCTTCCGCCTCCGCAAGAGAACGTGCACTAACCCGGAGCCC
CGCAACGGGGGCCTGCCCTGCGTGGCCGATGCTGCCGAGTACCAGGACTGCAACCCCCAGGCTTGCCCAGTTCGG
GGTCCTTGGTCCTGCTGGACCTCATGGTCTCCATGCTCAGCTTCCTCTGGTGCGGGTCACTATCAACGCACCCGT
TCCTCCACCAGCCCCGCACCCTCCCCAGGTGAGGACATCTGTCTCGGGCTGCACACGGACCACGCACTATCTGCC
ACACAGGCCTCCCCAGAAGGCTGGTCCCCCTGGTCTGAGTGGAGTAAGTGCACTCACGACGGAGCCCACAGCCGA
AGCCCGCACTGTGAGGAGCTCCTCCCAGGGTCCAGCCCCTCTGCTGGAAACAGCAGCCAGAGCCGCCCCTGCGTC
GAC

The 401 amino acid MOL4g polypeptide (SEQ ID NO:118) encoded by SEQ ID NO:117 is presented using the one-letter amino acid code in Table 4R.

TABLE 4R
MOL4g protein sequence

(SEQ ID NO:118)

GSGPWSPWQFCEHLDCDNSCSCLCRARSCDSPRPRCGGLDCLGPTIHIAN
CSRNCAWTPWSSWALCSTSCCIGFQVRQRSCSNPAPRHGGRICVGKSREE
RFCNENTPCPVPIFWASWGSWSKCGSNCGGGMQSRRRACENGNSCLGCGV
EFKTCNPEGCPEVRRNTPWTPWLPVNVTQGGARQEQRFRFTCRAPLADPH
CLQFCRRRTETRTCPADGSGSCDTDALVEVLLRSGSTSPHTVSGGWAAWG
PWSSCSRDCELGFRVRKRTCTNPEPRNGGLPCVGDAAEYQDCNPQACPVR
GAWSCWTSWSPCSASCGGGHYQRTRSCTSFAPSPGEDICLGLHTEEALCA
TQACPEGWSPWSEWSKCTDDGAQSRSRHCEELLPCSSACACNSSQSRPCV
D

Table 4S shows a ClustalW alignment of the MOL4 variants

[image]

[image]

[image]

[image]

MOL4a has homology to the proteins decribed in Table 4T.

TABLE 4T
BLASTP results for MOL4a

| Gene Index/ | | Length | Identity | Positives | |
|---|---|---|---|---|---|
| Identifier | Protein/Organism | (aa) | (%) | (%) | Expect |
| gi\|7305473\| | sema domain, seven | 1093 | 1021/1093 | 1053/1093 | 0.0 |
| ref\|NP_038689.1\| | thrombospondin repeats | | (93%) | (95%) | |
| | (type 1 and type 1 - | | | | |
| | like), transmembrane | | | | |
| | domain (TM) and short | | | | |
| | cytoplasmic domain, | | | | |
| | (sem [Mus musculus] | | | | |
| gi\|7959149\| | KIAA1445 protein | 1202 | 1202/1202 | 1202/1202 | 0.0 |
| dbj\|BAA95969.1\| | [Homo sapiens] | | (100%) | (100%) | |
| (AB040878) | | | | | |
| gi\|4506881\| | sema domain, seven | 1074 | 616/1043 | 781/1043 | 0.0 |
| ref\|NP_003957.1\| | thrombospondin repeats | | (59%) | (74%) | |
| | (type 1 and type 1 - | | | | |
| | like), transmembrane | | | | |
| | domain (TM) and short | | | | |
| | cytoplasmic domain, | | | | |
| | (semaphorin) 5A; | | | | |
| | semaphorin F; sema | | | | |
| | domain, seven | | | | |
| | thrombospondin repeats | | | | |
| | (type 1 and type 1 - | | | | |
| | like), transmembrane | | | | |
| | domain (TM) | | | | |
| gi\|12731706\| | sema domain, seven | 1074 | 617/1043 | 781/1043 | 0.0 |
| ref\|XP_004042.2\| | thrombospondin repeats | | (59%) | (74%) | |
| | (type 1 and type 1 - | | | | |
| | like), transmembrane | | | | |
| | domain (TM) and short | | | | |
| | cytoplasmic domain, | | | | |
| | (semaphorin) 5A [Homo | | | | |
| | sapiens] | | | | |
| gi\|6677915\| | sema domain, seven | 1077 | 617/1046 | 776/1046 | 0.0 |
| ref\|NP_033180.1\| | thrombospondin repeats | | (58%) | (73%) | |
| | (type 1 and type 1 - | | | | |
| | like), transmembrane | | | | |
| | domain (TM) and short | | | | |
| | cytoplasmic domain, | | | | |
| | (sem; M-Sema D [Mus | | | | |
| | musculus] | | | | |

This information is presented graphically in the multiple sequence alignment given in Table 4U (with MOL4 being shown on line 1) as a ClustalW analysis comparing MOL4 with related sequences.

[image]

[image]

[image]

[image]

Tables 4V-4AA list the domain descriptions from DOMAIN analysis results against MOL4a. The region from amino acid residue 327 through 725 (SEQ ID NO:12) most probably (E=2e⁻¹¹⁸) contains a Sema domain found in Semaphorins, aligned here in Table 4V. Semaphorins are involved in growth cone guidance, axonal pathfinding, and other developmental processes. The region from amino acid residue 1057 through 1109 (SEQ ID NO:12) most probably (E=3e⁻⁹) contains a Thrombospondin type-1 repeat found in thrombospondin-1 that binds to and activates TGF-beta, aligned here in Table 4W. TGF-beta is involved in the modulation of proliferation in many cell types. The region from amino acid residue 868 through 921 (SEQ ID NO:12) most probably (E=4e⁻⁸) also contains a Thrombospondin type-1 repeat found in thrombospondin-1 that binds to and activates TGF-beta, aligned here in Table 4X. The region from amino acid residue 926 through 972 (SEQ ID NO:12) most probably (E=6e⁻⁷) also contains a Thrombospondin type-1 repeat found in thrombospondin-1 that binds to and activates TGF-beta, aligned here in Table 4Y. The region from amino acid residue 1169 through 1210 (SEQ ID NO:12) most probably (E=0.001) also contains a Thrombospondin type-1 repeat found in thrombospondin-1 that binds to and activates TGF-beta, aligned here in Table 4Z. The region from amino acid residue 756 through 803 (SEQ ID NO:12) most probably (E=1e⁻⁵) also contains a Thrombospondin type-1 repeat found in thrombospondin-1 that binds to and activates TGF-beta, aligned here in Table 4AA. The presence of these domains indicates that the MOL4a sequence has properties similar to those of other proteins known to contain these domains.

[image]

[image]

[image]

The above defined information for MOL4 suggests that this semaphorin-like protein may function as a member of a “Semaphorin family”. Therefore, the novel nucleic acids and proteins identified here may be useful in potential therapeutic applications implicated in (but not limited to) various pathologies and disorders as indicated below. The potential therapeutic applications for MOL4 include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The thrombospondin type 1 repeat (TSR) superfamily: diverse proteins with related roles in neuronal development.

Adams J C, Tucker R P. Dev Dyn 2000 June;218(2):280-99

The semaphorins are a gene family characterized by the presence of a phylogenetically conserved 500-amino acid domain (Kolodkin et al., [1993]). Some are secreted, some are associated with the cell surface via a GPI Iinkage, and others are transmembrane proteins. Many are expressed in the developing nervous system, and at least some of these have repulsive properties (e.g., Raper and Kapfhammer, [1990]; Luo et al., [1993]; Pueschel et al., [1995]). Adams et al. ([1996]) cloned two novel semaphorins from murine cDNA libraries that they designated SemF and SemG. SemF and SemG are 72% similar to each other and share a common domain organization: a relatively short cytoplasmic tail with proline-rich SH3 domains (analyzed further by Wang et al., [1999]), a single hydrophobic transmembrane domain, seven TSRs that contain WSXW motifs but lack the CSVTCG motif, and finally the large semaphorin domain. Northern blotting revealed semG expression in the early (E11) mouse embryo, when in situ hybridization showed semG expression in the neuroepithelium (Adams et al., [1996]; Skaliora et al., [1998]). Thus, SemG could play a role in neuroblast proliferation. In the adult, SemG mRNA was detected in brain but not in any other tissue examined (Adams et al., [1996]).

Adams R H, Betz H, Puschel A W. 1996. A novel class of murine semaphorins with homology to thrombospondin is differentially expressed during early embryogenesis. Mech Dev 57: 33-45.

Skaliora I, Singer W, Betz H, Puschel A W. 1998. Differential patterns of semaphorin expression in the developing rat brain. Eur Neurosci 10: 1215-1229.

The MOL4 nucleic acids and proteins are useful in potential therapeutic applications implicated in Parkinson's disease, psychotic and neurological disorders, Alzheimers disease, cancer including but not limited to lung or breast cancer, endocrine disorders, inflammatory disorders, gastro-intestinal disorders and disorders of the respiratory system, and/or other pathologies and disorders. For example, a cDNA encoding the semaphorin-like protein may be useful in gene therapy, and the semaphorin-like protein may be useful when administered to a subject in need thereof. By way of nonlimiting example, the compositions of the present invention will have efficacy for treatment of patients suffering from Parkinson's disease, psychotic and neurological disorders, Alzheimers disease, cancer including but not limited to lung or breast cancer, endocrine disorders, inflammatory disorders, gastro-intestinal disorders and disorders of the respiratory system. MOL4, or fragments thereof, may further be useful in diagnostic applications, wherein the presence or amount of the nucleic acid or the protein are to be assessed.

These materials are further useful in the generation of antibodies that bind immuno-specifically to the novel MOL4 substances for use in therapeutic or diagnostic methods. These antibodies may be generated according to methods known in the art, using prediction from hydrophobicity charts, as described in the “Anti-MOLX Antibodies” section below. The disclosed MOL4 protein has multiple hydrophilic regions, each of which can be used as an immunogen. In one embodiment, a contemplated MOL4 epitope is from about amino acids 5 to 75. In another embodiment, a MOL4 epitope is from about amino acids 100 to 200. In additional embodiments, MOL4 epitopes are from about amino acids 300 to 375, 420 to 600, 600 to 675, 775 to 850, 900 to 1150, and from about amino acids 1250 to 1350. These novel proteins can also be used to develop assay systems for functional analysis.

### MOL5

### MOL5a

The disclosed novel semaphorin 4C-like nucleic acid of 3868 nucleotides, MOL5a, (also referred to as SC20422974-A) is shown in Table 5A. An ORF begins with an ATG initiation codon at nucleotides 453-455 and ends with a TGA codon at nucleotides 2952-2954. A putative untranslated region upstream from the initiation codon and downstream from the termination codon is underlined in Table 5A, and the start and stop codons are in bold letters.

TABLE 5A

MOL5a Nucleotide Sequence

(SEQ ID NO:13)

CGACTATCCATGAAGCCCGGAGCCCCAGTGGCTGCAAGGCCTGCTGCCTGAGGTTCTTTCAAGAAACTCAAACCT
CTTAGGCCTGAGTGTGTATGTTGGGCGGGGGTCCCCTTTTTATTTCTCAAATGATTTCCTGTTGCGCAGAGGTAG
TGGTGGGTCTGGAGGCCAGGGAGGGCTTCCCGGAGCCTGTTTAGCCTTCAGCCAACTCAACTCCTCCCCGCTTCC
CAGGGAGACCTGTGGTCTTTTAGGCAGAGGCCAAGTGTGGGGACTTAGGTCCACCTCCAAAGAGAAGGGGAAGGA
GGGCACCGGGGCTCCTGGAAGGCCTGATGAGGAGTCCTGTGGCCTCTCCTGCTGCGGGCCCCTCTGGTTTGCTTT
CTCTGGCTGTGATTTCTGACCATGTCTTTTCCCTCAGCAGGACACCTCCCCTGAAGCTCACAGCCGCGCCGTGCG
CCATGGCCCCACACTGGGCTGTCTGGCTGCTGGCAGCAAGGCTGTGGGGCCTGGGCATTGGGGCTGAGGTGTGGT
GGAACCTTGTGCCGCGTAAGACAGTGTCTTCTCGGGAGCTGGCCACGGTAGTACGCCGGTTCTCCCAGACCCGCA
TCCAGGACTTCCTGACACTGACCCTCACGGACCCCACTGGGCTTCTGTACGTCCGCGCCCGAGAGGCCCTGTTTG
CCTTCAGTGTAGAGGCTCTGGAGCTGCAAGGAGCGATCTCCTGGGAGGCCCCCGTGGAGAAGAAGACTGAGTGTA
TCCAGAAAGCGAAGAACAACCACACCGAGTCCTTCAACTTCATCCGCTTCCTGCACCCCTACAATCCCTCCCACC
TGTACGTCTCTGGCACCTACGCCTTCCAGCCCAAGTGCACCTACGTCAACATCCTCACCTTCACTTTCGAGCATG
GAGAGTTTGAAGATGGCAAGGGCAAGTGTCCCTATGACCCAGCTAAGGCCCATGCTGGCCTTCTTGTGOATGGTG
AGCTCTACTCGGCCACACTCAACAACTTCCTGGGCACGGAACCCATTATCCTGCGTAACATGGGCCCCCACCACT
CCATGAAGACAGACTACCTGGCCTTTTGGCTCAACCAACCTCACTTTGTAGGCTCTCCCTATGTACCTGAGAGTG
TGGGCAGCTTCACGGGGGACGACGACAAGGTCTACTTCCTCTTCAGGGAGCGCGCACTGGAGTCCGCCTCCTATG
CCGAGCAGGTGGTGGCTCCTGTGGCCCCTGTCTGCAAGGGCCATATGGGCGCCGCACGGACCCTGCAGAGGAAGT
GGACCACGTTCCTGAACCCGCCGCTGGCATCCTCTCCCCCCAACTGGCAGCTCTACTTCAACCACCTGCAGGCGA
TGCACACCCTGCAGGACACCTCCTGCCACAACACCACCTTCTTTGCGGTTTTTCAAGCACAGTCGGCTCACATGT
ACCTGTCGGCCATCTGTCACTACCAGTTCGAAGAGATCCAGCGGGTGTTTGAGGGCCCCTALAAGGAGTACCATG
AGGAACCCCACAAGTGGGACCCCTACACTGACCCTGTACCCAGCCCTCGGCCTGGCTCGTGCATTAACAACTGGC
ATCGGCGCCACGGCTACACCAGCTCCCTGCAGCTACCCCACAACATCCTCAACTTCGTCAAGAAGCACCCGCTGA
TGGAGGAGCAGGTGGGGCCTCGGTGCACCCCCCCCCTGCTCGTGAAGAACGCCACCAACTTCACCCACCTCCTCG
CCGACCGGGTTACAGGACTTCATGGAGCCACCTATACAGTGCTGTTCATTGGCACACGTCACGCATGCCTGCTCA
AGGCTCTGAGCCTGGGGCCCTCCCTTCACCTGATTGAGGAGCTGCAGCTGTTTGACCAGGAGCCCATGAGAAGCC
TGGTGCTATCTCAcTCGCAGAAGCTGCTCTTTGCCGGCTCCCGCTCTCACCTGGTGCAGCTCCCCCTGGCCGACT
GCATCAACTATCGCTCCTCTGCAGACTGTGTCCTCCCCCGGGACCCCTATTGCGCCTGCACCGTCAACACCAGCC
GCTGTGTGGCCGTGGGTGGCCACTCTGGGTCCTTTCTGATCCAGCATGTGATCACCTCGGACACTTCACGCATCT
GCAACCTCCGTGGCACTAAGAAAGTCAGGCCCACTCCCPAAAACATCACGGTGGTGGCGGGCACAGACCTGGTCC
TGCCCTGCCACCTCTCCTCCAACTTGGCCCATGCCCGCTGGACCTTTCGCGGCCGGGACCTGCCTGCGGAACAGC
CCGGGTCCTTCCTCTACGATGCCCGGCTCCAGCCCCTGGTTGTGATGGCTGCCCAGCCCCGCCATCCCGGCGCCT
ACCACTGCTTTTCAGAGGAGCACGGGGCGCCCCTGGCTGCTGAAGGCTACCTTGTGGCTGTCGTGCCAGGCCCGT
CGGTGACCTTGGAGGCCCGGCCCCCCCTGGAAAACCTGGGGCTGGTGTGGCTCCCGGTGCTGCCCCTGGGGGCTG
TGTGCCTGGTGCTGCTCCTGCTGGTGCTGTCATTGCGCCCCCGGCTGCGGGAAGAGCTGGAGAAAGGGGCCAACC
CTACTGAGAGGACCTTGGTGTACCCCCTGGAGCTCCCCAAGGACCCCACCAGTCCCcCCTTCCGGCCCTCTCCTG
AACCACATGAGAAACTTTGGCATCCTGTCGCTTACTACTATTCAGATGGCTCCCTTAAGATACTACCTGGGCATG
CCCGGTGCCAGCCCGGTCGCGGGCCCCCTTCGCCACCTCCAGGCATCCCAGGCCAGCCTCTGCCTTCTCCAACTC
GGCTTCACCTGCGGGGTGGGCGGAACTCAAATGCCAATGGTTACGTGCGCTTACAACTAGGAGCGGAGCACCGGG
GAGGGCTCGGGCACCCCCTGCCTGAGCTCGCGGATGAACTGAGACGCAAACTGCAGCPACCCCAGCCACTGCCCC
ACTCCAACCCCGACGAGTCATCAGTATGAGGGGAACCCCCACCGCGTCGCCGGGAAGCCTGGGAGGTGTAGCTCC
TACTTTTGCACAGGCACCACCTATCTCAGGGACATCGCACGGGCACCTCCTCTGTCTGGGACAGATACTCCCCAG
CACCCACCCGGCCATGACGACCTGCTCTCCTCAGCACCGGCACTGCCACTTGGTGTGGCTCACCACGGCACCAGC
CTCGCAGAAGGCATCTTCCTCCTCTCTGTGAATCACAGACACGCGGGACCCCAGCCGCCAAAACTTTTCAAGGCA
GAAGTTTCAAGATGTGTCTTTGTCTCTATTTGCACATGTCTTTGTGTGTCTGTCTATGTCTGTCTGCACGCGCGT
GCGCGCTTGTGGCATAGCTTCCTGTTTCTGTCAAGTCTTCCCTTGGCCTGGGTCCTCCTGGTGAGTCATTGGAGC
TATGAAGGGGAAGCGGTCGTATCACTTTGTCTCTCCTACCCCCACTCCCCCGAGTGTCGGGCAGCGATGTACATA
TGGAGGTGGGGTGGACAGGGTGCTCTGCCCCTTCAGACGGAGTGCAGCGCTTGCGGTOGGCCTAGTCCTGCTCCT
AGGGCTGTGAATGTTTTCACGGTGCGGGGAGGCAGATGGAGCCTCCTGTGTGTTTGGGGGGAAGGGTCGGTGGGG
CCTCCCACTTGGCCCCGCGGTTCAGTGGTATTTTATACTTGCCTTCTTCCTGTACAGGGCTGGGAAAGGCTGTGT
GAGGGGAGAGAAGGGAGAGGGTGGGCCTGCTGTCGACAATCGCATACTCTCTTCCACCCCTACGACGAGGGCTCC
TAACAGTGTAACTTATTGTGTCCCCGCGTATTTATTTGTTGTAAATATTTGAGTATTTTTATATTGACAAATAAA
ATGGAGAAAAAAAAAAAAAAAAAAAAAAAAGTCGTATCGATGT

The MOL5a protein encoded by SEQ ID NO:13 has 833 amino acid residues and is presented using the one-letter code in Table 5B. The Psort profile for MOL5a predicts that this sequence is likely to be localized at the mitochondrial inner membrane with a certainty of 0.8000 or plasma membrane with a certainty of 0.7000. MOL5a has a cleavage site between amino acods 20 and 21 (GIG-AE), and a molecular weight of 92617.0 Daltons.

TABLE 5B
Encoded MOL5a protein sequence

(SEQ ID NO:14)

MAPHWAVWLLAARLWGLGIGAEVWWNLVPRKTVSSGELATVVRRFSQTGIQDFLTLTLTEPTGLLYVGAREAL
FAFSVEALELQGAISWEAPVEKKTECIQKGKNNQTECFNFIRFLQPYNASHLYVCGTYAFQPKCTYVNMLTFT
LEHGEFEDGKGKCPYDPAKGHAGLLVDGELYSATLNNFLGTEPIILRNMGPHHSMKTEYLAFWLNEPHFVGSA
YVPESVGSFTGDDDKVYFLFRERAVESDCYAEQVVARVARVCKCDMGGARTLQRKWTTFLKARLACSAPNWQL
YFNQLQAMHTLQDTSWHNTTFTGVFQAQWGDMYLSAICEYQLEETQRVFECFYKEYHEEAQKWDRYTDPVPSP
RPGSCINNWHRRHGYTSSLELPDNILNFVKKHPLMEEQVGFRWSRPLLVKKGTNFTHLVADRVTGLDCATYTV
LFIGTGQAWLLKAVSLGPWVHLIEELQLFDQEPMRSLVLSQSQKLLFAGSRSQLVQLPVADCMKYRSCADCVL
ARDPYCAWSVNTSRCVAVGGHSGSFLTQHVMTSDTSCICNLRGSKKVRPTPKNITVVAGTDLVLPCHLSSNLA
HARWTFGCRDLFAEQPGSFLYDARLQALVVMAAQRRHAGAYHCFSEEQGARLAAEGYLVAVVAGPSVTLEARA
PLENLGLVWLAVVALGAVCLVLLLLVLSLRRRLREELEKGAKATERTLVYPLELPKEPTSPPFRPCPEPDEKL
WDPVGYYYSDGSLKIVPGHARCQPGCCPPSPPPGIPGQPLPSPTRLHLGGGRNSNANGYVRLQLGGEDRGGLG
HPLPELADELRRKLQQRQPLPDSNPEESSV

The disclosed nucleic acid sequence for MOL5a has 2917 of 3443 bases (84%) identical to a semaphorin 4C mRNA (GENBANK-ID: S79463|acc:S79463) (E=0.0).

The full MOL5a amino acid sequence has 729 of 834 amino acid residues (87%) identical to, and 772 of 834 residues (92%) positives with, the 834 amino acid semaphorin 4C Precursor protein from Mus musculus (Mouse) (ptnr:SPTREMBL-ACC: Q64151) (E=0.0). In addition, this protein contains the following protein domains (as defined by Interpro) at the indicated nucleotide positions: Sema domain (a.a. 53-481; IPR001627), integrin_B (a.a. 505-519; IPR000413), Plexin_repeat (a.a. 499-551; IPR002165), ig (a.a. 570-629; IPR000353)

MOL5a expression in different tissues was examined through TaqMan as described below in Example 1.

### Chromosomal Localization

MOL5a has been localized to human chromosome 2.

### MOL5b

Another disclosed novel semaphorin 4C-like nucleic acid of 2558 nucleotides, MOL5b, (also referred to as SC14998905_EXT) is shown in Table 5C. An ORF begins with an ATG initiation codon at nucleotides 21-23 and ends with a TGA codon at nucleotides 2520-2522. A putative untranslated region upstream from the initiation codon and downstream from the termination codon is underlined in Table 5C, and the start and stop codons are in bold letters.

TABLE 5C

MOL5b Nucleotide Sequence

(SEQ ID NO:15)

TCAGAGCCGGGGCGTCCGCCATGGCCCCACACTGGGCTGTCTGCCTGCTGGCAGCAAGGCTGTGCGGCCTCCGCA
TTGCGGCTGAGGTGTGGTGGAACCTTGTGCCCCGTAAGACACTCTCTTCTGGGGACCTCGCCACGGTAGTACGGC
GGTTCTCCCAGACCCCCATCCAGGACTTCCTCACACTGACCCTGACGGAGCCCACTGGGCTTCTGTACGTGCGCC
CCACGGACCATGCCTCTGCACTGGGCGTCCCTGTGTTCCTGCTGCAGGCTGTGATCTCCTGGGAGGCCCCCGTCG
AGAAGAACACTCAGTGTATCCACAAAGGGAAGAACAACCAGACCGACTGCTTCPACTTCATCCCCTTCCTGCAGC
CCTACAATGCCTCCCACCTGTACGTCTGTGGCACCTACGCCTTCCAGCCCAAGTGCACCTACGTCAACATGCTCA
CCTTCACTTTGGAGCATGGAGAGTTTGAAGATGGGAAGGGCAAGTGTCCCTATGACCCAGCTAAGGGCCATGCTG
GCCTTCTTGTGGATGGTGAGCTGTACTCGGCCACACTCAACAACTTCCTGGGCACGGAACCCATTATCCTGCGTA
ACATGGGGCCCCACCACTCCATGAAGACAGAGTACCTGGCCTTTTGGCTCAACGAACCTCACTTTGTAGGCTCTG
CCTATGTACCTGAGAGTGTGGGCAGCTTCACGGGGGACGACGACAACGTCTACTTCTTCTTCAGGGAGCCGGCAG
TGCAGTCCCACTGCTATGCCGAGCAGGTCGTGGCTCGTCTGGCCCGTGTCTGCAAGGGCGATATCGGGGGCCCAC
GGACCCTGCAGAGGAAGTGGACCACGTTCCTGAAGGCGCGGCTGGCATGCTCTCCCCCGAACTGGCAGCTCTACT
TCAACCAGCTGCAGGCGATGCACACCCTGCAGGACACCTCCTGGCACAACACCACCTTCTTTGGGGTTTTTCAAG
CACAGTGGCGTCACATGTACCTGTCGGCCATCTGTGAGTACCAGTTGGAAGAGATCCAGCGCGTGTTTCAGGGCC
CCTATAAGGAGTACCATGACGAAGCCCAGAAGTCCGACCCCTACACTGACCCTGTACCCAGCCCTCGCCCTGGCT
CGTGCATTAACAACTGGCATCGGCGCCACGGCTACACCAGCTCCCTGGAGCTACCCGACAACATCCTCAACTTCG
TCAAGAAGCACCCGCTGATGGAGGAGCAGGTGGGGCCTCGGTGGAGCCGCCCCCTGCTCGTGAAGAAGGGCACCA
ACTTCACCCACCTGCTCGCCGACCGGCTTACAGGACTTGATGGAGCCACCTATACAGTCCTGTTCATTCGCACAG
GACACGGCTCGCTCCTCAAGGCTCTGAGCCTCCGGCCCTGGGTTCACCTGATTGACGAGCTCCAGCTCTTTGACC
AGGAGCCCATGAGAAGCCTGGTCCTATCTCAGAGCAAGAACCTCCTCTTTGCCGCCTCCCGCTCTCAGCTGGTCC
AGCTGCCCGTGGCCGACTGCATGAAGTATCGCTCCTGTGCAGACTGTGTCCTCGCCCGGGACCCCTATTCCGCCT
GGAGCGTCAACACCACCCCCTGTGTGCCCGTGGGTGCCCACTCTGGATCTCTACTGATCCAGCATGTGATGACCT
CGGACACTTCAGGCATCTGCAACCTCCGTGGCAGTAAGAAAGTCAGGCCCACTCCCAAAAACATCACGGTGGTGG
CGGGCACAGACCTGGTCCTCCCCTGCCACCTCTCCTCCAACTTGGCCCATGCCCGCTGGACCTTTGGGGGCCGGG
ACCTGCCTGCGGAACACCCCGGGTCCTTCCTCTACCATGCCCGGCTCCAGGCCCTCCTTGTGATGGCTCCCCAGC
CCCCCCATGCCGGGCCCTACCACTGCTTTTCACACGAGCAGGGGGCCCCGCTCGCTGCTGAAGGCTACCTTCTCC
CTGTCGTGCCAGGCCCGTCGGTCACCTTCGAGGCCCGGGCCCCCCTGGAAAACCTGGCGCTGCTGTCGCTCGCGG
TGGTCGCCCTGGGGGCTCTGTGCCTGGTGCTGCTGCTGCTCGTGCTCTCATTGCCCCGGCCGCTGCGGGAAGAGC
TGGAGAAAGGGGCCAAGGCTACTGACACGACCTTGCTCTACCCCCTGGAGCTCCCCAAGGAGCCCACCAGTCCCC
CCTTCCGGCCCTGTCCTGAACCACATGACAPACTTTGCCATCCTGTCGGTTACTACTATTCAGATGGCTCCCTTA
AGATAGTACCTCGCCATGCCCGGTCCCAGCCCGGTCGGGGGCCCCCTTCGCCACCTCCACGCATCCCAGGCCAGC
CTCTGCCTTCTCCAACTCGGCTTCACCTGGGGGGTGGGCGGAACTCAAATGCCAATGGTTACGTGCGCTTACAAC
TAGGACCGGAGGACCGGGGAGGGCTCGGGCACCCCCTCCCTGAGCTCGCGGATGPACTGAGACGCAAACTGCAGC
AACCCCAGCCACTGCCCGACTCCAACCCCCAGGAGTCATCAGTATGACGGCAACCCCCACCGCGTCGCCGGCAAG
CGTGGGAC

The MOL5b protein encoded by SEQ ID NO:16 has 833 amino acid residues and is presented using the one-letter code in Table 5D. The Psort profile for MOL5b predicts that this sequence is likely to be localized at the plasma membrane with a certainty of 0.7000.

TABLE 5D

Encoded MOL5b protein sequence

(SEQ ID NO:16)

MAPHWAVWLLAARLWGLGIGAEVWWNLVPRKTVSSGELATVVRRFSQTGIQDFLTLTLTEPTGLLYVGARDHA
SALGVPVLLLQAVISWEAPVEKKTECIQKGKNNQTECFNFIRFLQPYNASHLYVCGTYAFQPKCTYVNMLTFT
LEHGEFEDGKGKCPYDPAKGHAGLLVDGELYSATLNNFLGTEPIILRNMGPHHSMKTEYLAFWLNEPHFVGSA
YVPESVGSFTGDDDKVYFFFRERAVESDCYAEQVVARVARVCKGDMGGARTLQRKWTTFLKARLACSAPNWQL
YFNQLQAMHTLQDTSWHNTTFFCVFQAQWCDMYLSAICEYQLEEIQRVFEGPYKEYHEEAQKWDRYTDPVPSP
RPGSCTNNWHRRHGYTSSLELPDNILNFVKKHPLMEEQVGPRWSRPLLVKKGTNFTHLVADRVTGLDGATYTV
LFIGTGDGWLLKAVSLGPWVHLIEELQLFDQEFMRSLVLSQSKKLLFAGSRSQLVQLPVADCMKYRSCADCVL
ARDPYCAWSVNTSRCVAVGGHSGSLLIQHVMTSDTSGTCNLRGSKKVRPTPKNITVVAGTDLVLPCHLSSNLA
HARWTFGGRDLPAEQPGSFLYDARLQALVVMAAQPRHAGAYHCFSEEQGARLAAEGYLVAVVAGPSVTLEARA
PLENLGLVWLAVVALGAVCLVLLLLVLSLRRRLREELEKGAKATERTLVYPLELPKEPTSPPFRPCPEPDEKL
WDPVGYYYSDCSLKIVPGHARCQPGGCPPSPPPGTPCQFLPSPTRLHLCGGRNSNANGYVRLQLGGEDRGGLG
HPLPELADELRRKLQQRQPLPDSNPEESSV

The disclosed nucleic acid sequence for MOL5b has 1695 of 2019 bases (83%) identical to a mouse Semaphorin4C mRNA (GENBANK-ID: S79463) (E=0.0).

The full MOL5b amino acid sequence has 722 of 834 amino acid residues (86%) identical to, and 765 of 834 residues (91%) positive with the amino acid Semaphorin4C HOMOLOG protein from Mouse (S79463_SEMA_{—}4C_MOUSE) (E=0.0). The global sequence homology (as defined by FASTA alignment with the full length sequence of this protein) is 91% amino acid homology and 86% amino acid identity.

### Chromosomal Localization

MOL5b has been localized to human chromosome 2.

### MOL5c

In the present invention, the target sequence identified previously, MOL5b, was subjected to the exon linking process to confirm the sequence. PCR primers were designed by starting at the most upstream sequence available, for the forward primer, and at the most downstream sequence available for the reverse primer. In each case, the sequence was examined, walking inward from the respective termini toward the coding sequence, until a suitable sequence that is either unique or highly selective was encountered, or, in the case of the reverse primer, until the stop codon was reached. Such primers were designed based on in silico predictions for the full length cDNA, part (one or more exons) of the DNA or protein sequence of the target sequence, or by translated homology of the predicted exons to closely related human sequences sequences from other species. These primers were then employed in PCR amplification based on the following pool of human cDNAs: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea, uterus. Usually the resulting amplicons were gel purified, cloned and sequenced to high redundancy. The resulting sequences from all clones were assembled with themselves, with other fragments in CuraGen Corporation's database and with public ESTs. Fragments and ESTs were included as components for an assembly when the extent of their identity with another component of the assembly was at least 95% over 50 bp. In addition, sequence traces were evaluated manually and edited for corrections if appropriate. These procedures provide the sequence reported below, which is designated MOL5c (Accession Number CG50907-02). This differs from the previously identified sequence, MOL5b, in having 17 different amino acids.

The disclosed novel semaphorin 4C-like nucleic acid of 3112 nucleotides, MOL5c, (also referred to as CG50907-02) is shown in Table 5E. An ORF begins with an ATG initiation codon at nucleotides 104-106 and ends with a TGA codon at nucleotides 2603-2605. A putative untranslated region upstream from the initiation codon and downstream from the termination codon is underlined in Table 5E, and the start and stop codons are in bold letters.

TABLE 5E

MOL5c Nucleotide Sequence

(SEQ ID NO:17)

TGCTGCGGGCCCCTCTGGTTTGCTTTCTCTGGCTGTGATTTCTGACCATGTCTTTTCCCTCAGCAGGACACCTGG
CCTGAAGCTCAGAGCCGGGGCGTCCCCCATGGCCCCACACTGCCCTGTCTGGCTGCTGGCAGCAAGGCTGTGGGG
CCTGGGCATTGGGGCTCAGGTCTGGTGGAACCTTGTGCCCCGTAAGACAGTGTCTTCTGGGGAGCTGGCCACGCT
AGTACGGCGGTTCTCCCACACCGGCATCCAGGACTTCCTGACACTGACCCTGACGGAGCCCACTGGCCTTCTCTA
CGTGCGCGCCAGCGACCATGCCTCTGCACTGGGCGTCCCTGTGTTCCTGCTGCACGCTGTGATCTCCTCGGAGCC
CCCCGTGGAGAAGAAGACTGAGTGTATCCAGAAAGGGAAGAACAACCAGACCGAGTGCTTCAACTTCATCCGCTT
CCTGCAGCCCTACAATGCCTCCCACCTGTACGTCTGTGGCACCTACGCCTTCCAGCCCAAGTGCACCTACGTCAA
CATGCTCACCTTCACTTTGGAGCATGGAGAGTTTGAAGATGGGAAGGGCAAGTGTCCCTATGACCCAGCTAAGGG
CCATGCTGCCCTTCTTGTGCATGGTGAGCTGTACTCCGCCACACTCAACAACTTCCTCGGCACGGAACCCATTAT
CCTGCGTAACATGGGGCCCCACCACTCCATGAAGACAGAGTACCTGGCCTTTTGGCTCAACGAACCTCACTTTGT
AGGCTCTGCCTATGTACCTGAGAGTGTCGGCAGCTTCACGGGGCACGACGACAACGTCTACTTCTTCTTCAGGGA
GCCGGCAGTGGAGTCCGACTCCTATGCCGAGCAGGTCGTGCCTCGTGTGGCCCGTCTCTGCAAGCGCGATATGGC
GGGCGCACGGACCCTGCAGAGGAAGTGGACCACGTTCCTGAAGGCGCGGCTGGCATGCTCTGCCCCGAACTGGCA
GCTCTACTTCAACCAGCTGCAGCCGATGCACACCCTGCAGGACACCTCCTGGCACAACACCACCTTCTTTGGGGT
TTTTCAAGCACACTCGGGTGACATGTACCTGTCGGCCATCTCTCAGTACCAGTTGGAAGAGATCCAGCGGGTGTT
TGACGGCCCCTATAACGAGTACCATGAGGPAGCCCAGAAGTGGGACCGCTACACTGACCCTGTACCCAGCCCTCC
GCCTGGCTCGTGCATTAACAACTGGCATCCGCGCCACGGCTACACCAGCTCCCTGCACCTACCCGACAACATCCT
CAACTTCGTCAAGAAGCACCCGCTCATGGAGCACCAGGTCGGGCCTCGGTCGAGCCGCCCCCTCCTCGTGAAGAA
GGGCACCAACTTCACCCACCTCGTGCCCGACCGGGTTACAGGACTTCATGGAGCCACCTATACAGTGCTGTTCAT
TGGCACAGGAGACGGCTGGCTGCTCAAGGCTGTGACCCTGCGCCCCTGGCTTCACCTGATTGAGCAGCTGCAGCT
GTTTCACCAGGAGCCCATCACAAGCCTGGTCCTATCTCAGAGCAAGAAGCTGCTCTTTGCCGGCTCCCGCTCTCA
GCTGGTGCAGCTGCCCGTGGCCGACTGCATGPAGTATCGCTCCTGTGCACACTCTGTCCTCGCCCGCGACCCCTA
TTGCGCCTGCACCGTCAACACCAGCCGCTGTGTGGCCGTGGGTGGCCACTCTGGATCTCTACTGATCCAGCATCT
GATGACCTCGGACACTTCAGGCATCTGCAACCTCCGTGGCAGTAAGAAAGTCAGGCCCACTCCCAAAAACATCAC
GGTGGTGGCGGGCACAGACCTGGTGCTGCCCTGCCACCTCTCCTCCAACTTGGCCCATGCCCGCTGGACCTTTGC
GGGCCGCCACCTGCCTGCGGAACAGCCCGGGTCCTTCCTCTACCATGCCCCGCTCCAGGCCCTCGTTGTGATGGC
TGCCCAGCCCCGCCATGCCGGGCCCTACCACTGCTTTTCAGACGAGCAGGGGGCGCGGCTGGCTGCTGAACGCTA
CCTTGTGGCTCTCGTGGCACCCCCGTCGGTCACCTTGGAGGCCCGGGCCCCCCTGGAAAACCTGGGGCTCGTGTG
GCTGGCGGTGGTGGCCCTGGGGOCTGTGTGCCTGGTGCTGCTGCTGCTGGTGCTGTCATTCCGCCGCCGGCTGCG
GGAAGAGCTGGAGAAAGGGGCCAACGCTACTCAGAGCACCTTGGTGTACCCCCTGGAGCTGCCCAAGGAGCCCAC
CAGTCCCCCCTTCCGGCCCTGTCCTGAACCAGATGAGAAACTTTGGGATCCTGTCGGTTACTACTATTCAGATGG
CTCCCTTAAGATAGTACCTGGGCATCCCCGCTGCCAGCCCGCTGCCGGGCCCCCTTCGCCACCTCCAGGCATCCC
AGGCCAGCCTCTGCCTTCTCCAACTCGGCTTCACCTGGGGGGTGGGCGGAACTCAAATGCCAATGGTTACGTGCG
CTTACAACTAGGAGGGCAGGACCGGCCAGGGCTCGGGCACCCCCTGCCTGAGCTCGCGGATGAACTGAGACGCAA
ACTCCAGCAACGCCAGCCACTGCCCGACTCCAACCCCGAGGAGTCATCAGTATGAGGGGAACCCCCACCGCGTCG
GCGCCAAGCCTGGGACCTGTACCTCCTACTTTTGCACAGCCACCACCTACCTCACCCACATGCCACGGCCACCTG
CTCTCTCTGGCACAGATACTGCCCAGCACCCACCCCGCCATGAGGACCTCCTCTGCTCAGCACGCGCACTGCCAC
TTGCTGTGCCTCACCACGOCACCACCCTCGCAGAACGCATCTTCCTCCTCTCTGTGAATCACACACACCCGCGAC
CCCACCCCCCAAAACTTTTCAAGCCAGAACTTTCAACATGTGTGTTTCTCTCTATTTGCACATGTGTTTGTGTGT
GTGTGTATCTGTGTCTGCACCCGCCTCCCCCCTTGTGGCATAGCCTTCCTGTTTCTGTCAAGTCTTCCCTTGCCC
TGGGTCCTCCTGGTGAGTCATTGGAGCTATGAAGGGGAAGGGGTCGTATCACTTTGTCTCTCCTACCCCCACTGC
CCCCAGTGTCCCCCACCOATCTACATATGCAGCTGGG

The MOL5c protein encoded by SEQ ID NO:17 has 833 amino acid residues and is presented using the one-letter code in Table 5F. The Psort profile for MOL5c predicts that this sequence has a signal peptide and the signal peptide is predicted by SignalP to be cleaved between amino acid 20 and 21: GIG-AE. This sequence is likely to be localized at the mitochondrial inner membrane with a certainty of 0.8000 and the plasma membrane with a certainty of 0.7000.

TABLE 5F

Encoded MOL5c protein sequence

(SEQ ID NO:18)

MAPHNAVWLLAARLWGLGIGAEVWWNLVPRKTVSSGELATVVRRFSQTGIQDFLTLTLTEPTGLLYVCARDHA
SALGVPVLLLQAVISWEAPVEKRTECIQKGKNNQTECFNFIRFLQPYNASHLYVCGTYAEQPKCTYVNMLTFT
LEHCEFEDGKGKCPYDPAKGHAGLLVDGELYSATLNNFLGTEPIILRNMGPHHSMKTEYLAFWLNEPHFVGSA
YVRESVGSFTGDDDKVYFFFRERAVESDCYAEQVVARVARVCKGDMGGARTLQRKWTTFLKARLACSAPNWQL
YFNQLQAMHTLQDTSWHNTTFFGVFQAQWGDMYLSAICEYQLEEIQRVFEGRYKEYHEEAQKWDRYTDPVPSF
RPGSCINNWHRRHGYTSSLELPDNILNFVKKHPLMEEQVGPRWSRPLLVKKGTNFTHLVADRVTGLDGATYTV
LFIGTGDGWLLKAVSLGPWVHLIEELQLFDQEPMRSLVLSQSKKLLFAGSRSQLVQLPVADCMKYRSCADCVL
ARDPYCAWSVNTSFCVAVGGHSCSLLIQHVMTSDTSGTCNLRGSKKVRPTPKNITVVAGTDLVLPCHLSSNLA
HARWTFGCRDLPAEQPGSFLYDARLQALVVMAAQPRHAGAYHCFSEEQGARLAAEGYLVAVVACPSVTLEARA
PLENLGLVWLAVVALGAVCLVLLLLVLSLRRRLREELEKGAKATERTLVYPLELPKEPTSPPFRPCPEPDEKL
WDPVGYYYSDGSLKIVPGHARCQPCCGPPSPPPGIPCQPLPSPTRLHLGGGRNSNANGYVRLQLGGEDRGGLG
HPLPELADELRRKLQQRQPLPDSNPEESSV

The disclosed nucleic acid sequence for MOL5c has 2879 of 2906 bases (99%) identical to a gb:GENBANK-ID:AB051526|acc:AB051526.1 mRNA from Homo sapiens (Homo sapiens mRNA for KIAA1739 protein, partial cds) (E=0.0).

The full MOL5 amino acid sequence has 722 of 834 amino acid residues (86%) identical to, and 765 of 834 amino acid residues (91%) similar to, the 834 amino acid residue ptnr:SWISSPROT-ACC:Q64151 protein from Mus musculus (Mouse) (SEMAPHORIN 4C PRECURSOR (SEMAPHORIN I) (SEMA I) (SEMAPHORIN C-LIKE 1) (M-SEMA F)) (E=0.0). The global sequence homology (as defined by FASTA alignment with the full length sequence of this protein) is 91% amino acid homology and 86% amino acid identity.

The presence of identifiable domains in the protein disclosed herein was determined by searches versus domain databases such as Pfam, PROSITE, ProDom, Blocks or Prints and then identified by the Interpro domain accession number. Significant domains are summarized in Table 5G.

TABLE 5G
Domain similarities for MOL5c
Scores for sequence family classification (score includes
all domains):

| Model | Description | Score | E-value | N |
|---|---|---|---|---|
| Sema | Sema domain | 664.4 | 5.8e−196 | 1 |
| Plexin_repeat | Plexin repeat | 25.8 | 0.001 | 1 |
| ig | Immunoglobulin domain | 8.5 | 0.44 | 1 |
| integrin_B | Integrins, beta chain | 7.0 | 0.04 | 1 |

Parsed for domains:

| | | | | hmm- | | | |
|---|---|---|---|---|---|---|---|
| Model | Domain | seq-f | seq-t | f | hmm-t | score | E-value |
| Sema | 1/1 | 53 | 481 . . . | 1 | 490 [ ] | 664.4 | 5.8e−196 |
| integrin_{—} | 1/1 | 505 | 519 . . . | 1 | 14 [ . | 7.0 | 0.04 |
| B | | | | | | | |
| Plexin_{—} | 1/1 | 499 | 551 . . . | 1 | 67 [ ] | 25.8 | 0.001 |
| repeat | | | | | | | |
| ig | 1/1 | 570 | 629 . . . | 1 | 45 [ ] | 8.5 | 0.44 |

The Sema domain occurs in semaphorins, which are a large family of secreted and transmembrane proteins, some of which function as repellent signals during axon guidance. Sema domains also occur in a hepatocyte growth factor receptor, in SEX protein (Goodman et al., 1998, Cell 95: 903-916) and in viral proteins.

The presence of these domains indicates that MOL5c likely has properties similar to those of other proteins known to contain this/these domain(s) and similar to the properties of these domains.

### Chromosomal Localization

MOL5c maps to chromosome 2. This assignment was made using mapping information associated with genomic clones, public genes and ESTs sharing sequence identity with the disclosed sequence and CuraGen Corporation's Electronic Northern bioinformatic tool.

### Tissue Expression

MOL5c is expressed in at least the following tissues: adrenal gland, bone marrow, brain—amygdala, brain—cerebellum, brain—hippocampus, brain—substantia nigra, brain—thalamus, brain—whole, fetal brain, fetal kidney, fetal liver, fetal lung, heart, kidney, lymphoma—Raji, mammary gland, pancreas, pituitary gland, placenta, prostate, salivary gland, skeletal muscle, small intestine, spinal cord, spleen, stomach, testis, thyroid, trachea and uterus. Expression information was derived from the tissue sources of the sequences that were included in the derivation of the sequence of MOL5c.

### MOL5d

The disclosed novel semaphorin 4C-like nucleic acid of 1914 nucleotides, MOL5d, (also referred to as CG50907-03) is shown in Table 5H. An ORF begins with an ATG initiation codon at nucleotides 104-106 and ends with a TGA codon at nucleotides 2603-2605. A putative untranslated region upstream from the initiation codon and downstream from the termination codon is underlined in Table 5H, and the start and stop codons are in bold letters.

TABLE 5H

MOL5d Nucleotide Sequence

(SEQ ID NO:119)

CTCGAGCTCCAAGGTCACCGACCGGCCTCCCACGACAGCCACAACGTACCCTTCAGCAGCCACCCGCGCCCCCTG
CTCCTCTCAAAAGCAGTGGTAGGCCCCGGCATCGCGCCCCTGGCCAGCCATCACAACCAGGCCCTGGAGCCGCGC
ATCGTAGAGGAAGGACCCGGGCTGTTCCGCAGGCAGGTCCCGGCCCCCAAAGGTCCAGCGGGCATGGGCCAAGTT
GGACGAGAGGTCGCACCGCAGCACCAGGTCTGTGCCCGCCACCACCGTGATGTTTTTGGGAGTGGGCCTGACTTT
CTTACTGCCACCGAGGTTGCAGATGCCTGAAGTGTCCCAGGTCATCACATGCTGGATCAGTAGACATCCAGAGTG
GCCACCCACGGCCACACAGCGGCTCCTGTTGACGCTCCACGCGCAATAGGCGTCCCGCCCCAGGACACACTCTGC
ACAGGAGCGATACTTCATGCAGTCGGCCACGGGCAGCTGCACCAGCTGAGAGCGGGAGCCGGCAAAGAGCAGCTT
CTTGCTCTGAGATAGCACCAGGCTTCTCATGGGCTCCTGGTCAAACAGCTGCAGCTCCTCAATCAGGTGAACCCA
GGGCCCCAGGCTCACAGCCTTGAGCAGCCAGCCGTCTCCTCTGCCAATGAACAGCACTGTATAGGTGGCTCCATC
AAGTCCTGTAACCCGCTCCGCCACCAGGTCGGTGAAGTTGGTGCCCTTCTTCACGAGCAGCGGGCGGCTCCACCG
AGGCCCCACCTGCTCCTCCATCAGCGGGTGCTTCTTGACGAAGTTGAGGATGTTGTCGGGTAGCTCCAGGGAGCT
GGTGTACCCGTGCCGCCGATGCCAGTTGTTAATGCACGAGCCAGGCCGAGGGCTCGGTACAGGGTCAGTGTAGCG
GTCCCACTTCTCGGCTTCCTCATGGTACTCCTTATAGGCGCCCTCAAACACCCGCTGCATCTCTTCCAACTGCTA
CTCACAGATGGCCGACAGCTACATGTCACCCCACTGTGCTTGAAAAACCCCAAAGAAGGTGGTGTTGTGCCAGCA
GGTGTCCTGCAGGGTGTGCATCCCCTGCACCTGGTTGAAGTAGAGCTGCCACTTCCCGGCAGAGCATGCCAGCCG
CGCCTTCAGGAACGTGCTCCACTTCCTCTGCAGGGTCCCTGCGCCCCCCATATCGCCCTTGCAGACACGCGCCAC
ACGAGCCACCACCTGCTCGGCATAGCAGTCGGACTCCACTGCCCGCTCCCTGAAGAAGAAGTAGACCTTGTCGTC
GTCCCCCCTGAAGCTGCCCACACTCTCAGGTACATAGCCAGAGCCTACAAAGTGACGTTCCTTGACCCAAAAGGC
CAGGTACTCTGTCTTCATGGAGTGGTGGGGCCCCATGTTACGCAGGATAATGGGTTCCGTGCCCAGGAAGTTGTT
GAGTGTGCCCCAGTACAGCTCACCATCCACAAGAAGGCCAGCATGGCCCTTACCTCGGTCATAGGGACACTTCCC
CTTCCCATCTTCAAACTCTCCATGCTCCAAAGTGAACGTGAGCATGTTCACCTAGGTCCACTTGGGCTCCAAGGC
GTAGGTGCCACAGACGTACAGCTGGGAGGCATTGTAGGGCTGCACGAAGCGGATGAAGTTGAAGCACTCGGTCTG
CTTGTTCTTCCCTTTCTGGATACACTCACTCTTCTCCTCCACCGGGGCCTCCCAGGAGATCGCTCCTTGCACCTC
CAGGGCCTCCATCCTGAACGCAAACAGGGCCTCTCGCGCCCCCACGTACAGPAGCCCAGTGGCCTCCGTCAGCGT
CAGTCTCAGGAAGTCCTCCATGCCGGTCTGCGAGAACCGCCGTACTACCGTGGCCAGCTCCCCACPAGACACTGT
CTTACGCGGCACAAGGTTCCACCACACCTCAGCAGATCT

The nucleic acid sequence for MOL5d is 99% identical to the 2156 sequence disclosed in WO200078802-A2 with a priority date of Jun. 23, 1999.

The MOL5d protein encoded by SEQ ID NO:120 has 634 amino acid residues and is presented using the one-letter code in Table 5I. The Psort profile for MOL5d predicts that this sequence has a signal peptide and the signal peptide is predicted by SignalP to be cleaved between amino acid 20 and 21: GIG-AE. This sequence is likely to be a type 1 membrane protein (ECD proposed for immunization) localized at the mitochondrial inner membrane with a certainty of 0.8000 and the plasma membrane with a certainty of 0.7000

TABLE 5I
Encoded MOL5d rrotein sequence

(SEQ ID NO:120)

AEVWWNLVPRKTVSSGELATVVRRFSQTGIQDFLTLTLTEPTGLLYVGAREALFAFSMEALELQGAISWEAPV
EEKTECIQKCKNNQTECFNFIRFLQPYNASHLYVCGTYAFQPKCTYVNMLTFTLEHGEFEDGKGKCPYDPAKG
HAGLLVDGELYSATLNNFLGTEPITLRNMCPHHSMKTEYLAFWLNEPHFVGSAYVPESVGSFTCDDDKVYFFF
RERAVESDCYAEQVVARVARVCKGDMGGARTLQRKWTTFLKARLACSAPNWQLYFNQLQAMHTLQDTSWHNTT
FFGVFQAQWGDMYLSATCEYQLEEIQRVFECPYKEYHEEAQKWDRYTDPVPSFRPGSCINNWHRRHCYTSSLE
LPDNILNFVKKHPLMEEQVGPRWSRPLLVKRGTNFTHLVADRVTGLDCATYTVLFICTGDGWLLRAVSLGRWV
HLIEELQLFDQEPMRSLVLSQSKKLLFAGSRSQLVQLPVADCMKYRSCADCVLARDPYCAWSVNTSRCVAVGG
HSGSLLIQHVMTSDTSGICNLRGSKKVRPTPKNITVVAGTDLVLPCHLSSNLAHARWTFGGRDLPAEQPGSFL
YDARLQALVVMAAQPRHAGAYHCFSEEQGARLAAEGYLVAVVAGPSVTLE

The disclosed amino acid sequence for MOL5d is 99% identical to Q9C0C4, June 2001, KIAA1739 PROTEIN—Homo scipiens (human), 963 aa (fragment). The amino acid sequence for MOL5d is also 99% identical to the 624 aa sequence with Accession number: AAB48378 disclosed in WO200078802-A2 with a priority date of Jun. 23, 1999. The amino acid sequence for MOL5d is also 98.9% identical to the 833 aa sequence with Accession number: AAE03640 disclosed in WO200142285-A2 with a priority date of Dec. 10, 1999.

MOL5d has been found to contain the following domains from the Pfam library: Sema domain, Plexin repeat, Immunoglobulin domain, Integrins, and beta chain domains.

Potential Role(s) of MOL5d in Tumorgenesis: Semaphorin are involved in neuronal axonal migration. Recently they have been associated with migration, invasion and apoptosis of tumor cells and endothelial cells. MOL5d (Semaphorin 4C-like protein) is overexpressed in the metastatic variant SW620 compared with SW480. It is also generally more expressed in cell lines derived from metastasis like the melanomas SK-Mel5 and HS688b and the liver met of gastric NCIN87. In the panel of tumor tissues, it is strongly induced in lung tumors and show overall overexpression in all other tumors. It potential role in tumorogenesis is likely to be to stimulate migration of tumor cells and therefore increase their ability to metastatize.

Impact of therapeutic targeting of MOL5d: Targeting with a human monoclonal antibody of MOL5d that results in an inhibition of the activity of this protein, preferably as it relates to its stimulation of migration and/or apoptotic/survival activity in tumor cells, specifically lung tumor cells, will have therapeutic effect on all solid tumor that depend on its activity, preferably on lung tumors.

### MOL5e

The disclosed novel semaphorin 4C-like nucleic acid of 1914 nucleotides, MOL5e, (also referred to as 170645595) is shown in Table 5J. An ORF begins with an AGA initiation codon at nucleotides 1-3 and ends with a GAG codon at nucleotides 1912-1914. The start and stop codons are in bold letters. Because the start and stop codons are not traditional initiation or termination codons, MOL5e could be a partial reading frame extending further in the 5′ and/or 3′ directions.

TABLE 5J

MOL5e Nucleotide Sequence

(SEQ ID NO:121)

AGATCTGCTGAGGTGTGGTGGAACCTTGTCCCCCGTAAGACAGTGTCTTCTGGGGACCTCGCCACGCTAGTACCG
CGGTTCTCCCAGACCCGCATCCAGGACTTCCTGACACTGACCCTGACGGAGCCCACTGGGCTTCTCTACCTGGGC
GCCCGAGAGGCCCTGTTTGCCTTCAGCATGGAGGCCCTGGAGCTGCAAGGACCGATCTCCTGGGAGCCCCCCGTG
GAGCAGAAGACTGAGTGTATCCAGAAAGGCAACPACAACCAGACCCACTCCTTCAACTTCATCCCCTTCCTGCAG
CCCTACAATGCCTCCCACCTGTACGTCTCTGGCACCTACGCCTTCCAGCCCAAGTCCACCTACCTCAACATGCTC
ACCTTCACTTTGGAGCATGGAGAGTTTGAAGATGGGAAGGGCAAGTGTCCCTATGACCCAGCTAAGGGCCATGCT
GGCCTTCTTGTGGATGCTCACCTGTACTCGGCCACACTCAACAACTTCCTGGGCACGGPACCCATTATCCTGCGT
AACATGGGGCCCCACCACTCCATGAAGACAGAGTACCTGGCCTTTTGGCTCAACGAACCTCACTTTGTAGGCTCT
GCCTATCTACCTGAGAGTGTGGCCAGCTTCACCCGGGACGACGACAAGGTCTACTTCTTCTTCAGGGACCGGGCA
GTGGAGTCCGACTGCTATCCCGAGCACGTGCTGGCTCGTGTGGCCCGTGTCTGCAACGCCGATATGGGGGGCCCA
CGGACCCTCCAGAGGAAGTGGACCACGTTCCTGAAGGCGCGCCTGGCATGCTCTGCCCCGAACTGGCAGCTCTAC
TTCAACCAGCTGCAGGCGATGCACACCCTGCAGGACACCTCCTGGCACAACACCACCTTCTTTGGGGTTTTTCAA
GCACAGTCGGGTGACATGTACCTGTCGGCCATCTGTGAGTACCACTTGGAAGAGATCCAGCGGGTGTTTGAGGGC
CCCTATAAGGAGTACCATGAGGAAGCCCAGAAGTGGCACCGCTACACTGACCCTGTACCCACCCCTCGGCCTGGC
TCGTGCATTAACAACTGGCATCCGCCCCACGGCTACACCAGCTCCCTGCAGCTACCCGACAACATCCTCAACTTC
GTCAAGAAGCACCCGCTGATGGAGGACCAGGTGGGGCCTCGGTGGAGCCCCCCCCTCCTCGTGAAGAAGGGCACC
AACTTCACCCACCTCGTGGCCGACCGGGTTACAGGACTTGATGGAGCCACCTATACACTGCTGTTCATTGGCACA
GGAGACGGCTGCCTGCTCAAGGCTCTGAGCCTGGGGCCCTGGGTTCACCTGATTCAGGAGCTGCAGCTGTTTGAC
CAGGAGCCCATGAGAAGCCTGGTGCTATCTCAGAGCAAGAAGCTGCTCTTTGCCGGCTCCCGCTCTCAGCTGGTG
CAGCTGCCCGTGCCCGACTGCATGAAGTATCGCTCCTGTGCACACTGTGTCCTCGCCCGGGACCCCTATTGCGCC
TGGACCGTCAACACCAGCCGCTGTGTGCCCCTGGGTGGCCACTCTGGATCTCTACTGATCCAGCATGTGATGACC
TCGGACACTTCAGGCATCTGCAACCTCCGTGGCAGTAAGAAAGTCAGGCCCACTCCCAAAAACATCACGGTGGTG
GCGGGCACAGACCTCGTGCTGCCCTGCCACCTCTCCTCCAACTTGGCCCATGCCCGCTGGACCTTTGGGGGCCCC
GACCTGCCTCCGGAACACCCCGGCTCCTTCCTCTACGATGCCCCGCTCCAGGCCCTGGTTCTGATGGCTGCCCAG
CCCCGCCATGCCGGGCCCTACCACTGCTTTTCAGAGGAGCACGGCGCGCGCCTGGCTGCTGAAGGCTACCTTGTC
GCTGTCCTGGCAGGCCCGTCGGTGACCTTGGAGCTCGAG

The reverse complement of MOL5e is shown in Table 5K.

TABLE 5K

MOLSe Rcverse Complement Nucleotide Sequence

(SEQ ID NO:122)

CTCGACCTCCAAGCTCACCGACGGGCCTGCCACGACAGCCACAAGGTAGCCTTCAGCACCCACCCGCGCCCCCTG
CTCCTCTGAAAAGCAGTGCTAGGCCCCGGCATGGCGGGGCTCGGCAGCCATCACPACCAGCGCCTGGAGCCGGGC
ATCGTAGAGGAAGGACCCGGGCTGTTCCGCAGGCAGGTCCCGGCCCCCAAAGGTCCAGCGGGCATGGGCCAAGTT
GGAGGACAGGTGCCAGGGCACCACCAGGTCTGTGCCCGCCACCACCGTGATGTTTTTCGGAGTGGGCCTGACTTT
CTTACTGCCACGGAGCTTCCACATGCCTGAAGTGTCCCAGGTCATCACATCCTGGATCAGTAGAGATCCAGACTC
GCCACCCACGGCCACACAGCGGCTGGTGTTGACCCTCCACGCGCAATAGGGGTCCCGGCCGACGACACAGTCTGC
ACAGGAGCGATACTTCATCCACTCCCCCACGCCCAGCTGCACCAGCTGAGAGCCGGACCCCGCAAAGAGCACCTT
CTTGCTCTGAGATAGCACCAGGCTTCTCATGGGCTCCTGGTCAAACAGCTGCAGCTCCTCAATCAGGTGAACCCA
GGGCCCCAGGCTCACAGCCTTGAGCAGCCAGCCGTCTCCTGTGCCAATGAACAGCACTGTATAGGTGGCTCCATC
AAGTCCTGTAACCCCGTCGGCCACCACGTCGGTGAAGTTGGTGCCCTTCTTCACGAGCACCGGGCCCCTCCACCG
AGGCCCCACCTGCTCCTCCATCAGCGCGTGCTTCTTGACGAAGTTGAGGATCTTGTCGCGTACCTCCAGGGACCT
GGTGTAGCCGTCGCGCCGATGCCAGTTGTTAATGCACGACCCAGGCCGAGGGCTGGGTACAGGGTCACTGTAGCG
GTCCCACTTCTGCCCTTCCTCATGGTACTCCTTATAGGGGCCCTCAAACACCCGCTGGATCTCTTCCAACTGGTA
CTCACAGATGGCCGACAGGTACATGTCACCCCACTGTGCTTGAAAAACCCCAAAGAAGGTGGTGTTGTGCCAGGA
GGTGTCCTGCAGGCTGTGCATCGCCTGCAGCTGGTTGAAGTAGAGCTGCCAGTTCGGGGCACAGCATGCCAGCCG
CCCCTTCAGGAACGTGGTCCACTTCCTCTGCAGGGTCCCTGCGCCCCCCATATCGCCCTTGCAGACACGCCCCAC
ACGAGCCACCACCTGCTCGGCATAGCAGTCGGACTCCACTGCCCGCTCCCTGAAGAAGAAGTAGACCTTGTCGTC
CTCCCCCGTCAAGCTGCCCACACTCTCAGGTACATAGGCAGAGCCTACAAAGTGAGGTTCGTTGACCCAAAACGC
CACGTACTCTGTCTTCATGGAGTGCTCGGGCCCCATGTTACGCAGGATAATGGGTTCCGTGCCCAGGAAGTTCTT
GAGTGTGGCCCAGTACACCTCACCATCCACAAGAAGCCCAGCATGGCCCTTAGCTGGGTCATAGCCACACTTGCC
CTTCCCATCTTCAAACTCTCCATGCTCCAAAGTGAAGGTGAGCATGTTGACGTAGGTGCACTTGGGCTGGAAGGC
GTAGGTGCCACAGACGTACAGGTGGGAGGCATTGTAGGGCTGCAGGAAGCGGATGAAGTTGAAGCACTCGGTCTG
GTTGTTCTTCCCTTTCTGGATACACTCAGTCTTCTCCTCCACGGGGGCCTCCCAGCAGATCGCTCCTTGCAGCTC
CACGGCCTCCATGCTCAAGGCAAACAGCGCCTCTCGGCCGCCCACGTACAGAAGCCCAGTGCGCTCCGTCAGCGT
CAGTGTCAGGAAGTCCTCGATGCCGGTCTGGGAGAACCGCCGTACTACCGTCGCCAGCTCCCCAGAAGACACTGT
CTTACGCGGCACAACGTTCCACCACACCTCAGCAGATCT

The MOL5e protein encoded by SEQ ID NO:123 has 638 amino acid residues and is presented using the one-letter code in Table 5L.

TABLE 5L
Encoded MOL5e protein sequence

(SEQ ID NO:123)

RSAEVWWNLVPRKTVSSGELATVVRRFSQTGIQDFLTLTLTEPTGLLYVGAREALFAFSMEALELQGAISWEA
PVEEKTECIQKGKNNQTECFNFIRFLQPYNASHLYVCGTYAFQPKCTYVNMLTFTLEHGEFEDGKGKCPYDPA
KGHAGLLVDGELYSATLNNFLGTEPIILRNMGPHHSMKTEYLAFWLNEPHFVGSAYVPESVGSFTGDDDKVYF
FFRERAVESDCYAEQVVARVARVCKGDMGGARTLQRKWTTFLKARLACSAPNWQLYFNQLQAMHTLQDTSWHN
TTFFGVFQAQWGDMYLSAICEYQLEEIQRVFEGPYKEYHEEAQKWDRYTDPVPSPRPGSCINNWHRRHGYTSS
LELPDNILNFVKKHPLMEEQVGPRWSRPLLVKKGTNFTHLVADRVTGLDGATYTVLFIGTGDGWLLKAVSLGP
WVHLIEELQLFDQEPMRSLVLSQSKKLLFAGSRSQLVQLPVADCMKYRSCADCVLARDPYCAWSVNTSRCVAV
GGHSGSLLIQHVMTSDTSGICNLRGSKKVRPTPKNITVVAGTDLVLPCHLSSNLAHARWTFGGRDLPAEQPGS
FLYDARLQALVVMAAQPRHAGAYHCFSEEQGARLAAEGYLVAVVAGPSVTLELE

### MOL5f

The disclosed novel semaphorin 4C-like nucleic acid of 1914 nucleotides, MOL5f, (also referred to as 170645599) is shown in Table 5M. An ORF begins with an AGA initiation codon at nucleotides 1-3 and ends with a GAG codon at nucleotides 1912-1914. The start and stop codons are in bold letters. Because the start and stop codons are not traditional initiation or termination codons, MOL5f could be a partial reading frame extending further in the 5′ and/or 3′ directions.

TABLE 5M
MOL5f Nucleotide Sequence

(SEQ ID NO:124)

AGATCTGCTGAGGTGTGGTGGAACCTTGTGCCGCGTAAGACAGTGTCTTCTGGGGAGCTGGCCACGGTAGTACGG
CGGTTCTCCCAGACCGGCATCCAGGACTTCCTGACACTGACGCTGACGGAGCCCACTGGGCTTCTGTACGTGGGC
GCCCGAGAGGCCCTGTTTGCCTTCAGCATGGAGGCCCTGGAGCTGCAAGGAGCGATCTCCTGGGAGGCCCCCGTG
GAGAAGAAGACTGAGTGTATCCAGAAAGGGAAGAACAGCCAGACCGAGTGCTTCAACTTCATCCGCTTCCTGCAG
CCCTACAATGCCTCCCACCTGTACGTCTGTGGCACCTACGCCTTCCAGCCCAAGTGCACCTACGTCAACATGCTC
ACCTTCACTTTGGAGCATGGAGAGTTTGAAGATGGGAAGGGCAAGTGTCCCTATGACCCAGCTAAGGGCCATGCT
GGCCTTCTTGTGGATGGTGAGCTGTACTCGGCCACACTCAACAACTTCCTGGGCACGGAACCCATTATCCTGCGT
AACATGGGGCCCCACCACTCCATGAAGACAGAGTACCTGGCCTTTTGGCTCAACGAACCTCACTTTGTAGGCTCT
GCCTATGTACCTGAGAGTGTGGGCAGCTTCACGGGGGACGACGACAAGGTCTACTTCTTCTTCAGGGAGCGGGCA
GTGGAGTCCGACTGCTATGCCGAGCAGGTGGTGGCTCGTGTGGCCCGTGTCTGCAAGGGCGATATGGGGGGCGCA
CGGACCCTGCAGAGGAAGTGGACCACGTTCCTGAAGGCGCGGCTGGCATGCTCTGCCCCGAACTGGCAGCTCTAC
TTCAACCAGCTGCAGGCGATGCACACCCTGCAGGACACCTCCTGGCACAACACCACCTTCTTTGGGGTTTTTCAA
GCACAGTGGGGTGACATGTACCTGTCGGCCATCTGTGAGTACCAGTTGGAAGAGATCCAGCGGGTGTTTGAGGGC
CCCTATAAGGAGTACCATGAGGAAGCCCAGAAGTGGGACCGCTACACTGACCCTGTACCCAGCCCTCGGCCTGGC
TCGTGCATTAACAACTGGCATCGGCGCCACGGCTACACCAGCTCCCTGGAGCTACCCGACAACATCCTCAACTTC
GTCAAGAAGCACCCGCTGATGGAGGAGCAGGTGGGGCCTCGGTGGAGCCGCCCCCTGCTCGTGAAGAAGGGCACC
AACTTCACCCACCTGGTGGCCGACCGGGTTACAGGACTTGATGGAGCCACCTATACAGTGCTGTTCATTGGCACA
GGAGACGGCTGGCTGCTCAAGGCTGTGAGCCTGGGGCCCTGGGTTCACCTGATTGAGGAGCTGCAGCTGTTTGAC
CAGGAGCCCATGAGAAGCCTGGTGCTATCTCAGAGCAAGAAGCTGCTCTTTGCCGGCTCCCGCTCTCAGCTGGTG
CAGCTGCCCGTGGCCGACTGCATGAAGTATCGCTCCTGTGCAGACTGTGTCCTCGCCCGGGACCCCTATTGCGCC
TGGAGCGTCAACACCAGCCGCTGTGTGGCCGTGGGTGGCCACTCTGGATCTCTACTGATCCAGCATGTGATGACC
TCGGACACTTCAGGCATCTGCAACCTCCGTGGCAGTAAGAAAGTCAGGCCCACTCCCAAAAACATCACGGTGGTG
GCGGGCACAGACCTGGTGCTGCCCTGCCACCTCTCCTCCAACTTGGCCCATGCCCGCTGGACCTTTGGGGGCCGG
GACCTGCCTGCGGAACAGCCCGGGTCCTTCCTCTACGATGCCCGGCTCCAGGCCCTGGTTGTGATGGCTGCCCAG
CCCCGCCATGCCGGGGCCTACCACTGCTTTTCAGAGGAGCAGGGGGCGCGGCTGGCTGCTGAAGGCTACCTTGTG
GCTGTCGTGGCAGGCCCGTCGGTGACCTTGGAGCTCGAG

The MOL5f protein encoded by SEQ ID NO:125 has 638 amino acid residues and is presented using the one-letter code in Table 5N.

TABLE 5N
Encoded MOL5f protein sequence

(SEQ ID NO:125)

RSAEVWWNLVPRKTVSSGELATVVRRFSQTGIQDFLTLTLTEPTGLLYVGAREALFAFSMEALELQGAISWEA
PVEKKTECIQKGKNSQTECFNFIRFLQPYNASHLYVCGTYAFQPKCTYVNMLTFTLEHGEFEDGKGKCPYDPA
KGHAGLLVDGELYSATLNNFLGTEPIILRNMGPHHSMKTEYLAFWLNEPHFVGSAYVPESVGSFTGDDDKVYF
FFRERAVESDCYAEQVVARVARVCKGDMGGARTLQRKWTTFLKARLACSAPNWQLYFNQLQAMHTLQDTSWHN
TTFFGVFQAQWGDMYLSAICEYQLEEIQRVFEGPYKEYHEEAQKWDRYTDPVPSPRPGSCINNWHRRHGYTSS
LELPDNILNFVKKHPLMEEQVGPRWSRPLLVKKGTNFTHLVADRVTGLDGATYTVLFIGTGDGWLLKAVSLGP
WVHLIEELQLFDQEPMRSLVLSQSKKLLFAGSRSQLVQLPVADCMKYRSCADCVLARDPYCAWSVNTSRCVAV
GGHSGSLLIQHVMTSDTSGICNLRGSKKVRPTPKNITVVAGTDLVLPCHLSSNLAHARWTFGGRDLPAEQPGS
FLYDARLQALVVMAAQPRHAGAYHCFSEEQGARLAAEGYLVAVVAGPSVTLELE

Table 5O shows a ClustalW alignment of the MOL5 variants.

[image]

[image]

[image]

MOL5a also has homology to other proteins as shown in BLAST alignment results in Table 5P

TABLE 5P
BLAST results for MOL5a

| Gene Index/ | | Length | Identity | Positives | |
|---|---|---|---|---|---|
| Identifier | Protein/Organism | (aa) | (%) | (%) | Expect |
| gi\|12698023\|dbj\| | KIAA1739 protein | 963 | 785/801 | 789/801 | 0.0 |
| BAB21830.1\| (AB051526) | [Homo sapiens] | | (98%) | (98%) | |
| gi\|8134699\|sp\|Q64151\| | SEMAPHORIN 4C | 834 | 722/834 | 765/834 | 0.0 |
| SM4C_MOUSE | PRECURSOR | | (86%) | (91%) | |
| | (SEMAPHORIN I) | | | | |
| | (SEMA I) | | | | |
| | (SEMAPHORIN C- | | | | |
| | LIKE 1) (M-SEMA | | | | |
| | F) | | | | |
| gi\|13637386\|ref\| | hypothetical | 510 | 510/510 | 510/510 | 0.0 |
| XP_002614.2\| | protein FLJ20369 | | (100%) | (100%) | |
| | [Homo sapiens] | | | | |
| gi\|8923346\|ref\| | sema domain, | 510 | 509/510 | 509/510 | 0.0 |
| NP_060259.1\| | immunoglobulin | | (99%) | (99%) | |
| | domain (Ig), | | | | |
| | transmembrane | | | | |
| | domain TM; | | | | |
| | cytokeratin 14; | | | | |
| | adipocyte-derived | | | | |
| | leucine | | | | |
| | aminopeptidase; | | | | |
| | hypothetical | | | | |
| | protein MGC10851; | | | | |
| | hypothetical | | | | |
| | protein FLJ14662; | | | | |
| | sphingomyelin | | | | |
| | phosphodiesterase | | | | |
| | -1, acid | | | | |
| | lysosomal; Pro- | | | | |
| | platelet ba> | | | | |
| gi\|13633937\|sp\| | SEMAPHORIN 4G | 838 | 292/673 | 381/673 | e−138 |
| Q9NTN9\|SM4G_HUMAN | PRECURSOR | | (43%) | (56%) | |

This information is presented graphically in the multiple sequence alignment given in Table 5Q (with MOL5a being shown on line 1, and MOL5b on line 2) as a ClustalW analysis comparing MOL5 with related protein sequences.

[image]

[image]

[image]

[image]

MOL5b and MOL5c share close homology to each other and therefore to other proteins as is shown in the BLAST alignment in Table 5R

[image]

[image]

[image]

[image]

[image]

As used herein, any reference to MOL5 encompasses MOL5a, MOL5b, and MOL5c, unless otherwise indicated.

Table 5S and 5T list the domain descriptions from DOMAIN analysis results against MOL5. The region from amino acid residue 66 through 487 (SEQ ID NO:14) most probably (E=3e⁻¹²⁵) contains a Sema domain found in Semaphorins, described above under MOL4, and aligned here in Table 5S The region from amino acid residue 562 through 627 (SEQ ID NO:14) most probably (E=1e⁻⁴) also contains a Sema domain found in Semaphorins, aligned here in Table 5T This indicates that the MOL5 sequence has properties similar to those of other proteins known to contain this domain.

[image]

[image]

The protein similarity information, expression pattern, cellular localization, and map location for MOL5 suggest that this Semaphorin 4C-like protein may have important structural and/or physiological functions characteristic of the Semaphorin family. These functions include growth cone guidance, axonal pathfindin, and embryonic development. Therefore, the MOL5 nucleic acids and proteins are useful in potential diagnostic and therapeutic applications and as a research tool. These include serving as a specific or selective nucleic acid or protein diagnostic and/or prognostic marker, wherein the presence or amount of the nucleic acid or the protein are to be assessed. These also include potential therapeutic applications such as the following: (i) a protein therapeutic, (ii) a small molecule drug target, (iii) an antibody target (therapeutic, diagnostic, drug targeting/cytotoxic antibody), (iv) a nucleic acid useful in gene therapy (gene delivery/gene ablation), (v) an agent promoting tissue regeneration in vitro and in vivo, and (vi) a biological defense weapon.

The MOL5 nucleic acids and proteins have applications in the diagnosis and/or treatment of various diseases and disorders. For example, the compositions of the present invention will have efficacy for the treatment of patients suffering from: Rheumatoid arthritis (RA), CNS disorders, Alzheimer, Down syndrome, Schizophrenia, Parkinsons diseases as well as other diseases, disorders and conditions.

MOL5 is a Semaphorin 4C like protein. Semaphorin 4C (S4C, previously called M-SemaF) was recently identified as a brain rich transmembrane member of semaphorin family of the vertebrate. In the cytoplasmic domain of S4C there is a proline-rich region suggesting that the cytoplasmic domain may play an important role in Sema4C function. The cytoplasmic domain (cd) of M-SemaF(S4C)-associating protein has been identified with a MW of 75 kDa, named SFAP75, from mouse brain. SFAP75 turned out to be the same as the recently reported neurite-outgrowth-related protein named Norbin. Deletion mutants analyses of S4C and SFAP75 have revealed that the membrane-proximal region of S4Ccd binds to the intermediate region of SFAP75. Western blot and immunohistochemical analyses with anti-Sema4C and anti-SFAP75 antibodies indicated that S4C and SFAP75 were specially enriched in the brain with a similar distribution pattern to each other. These results suggest that S4C interacts with SFAP75 and plays a role in neural function in brain.

Semaphorins are also known to act as chemorepulsive molecules that guide axons during neural development. Sema4C, a group 4 semaphorin, is a transmembrane semaphorin of unknown function. The cytoplasmic domain of Sema4C contains a proline-rich region that may interact with some signaling proteins. It has been demonstrated that Sema4C is enriched in the adult mouse brain and associated with PSD-95 isoforms containing PDZ (PSD-95/DLG/ZO-1) domains, such as PSD-95/SAP90, PSD-93/chapsin110, and SAP97/DLG-1, which are concentrated in the post-synaptic density of the brain. In the neocortex, S4C is enriched in the synaptic vesicle fraction and Triton X-100 insoluble post-synaptic density fraction. Immunostaining for Sema4C overlaps that for PSD-95 in superficial layers I-IV of the neocortex. In neocortical culture, S4C is colocalized with PSD-95 in neurons, with a dot-like pattern along the neurites. Sema4C thus may function in the cortical neurons as a bi-directional transmembrane ligand through interacting with PSD-95.

These materials are further useful in the generation of antibodies that bind immuno-specifically to the novel MOL5 substances for use in therapeutic or diagnostic methods. These antibodies may be generated according to methods known in the art, using prediction from hydrophobicity charts, as described in the “Anti-MOLX Antibodies” section below. The disclosed MOL5 protein has multiple hydrophilic regions, each of which can be used as an immunogen. In one embodiment, a contemplated MOL5 epitope is from about amino acids 30 to 70. In another embodiment, a MOL5 epitope is from about amino acids 100 to 150. In additional embodiments, MOL5 epitopes are from about amino acids 175 to 200, 220 to 450, 550 to 575, 590 to 610, and from about amino acids 675 to 850. These novel proteins can also be used to develop assay systems for functional analysis.

### MOL6

The disclosed novel kappa casein precursor-like MOL6 nucleic acid of 603 nucleotides (also referred to as GMAC060288_A) is shown in Table 6A. An open reading begins with an ATG initiation codon at nucleotides 31-33 and ends with a TAA codon at nucleotides 574-576. A putative untranslated region upstream from the initiation codon and downstream from the termination codon are underlined in Table 6A, and the start and stop codons are in bold letters.

TABLE 6A
MOL6 Nucleotide Sequence

(SEQ ID NO:19)

TTTTTTTTAAATTTATCTTTAGGTGCAATAATGAAGAGTTTTCTTCTAGTTGTCAATGCCCTGGCATTAACCCTG
CCTTTTTTGCTAGTGGAGGTTCAAAACCAGAAACAACCAGCATGCCATGAGAATGATGAAAGACCATTCTATCAG
AAAACGTTCACATATGTCCCAATGTATTATGTGCAAAATAGCTATCTTTATTATGGACCCAATTTGTACAAACGT
AGACCAGCTATAGCATTAAATAATCAATATGGGCTTCGCACATATTATGCAACCCAAGCTGTAGTTAGGGCACAT
GCCCAAATTCCTCAGCGGCAATACCTGCCAAATAGCCACCACACTGTGGTACGTCGCCCAAACCTGCATCCATCA
TTTATTGCAATCCCCCCAAAGAAAATTCAGGATAAAATAATCATCCCTACCATCAATACCATTGCTACTGTTGAA
CCTACACCAGCTCCTGCCACTGAACCAACGGTGGACAGTGTAATCACTCCAGAAGCTTTTTCAGAGTCCATCATC
ACGAGCACCCCTGAGACAACCACAGTTGCAGTTACTCCACCTACGGCATAAAAACACCAAGGAAATATCAAAGAA
CAC

The MOL6 protein encoded by SEQ ID NO:20 has 181 amino acid residues, and is presented using the one-letter code in Table 6B (SEQ ID NO:20). The Psort profile for MOL6 predicts that this sequence has a signal peptide and is likely to be localized outside the cell with a certainty of 0.8200. The most likely cleavage site for a peptide is between amino acids 24 and 25: VQN-QK based on the SignalP result. The molecular weight of the MOL6 protein is 20424.3 Daltons.

TABLE 6B
Encoded MOL6 protein sequence.

(SEQ ID NO:20)

MKSFLLVVNALALTLPFLLVEVQNQKQPACHENDERPFYQKTFTYVPMYYVQNSYLYYGPNLYKRRPAIALNNQYG
LRTYYATQAVVRAHAQIPQRQYLPNSHHTVVRRPNLHPSFIAIPPKKIQDKIIIPTINTIATVEPTPAPATEPTVD
SVITPEAFSESIITSTPETTTVAVTPPTA

The disclosed nucleic acid sequence has 566 of 586 bases (96%) identical to a Homo sapiens kappa casein precursor mRNA (GENBANK-ID: ACC: I29004) (E value=9.8e−¹¹⁶).

The full amino acid sequence of MOL6 was found to have 165 of 182 amino acid residues (90%) identical to, and 168 of 182 residues (92%) positive with, the 182 amino acid residue kappa casein precursor protein from Homo sapiens (ptnr: SWISSPROT-ACC:P07498) (E value=3.0e−⁸³), 165 of 182 amino acid residues (90%) identical to, and 168 of 182 residues (92%) positive with patp:AAR39351 Recombinant human kappa casein—Homo sapiens having 182 aa (E value=3.0e−⁸³), and 165 of 182 amino acid residues (90%) identical to, and 168 of 182 residues (92%) positive with patp:AAR92150 Human milk kappa-casein having 182 amino acids (E value=3.0e−⁸³).

The global sequence homology (as defined by FASTA alignment with the full length sequence of this protein) is 92.265% amino acid homology and 91.160% amino acid identity. In addition, this protein contains the following protein domains (as defined by Interpro) at the indicated nucleotide positions: casein_kappa (IPR000117) at amino acid positions1 to 181.

The full amino acid sequence of MOL6 was found to have homology with several proteins, including those disclosed in the BLASTP data in Table 6C.

TABLE 6C
BLAST results for MOL6

| Gene Index/ | | | | Positives | |
|---|---|---|---|---|---|
| Identifier | Protein/Organism | Length (aa) | Identity (%) | (%) | Expect |
| gi\|1705606\| | KAPPA CASEIN | 182 | 154/171 | 157/171 | 3e−59 |
| sp\|P07498\| | PRECURSOR | | (90%) | (91%) | |
| CASK_HUMAN | | | | | |
| gi\|4885161\|ref\| | casein, kappa | 182 | 153/171 | 156/171 | 9e−59 |
| NP_005203.1\| | [Homo sapiens] | | (89%) | (90%) | |
| gi\|186655\|gb\| | kappa-casein | 182 | 153/171 | 156/171 | 6e−55 |
| AAA59456.1\| | [Homo sapiens] | | (89%) | (90%) | |
| gi\|13633560\|ref\| | casein, kappa | 182 | 144/171 | 147/171 | 3e−54 |
| XP_003538.3\| | [Homo sapiens] | | (84%) | (85%) | |
| gi\|2493502\|sp\|P7913 | KAPPA CASEIN | 182 | 102/178 | 118/178 | 2e−31 |
| 9\|CASK_CAMDR | PRECURSOR | | (57%) | (65%) | |

This information is presented graphically in the multiple sequence alignment given in Table 6D (with MOL6 being shown on line 1) as a ClustalW analysis comparing MOL6 with related protein sequences.

[image]

[image]

Table 6E lists the domain description from DOMAIN analysis results against MOL6. The region from amino acid residue 1 through 116 (SEQ ID NO:20) most probably (E=2e⁻³⁶) contains a casein kappa domain found in Kappa casein, aligned here in Table 6E. This indicates that the MOL6 sequence has properties similar to those of other proteins known to contain this domain.

[image]

The above defined information for MOL6 suggests that this kappa casein precursor-like protein may function as a member of a “Kappa Casein Precursor family”. Members of this family is found as a nutritional component of human milk. Therefore, the novel nucleic acids and proteins identified here may be useful in potential therapeutic applications implicated in (but not limited to) various pathologies and disorders as indicated below. The potential therapeutic applications for MOL6 include, but are not limited to: protein therapeutic, small molecule drug target, antibody target (therapeutic, diagnostic, drug targeting/cytotoxic antibody), diagnostic and/or prognostic marker, kappa casein precursor therapy (kappa casein precursor delivery/kappa casein precursor ablation), research tools, tissue reKappa Casein Precursor ration in vivo and in vitro of all tissues and cell types composing (but not limited to) those defined here.

The MOL6 nucleic acids and proteins are useful in potential therapeutic applications implicated in nutritional deficiencies. It is used as a nutrient supplement in milk based products to provide a substantial improvement of the nutritional and biological value of the formulae, making it closer in similarity to human milk. Kappa casein can also be used as a pharmaceutical and/or other pathologies and disorders. For example, a cDNA encoding the kappa casein precursor-like protein may be useful in kappa casein precursor therapy, and the kappa casein precursor-like protein may be useful when administered to a subject in need thereof. By way of nonlimiting example, the compositions of the present invention will have efficacy for treatment of patients suffering from nutritional deficiencies. MOL6, or fragments thereof, may further be useful in diagnostic applications, wherein the presence or amount of the nucleic acid or the protein are to be assessed.

These materials are further useful in the generation of antibodies that bind immuno-specifically to the novel MOL6 substances for use in therapeutic or diagnostic methods. These antibodies may be generated according to methods known in the art, using prediction from hydrophobicity charts, as described in the “Anti-MOLX Antibodies” section below. The disclosed MOL6 protein has multiple hydrophilic regions, each of which can be used as an immunogen. In one embodiment, a contemplated MOL6 epitope is from about amino acids 30 to 125. In another embodiment, a MOL6 epitope is from about amino acids 140 to 160. These novel proteins can also be used to develop assay systems for functional analysis.

### MOL7

A novel nucleic acid encoding a human Rh type B glycoprotein-like-protein MOL7 was identified by TblastN using CuraGen Corporation's sequence file for MOL7 probes or homologs, and run against the Genomic Daily Files made available by GenBank. The disclosed novel MOL7 nucleic acid of 1765 nucleotides (also referred to as AF193808A) is shown in Table 7A. An open reading frame begins with an ATG initiation codon at nucleotides 39-41 and ends with a TAA codon at nucleotides 1383-1385. A putative untranslated region upstream from the initiation codon and downstream from the termination codon are underlined in Table 7A, and the start and stop codons are in bold letters.

TABLE 7A
MOL7 Nucleotide Sequence

(SEQ ID NO:21)

AAAGCCTGCGAGCGCCAGCCGAGATCGCATCCCAACCCATGGCCGGGTCTCCTAGCCGCGCCGCGGGCCGGCGACTGC
AGCTTCCCCTGCTGTGCCTCTTCCTCCAGGGCGCCACTGCCGTCCTCTTTGCTGTCTTTGTCCGCTACAACCACAAAA
CCGACGCTGCCCTCTGGCACCGGAGCAACCACAGTAACGCGGACAATGAATTTTACTTTCGCTACCCAAGTTTCCAGG
ACGTGCATGCCATGGTCTTCGTGGGCTTTGACTTCCTCATGGTCTTCCTGCAGCGTTACGGCTTCAGCAGCGTGGGCT
TCACCTTCCTCCTGGCCGCCTTTGCCCTGCAGTGGTCCACACTGGTCCAGGGCTTTCTCCACTCCTTCCACGGTGGCC
ACATCCATGTTGGCGTGGAGAGCATGATCAATGCTGACTTTTGTGCGGGGGCCGTGCTCATCTCCTTTGGTGCCGTCC
TGGGCAAGACCGGGCCTACCCAGCTGCTGCTCATGGCCCTGCTGGAGGTGGTGCTGTTTGGCATCAATGAGTTTGTGC
TCCTTCATCTCCTGGGGGTGAGAGTCTGGGGAGGGATTTCTAGGGTTATGTCTAGTACCATGCTGGAGAAGAGCAAGC
ACCGCCAGGGCTCCGTCTACCATTCAGACCTCTTCGCCATGATTGGTGGGACCATCTTCCTGTGGATCTTCTGGCCTA
GCTTCAATGCTGCACTCACAGCGCTGGGGGCTGGGCAGCATCGGACGGCCCTCAACACATACTACTCCCTGGCTGCCA
GCACCCTTGGCACCTTTGCCTTGTCAGCCCTTGTAGGGGAAGATGGGAGGCTTGACATGGTAGTCCACATCCAAAATG
CAGCGCTGGCTGGAGGGGTTGTGGTGGGGACCTCAAGTGAAATGATGCTGACACCCTTTGGGGCTCTGGCAGCTGGCT
TCTTGGCTGGGACTGTCTCCACGCTGGGGTACAAGTTCTTCACGCCCATCCTTGAATCAAAATTCAAAGTCCAAGACA
CATGTGGAGTCCACAACCTCCATGGGATGCCGGGGGTCCTGGGGGCCCTCCTGGGGGTCCTTGTGGCTGGACTTGCCA
CCCATGAAGCTTACGGAGATGGGCTGGAGAGTGTGTTTCCACTCATAGCCGAGGGCCAGCGCAGTGCCACGTCACAGG
CCATGCACCAGCTCTTCGGGCTGTTTGTCACACTGATGTTTGCCTCTGTGGGCGGGGGCCTTGGAGGTGGGCTCCTGC
TGAAGCTACCCTTTCTGGACTCCCCCCCCGACTCCCAGCACTACGAGGACCAAGTTCACTGGCAGGTGGTGCCTGGCG
AGCATGAGGATAAAGCCCAGAGACCTCTGAGGGTGGAGGAGGCAGACACTCAGGCCTAACCCACTGCCAGCCCCTGAG
AGGACACGCTCCTTTTCGAAGATGCTGACTGGCTGCTACTAGGAAGTTCTTTTTGAGCTCCCATTCCTCCAGCTGCAA
GAAGGGAGCCATGAGCCAGAAGGAGGCCCCTTTCCACAGGCAGCGTCTCCACAGGGAGAGGGGCAACAGGAGGCTGGG
AAATGGTGGGGAGTGGGGCCGTAACTGGGTACAATAGGGGGAACCTCACCAGATGCCCAACCCGACTGCCCTACCAGC
CTGCACATGGGTAGAAGAGGCCAAATTGAGGCACCCAAGTGATCCACTGGCCCCACGTCACACAGTTACAGTGAAGCC
CAAGCCAGGCCTGGTTGAGGGTGATAAACGCCACTGTCTTTAAGGAAAA

The MOL7 protein encoded by SEQ ID NO:21 has 448 amino acid residues, and is presented using the one-letter code in Table 7B (SEQ ID NO:22). The SignalP, Psort and/or Hydropathy profile for MOL7 predict that MOL7 has a signal peptide and is likely to be localized at the plasma membrane with a certainty of 0.6400. The SignalP shows a signal sequence is coded for with the most likely cleavage site being between amino acids 27 and 28: ATA-VL. This is typical of this type of membrane protein. The molecular weight of the MOL7 protein is 48304.3 Daltons.

TABLE 7B
Encoded MOL7 protein sequence.

| | |
|---|---|
| MAGSPSRAAGRRLQLPLLCLFLQGATAVLFAVFVRYNHKTDAALWHRSNHSNADNEFYFRYPSFQDV | (SEQ ID NO:22) |
| HAMVFVGFDFLMVFLQRYGFSSVGFTFLLAAFALQWSTLVQGFLHSFHGGHIHVGVESMINADFCAG | |
| AVLISFGAVLGKTGPTQLLLMALLEVVLFGINEFVLLHLLGVRVWGGISRVMSSTMLEKSKHRQGSV | |
| YHSDLFAMIGGTIFLWIFWPSFNAALTALGAGQHRTALNTYYSLAASTLGTFALSALVGEDGRLDMV | |
| VHIQNAALAGGVVVGTSSEMMLTPFGALAAGFLAGTVSTLGYKFFTPILESKFKVQDTCGVHNLHGM | |
| PGVLGALLGVLVAGLATHEAYGDGLESVFPLIAEGQRSATSQAMHQLFGLFVTLMFASVGGGLGGGL | |
| LLKLPFLDSPPDSQHYEDQVHWQVVPGEHEDKAQRPLRVEEADTQA | |

The nucleic acid sequence of MOL7 was found to have 680 of 815 bases (83) identical to a mouse Rh type b glycoprotein mRNA (GENBANK-ID:AF1938081|acc:AF193808).

The full amino acid sequence of MOL7 was found to have 363 of 448 amino acid residues (81%) identical to, and 399 of 448 residues (89%) positive with, the 455 amino acid residue mouse RH TYPE B GLYCOPROTEIN (ptnr: SPTREMBL-ACC:Q9QXP1)

The full amino acid sequence of MOL7 was found to have homology with several proteins, including those disclosed in the BLASTP data in Table 7C.

TABLE 7C
BLAST results for MOL7

| Gene Index/ | | Length | Identity | Positives | |
|---|---|---|---|---|---|
| Identifier | Protein/Oragnism | (aa) | (%) | (%) | Expect |
| gi\|9966891\|ref\|NP_065140.1\| | Rh type B | 458 | 433/462 | 435/462 | 0.0 |
| | glycoprotein | | (93%) | (93%) | |
| | [Homo sapiens] | | | | |
| gi\|14346006\|gb\|AKK15395.1\| | Rh type B | 458 | 429/462 | 431/462 | 0.0 |
| (AY013268) | glycoprotein [Pan | | (92%) | (92%) | |
| | troglodytes] | | | | |
| gi\|14486159\|gb\|AAK14651.1\| | Rh type B | 458 | 384/462 | 409/462 | 0.0 |
| (AY013261) | glycoprotein [Sus | | (83%) | (88%) | |
| | scrofa] | | | | |
| gi\|10946710\|ref\|NP_067350.1\| | Rhesus blood | 455 | 362/452 | 398/452 | e-177 |
| | froup-associated | | (80%) | (87%) | |
| | B glycoprotein; | | | | |
| | Rh type B | | | | |
| | glycoprotein [Mus | | | | |
| | musculus] | | | | |
| gi\|14486161\|gb\|AAK14652.1\| | Rh type B | 458 | 373/462 | 404/462 | e-176 |
| (AY013262) | glycoprotein | | (80%) | (86%) | |
| | cuniculus] | | | | |

This information is presented graphically in the multiple sequence alignment given in Table 7D (with MOL7 being shown on line 1) as a ClustalW analysis comparing MOL7 with related protein sequences.

[image]

[image]

[image]

Table 7E lists the domain description from DOMAIN analysis results against MOL7. The region from amino acid residue 25 through 336 (SEQ ID NO:22) most probably (E=1e⁻³³) contains an ammonium transporter domain found in Ammonium transporters, aligned here in Table 7E. This indicates that the MOL7 sequence has properties similar to those of other proteins known to contain this domain.

[image]

[image]

### TaqMan Data

Example 2 shows a TaqMan expression profile in 41 normal human tissues and 55 human cancer cell lines. The MOL7 gene is expressed in normal tissues, specifically lung, colon, small intestine, and prostate, and is lost in cancer cell lines.

Example 2 also shows replicate TaqMan expression results in tumor tissues that are often matched with normal adjacent tissue (NAT), as defined by the operating surgeon. The results reveal that the MOL7 human Rh type B glycoprotein is overexpressed in kidney tumors compared with their NAT and normal tissues.

### Chromosomal localization:

This gene belongs to genomic DNA GenBank AL139130 which maps to chromosome 1.

### Tissue expression:

MOL7 has been found to be expressed in Renal clear cell carcinoma by EST analysis. Genbank EST AI310325 has 100% identity with novel Rh type B glycoprotein and was obtained from 2 pooled tumors (clear cell type). Kidney, AI925934 has 100% identity with novel Rh type B glycoprotein and was obtained from Kidney. Fetal spleen R83833 and AI022447 have 96% identity to novel Rh type B glycoprotein and were obtained from Fetal spleen. The tissue expression profile of was also determined by TaqMan.

### Uses of the Compositions of the Invention

The expression pattern, map location and protein similarity information for the MOL7 suggest that this gene may function as “an Rh family” member. Therefore, the MOL7 nucleic acids and proteins are useful in potential therapeutic applications implicated in various pathologies/disorders described and/or other pathologies/disorders

Potential therapeutic uses for MOL7 include: Protein therapeutic, Small molecule drug target, Antibody target (Therapeutic, Diagnostic, Drug targeting/Cytotoxic antibody), Diagnostic and/or prognostic marker, Gene therapy (gene delivery/gene ablation), Research tools, Tissue regeneration in vitro and in vivo (regeneration for all these tissues and cell types composing these tissues and cell types derived from these tissues)

The MOL7 nucleic acids and proteins are useful in potential therapeutic applications implicated in various names of pathologies/disorders described below and/or other pathologies disorders. For example, a cDNA encoding the RH TYPE B GLYCOPROTEIN-like protein may be useful in gene therapy, and the RH TYPE B GLYCOPROTEIN-like protein may be useful when administered to a subject in need thereof. By way of nonlimiting example, the compositions of the present invention will have efficacy for treatment of patients suffering from the pathologies described above. The novel nucleic acid encoding MOL7, or fragments thereof, may further be useful in diagnostic applications, wherein the presence or amount of the nucleic acid or the protein are to be assessed.

These materials are further useful in the generation of antibodies that bind immuno-specifically to the novel MOL7 substances for use in therapeutic or diagnostic methods. These antibodies may be generated according to methods known in the art, using prediction from hydrophobicity charts, as described in the “Anti-MOLX Antibodies” section below. The disclosed MOL7 protein has multiple hydrophilic regions, each of which can be used as an immunogen. In one embodiment, a contemplated MOL7 epitope is from about amino acids 40 to 80. In another embodiment, a MOL7 epitope is from about amino acids 160 to 190. In additional embodiments, MOL7 epitopes are from about amino acids 175 to 225, 235 to 250, 325 to 345, 360 to 380, and from about amino acids 400 to 450. These novel proteins can also be used to develop assay systems for functional analysis.

### MOL8

A novel human Noelin-2-like nucleic acid was identified by TblastN using CuraGen Corporation's sequence file. The disclosed novel MOL8 nucleic acid of 1399 nucleotides (also referred to as SC84366578_A) is shown in Table 8A. An open reading frame begins with an ATG initiation codon at nucleotides 14-16 and ends with a TAG codon at nucleotides 1391-1393. A putative untranslated region downstream from the termination codon are underlined in Table 8A, and the start and stop codons are in bold letters.

TABLE 8A
MOL8 Nucleotide Sequence

(SEQ ID NO:23)

TGTTTTACTTGAAATGCTACAAACCAACACTCTTTTTATCCTAAAACAGGAGTCTGTGTTTTATGTTTCCCTTTGG
TTTCCTCAGACTCAGATTAGTCCTAAAGAAGGGTGGCAGGTGTACAGCTCAGCTCAGGATCCTGATGGGCGGTGCA
TTTGCACAGTTGTTGCTCCAGAACAAAACCTGTGTTCCCGGGATGCCAAAAGCAGGCAACTTCGCCAACTACTGGA
AAAGGTACAGAACATGTCCCAGTCTATTGAAGTCTTAAACTTGAGAACTCAGAGAGATTTCCAATATGTTTTAAAA
ATGGAAACCCAAATGAAAGGGCTGAAGGCAAAATTTCGGCAGATTGAAGATGATCGAAAGACACTTATGACCAAGC
ATTTTCAGCAGGAGTTGAAAGAGAAAATGGACGAGCTCCTGCCTTTGATCCCCGTGCTGGAACAGTGCAAAACAGA
TGCTAAGTTCATCACCCAGTTCAAGGAGGAAATAAGGAATCTGTCTGCTGTCCTCACTGGTATTCAGGAGGAAATT
GGTGCCTATGACTACGAGGAACTACACCAAAGAGTGCTGAGCTTGGAAACAAGACTTCGTGACTGCATGAAAAAGC
TATGTGGCAAACTGATGAAAATCACAGGCCCAGTTACAGTCAAGACATCTGGAACCCGATTTGGTGCTTGGATGAC
AGACCCTTTAGCATCTGAGAAAAACAACAGAGTATGGTACATGGACAGTTATACTAACAATAAAATTGTTCGTGAA
TACAAATCAATTGCAGACTTTGTCAGTGGGGCTGAATCAAGGACATACAACCTTCCTTTCAAGTGGGCAGGAACTA
ACCATGTTGTCTACAATGGCTCACTCTATTTTAACAAGTATCAGAGTAATATCATCATCAAATACAGCTTTGATAT
GGGGAGAGTGCTTGCCCAACGAAGCCTGGAGTATGCTGGTTTTCATAATGTTTACCCCTACACATGGGGTGGATTC
TCTGACATCGACCTAATGGCTGATGAAATCGGGCTGTGGGCTGTGTATGCAACTAACCAGAATGCAGGCAATATTG
TCATCAGCCAACTTAACCAAGATACCTTGGAGGTGATGAAGAGCTGGAGCACTGGCTACCCCAAGAGAAGTGCAGG
GGAATCTTTCATGATCTGTGGGACACTGTATGTCACCAACTCCCACTTAACTGGAGCCAAGGTGTATTATTCCTAT
TCCACCAAAACCTCCACATATGAGTACACAGACATTCCCTTCCATAACCAATACTTTCACATATCCATGCTTGACT
ACAATGCAAGAGATCGAGCTCTCTATGCCTGGAACAATGGCCACCAGGTGCTGTTCAATGTCACCCTTTTCCATAT
CATCAAGACAGAGGATGACACATAGGCAAAT

The MOL8 protein encoded by SEQ ID NO:23 has 459 amino acid residues, and is presented using the one-letter code in Table 8B (SEQ ID NO:24). The SignalP, Psort and/or Hydropathy profile for MOL8 predict that MOL8 has no signal peptide and is likely to be localized at the microbody (peroxisome) with a certainty of 0.5616. The molecular weight of the MOL8 protein is 53275.2 Daltons.

TABLE 8B
Encoded MOL8 protein sequence.

(SEQ ID NO:24)

MLQTNTLFILKQESVFYVSLWFPQTQISPKEGWQVYSSAQDPDGRCICTVVAPEQNLCSRDAKSRQLRQLLEKVQN
MSQSIEVLNLRTQRDFQYVLKMETQMKGLKAKFRQIEDDRKTLMTKHFQQELKEKMDELLPLIPVLEQCKTDAKFI
TQFKEEIRNLSAVLTGIQEEIGAYDYEELHQRVLSLETRLRDCMKKLCGKLMKITGPVTVKTSGTRFGAWMTDPLA
SEKNNRVWYMDSYTNNKIVREYKSIADFVSGAESRTYNLPFKWAGTNHVVYNGSLYFNKYQSNIIIKYSFDMGRVL
AQRSLEYAGFHNVYPYTWGGFSDIDLMADEIGLWAVYATNQNAGNIVISQLNQDTLEVMKSWSTGYPKRSAGESFM
ICGTLYVTNSHLTGAKVYYSYSTKTSTYEYTDIPFHNQYFHISMLDYNARDRALYAWNNGHQVLFNVTLFHIIKTE
DDT

The nucleotide sequence of MOL8 has 889 of 1286 bases (69%) identical to a Gallus gallus NOELIN-2 mRNA (GENBANK-ID: AF239804). The full amino acid sequence of the protein of the invention was found to have 288 of 448 amino acid residues (64%) identical to, and 367 of 448 residues (80%) positive with, the 457 amino acid residue NOELIN-2 protein from Gallus gallus (Chicken) (ptnr:SPTREMBL-ACC: AAF43715), and 439 of 459 amino acid residues (95%) identical to, and 442 of 459 residues (96%) positive with, the 458 amino acid residue patp:AAB74696 Human membrane associated protein MEMAP-2.

The global sequence homology (as defined by FASTA alignment with the full length sequence of this protein) is 74% amino acid homology and 65% amino acid identity. In addition, this protein contains the following protein domain (as defined by Pfam) at the indicated nucleotide positions: Olfactomedin-like domain (PF02191) at amino acid positions 201 to 451.

The full amino acid sequence of MOL8 was found to have homology with several proteins including those disclosed in the BLASTP data in Table 8C.

TABLE 8C
BLAST results for MOL8

| Gene Index/ | | Length | Identity | Positives | |
|---|---|---|---|---|---|
| Identifier | Protein/Organism | (aa) | (%) | (%) | Expect |
| gi\|3024210\|sp\|Q62609\|NOE1_RAT | NOELIN PRECURSOR | 485 | 285/431 | 355/431 | e-172 |
| | (NEURONAL | | | | |
| | OLFACTOMEDIN- | | | | |
| | RELATED ER | | | | |
| | LOCALIZED | | | | |
| | PROTEIN) | | | | |
| | (PANCORTIN) | | | | |
| | (1B426B) | | | | |
| gi\|13124385\|sp\|Q91AK4\|NOE1_CHICK | NOELIN PRECURSOR | 485 | 284/431 | 357/431 | e-171 |
| | (NEURONAL | | (65%) | (81%) | |
| | OLFACTOMEDIN- | | | | |
| | RELATED ER | | | | |
| | LOCALIZED | | | | |
| | PROTEIN) | | | | |
| | (PANCORTIN) | | | | |
| gi\|9506929\|ref\|NP_062371.1\| | olfactomedin | 485 | 284/431 | 354/431 | e-171 |
| | related ER | | (65%) | (81%) | |
| | localized protein | | | | |
| | [Mus musculus] | | | | |
| gi\|7248902\|gb\|AAF43715.1\|AF239804_1 | MOELIN-2 [Gallus | 457 | 284/431 | 357/431 | e-171 |
| (AF239804) | gallus] | | (65%) | (81%) | |
| gi\|2143875\|pir\|\|I73636 | neuronal | 457 | 285/431 | 355/431 | e-171 |
| | olfactomedin- | | (66%) | (82%) | |
| | related ER | | | | |
| | localized protein- | | | | |
| | rat | | | | |

Homology between MOL8 and other proteins are presented graphically in the multiple sequence alignment given in Table 8D (with MOL8 being shown on line 1) as a ClustalW analysis comparing MOL8 with related protein sequences.

[image]

[image]

[image]

Table 8E lists the domain description from DOMAIN analysis results against MOL8. The region from amino acid residue 201 through 457 (SEQ ID NO:24) most probably (E=4e⁻⁸⁵) contains a Olfactomedin-like domain, aligned in Table 8E. This indicates that the MOL8 sequence has properties similar to those of other proteins known to contain this domain.

[image]

### Uses of the Compositions of the Invention

The above defined information for MOL8 suggests that this Noelin-2-like protein may function as a member of a “Noelin-2 family”. This family is involved in neural crest development, and other developmental processes. Therefore, the novel nucleic acids and proteins identified here may be useful in potential therapeutic applications implicated in (but not limited to) various pathologies and disorders as indicated below. The potential therapeutic applications for MOL8 include, but are not limited to: protein therapeutic, small molecule drug target, antibody target (therapeutic, diagnostic, drug targeting/cytotoxic antibody), diagnostic and/or prognostic marker, gene therapy (gene delivery/gene ablation), research tools, tissue regeneration in vivo and in vitro of all tissues and cell types composing (but not limited to) those defined here.

The MOL8 nucleic acids and proteins are useful in potential therapeutic applications implicated in neural crest development in early embryonic stage. For example, a cDNA encoding the Noelin-2-like protein may be useful in gene therapy, and the Noelin-2-like protein may be useful when administered to a subject in need thereof. By way of nonlimiting example, the compositions of the present invention will have efficacy for treatment of patients suffering from primary open-angle glaucoma (POAG), and bone disorders, hematopoietic disorders, neuro-developmental disorders, cancer, autoimmune disorders, psychiatric disorders. The novel nucleic acid encoding MOL8, or fragments thereof, may further be useful in diagnostic applications, wherein the presence or amount of the nucleic acid or the protein are to be assessed.

These materials are further useful in the generation of antibodies that bind immuno-specifically to the novel MOL8 substances for use in therapeutic or diagnostic methods. These antibodies may be generated according to methods known in the art, using prediction from hydrophobicity charts, as described in the “Anti-MOLX Antibodies” section below. The disclosed MOL8 protein has multiple hydrophilic regions, each of which can be used as an immunogen. In one embodiment, a contemplated MOL8 epitope is from about amino acids 20 to 50. In another embodiment, a MOL8 epitope is from about amino acids 50 to 125. in additional embodiments, MOL8 epitopes are from about amino acids 140 to 210, 225 to 320, 350 to 375, and from about amino acids 380 to 440. These novel proteins can also be used to develop assay systems for functional analysis.

TABLE 9
Summary Of Nucleic Acids And Proteins Of The Invention

| | | | Nucleic | Amino |
|---|---|---|---|---|
| | | | Acid | Acid |
| | | | SEQ ID | SEQ ID |
| Name | Tables | Clone; Description of Homolog | NO | NO |
| MOL1 | 1A, 1B, | MOL1a: SC29674552_EXT | 1 | 2 |
| | 1D, 1E | MOL1b: CG56250-02 | 3 | 4 |
| MOL2 | 2A, 2B | MOL2a: SC98428706_EXT | 5 | 6 |
| | 2C, 2D | MOL2b: 191999007 | 101 | 102 |
| | 2E, 2F | MOL2c: 192586956 | 103 | 104 |
| MOL3 | 3A, 3B | MOL3a: SC85516573_EXT | 7 | 8 |
| | 3D, 3E | MOL3b: CG53027-02 | 9 | 10 |
| MOL4 | 4A, 4B, | MOL4a: SC_111750277_A | 11 | 12 |
| | 4D, 4E | MOL4b: CG106951-02 | 105 | 106 |
| | 4F, 4G | MOL4c: CG106951-04 | 107 | 108 |
| | 4I, 4K | MOL4d: 209829549 | 109 | 111 |
| | 4L, 4N | MOL4e: 209829553 | 112 | 114 |
| | 4O, 4P | MOL4f: 209829642 | 115 | 116 |
| | 4Q, 4R | MOL4g: 209829670 | 117 | 118 |
| MOL5 | 5A, 5B, | MOL5a: SC20422974_A | 13 | 14 |
| | 5C, 5D, | MOL5b: SC14998905_EXT | 15 | 16 |
| | 5E, 5F | MOL5c: CG50907-02 | 17 | 18 |
| | 5H, 5I | MOL5d: CG50907-03 | 119 | 120 |
| | 5J, SL | MOL5e: 170645595 | 121 | 123 |
| | 5M, 5N | MOL5f: 170645599 | 124 | 125 |
| MOL6 | 6A, 6B | MOL6: GMAC060288_A | 19 | 20 |
| MOL7 | 7A, 7B | MOL7: AF193808A | 21 | 22 |
| MOL8 | 8A, 8B | MOL8: SC84366578_A | 23 | 24 |

### MOLX Nucleic Acids and Polypeptides

One aspect of the invention pertains to isolated nucleic acid molecules that encode MOLX polypeptides or biologically active portions thereof. Also included in the invention are nucleic acid fragments sufficient for use as hybridization probes to identify MOLX-encoding nucleic acids (e.g., MOLX mRNAs) and fragments for use as PCR primers for the ampliflcation and/or mutation of MOLX nucleic acid molecules. As used herein, the term “nucleic acid molecule” is intended to include DNA molecules (e.g, cDNA or genomic DNA), RNA molecules (e.g., mRNA), analogs of the DNA or RNA generated using nucleotide analogs, and derivatives, fragments and homologs thereof. The nucleic acid molecule may be single-stranded or double-stranded, but preferably is comprised double-stranded DNA.

An MOLX nucleic acid can encode a mature MOLX polypeptide. As used herein, a “mature” form of a polypeptide or protein disclosed in the present invention is the product of a naturally occurring polypeptide or precursor form or proprotein. The naturally occurring polypeptide, precursor or proprotein includes, by way of nonlimiting example, the full-length gene product, encoded by the corresponding gene. Alternatively, it may be defined as the polypeptide, precursor or proprotein encoded by an ORF described herein. The product “mature” form arises, again by way of nonlimiting example, as a result of one or more naturally occurring processing steps as they may take place within the cell, or host cell, in which the gene product arises. Examples of such processing steps leading to a “mature” form of a polypeptide or protein include the cleavage of the N-terminal methionine residue encoded by the initiation codon of an ORF, or the proteolytic cleavage of a signal peptide or leader sequence. Thus a mature form arising from a precursor polypeptide or protein that has residues 1 to N, where residue1 is the N-terminal methionine, would have residues 2 through N remaining after removal of the N-terminal methionine. Alternatively, a mature form arising from a precursor polypeptide or protein having residues 1 to N, in which an N-terminal signal sequence from residue 1 to residue M is cleaved, would have the residues from residue M+1 to residue N remaining. Further as used herein, a “mature” form of a polypeptide or protein may arise from a step of post-translational modification other than a proteolytic cleavage event. Such additional processes include, by way of non-limiting example, glycosylation, myristoylation or phosphorylation. In general, a mature polypeptide or protein may result from the operation of only one of these processes, or a combination of any of them.

The term “probes”, as utilized herein, refers to nucleic acid sequences of variable length, preferably between at least about 10 nucleotides (nt), 100 nt, or as many as approximately, e.g., 6,000 nt, depending upon the specific use. Probes are used in the detection of identical, similar, or complementary nucleic acid sequences. Longer length probes are generally obtained from a natural or recombinant source, are highly specific, and much slower to hybridize than shorter-length oligomer probes. Probes may be single- or double-stranded and designed to have specificity in PCR, membrane-based hybridization technologies, or ELISA-like technologies.

The term “isolated” nucleic acid molecule, as utilized herein, is one, which is separated from other nucleic acid molecules which are present in the natural source of the nucleic acid. Preferably, an “isolated” nucleic acid is free of sequences which naturally flank the nucleic acid (i.e., sequences located at the 5′- and 3′-termini of the nucleic acid) in the genomic DNA of the organism from which the nucleic acid is derived. For example, in various embodiments, the isolated MOLX nucleic acid molecules can contain less than about 5 kb, 4 kb, 3 kb, 2 kb, 1 kb, 0.5 kb or 0.1 kb of nucleotide sequences which naturally flank the nucleic acid molecule in genomic DNA of the cell/tissue from which the nucleic acid is derived (e.g., brain, heart, liver, spleen, etc.). Moreover, an “isolated” nucleic acid molecule, such as a cDNA molecule, can be substantially free of other cellular material or culture medium when produced by recombinant techniques, or of chemical precursors or other chemicals when chemically synthesized.

A nucleic acid molecule of the invention, e.g., a nucleic acid molecule having the nucleotide sequence of SEQ ID NOS:1, 3, 5, 7, 9, 11, 13, 15, 17, 19, 21, 23, 101, 103, 105, 107, 109, 112, 115, 117, 119, 121, and 124, or a complement of this aforementioned nucleotide sequence, can be isolated using standard molecular biology techniques and the sequence information provided herein. Using all or a portion of the nucleic acid sequence of SEQ ID NOS:1, 3, 5, 7, 9, 11, 13, 15, 17, 19, 21, 23, 101, 103, 105, 107, 109, 112, 115, 117, 119, 121, and 124 as a hybridization probe, MOLX molecules can be isolated using standard hybridization and cloning techniques (e.g., as described in Sambrook, et al., (eds.), MOLECULAR CLONING: A LABORATORY MANUAL 2^{nd} Ed., Cold Spring Harbor Laboratory Press, Cold Spring Harbor, N.Y., 1989; and Ausubel, et al., (eds.), CURRENT PROTOCOLS IN MOLECULAR BIOLOGY, John Wiley & Sons, New York, N.Y., 1993.)

A nucleic acid of the invention can be amplified using cDNA, mRNA or alternatively, genomic DNA, as a template and appropriate oligonucleotide primers according to standard PCR amplification techniques. The nucleic acid so amplified can be cloned into an appropriate vector and characterized by DNA sequence analysis. Furthermore, oligonucleotides corresponding to MOLX nucleotide sequences can be prepared by standard synthetic techniques, e.g, using an automated DNA synthesizer.

As used herein, the term “oligonucleotide” refers to a series of linked nucleotide residues, which oligonucleotide has a sufficient number of nucleotide bases to be used in a PCR reaction. A short oligonucleotide sequence may be based on, or designed from, a genomic or cDNA sequence and is used to amplify, confirm, or reveal the presence of an identical, similar or complementary DNA or RNA in a particular cell or tissue. Oligonucleotides comprise portions of a nucleic acid sequence having about 10 nt, 50 nt, or 100 nt in length, preferably about 15 nt to 30 nt in length. In one embodiment of the invention, an oligonucleotide comprising a nucleic acid molecule less than 100 nt in length would further comprise at least 6 contiguous nucleotides of SEQ ID NOS:1, 3, 5, 7, 9, 11, 13, 15, 17, 19, 21, 23, 101, 103, 105, 107, 109, 112, 115, 117, 119, 121, and 124, or a complement thereof. Oligonucleotides may be chemically synthesized and may also be used as probes.

In another embodiment, an isolated nucleic acid molecule of the invention comprises a nucleic acid molecule that is a complement of the nucleotide sequence shown in SEQ ID NOS:1, 3, 5, 7, 9, 11, 13, 15, 17, 19, 21, 23, 101, 103, 105, 107, 109, 112, 115, 117, 119, 121, and 124, or a portion of this nucleotide sequence (e.g., a fragment that can be used as a probe or primer or a fragment encoding a biologically-active portion of an MOLX polypeptide). A nucleic acid molecule that is complementary to the nucleotide sequence shown in SEQ ID NOS:1, 3, 5, 7, 9, 11, 13, 15, 17, 19, 21, 23, 101, 103, 105, 107, 109, 112, 115, 117, 119, 121, and 124 is one that is sufficiently complementary to the nucleotide sequence shown in SEQ ID NOS:1, 3, 5, 7, 9, 11, 13, 15, 17, 19, 21, 23, 101, 103, 105, 107, 109, 112, 115, 117, 119, 121, and 124 that it can hydrogen bond with little or no mismatches to the nucleotide sequence shown SEQ ID NOS:1, 3, 5, 7, 9, 11, 13, 15, 17, 19, 21, 23, 101, 103, 105, 107, 109, 112, 115, 117, 119, 121, and 124, thereby forming a stable duplex.

As used herein, the term “complementary” refers to Watson-Crick or Hoogsteen base pairing between nucleotides units of a nucleic acid molecule, and the term “binding” means the physical or chemical interaction between two polypeptides or compounds or associated polypeptides or compounds or combinations thereof. Binding includes ionic, non-ionic, van der Waals, hydrophobic interactions, and the like. A physical interaction can be either direct or indirect. Indirect interactions may be through or due to the effects of another polypeptide or compound. Direct binding refers to interactions that do not take place through, or due to, the effect of another polypeptide or compound, but instead are without other substantial chemical intermediates.

Fragments provided herein are defined as sequences of at least 6 (contiguous) nucleic acids or at least 4 (contiguous) amino acids, a length sufficient to allow for specific hybridization in the case of nucleic acids or for specific recognition of an epitope in the case of amino acids, respectively, and are at most some portion less than a full length sequence. Fragments may be derived from any contiguous portion of a nucleic acid or amino acid sequence of choice. Derivatives are nucleic acid sequences or amino acid sequences formed from the native compounds either directly or by modification or partial substitution. Analogs are nucleic acid sequences or amino acid sequences that have a structure similar to, but not identical to, the native compound but differs from it in respect to certain components or side chains. Analogs may be synthetic or from a different evolutionary origin and may have a similar or opposite metabolic activity compared to wild type. Homologs are nucleic acid sequences or amino acid sequences of a particular gene that are derived from different species.

Derivatives and analogs may be full length or other than full length, if the derivative or analog contains a modified nucleic acid or amino acid, as described below. Derivatives or analogs of the nucleic acids or proteins of the invention include, but are not limited to, molecules comprising regions that are substantially homologous to the nucleic acids or proteins of the invention, in various embodiments, by at least about 70%, 80%, or 95% identity (with a preferred identity of 80-95%) over a nucleic acid or amino acid sequence of identical size or when compared to an aligned sequence in which the alignment is done by a computer homology program known in the art, or whose encoding nucleic acid is capable of hybridizing to the complement of a sequence encoding the aforementioned proteins under stringent, moderately stringent, or low stringent conditions. See e.g Ausubel, et al., CURRENT PROTOCOLS IN MOLECULAR BIOLOGY, John Wiley & Sons, New York, N.Y., 1993, and below.

A “homologous nucleic acid sequence” or “homologous amino acid sequence,” or variations thereof, refer to sequences characterized by a homology at the nucleotide level or amino acid level as discussed above. Homologous nucleotide sequences encode those sequences coding for isoforms of MOLX polypeptides. Isoforms can be expressed in different tissues of the same organism as a result of, for example, alternative splicing of RNA. Alternatively, isoforms can be encoded by different genes. In the invention, homologous nucleotide sequences include nucleotide sequences encoding for an MOLX polypeptide of species other than humans, including, but not limited to: vertebrates, and thus can include, e.g., frog, mouse, rat, rabbit, dog, cat cow, horse, and other organisms. Homologous nucleotide sequences also include, but are not limited to, naturally occurring allelic variations and mutations of the nucleotide sequences set forth herein. A homologous nucleotide sequence does not, however, include the exact nucleotide sequence encoding human MOLX protein. Homologous nucleic acid sequences include those nucleic acid sequences that encode conservative amino acid substitutions (see below) in SEQ ID NOS:1, 3, 5, 7, 9, 11, 13, 15, 17, 19, 21, 23, 101, 103, 105, 107, 109, 112, 115, 117, 119, 121, and 124, as well as a polypeptide possessing MOLX biological activity. Various biological activities of the MOLX proteins are described below.

An MOLX polypeptide is encoded by the open reading frame (“ORF”) of an MOLX nucleic acid. An ORF corresponds to a nucleotide sequence that could potentially be translated into a polypeptide. A stretch of nucleic acids comprising an ORF is uninterrupted by a stop codon. An ORF that represents the coding sequence for a full protein begins with an ATG “start” codon and terminates with one of the three “stop” codons, namely, TAA, TAG, or TGA. For the purposes of this invention, an ORF may be any part of a coding sequence, with or without a start codon, a stop codon, or both. For an ORF to be considered as a good candidate for coding for a bona fide cellular protein, a minimum size requirement is often set, e.g., a stretch of DNA that would encode a protein of 50 amino acids or more.

The nucleotide sequences determined from the cloning of the human MOLX genes allows for the generation of probes and primers designed for use in identifying and/or cloning MOLX homologues in other cell types, e.g. from other tissues, as well as MOLX homologues from other vertebrates. The probe/primer typically comprises substantially purified oligonucleotide. The oligonucleotide typically comprises a region of nucleotide sequence that hybridizes under stringent conditions to at least about 12, 25, 50, 100, 150, 200, 250, 300, 350 or 400 consecutive sense strand nucleotide sequence of SEQ ID NOS:1, 3, 5, 7, 9, 11, 13, 15, 17, 19, 21, 23, 101, 103, 105, 107, 109, 112, 115, 117, 119, 121, and 124; or an anti-sense strand nucleotide sequence of SEQ ID NOS:1, 3, 5, 7, 9, 11, 13, 15, 17, 19, 21, 23, 101, 103, 105, 107, 109, 112, 115, 117, 119, 121, and 124; or of a naturally occurring mutant of SEQ ID NOS:1, 3, 5, 7, 9, 11, 13, 15, 17, 19, 21, 23, 101, 103, 105, 107, 109, 112, 115, 117, 119, 121, and 124.

Probes based on the human MOLX nucleotide sequences can be used to detect transcripts or genomic sequences encoding the same or homologous proteins. In various embodiments, the probe further comprises a label group attached thereto, e.g. the label group can be a radioisotope, a fluorescent compound, an enzyme, or an enzyme co-factor. Such probes can be used as a part of a diagnostic test kit for identifying cells or tissues which mis-express an MOLX protein, such as by measuring a level of an MOLX-encoding nucleic acid in a sample of cells from a subject e.g., detecting MOLX mRNA levels or determining whether a genomic MOLX gene has been mutated or deleted.

“A polypeptide having a biologically-active portion of an MOLX polypeptide” refers to polypeptides exhibiting activity similar, but not necessarily identical to, an activity of a polypeptide of the invention, including mature forms, as measured in a particular biological assay, with or without dose dependency. A nucleic acid fragment encoding a “biologically-active portion of MOLX” can be prepared by isolating a portion SEQ ID NOS:1, 3, 5, 7, 9, 11, 13, 15, 17, 19, 21, 23, 101, 103, 105, 107, 109, 112, 115, 117, 119, 121, and 124 that encodes a polypeptide having an MOLX biological activity (the biological activities of the MOLX proteins are described below), expressing the encoded portion of MOLX protein (e.g., by recombinant expression in vitro) and assessing the activity of the encoded portion of MOLX.

### MOLX Nucleic Acid and Polypeptide Variants

The invention further encompasses nucleic acid molecules that differ from the nucleotide sequences shown SEQ ID NOS:1, 3, 5, 7, 9, 11, 13, 15, 17, 19, 21, 23, 101, 103, 105, 107, 109, 112, 115, 117, 119, 121, and 124 due to degeneracy of the genetic code and thus encode the same MOLX proteins as that encoded by the nucleotide sequences shown in SEQ ID NOS:1, 3, 5, 7, 9, 11, 13, 15, 17, 19, 21, 23, 101, 103, 105, 107, 109, 112, 115, 117, 119, 121, and 124. In another embodiment, an isolated nucleic acid molecule of the invention has a nucleotide sequence encoding a protein having an amino acid sequence shown in SEQ ID NOS:2, 4, 6, 8, 10, 12, 14, 16, 18, 20, 22, 24, 102, 104, 106, 108, 111, 114, 116, 118, 120, 123, and 125.

In addition to the human MOLX nucleotide sequences shown in SEQ ID NOS:1, 3, 5, 7, 9, 11, 13, 15, 17, 19, 21, 23, 101, 103, 105, 107, 109, 112, 115, 117, 119, 121, and 124 it will be appreciated by those skilled in the art that DNA sequence polymorphisms that lead to changes in the amino acid sequences of the MOLX polypeptides may exist within a population (e g., the human population). Such genetic polymorphism in the MOLX genes may exist among individuals within a population due to natural allelic variation. As used herein, the terms “gene” and “recombinant gene” refer to nucleic acid molecules comprising an open reading frame (ORF) encoding an MOLX protein, preferably a vertebrate MOLX protein. Such natural allelic variations can typically result in 1-5% variance in the nucleotide sequence of the MOLX genes. Any and all such nucleotide variations and resulting amino acid polymorphisms in the MOLX polypeptides, which are the result of natural allelic variation and that do not alter the functional activity of the MOLX polypeptides, are intended to be within the scope of the invention.

Moreover, nucleic acid molecules encoding MOLX proteins from other species, and thus that have a nucleotide sequence that differs from the human sequence SEQ ID NOS:1, 3, 5, 7, 9, 11, 13, 15, 17, 19, 21, 23, 101, 103, 105, 107, 109, 112, 115, 117, 119, 121, and 124 are intended to be within the scope of the invention. Nucleic acid molecules corresponding to natural allelic variants and homologues of the MOLX cDNAs of the invention can be isolated based on their homology to the human MOLX nucleic acids disclosed herein using the human cDNAs, or a portion thereof, as a hybridization probe according to standard hybridization techniques under stringent hybridization conditions.

Accordingly, in another embodiment, an isolated nucleic acid molecule of the invention is at least 6 nucleotides in length and hybridizes under stringent conditions to the nucleic acid molecule comprising the nucleotide sequence of SEQ ID NOS:1, 3, 5, 7, 9, 11, 13, 15, 17, 19, 21, 23, 101, 103, 105, 107, 109, 112, 115, 117, 119, 121, and 124. In another embodiment, the nucleic acid is at least 10, 25, 50, 100, 250, 500, 750, 1000, 1500, or 2000 or more nucleotides in length. In yet another embodiment, an isolated nucleic acid molecule of the invention hybridizes to the coding region. As used herein, the term “hybridizes under stringent conditions” is intended to describe conditions for hybridization and washing under which nucleotide sequences at least 60% homologous to each other typically remain hybridized to each other.

Homologs (i.e., nucleic acids encoding MOLX proteins derived from species other than human) or other related sequences (e.g, paralogs) can be obtained by low, moderate or high stringency hybridization with all or a portion of the particular human sequence as a probe using methods well known in the art for nucleic acid hybridization and cloning.

As used herein, the phrase “stringent hybridization conditions” refers to conditions under which a probe, primer or oligonucleotide will hybridize to its target sequence, but to no other sequences. Stringent conditions are sequence-dependent and will be different in different circumstances. Longer sequences hybridize specifically at higher temperatures than shorter sequences. Generally, stringent conditions are selected to be about 5° C. lower than the thermal melting point (Tm) for the specific sequence at a defined ionic strength and pH. The Tm is the temperature (under defined ionic strength, pH and nucleic acid concentration) at which 50% of the probes complementary to the target sequence hybridize to the target sequence at equilibrium. Since the target sequences are generally present at excess, at Tm, 50% of the probes are occupied at equilibrium. Typically, stringent conditions will be those in which the salt concentration is less than about 1.0 M sodium ion, typically about 0.01 to 1.0 M sodium ion (or other salts) at pH 7.0 to 8.3 and the temperature is at least about 30° C. for short probes, primers or oligonucleotides (e g., 10 nt to 50 nt) and at least about 60° C. for longer probes, primers and oligonucleotides. Stringent conditions may also be achieved with the addition of destabilizing agents, such as formamide.

Stringent conditions are known to those skilled in the art and can be found in Ausubel, et al., (eds.), CURRENT PROTOCOLS IN MOLECULAR BIOLOGY, John Wiley & Sons, N.Y. (1989), 6.3.1-6.3.6. Preferably, the conditions are such that sequences at least about 65%, 70%, 75%, 85%, 90%, 95%, 98%, or 99% homologous to each other typically remain hybridized to each other. A non-limiting example of stringent hybridization conditions are hybridization in a high salt buffer comprising 6×SSC, 50 mM Tris-HCl (pH 7.5), 1 mM EDTA, 0.02% PVP, 0.02% Ficoll, 0.02% BSA, and 500 mg/ml denatured salmon sperm DNA at 65° C., followed by one or more washes in 0.2×SSC, 0.01% BSA at 50° C. An isolated nucleic acid molecule of the invention that hybridizes under stringent conditions to the sequences of SEQ ID NOS:1, 3, 5, 7, 9, 11, 13, 15, 17, 19, 21, 23, 101, 103, 105, 107, 109, 112, 115, 117, 119, 121, and 124 corresponds to a naturally-occurring nucleic acid molecule. As used herein, a “naturally-occurring” nucleic acid molecule refers to an RNA or DNA molecule having a nucleotide sequence that occurs in nature (e.g., encodes a natural protein).

In a second embodiment, a nucleic acid sequence that is hybridizable to the nucleic acid molecule comprising the nucleotide sequence of SEQ ID NOS:1, 3, 5, 7, 9, 11, 13, 15, 17, 19, 21, 23, 101, 103, 105, 107, 109, 112, 115, 117, 119, 121, and 124 or fragments, analogs or derivatives thereof, under conditions of moderate stringency is provided. A non-limiting example of moderate stringency hybridization conditions are hybridization in 6×SSC, 5× Denhardt's solution, 0.5% SDS and 100 mg/ml denatured salmon sperm DNA at 55° C., followed by one or more washes in 1×SSC, 0.1% SDS at 37° C. Other conditions of moderate stringency that may be used are well-known within the art. See, e.g., Ausubel, et al. (eds.), 1993, CURRENT PROTOCOLS IN MOLECULAR BIOLOGY, John Wiley & Sons, NY, and Kriegler, 1990; GENE TRANSFER AND EXPRESSION, A LABORATORY MANUAL, Stockton Press, NY.

In a third embodiment, a nucleic acid that is hybridizable to the nucleic acid molecule comprising the nucleotide sequences of SEQ ID NOS:1, 3, 5, 7, 9, 11, 13, 15, 17, 19, 21, 23, 101, 103, 105, 107, 109, 112, 115, 117, 119, 121, and 124 or fragments, analogs or derivatives thereof, under conditions of low stringency, is provided. A non-limiting example of low stringency hybridization conditions are hybridization in 35% formamide, 5×SSC, 50 mM Tris-HCl (pH 7.5), 5 mM EDTA, 0.02% PVP, 0.02% Ficoll, 0.2% BSA, 100 mg/ml denatured salmon sperm DNA, 10% (wt/vol) dextran sulfate at 40° C., followed by one or more washes in 2×SSC, 25 mM Tris-HCl (pH 7.4), 5 mM EDTA, and 0.1% SDS at 50° C. Other conditions of low stringency that may be used are well known in the art (e.g, as employed for cross-species hybridizations). See, e g, Ausubel, et al. (eds.), 1993, CURRENT PROTOCOLS IN MOLECULAR BIOLOGY, John Wiley & Sons, NY, and Kriegler, 1990, GENE TRANSFER AND EXPRESSION, A LABORATORY MANUAL, Stockton Press, NY; Shilo and Weinberg, 1981. Proc Natl Acad Sci USA 78: 6789-6792.

### Conservative Mutations

In addition to naturally-occurring allelic variants of MOLX sequences that may exist in the population, the skilled artisan will further appreciate that changes can be introduced by mutation into the nucleotide sequences of SEQ ID NOS:1, 3, 5, 7, 9, 11, 13, 15, 17, 19, 21, 23, 101, 103, 105, 107, 109, 112, 115, 117, 119, 121, and 124 thereby leading to changes in the amino acid sequences of the encoded MOLX proteins, without altering the functional ability of said MOLX proteins. For example, nucleotide substitutions leading to amino acid substitutions at “non-essential” amino acid residues can be made in the sequence of SEQ ID NOS:2, 4, 6, 8, 10, 12, 14, 16, 18, 20, 22, 24, 102, 104, 106, 108, 111, 114, 116, 118, 120, 123, and 125. A “non-essential” amino acid residue is a residue that can be altered from the wild-type sequences of the MOLX proteins without altering their biological activity, whereas an “essential” amino acid residue is required for such biological activity. For example, amino acid residues that are conserved among the MOLX proteins of the invention are predicted to be particularly non-amenable to alteration. Amino acids for which conservative substitutions can be made are well-known within the art.

Another aspect of the invention pertains to nucleic acid molecules encoding MOLX proteins that contain changes in amino acid residues that are not essential for activity. Such MOLX proteins differ in amino acid sequence from SEQ ID NOS:2, 4, 6, 8, 10, 12, 14, 16, 18, 20, 22, 24, 102, 104, 106, 108, 111, 114, 116, 118, 120, 123, and 125 yet retain biological activity. In one embodiment, the isolated nucleic acid molecule comprises a nucleotide sequence encoding a protein, wherein the protein comprises an amino acid sequence at least about 45% homologous to the amino acid sequences of SEQ ID NOS:2, 4, 6, 8, 10, 12, 14, 16, 18, 20, 22, 24, 102, 104, 106, 108, 111, 114, 116, 118, 120, 123, and 125. Preferably, the protein encoded by the nucleic acid molecule is at least about 60% homologous to SEQ ID NOS:2, 4, 6, 8, 10, 12, 14, 16, 18, 20, 22, 24, 102, 104, 106, 108, 111, 114, 116, 118, 120, 123, and 125; more preferably at least about 70% homologous to SEQ ID NOS:2, 4, 6, 8, 10, 12, 14, 16, 18, 20, 22, 24, 102, 104, 106, 108, 111, 114, 116, 118, 120, 123, and 125; still more preferably at least about 80% homologous to SEQ ID NOS:2, 4, 6, 8, 10, 12, 14, 16, 18, 20, 22, 24, 102, 104, 106, 108, 111, 114, 116, 118, 120, 123, and 125; even more preferably at least about 90% homologous to SEQ ID NOS:2, 4, 6, 8, 10, 12, 14, 16, 18, 20, 22, 24, 102, 104, 106, 108, 111, 114, 116, 118, 120, 123, and 125; and most preferably at least about 95% homologous to SEQ ID NOS:2, 4, 6, 8, 10, 12, 14, 16, 18, 20, 22, 24, 102, 104, 106, 108, 111, 114, 116, 118, 120, 123, and 125.

An isolated nucleic acid molecule encoding an MOLX protein homologous to the protein of SEQ ID NOS:2, 4, 6, 8, 10, 12, 14, 16, 18, 20, 22, 24, 102, 104, 106, 108, 111, 114, 116, 118, 120, 123, and 125 can be created by introducing one or more nucleotide substitutions, additions or deletions into the nucleotide sequence of SEQ ID NOS:1, 3, 5, 7, 9, 11, 13, 15, 17, 19, 21, 23, 101, 103, 105, 107, 109, 112, 115, 117, 119, 121, and 124 such that one or more amino acid substitutions, additions or deletions are introduced into the encoded protein.

Mutations can be introduced into SEQ ID NOS:2, 4, 6, 8, 10, 12, 14, 16, 18, 20, 22, 24, 102, 104, 106, 108, 111, 114, 116, 118, 120, 123, and 125 by standard techniques, such as site-directed mutagenesis and PCR-mediated mutagenesis. Preferably, conservative amino acid substitutions are made at one or more predicted, non-essential amino acid residues. A “conservative amino acid substitution” is one in which the amino acid residue is replaced with an amino acid residue having a similar side chain. Families of amino acid residues having similar side chains have been defined within the art. These families include amino acids with basic side chains (e.g, lysine, arginine, histidine), acidic side chains (e.g., aspartic acid, glutamic acid), uncharged polar side chains (e g., glycine, asparagine, glutamine, serine, threonine, tyrosine, cysteine), nonpolar side chains (e.g, alanine, valine, leucine, isoleucine, proline, phenylalanine, methionine, tryptophan), beta-branched side chains (e.g., threonine, valine, isoleucine) and aromatic side chains (e g., tyrosine, phenylalanine, tryptophan, histidine). Thus, a predicted non-essential amino acid residue in the MOLX protein is replaced with another amino acid residue from the same side chain family. Alternatively, in another embodiment, mutations can be introduced randomly along all or part of an MOLX coding sequence, such as by saturation mutagenesis, and the resultant mutants can be screened for MOLX biological activity to identify mutants that retain activity. Following mutagenesis of SEQ ID NOS:1, 3, 5, 7, 9, 11, 13, 15, 17, 19, 21, 23, 101, 103, 105, 107, 109, 112, 115, 117, 119, 121, and 124, the encoded protein can be expressed by any recombinant technology known in the art and the activity of the protein can be determined.

The relatedness of amino acid families may also be determined based on side chain interactions. Substituted amino acids may be fully conserved “strong” residues or fully conserved “weak” residues. The “strong” group of conserved amino acid residues may be any one of the following groups: STA, NEQK, NHQK, NDEQ, QHRK, MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLIM, HFY, wherein the letters within each group represent the single letter amino acid code.

In one embodiment, a mutant MOLX protein can be assayed for (i) the ability to form protein:protein interactions with other MOLX proteins, other cell-surface proteins, or biologically-active portions thereof, (ii) complex formation between a mutant MOLX protein and an MOLX ligand; or (iii) the ability of a mutant MOLX protein to bind to an intracellular target protein or biologically-active portion thereof; (e.g avidin proteins).

In yet another embodiment, a mutant MOLX protein can be assayed for the ability to regulate a specific biological function (e g. regulation of insulin release).

### Antisense Nucleic Acids

Another aspect of the invention pertains to isolated antisense nucleic acid molecules that are hybridizable to or complementary to the nucleic acid molecule comprising the nucleotide sequence of SEQ ID NOS:1, 3, 5, 7, 9, 11, 13, 15, 17, 19, 21, 23, 101, 103, 105, 107, 109, 112, 115, 117, 119, 121, and 124, or fragments, analogs or derivatives thereof. An “antisense” nucleic acid comprises a nucleotide sequence that is complementary to a “sense” nucleic acid encoding a protein (e.g., complementary to the coding strand of a double-stranded cDNA molecule or complementary to an mRNA sequence). In specific aspects, antisense nucleic acid molecules are provided that comprise a sequence complementary to at least about 10, 25, 50, 100, 250 or 500 nucleotides or an entire MOLX coding strand, or to only a portion thereof. Nucleic acid molecules encoding fragments, homologs, derivatives and analogs of an MOLX protein of SEQ ID NOS:2, 4, 6, 8, 10, 12, 14, 16, 18, 20, 22, 24, 102, 104, 106, 108, 111, 114, 116, 118, 120, 123, and 125, or antisense nucleic acids complementary to an MOLX nucleic acid sequence of SEQ ID NOS:1, 3, 5, 7, 9, 11, 13, 15, 17, 19, 21, 23, 101, 103, 105, 107, 109, 112, 115, 117, 119, 121, and 124, are additionally provided.

In one embodiment, an antisense nucleic acid molecule is antisense to a “coding region” of the coding strand of a nucleotide sequence encoding an MOLX protein. The term “coding region” refers to the region of the nucleotide sequence comprising codons which are translated into amino acid residues. In another embodiment, the antisense nucleic acid molecule is antisense to a “noncoding region” of the coding strand of a nucleotide sequence encoding the MOLX protein. The term “noncoding region” refers to 5′ and 3′ sequences which flank the coding region that are not translated into amino acids (i.e., also referred to as 5′ and 3′ untranslated regions).

Given the coding strand sequences encoding the MOLX protein disclosed herein, antisense nucleic acids of the invention can be designed according to the rules of Watson and Crick or Hoogsteen base pairing. The antisense nucleic acid molecule can be complementary to the entire coding region of MOLX mRNA, but more preferably is an oligonucleotide that is antisense to only a portion of the coding or noncoding region of MOLX mRNA. For example, the antisense oligonucleotide can be complementary to the region surrounding the translation start site of MOLX mRNA. An antisense oligonucleotide can be, for example, about 5, 10, 15, 20, 25, 30, 35, 40, 45 or 50 nucleotides in length. An antisense nucleic acid of the invention can be constructed using chemical synthesis or enzymatic ligation reactions using procedures known in the art. For example, an antisense nucleic acid (e.g., an antisense oligonucleotide) can be chemically synthesized using naturally-occurring nucleotides or variously modified nucleotides designed to increase the biological stability of the molecules or to increase the physical stability of the duplex formed between the antisense and sense nucleic acids (e.g, phosphorothioate derivatives and acridine substituted nucleotides can be used).

Examples of modified nucleotides that can be used to generate the antisense nucleic acid include: 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, and 2,6-diaminopurine. Alternatively, the antisense nucleic acid can be produced biologically using an expression vector into which a nucleic acid has been subcloned in an antisense orientation (i.e., RNA transcribed from the inserted nucleic acid will be of an antisense orientation to a target nucleic acid of interest, described further in the following subsection).

The antisense nucleic acid molecules of the invention are typically administered to a subject or generated in situ such that they hybridize with or bind to cellular mRNA and/or genonic DNA encoding an MOLX protein to thereby inhibit expression of the protein (e g, by inhibiting transcription and/or translation). The hybridization can be by conventional nucleotide complementarity to form a stable duplex, or, for example, in the case of an antisense nucleic acid molecule that binds to DNA duplexes, through specific interactions in the major groove of the double helix. An example of a route of administration of antisense nucleic acid molecules of the invention includes direct injection at a tissue site. Alternatively, antisense nucleic acid molecules can be modified to target selected cells and then administered systemically. For example, for systemic administration, antisense molecules can be modified such that they specifically bind to receptors or antigens expressed on a selected cell surface (e g., by linking the antisense nucleic acid molecules to peptides or antibodies that bind to cell surface receptors or antigens). The antisense nucleic acid molecules can also be delivered to cells using the vectors described herein. To achieve sufficient nucleic acid molecules, vector constructs in which the antisense nucleic acid molecule is placed under the control of a strong pol II or pol III promoter are preferred.

In yet another embodiment, the antisense nucleic acid molecule of the invention is an α-anomeric nucleic acid molecule. An α-anomeric nucleic acid molecule forms specific double-stranded hybrids with complementary RNA in which, contrary to the usual β-units, the strands run parallel to each other. See, e.g., Gaultier, et al., 1987. Nucl. Acids Res. 15: 6625-6641. The antisense nucleic acid molecule can also comprise a 2′-o-methylribonucleotide (see, e.g., Inoue, et al. 1987. Nucl. Acids Res. 15: 6131-6148) or a chimeric RNA-DNA analogue (see, e.g., Inoue, et al., 1987. FEBS Lett. 215: 327-330.

### Ribozymes and PNA Moieties

Nucleic acid modifications include, by way of non-limiting example, modified bases, and nucleic acids whose sugar phosphate backbones are modified or derivatized. These modifications are carried out at least in part to enhance the chemical stability of the modified nucleic acid, such that they may be used, for example, as antisense binding nucleic acids in therapeutic applications in a subject.

In one embodiment, an antisense nucleic acid of the invention is a ribozyme. Ribozymes are catalytic RNA molecules with ribonuclease activity that are capable of cleaving a single-stranded nucleic acid, such as an mRNA, to which they have a complementary region. Thus, ribozymes (e.g, hammerhead ribozymes as described in Haselhoff and Gerlach 1988. Nature 334: 585-591) can be used to catalytically cleave MOLX mRNA transcripts to thereby inhibit translation of MOLX mRNA. A ribozyme having specificity for an MOLX-encoding nucleic acid can be designed based upon the nucleotide sequence of an MOLX cDNA disclosed herein (i.e., SEQ ID NOS:1, 3, 5, 7, 9, 11, 13, 15, 17, 19, 21, 23, 101, 103, 105, 107, 109, 112, 115, 117, 119, 121, and 124). For example, a derivative of a Tetrahymena L-19 IVS RNA can be constructed in which the nucleotide sequence of the active site is complementary to the nucleotide sequence to be cleaved in an MOLX-encoding mRNA. See, e.g., U.S. Pat. No. 4,987,071 to Cech, et al. and U.S. Pat. No. 5,116,742 to Cech, et al. MOLX mRNA can also be used to select a catalytic RNA having a specific ribonuclease activity from a pool of RNA molecules. See, e.g., Bartel et al., (1993) Science 261:1411-1418.

Alternatively, MOLX gene expression can be inhibited by targeting nucleotide sequences complementary to the regulatory region of the MOLX nucleic acid (e g., the MOLX promoter and/or enhancers) to form triple helical structures that prevent transcription of the MOLX gene in target cells. See, e g., Helene, 1991. Anticancer Drug Des. 6: 569-84; Helene, et al. 1992. Ann. N.Y. Acad. Sci. 660: 27-36; Maher, 1992. Bioassays 14: 807-15.

In various embodiments, the MOLX nucleic acids can be modified at the base moiety, sugar moiety or phosphate backbone to improve, e.g., the stability, hybridization, or solubility of the molecule. For example, the deoxyribose phosphate backbone of the nucleic acids can be modified to generate peptide nucleic acids. (See, e.g., Hyrup, et al., 1996. Bioorg Med Chem 4: 5-23. As used herein, the terms “peptide nucleic acids” or “PNAs” refer to nucleic acid mimics (e.g., DNA mimics) in which the deoxyribose phosphate backbone is replaced by a pseudopeptide backbone and only the four natural nucleobases are retained. The neutral backbone of PNAs has been shown to allow for specific hybridization to DNA and RNA under conditions of low ionic strength. The synthesis of PNA oligomers can be performed using standard solid phase peptide synthesis protocols as described in Hyrup, et al., 1996. supra; Perry-O'Keefe, et al., 1996. Proc. Natl. Acad Sci USA 93: 14670-14675.

PNAs of MOLX can be used in therapeutic and diagnostic applications. For example, PNAs can be used as antisense or antigene agents for sequence-specific modulation of gene expression by, e.g., inducing transcription or translation arrest or inhibiting replication. PNAs of MOLX can also be used, for example, in the analysis of single base pair mutations in a gene (e.g., PNA directed PCR clamping; as artificial restriction enzymes when used in combination with other enzymes, e.g., S₁ nucleases (see, Hyrup, et al., 1996. supra); or as probes or primers for DNA sequence and hybridization (see, Hyrup, et al., 1996, supra; Perry-O'Keefe, et al., 1996. supra).

In another embodiment, PNAs of MOLX can be modified, e.g., to enhance their stability or cellular uptake, by attaching lipophilic or other helper groups to PNA, by the formation of PNA-DNA chimeras, or by the use of liposomes or other techniques of drug delivery known in the art. For example, PNA-DNA chimeras of MOLX can be generated that may combine the advantageous properties of PNA and DNA. Such chimeras allow DNA recognition enzymes (e.g., RNase H and DNA polymerases) to interact with the DNA portion while the PNA portion would provide high binding affinity and specificity. PNA-DNA chimeras can be linked using linkers of appropriate lengths selected in terms of base stacking, number of bonds between the nucleobases, and orientation (see, Hyrup, et al., 1996. supra). The synthesis of PNA-DNA chimeras can be performed as described in Hyrup, et al., 1996. supra and Finn, et al., 1996. Nucl Acids Res 24: 3357-3363. For example, a DNA chain can be synthesized on a solid support using standard phosphoramidite coupling chemistry, and modified nucleoside analogs, e.g., 5′-(4-methoxytrityl)amino-5′-deoxy-thymidine phosphoramidite, can be used between the PNA and the 5′ end of DNA. See, e.g., Mag, et al., 1989. Nucl Acid Res 17: 5973-5988. PNA monomers are then coupled in a stepwise manner to produce a chimeric molecule with a 5′ PNA segment and a 3′ DNA segment. (See, e g, Finn, et al., 1996. supra. Alternatively, chimeric molecules can be synthesized with a 5′ DNA segment and a 3′ PNA segment. See, e.g., Petersen, et al., 1975. Bioorg Med. Chem. Lett. 5: 1119-11124.

In other embodiments, the oligonucleotide may include other appended groups such as peptides (e.g, for targeting host cell receptors in vivo), or agents facilitating transport across the cell membrane (see, e.g, Letsinger, et al., 1989. Proc. Natl. Acad Sci. U.S.A. 86: 6553-6556; Lemaitre, et al., 1987. Proc. Natl. Acad. Sci. 84: 648-652; PCT Publication No. WO88/09810) or the blood-brain barrier (see, e.g., PCT Publication No. WO 89/10134). In addition, oligonucleotides can be modified with hybridization triggered cleavage agents (see, e.g., Krol, et al., 1988. BioTechniques 6:958-976) or intercalating agents (see, e.g., Zon, 1988. Pharm Res. 5: 539-549). To this end, the oligonucleotide may be conjugated to another molecule, e.g., a peptide, a hybridization triggered cross-linking agent, a transport agent, a hybridization-triggered cleavage agent, and the like.

### MOLX Polypeptides

A polypeptide according to the invention includes a polypeptide including the amino acid sequence of MOLX polypeptides whose sequences are provided in SEQ ID NOS:2, 4, 6, 8, 10, 12, 14, 16, 18, 20, 22, 24, 102, 104, 106, 108, 111, 114, 116, 118, 120, 123, and 125. The invention also includes a mutant or variant protein any of whose residues may be changed from the corresponding residues shown in SEQ ID NOS:2, 4, 6, 8, 10, 12, 14, 16, 18, 20, 22, 24, 102, 104, 106, 108, 111, 114, 116, 118, 120, 123, and 125 while still encoding a protein that maintains its MOLX activities and physiological functions, or a functional fragment thereof.

In general, an MOLX variant that preserves MOLX-like function includes any variant in which residues at a particular position in the sequence have been substituted by other amino acids, and further include the possibility of inserting an additional residue or residues between two residues of the parent protein as well as the possibility of deleting one or more residues from the parent sequence. Any amino acid substitution, insertion, or deletion is encompassed by the invention. In favorable circumstances, the substitution is a conservative substitution as defined above.

One aspect of the invention pertains to isolated MOLX proteins, and biologically-active portions thereof, or derivatives, fragments, analogs or homologs thereof. Also provided are polypeptide fragments suitable for use as immunogens to raise anti-MOLX antibodies. In one embodiment, native MOLX proteins can be isolated from cells or tissue sources by an appropriate purification scheme using standard protein purification techniques. In another embodiment, MOLX proteins are produced by recombinant DNA techniques. Alternative to recombinant expression, an MOLX protein or polypeptide can be synthesized chemically using standard peptide synthesis techniques.

An “isolated” or “purified” polypeptide or protein or biologically-active portion thereof is substantially free of cellular material or other contaminating proteins from the cell or tissue source from which the MOLX protein is derived, or substantially free from chemical precursors or other chemicals when chemically synthesized. The language “substantially free of cellular material” includes preparations of MOLX proteins in which the protein is separated from cellular components of the cells from which it is isolated or recombinantly-produced. In one embodiment, the language “substantially free of cellular material” includes preparations of MOLX proteins having less than about 30% (by dry weight) of non-MOLX proteins (also referred to herein as a “contaminating protein”), more preferably less than about 20% of non-MOLX proteins, still more preferably less than about 10% of non-MOLX proteins, and most preferably less than about 5% of non-MOLX proteins. When the MOLX protein or biologically-active portion thereof is recombinantly-produced, it is also preferably substantially free of culture medium, i e., culture medium represents less than about 20%, more preferably less than about 10%, and most preferably less than about 5% of the volume of the MOLX protein preparation.

The language “substantially free of chemical precursors or other chemicals” includes preparations of MOLX proteins in which the protein is separated from chemical precursors or other chemicals that are involved in the synthesis of the protein. In one embodiment, the language “substantially free of chemical precursors or other chemicals” includes preparations of MOLX proteins having less than about 30% (by dry weight) of chemical precursors or non-MOLX chemicals, more preferably less than about 20% chemical precursors or non-MOLX chemicals, still more preferably less than about 10% chemical precursors or non-MOLX chemicals, and most preferably less than about 5% chemical precursors or non-MOLX chemicals.

Biologically-active portions of MOLX proteins include peptides comprising amino acid sequences sufficiently homologous to or derived from the amino acid sequences of the MOLX proteins (e.g., the amino acid sequence shown in SEQ ID NOS:2, 4, 6, 8, 10, 12, 14, 16, 18, 20, 22, 24, 102, 104, 106, 108, 111, 114, 116, 118, 120, 123, and 125) that include fewer amino acids than the full-length MOLX proteins, and exhibit at least one activity of an MOLX protein. Typically, biologically-active portions comprise a domain or motif with at least one activity of the MOLX protein. A biologically-active portion of an MOLX protein can be a polypeptide which is, for example, 10, 25, 50, 100 or more amino acid residues in length.

Moreover, other biologically-active portions, in which other regions of the protein are deleted, can be prepared by recombinant techniques and evaluated for one or more of the functional activities of a native MOLX protein.

In an embodiment, the MOLX protein has an amino acid sequence shown in SEQ ID NOS:2, 4, 6, 8, 10, 12, 14, 16, 18, 20, 22, 24, 102, 104, 106, 108, 111, 114, 116, 118, 120, 123, and 125. In other embodiments, the MOLX protein is substantially homologous to SEQ ID NOS:2, 4, 6, 8, 10, 12, 14, 16, 18, 20, 22, 24, 102, 104, 106, 108, 111, 114, 116, 118, 120, 123, and 125, and retains the functional activity of the protein of SEQ ID NOS:2, 4, 6, 8, 10, 12, 14, 16, 18, 20, 22, 24, 102, 104, 106, 108, 111, 114, 116, 118, 120, 123, and 125, yet differs in amino acid sequence due to natural allelic variation or mutagenesis, as described in detail, below. Accordingly, in another embodiment, the MOLX protein is a protein that comprises an amino acid sequence at least about 45% homologous to the amino acid sequence SEQ ID NOS:2, 4, 6, 8, 10, 12, 14, 16, 18, 20, 22, 24, 102, 104, 106, 108, 111, 114, 116, 118, 120, 123, and 125, and retains the functional activity of the MOLX proteins of SEQ ID NOS:2, 4, 6, 8, 10, 12, 14, 16, 18, 20, 22, 24, 102, 104, 106, 108, 111, 114, 116, 118, 120, 123, and 125.

### Determining Homology Between Two or More Sequences

To determine the percent homology of two amino acid sequences or of two nucleic acids, the sequences are aligned for optimal comparison purposes (e.g., gaps can be introduced in the sequence of a first amino acid or nucleic acid sequence for optimal alignment with a second amino or nucleic acid sequence). The amino acid residues or nucleotides at corresponding amino acid positions or nucleotide positions are then compared. When a position in the first sequence is occupied by the same amino acid residue or nucleotide as the corresponding position in the second sequence, then the molecules are homologous at that position (i.e., as used herein amino acid or nucleic acid “homology” is equivalent to amino acid or nucleic acid “identity”).

The nucleic acid sequence homology may be determined as the degree of identity between two sequences. The homology may be determined using computer programs known in the art, such as GAP software provided in the GCG program package. See, Needleman and Wunsch, 1970. J Mol Biol 48: 443-453. Using GCG GAP software with the following settings for nucleic acid sequence comparison: GAP creation penalty of 5.0 and GAP extension penalty of 0.3, the coding region of the analogous nucleic acid sequences referred to above exhibits a degree of identity preferably of at least 70%, 75%, 80%, 85%, 90%, 95%, 98%, or 99%, with the CDS (encoding) part of the DNA sequence shown in SEQ ID NOS:1, 3, 5, 7, 9, 11, 13, 15, 17, 19, 21, 23, 101, 103, 105, 107, 109, 112, 115, 117, 119, 121, and 124.

The term “sequence identity” refers to the degree to which two polynucleotide or polypeptide sequences are identical on a residue-by-residue basis over a particular region of comparison. The term “percentage of sequence identity” is calculated by comparing two optimally aligned sequences over that region of comparison, determining the number of positions at which the identical nucleic acid base (e g., A, T, C, G, U, or I, in the case of nucleic acids) occurs in both sequences to yield the number of matched positions, dividing the number of matched positions by the total number of positions in the region of comparison (i.e., the window size), and multiplying the result by 100 to yield the percentage of sequence identity. The term “substantial identity” as used herein denotes a characteristic of a polynucleotide sequence, wherein the polynucleotide comprises a sequence that has at least 80 percent sequence identity, preferably at least 85 percent identity and often 90 to 95 percent sequence identity, more usually at least 99 percent sequence identity as compared to a reference sequence over a comparison region.

### Chimeric and Fusion Proteins

The invention also provides MOLX chimeric or fusion proteins. As used herein, an MOLX “chimeric protein” or “fusion protein” comprises an MOLX polypeptide operatively-linked to a non-MOLX polypeptide. An “MOLX polypeptide” refers to a polypeptide having an amino acid sequence corresponding to an MOLX protein (SEQ ID NOS:2, 4, 6, 8, 10, 12, 14, 16, 18, 20, 22, 24, 102, 104, 106, 108, 111, 114, 116, 118, 120, 123, and 125), whereas a “non-MOLX polypeptide” refers to a polypeptide having an amino acid sequence corresponding to a protein that is not substantially homologous to the MOLX protein, e.g, a protein that is different from the MOLX protein and that is derived from the same or a different organism. Within an MOLX fusion protein the MOLX polypeptide can correspond to all or a portion of an MOLX protein. In one embodiment, an MOLX fusion protein comprises at least one biologically-active portion of an MOLX protein. In another embodiment, an MOLX fusion protein comprises at least two biologically-active portions of an MOLX protein. In yet another embodiment, an MOLX fusion protein comprises at least three biologically-active portions of an MOLX protein. Within the fusion protein, the term “operatively-linked” is intended to indicate that the MOLX polypeptide and the non-MOLX polypeptide are fused in-frame with one another. The non-MOLX polypeptide can be fused to the N-terminus or C-terminus of the MOLX polypeptide.

In one embodiment, the fusion protein is a GST-MOLX fusion protein in which the MOLX sequences are fused to the C-terminus of the GST (glutathione S-transferase) sequences. Such fusion proteins can facilitate the purification of recombinant MOLX polypeptides.

In another embodiment, the fusion protein is an MOLX protein containing a heterologous signal sequence at its N-terminus. In certain host cells (e.g., mammalian host cells), expression and/or secretion of MOLX can be increased through use of a heterologous signal sequence.

In yet another embodiment, the fusion protein is an MOLX-immunoglobulin fusion protein in which the MOLX sequences are fused to sequences derived from a member of the immunoglobulin protein family. The MOLX-immunoglobulin fusion proteins of the invention can be incorporated into pharmaceutical compositions and administered to a subject to inhibit an interaction between an MOLX ligand and an MOLX protein on the surface of a cell, to thereby suppress MOLX-mediated signal transduction in vivo. The MOLX-immunoglobulin fusion proteins can be used to affect the bioavailability of an MOLX cognate ligand. Inhibition of the MOLX ligand/MOLX interaction may be useful therapeutically for both the treatment of proliferative and differentiative disorders, as well as modulating (e.g. promoting or inhibiting) cell survival. Moreover, the MOLX-immunoglobulin fusion proteins of the invention can be used as immunogens to produce anti-MOLX antibodies in a subject, to purify MOLX ligands, and in screening assays to identify molecules that inhibit the interaction of MOLX with an MOLX ligand.

An MOLX chimeric or fusion protein of the invention can be produced by standard recombinant DNA techniques. For example, DNA fragments coding for the different polypeptide sequences are ligated together in-frame in accordance with conventional techniques, e.g., by employing blunt-ended or stagger-ended termini for ligation, restriction enzyme digestion to provide for appropriate termini, filling-in of cohesive ends as appropriate, alkaline phosphatase treatment to avoid undesirable joining, and enzymatic ligation. In another embodiment, the fusion gene can be synthesized by conventional techniques including automated DNA synthesizers. Alternatively, PCR amplification of gene fragments can be carried out using anchor primers that give rise to complementary overhangs between two consecutive gene fragments that can subsequently be annealed and reamplified to generate a chimeric gene sequence (see, e.g., Ausubel, et al. (eds.) CURRENT PROTOCOLS IN MOLECULAR BIOLOGY, John Wiley & Sons, 1992). Moreover, many expression vectors are commercially available that already encode a fusion moiety (e.g, a GST polypeptide). An MOLX-encoding nucleic acid can be cloned into such an expression vector such that the fusion moiety is linked in-frame to the MOLX protein.

### MOLX Agonists and Antagonists

The invention also pertains to variants of the MOLX proteins that function as either MOLX agonists (i.e., mimetics) or as MOLX antagonists. Variants of the MOLX protein can be generated by mutagenesis (e.g., discrete point mutation or truncation of the MOLX protein). An agonist of the MOLX protein can retain substantially the same, or a subset of, the biological activities of the naturally occurring form of the MOLX protein. An antagonist of the MOLX protein can inhibit one or more of the activities of the naturally occurring form of the MOLX protein by, for example, competitively binding to a downstream or upstream member of a cellular signaling cascade which includes the MOLX protein. Thus, specific biological effects can be elicited by treatment with a variant of limited function. In one embodiment, treatment of a subject with a variant having a subset of the biological activities of the naturally occurring form of the protein has fewer side effects in a subject relative to treatment with the naturally occurring form of the MOLX proteins.

Variants of the MOLX proteins that function as either MOLX agonists (i.e., mimetics) or as MOLX antagonists can be identified by screening combinatorial libraries of mutants (e.g., truncation mutants) of the MOLX proteins for MOLX protein agonist or antagonist activity. In one embodiment, a variegated library of MOLX variants is generated by combinatorial mutagenesis at the nucleic acid level and is encoded by a variegated gene library. A variegated library of MOLX variants can be produced by, for example, enzymatically ligating a mixture of synthetic oligonucleotides into gene sequences such that a degenerate set of potential MOLX sequences is expressible as individual polypeptides, or alternatively, as a set of larger fusion proteins (e.g., for phage display) containing the set of MOLX sequences therein. There are a variety of methods which can be used to produce libraries of potential MOLX variants from a degenerate oligonucleotide sequence. Chemical synthesis of a degenerate gene sequence can be performed in an automatic DNA synthesizer, and the synthetic gene then ligated into an appropriate expression vector. Use of a degenerate set of genes allows for the provision, in one mixture, of all of the sequences encoding the desired set of potential MOLX sequences. Methods for synthesizing degenerate oligonucleotides are well-known within the art. See, e.g., Narang, 1983. Tetrahedron 39: 3; Itakura, et al., 1984. Annu. Rev. Biochem. 53: 323; Itakura, et al., 1984. Science 198: 1056; Ike, et al., 1983. Nucl. Acids Res. 11: 477.

### Polypeptide Libraries

In addition, libraries of fragments of the MOLX protein coding sequences can be used to generate a variegated population of MOLX fragments for screening and subsequent selection of variants of an MOLX protein. In one embodiment, a library of coding sequence fragments can be generated by treating a double stranded PCR fragment of an MOLX coding sequence with a nuclease under conditions wherein nicking occurs only about once per molecule, denaturing the double stranded DNA, renaturing the DNA to form double-stranded DNA that can include sense/antisense pairs from different nicked products, removing single stranded portions from reformed duplexes by treatment with S₁ nuclease, and ligating the resulting fragment library into an expression vector. By this method, expression libraries can be derived which encodes N-terminal and internal fragments of various sizes of the MOLX proteins.

Various techniques are known in the art for screening gene products of combinatorial libraries made by point mutations or truncation, and for screening cDNA libraries for gene products having a selected property. Such techniques are adaptable for rapid screening of the gene libraries generated by the combinatorial mutagenesis of MOLX proteins. The most widely used techniques, which are amenable to high throughput analysis, for screening large gene libraries typically include cloning the gene library into replicable expression vectors, transforming appropriate cells with the resulting library of vectors, and expressing the combinatorial genes under conditions in which detection of a desired activity facilitates isolation of the vector encoding the gene whose product was detected. Recursive ensemble mutagenesis (REM), a new technique that enhances the frequency of functional mutants in the libraries, can be used in combination with the screening assays to identify MOLX variants. See, e.g., Arkin and Yourvan, 1992. Proc. Natl. Acad. Sci. USA 89: 7811-7815; Delgrave, et al., 1993. Protein Engineering 6:327-331.

### Anti-MOLX Antibodies

The invention encompasses antibodies and antibody fragments, such as F_{ab} or (F_{ab})_{2.} that bind immunospecifically to any of the MOLX polypeptides of said invention.

An isolated MOLX protein, or a portion or fragment thereof, can be used as an immunogen to generate antibodies that bind to MOLX polypeptidles using standard techniques for polyclonal and monoclonal antibody preparation. The full-length MOLX proteins can be used or, alternatively, the invention provides antigenic peptide fragments of MOLX proteins for use as immunogens. The antigenic MOLX peptides comprises at least 4 amino acid residues of the amino acid sequence shown in SEQ ID NOS:2, 4, 6, 8, 10, 12, 14, 16, 18, 20, 22, 24, 102, 104, 106, 108, 111, 114, 116, 118, 120, 123, and 125 and encompasses an epitope of MOLX Such that an antibody raised against the peptide forms a specific immune complex with MOLX. Preferably, the antigenic peptide comprises at least 6, 8, 10, 15, 20, or 30 amino acid residues. Longer antigenic peptides are sometimes preferable over shorter antigenic peptides, depending on use and according to methods well known to someone skilled in the art.

In certain embodiments of the invention, at least one epitope encompassed by the antigenic peptide is a region of MOLX that is located on the surface of the protein (e.g, a hydrophilic region). As a means for targeting antibody production, hydropathy plots showing regions of hydrophilicity and hydrophobicity may be generated by any method well known in the art, including, for example, the Kyte Doolittle or the Hopp Woods methods, either with or without Fourier transformation (see, e.g., Hopp and Woods, 1981. Proc Nat Acad Sci. USA 78: 3824-3828; Kyte and Doolittle, 1982. J. Mol. Biol. 157: 105-142, each incorporated herein by reference in their entirety).

As disclosed herein, MOLX protein sequences of SEQ ID NOS:2, 4, 6, 8, 10, 12, 14, 16, 18, 20, 22, 24, 102, 104, 106, 108, 111, 114, 116, 118, 120, 123, and 125, or derivatives, fragments, analogs or homologs thereof, may be utilized as immunogens in the generation of antibodies that immunospecifically-bind these protein components. The term “antibody” as used herein refers to immunoglobulin molecules and immunologically-active portions of immunoglobulin molecules, i e., molecules that contain an antigen binding site that specifically-binds (immunoreacts with) an antigen, such as MOLX. Such antibodies include, but are not limited to, polyclonal, monoclonal, chimeric, single chain, F_{ab} and F(_{ab′)2} fragments, and an F_{ab} expression library. In a specific embodiment, antibodies to human MOLX proteins are disclosed. Various procedures known within the art may be used for the production of polyclonal or monoclonal antibodies to an MOLX protein sequence of SEQ ID NOS:2, 4, 6, 8, 10, 12, 14, 16, 18, 20, 22, 24, 102, 104, 106, 108, 111, 114, 116, 118, 120, 123, and 125, or a derivative, fragment, analog or homolog thereof. Some of these proteins are discussed below.

For the production of polygonal antibodies, various suitable host animals (e g, rabbit, goat, mouse or other mammal) may be immunized by injection with the native protein, or a synthetic variant thereof or a derivative of the foregoing. An appropriate immunogenic preparation can contain, for example, recombinantly-expressed MOLX protein or a chemically-synthesized MOLX polypeptide. The preparation can further include an adjuvant. Various adjuvants used to increase the immunological response include, but are not limited to, Freund's (complete and incomplete), mineral gels (e g., aluminum hydroxide), surface active substances (e.g., lysolecithin, pluronic polyols, polyanions, peptides, oil emulsions, dinitrophenol, etc.), human adjuvants such as Bacille Calmette-Guerin and Corynebacterium parvum, or similar immunostimulatory agents. If desired, the antibody molecules directed against MOLX can be isolated from the mammal (e g, from the blood) and further purified by well known techniques, such as protein A chromatography to obtain the IgG fraction.

The term “monoclonal antibody” or “monoclonal antibody composition”, as used herein, refers to a population of antibody molecules that contain only one species of an antigen binding site capable of immunoreacting with a particular epitope of MOLX. A monoclonal antibody composition thus typically displays a single binding affinity for a particular MOLX protein with which it immunoreacts. For preparation of monoclonal antibodies directed towards a particular MOLX protein, or derivatives, fragments, analogs or homologs thereof, any technique that provides for the production of antibody molecules by continuous cell line culture may be utilized. Such techniques include, but are not limited to, the hybridoma technique (see, e.g., Kohler & Milstein, 1975. Nature 256: 495-497), the trioma technique; the human B-cell hybridoma technique (see, e.g., Kozbor, et al., 1983. Immunol. Today 4: 72) and the EBV hybridoma technique to produce human monoclonal antibodies (see, e.g, Cole, et al., 1985. In: MONOCLONAL ANTIBODIES AND CANCER THERAPY, Alan R. Liss, Inc., pp. 77-96). Human monoclonal antibodies may be utilized in the practice of the invention and may be produced by using human hybridomas (see, e g, Cote, et al., 1983. Proc Natl Acad Sci USA 80: 2026-2030) or by transforming human B-cells with Epstein Barr Virus in vitro (see, e g., Cole, et al, 1985. In: MONOCLONAL ANTIBODIES AND CANCER THERAPY, Alan R. Liss, Inc., pp. 77-96). Each of the above citations is incorporated herein by reference in their entirety.

According to the invention, techniques can be adapted for the production of single-chain antibodies specific to an MOLX protein (see, e.g., U.S. Pat. No. 4,946,778). In addition, methods can be adapted for the construction of F_{ab} expression libraries (see, e.g., Huse, et al, 1989. Science 246: 1275-1281) to allow rapid and effective identification of monoclonal F_{ab} fragments with the desired specificity for an MOLX protein or derivatives, fragments, analogs or homologs thereof. Non-human antibodies can be “humanized” by techniques well known in the art. See, e g, U.S. Pat. No. 5,225,539. Antibody fragments that contain the idiotypes to an MOLX protein may be produced by techniques known in the art including, but not limited to: (i) an F_{(ab′)2} fragment produced by pepsin digestion of an antibody molecule; (ii) an F_{ab} fragment generated by reducing the disulfide bridges of an F_{(ab′)2} fragment; (iii) an F_{ab} fragment generated by the treatment of the antibody molecule with papain and a reducing agent; and (iv) Fᵥ fragments.

Additionally, recombinant anti-MOLX antibodies, such as chimeric and humanized monoclonal antibodies, comprising both human and non-human portions, which can be made using standard recombinant DNA techniques, are within the scope of the invention. Such chimeric and humanized monoclonal antibodies can be produced by recombinant DNA techniques known in the art, for example using methods described in International Application No. PCT/US86/02269; European Patent Application No. 184,187; European Patent Application No. 171,496; European Patent Application No. 173,494; PCT International Publication No. WO 86/01533; U.S. Pat. No. 4,816,567; U.S. Pat. No. 5,225,539; European Patent Application No. 125,023; Better, et al., 1988. Science 240: 1041-1043; Lui, et al., 1987. Proc Natl Acad. Sci USA 84: 3439-3443, Lui, et al., 1987. J. Immunol. 139: 3521-3526; Sun, et al., 1987. Proc. Natl Acad. Sci USA 84: 214-218; Nishimura, et al., 1987. Cancer Res 47: 999-1005; Wood, et al., 1985. Nature 314:446-449; Shaw, et al., 1988. J Natl. Cancer Inst. 80: 1553-1559); Morrison (1985) Science 229:1202-1207; Oi, et al. (1986) BioTechniques 4:214; Jones, et al., 1986. Nature 321: 552-525; Verhoeyan, et al., 1988. Science 239: 1534; and Beidler, et al., 1988. J. Immunol. 141: 4053-4060. Each of the above citations are incorporated herein by reference in their entirety.

In one embodiment, methods for the screening of antibodies that possess the desired specificity include, but are not limited to, enzyme-linked immunosorbent assay (ELISA) and other immunologically-mediated techniques known within the art. In a specific embodiment, selection of antibodies that are specific to a particular domain of an MOLX protein is facilitated by generation of hybridomas that bind to the fragment of an MOLX protein possessing such a domain. Thus, antibodies that are specific for a desired domain within an MOLX protein, or derivatives, fragments, analogs or homologs thereof are also provided herein.

Anti-MOLX antibodies may be used in methods known within the art relating to the localization and/or quantitation of an MOLX protein (e.g, for use in measuring levels of the MOLX protein within appropriate physiological samples, for use in diagnostic methods, for use in imaging the protein, and the like). In a given embodiment, antibodies for MOLX proteins, or derivatives, fragments, analogs or homologs thereof, that contain the antibody derived binding domain, are utilized as pharmacologically-active compounds (hereinafter “Therapeutics”).

An anti-MOLX antibody (e.g., monoclonal antibody) can be used to isolate an MOLX polypeptide by standard techniques, such as affinity chromatography or immunoprecipitation. An anti-MOLX antibody can facilitate the purification of natural MOLX polypeptide from cells and of recombinantly-produced MOLX polypeptide expressed in host cells. Moreover, an anti-MOLX antibody can be used to detect MOLX protein (e.g., in a cellular lysate or cell supernatant) in order to evaluate the abundance and pattern of expression of the MOLX protein. Anti-MOLX antibodies can be used diagnostically to monitor protein levels in tissue as part of a clinical testing procedure, e g, to, for example, determine the efficacy of a given treatment regimen. Detection can be facilitated by coupling (i.e., physically linking) the antibody to a detectable substance. Examples of detectable substances include various enzymes, prosthetic groups, fluorescent materials, luminescent materials, bioluminescent materials, and radioactive materials. Examples of suitable enzymes include horseradish peroxidase, alkaline phosphatase, β-galactosidase, or acetylcholinesterase; examples of suitable prosthetic group complexes include streptavidin/biotin and avidin/biotin; examples of suitable fluorescent materials include umbelliferone, fluorescein, fluorescein isothiocyanate, rhodamine, dichlorotriazinylamine fluorescein, dansyl chloride or phycoerythrin; an example of a luminescent material includes luminol; examples of bioluminescent materials include luciferase, luciferin, and aequorin, and examples of suitable radioactive material include ¹²⁵I, ¹³¹I, ³⁵S or ³H.

### MOLX Recombinant Expression Vectors and Host Cells

Another aspect of the invention pertains to vectors, preferably expression vectors, containing a nucleic acid encoding an MOLX protein, or derivatives, fragments, analogs or homologs thereof. As used herein, the term “vector” refers to a nucleic acid molecule capable of transporting another nucleic acid to which it has been linked. One type of vector is a “plasmid”, which refers to a circular double stranded DNA loop into which additional DNA segments can be ligated. Another type of vector is a viral vector, wherein additional DNA segments can be ligated into the viral genome. Certain vectors are capable of autonomous replication in a host cell into which they are introduced (e g, bacterial vectors having a bacterial origin of replication and episomal mammalian vectors). Other vectors (e.g., non-episomal mammalian vectors) are integrated into the genome of a host cell upon introduction into the host cell, and thereby are replicated along with the host genome. Moreover, certain vectors are capable of directing the expression of genes to which they are operatively-linked. Such vectors are referred to herein as “expression vectors”. In general, expression vectors of utility in recombinant DNA techniques are often in the form of plasmids. In the present specification, “plasmids” and “vector” can be used interchangeably as the plasmid is the most commonly used form of vector. However, the invention is intended to include such other forms of expression vectors, such as viral vectors (e g., replication defective retroviruses, adenoviruses and adeno-associated viruses), which serve equivalent functions.

The recombinant expression vectors of the invention comprise a nucleic acid of the invention in a form suitable for expression of the nucleic acid in a host cell, which means that the recombinant expression vectors include one or more regulatory sequences, selected on the basis of the host cells to be used for expression, that is operatively-linked to the nucleic acid sequence to be expressed. Within a recombinant expression vector, “operably-linked” is intended to mean that the nucleotide sequence of interest is linked to the regulatory sequence(s) in a manner that allows for expression of the nucleotide sequence (e.g, in an in vitro transcription/translation system or in a host cell when the vector is introduced into the host cell).

The term “regulatory sequence” is intended to includes promoters, enhancers and other expression control elements (e.g., polyadenylation signals). Such regulatory sequences are described, for example, in Goeddel, GENE EXPRESSION TECHNOLOGY: METHODS IN ENZYMOLOGY 185, Academic Press, San Diego, Calif. (1990). Regulatory sequences include those that direct constitutive expression of a nucleotide sequence in many types of host cell and those that direct expression of the nucleotide sequence only in certain host cells (e g., tissue-specific regulatory sequences). It will be appreciated by those skilled in the art that the design of the expression vector can depend on such factors as the choice of the host cell to be transformed, the level of expression of protein desired, etc. The expression vectors of the invention can be introduced into host cells to thereby produce proteins or peptides, including fusion proteins or peptides, encoded by nucleic acids as described herein (e.g, MOLX proteins, mutant forms of MOLX proteins, fusion proteins, etc.).

The recombinant expression vectors of the invention can be designed for expression of MOLX proteins in prokaryotic or eukaryotic cells. For example, MOLX proteins can be expressed in bacterial cells such as Escherichia coli, insect cells (using baculovirus expression vectors) yeast cells or mammalian cells. Suitable host cells are discussed further in Goeddel, GENE EXPRESSION TECHNOLOGY: METHODS IN ENZYMOLOGY 185, Academic Press, San Diego, Calif. (1990). Alternatively, the recombinant expression vector can be transcribed and translated in vitro, for example using T7 promoter regulatory sequences and T7 polymerase.

Expression of proteins in prokaryotes is most often carried out in Escherichia coli with vectors containing constitutive or inducible promoters directing the expression of either fusion or non-fusion proteins. Fusion vectors add a number of amino acids to a protein encoded therein, usually to the amino terminus of the recombinant protein. Such fusion vectors typically serve three purposes: (i) to increase expression of recombinant protein; (ii) to increase the solubility of the recombinant protein; and (iii) to aid in the purification of the recombinant protein by acting as a ligand in affinity purification. Often, in fusion expression vectors, a proteolytic cleavage site is introduced at the junction of the fusion moiety and the recombinant protein to enable separation of the recombinant protein from the fusion moiety subsequent to purification of the fusion protein. Such enzymes, and their cognate recognition sequences, include Factor Xa, thrombin and enterokinase. Typical fusion expression vectors include pGEX (Pharmacia Biotech Inc; Smith and Johnson, 1988. Gene 67: 31-40), pMAL (New England Biolabs, Beverly, Mass.) and pRIT5 (Pharmacia, Piscataway, N.J.) that fuse glutathione S-transferase (GST), maltose E binding protein, or protein A, respectively, to the target recombinant protein.

Examples of suitable inducible non-fusion E coli expression vectors include pTrc (Amrann et al., (1988) Gene 69:301-315) and pET 11d (Studier et al., GENE EXPRESSION TECHNOLOGY: METHODS IN ENZYMOLOGY 185, Academic Press, San Diego, Calif. (1990) 60-89).

One strategy to maximize recombinant protein expression in E. coli is to express the protein in a host bacteria with an impaired capacity to proteolytically cleave the recombinant protein. See, e.g., Gottesman, GENE EXPRESSION TECHNOLOGY: METHODS IN ENZYMOLOGY 185, Academic Press, San Diego, Calif. (1990) 119-128. Another strategy is to alter the nucleic acid sequence of the nucleic acid to be inserted into an expression vector so that the individual codons for each amino acid are those preferentially utilized in E coli (.see, e.g., Wada, et al., 1992. Nucl Acids Res. 20: 2111-2118). Such alteration of nucleic acid sequences of the invention can be carried out by standard DNA synthesis techniques.

In another embodiment, the MOLX expression vector is a yeast expression vector. Examples of vectors for expression in yeast Saccharomyces cerevisae include pYepSec1 (Baldari, et al., 1987. EMBO J 6: 229-234), pMFa (Kurjan and Herskowitz, 1982. Cell 30: 933-943), pJRY88 (Schultz et al., 1987. Gene 54: 113-123), pYES2 (Invitrogen Corporation, San Diego, Calif.), and picZ (InVitrogen Corp, San Diego, Calif.).

Alternatively, MOLX can be expressed in insect cells using baculovirus expression vectors. Baculovirus vectors available for expression of proteins in cultured insect cells (e.g, SF9 cells) include the pAc series (Smith, et al., 1983. Mol. Cell Biol 3: 2156-2165) and the pVL series (Lucklow and Summers, 1989. Virology 170: 31-39).

In yet another embodiment, a nucleic acid of the invention is expressed in mammalian cells using a mammalian expression vector. Examples of mammalian expression vectors include pCDM8 (Seed, 1987. Nature 329: 840) and pMT2PC (Kaufman, et al., 1987. EMBO J. 6: 187-195). When used in mammalian cells, the expression vector's control functions are often provided by viral regulatory elements. For example, commonly used promoters are derived from polyoma, adenovirus 2, cytomegalovirus, and simian virus 40. For other suitable expression systems for both prokaryotic and eukaryotic cells see, e g., Chapters 16 and 17 of Sambrook, et al., MOLECULAR CLONING: A LABORATORY MANUAL. 2nd ed., Cold Spring Harbor Laboratory, Cold Spring Harbor Laboratory Press, Cold Spring Harbor, N.Y., 1989.

In another embodiment, the recombinant mammalian expression vector is capable of directing expression of the nucleic acid preferentially in a particular cell type (e.g., tissue-specific regulatory elements are used to express the nucleic acid). Tissue-specific regulatory elements are known in the art. Non-limiting examples of suitable tissue-specific promoters include the albumin promoter (liver-specific; Pinkert, et al., 1987. Genes Dev. 1: 268-277), lymphoid-specific promoters (Calame and Eaton, 1988. Adv. Immunol. 43: 235-275), in particular promoters of T cell receptors (Winoto and Baltimore, 1989. EMBO J. 8: 729-733) and immunoglobulins (Banerji, et al., 1983. Cell 33: 729-740; Queen and Baltimore, 1983. Cell 33: 741-748), neuron-specific promoters (e g, the neurofilament promoter; Byrne and Ruddle, 1989. Proc Natl Acad. Sci. USA 86: 5473-5477), pancreas-specific promoters (Edlund, et al., 1985. Science 230: 912-916), and mammary gland-specific promoters (e.g, milk whey promoter; U.S. Pat. No. 4,873,316 and European Application Publication No. 264,166). Developmentally-regulated promoters are also encompassed, e.g., the murine hox promoters (Kessel and Gruss, 1990. Science 249: 374-379) and the α-fetoprotein promoter (Campes and Tilghman, 1989. Genes Dev. 3: 537-546).

The invention further provides a recombinant expression vector comprising a DNA molecule of the invention cloned into the expression vector in an antisense orientation. That is, the DNA molecule is operatively-linked to a regulatory sequence in a manner that allows for expression (by transcription of the DNA molecule) of an RNA molecule that is antisense to MOLX mRNA. Regulatory sequences operatively linked to a nucleic acid cloned in the antisense orientation can be chosen that direct the continuous expression of the antisenise RNA molecule in a variety of cell types, for instance viral promoters and/or enhancers, or regulatory sequences can be chosen that direct constitutive tissue specific or cell type specific expression of antisense RNA. The antisense expression vector can be in the form of a recombinant plasmid, phagemid or attenuated virus in which antisense nucleic acids are produced under the control of a high efficiency regulatory region, the activity of which can be determined by the cell type into which the vector is introduced. For a discussion of the regulation of gene expression using antisense genes see, e g, Weintraub, et al., “Antisense RNA as a molecular tool for genetic analysis,” Reviews-Trends in Genetics, Vol. I(1) 1986.

Another aspect of the invention pertains to host cells into which a recombinant expression vector of the invention has been introduced. The terms “host cell” and “recombinant host cell” are used interchangeably herein. It is understood that such terms refer not only to the particular subject cell but also to the progeny or potential progeny of such a cell. Because certain modifications may occur in succeeding generations due to either mutation or environmental influences, such progeny may not, in fact, be identical to the parent cell, but are still included within the scope of the term as used herein.

A host cell can be any prokaryotic or eukaryotic cell. For example, MOLX protein can be expressed in bacterial cells such as E coli, insect cells, yeast or mammalian cells (such as Chinese hamster ovary cells (CHO) or COS cells). Other suitable host cells are known to those skilled in the art.

Vector DNA can be introduced into prokaryotic or eukaryotic cells via conventional transformation or transfection techniques. As used herein, the terms “transformation” and “transfection” are intended to refer to a variety of art-recognized techniques for introducing foreign nucleic acid (e g, DNA) into a host cell, including calcium phosphate or calcium chloride co-precipitation, DEAE-dextran-mediated transfection, lipofection, or electroporation. Suitable methods for transforming or transfecting host cells can be found in Sambrook, et al (MOLECULAR CLONING: A LABORATORY MANUAL. 2nd ed., Cold Spring Harbor Laboratory, Cold Spring Harbor Laboratory Press, Cold Spring Harbor, N.Y., 1989), and other laboratory manuals.

For stable transfection of mammalian cells, it is known that, depending upon the expression vector and transfection technique used, only a small fraction of cells may integrate the foreign DNA into their genome. In order to identify and select these integrants, a gene that encodes a selectable marker (e g, resistance to antibiotics) is generally introduced into the host cells along with the gene of interest. Various selectable markers include those that confer resistance to drugs, such as G418, hygromycin and methotrexate. Nucleic acid encoding a selectable marker can be introduced into a host cell on the same vector as that encoding MOLX or can be introduced on a separate vector. Cells stably transfected with the introduced nucleic acid can be identified by drug selection (e.g., cells that have incorporated the selectable marker gene will survive, while the other cells die).

A host cell of the invention, such as a prokaryotic or eukaryotic host cell in culture, can be used to produce (i.e, express) MOLX protein. Accordingly, the invention further provides methods for producing MOLX protein using the host cells of the invention. In one embodiment, the method comprises culturing the host cell of invention (into which a recombinant expression vector encoding MOLX protein has been introduced) in a suitable medium such that MOLX protein is produced. In another embodiment, the method further comprises isolating MOLX protein from the medium or the host cell.

### Transgenic MOLX Animals

The host cells of the invention can also be used to produce non-human transgenic animals. For example, in one embodiment, a host cell of the invention is a fertilized oocyte or an embryonic stem cell into which MOLX protein-coding sequences have been introduced. Such host cells can then be used to create non-human transgenic animals in which exogenous MOLX sequences have been introduced into their genome or homologous recombinant animals in which endogenous MOLX sequences have been altered. Such animals are useful for studying the function and/or activity of MOLX protein and for identifying and/or evaluating modulators of MOLX protein activity. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA that is integrated into the genome of a cell from which a transgenic animal develops and that remains in the genome of the mature animal, thereby directing the expression of an encoded gene product in one or more cell types or tissues of the transgenic animal. As used herein, a “homologous recombinant animal” is a non-human animal, preferably a mammal, more preferably a mouse, in which an endogenous MOLX gene has been altered by homologous recombination between the endogenous gene and an exogenous DNA molecule introduced into a cell of the animal, e g, an embryonic cell of the animal, prior to development of the animal.

A transgenic animal of the invention can be created by introducing MOLX-encoding nucleic acid into the male pronuclei of a fertilized oocyte (e g, by microinjection, retroviral infection) and allowing the oocyte to develop in a pseudopregnant female foster animal. The hutman MOLX cDNA sequences of SEQ ID NOS:1, 3, 5, 7, 9, 11, 13, 15, 17, 19, 21, 23, 101, 103, 105, 107, 109, 112, 115, 117, 119, 121, and 124 can be introduced as a transgene into the genome of a non-human animal. Alternatively, a non-human homologue of the human MOLX gene, such as a mouse MOLX gene, can be isolated based on hybridization to the human MOLX cDNA (described further supra) and used as a transgene. Intronic sequences and polyadenylation signals can also be included in the transgene to increase the efficiency of expression of the transgene. A tissue-specific regulatory sequence(s) can be operably-linked to the MOLX transgene to direct expression of MOLX protein to particular cells. Methods for generating transgenic animals via embryo manipulation and microinjection, particularly animals such as mice, have become conventional in the art and are described, for example, in U.S. Pat. Nos. 4,736,866; 4,870,009; and 4,873,191; and Hogan, 1986. In: MANIPULATING THE MOUSE EMBRYO, Cold Spring Harbor Laboratory Press, Cold Spring Harbor, N.Y. Similar methods are used for production of other transgenic animals. A transgenic founder animal can be identified based upon the presence of the MOLX transgene in its genome and/or expression of MOLX mRNA in tissues or cells of the animals. A transgenic founder animal can then be used to breed additional animals carrying the transgene. Moreover, transgenic animals carrying a transgene-encoding MOLX protein can further be bred to other transgenic animals carrying other transgenes.

To create a homologous recombinant animal, a vector is prepared which contains at least a portion of an MOLX gene into which a deletion, addition or substitution has been introduced to thereby alter, e g, functionally disrupt, the MOLX gene. The MOLX gene can be a human gene (e.g, the cDNA of SEQ ID NOS:1, 3, 5, 7, 9, 11, 13, 15, 17, 19, 21, 23, 101, 103, 105, 107, 109, 112, 115, 117, 119, 121, and 124), but more preferably, is a non-human homologue of a human MOLX gene. For example, a mouse homologue of human MOLX gene of SEQ ID NOS:1, 3, 5, 7, 9, 11, 13, 15, 17, 19, 21, 23, 101, 103, 105, 107, 109, 112, 115, 117, 119, 121, and 124 can be used to construct a homologous recombination vector suitable for altering an endogenous MOLX gene in the mouse genome. In one embodiment, the vector is designed such that, upon homologous recombination, the endogenous MOLX gene is functionally disrupted (i e, no longer encodes a functional protein; also referred to as a “knock out” vector).

Alternatively, the vector can be designed such that, upon homologous recombination, the endogenous MOLX gene is mutated or otherwise altered but still encodes functional protein (e.g., the upstream regulatory region can be altered to thereby alter the expression of the endogenous MOLX protein). In the homologous recombination vector, the altered portion of the MOLX gene is flanked at its 5′- and 3′-termini by additional nucleic acid of the MOLX gene to allow for homologous recombination to occur between the exogenous MOLX gene carried by the vector and an endogenous MOLX gene in an embryonic stem cell. The additional flanking MOLX nucleic acid is of sufficient length for successful homologous recombination with the endogenous gene. Typically, several kilobases of flanking DNA (both at the 5′- and 3′-termini) are included in the vector. See, e g., Thomas, et al., 1987. Cell 51: 503 for a description of homologous recombination vectors. The vector is ten introduced into an embryonic stem cell line (e.g., by electroporation) and cells in which the introduced MOLX gene has homologously-recombined with the endogenous MOLX gene are selected. See, e g., Li, et al., 1992. Cell 69:915.

The selected cells are then injected into a blastocyst of an animal (e g, a mouse) to form aggregation chimeras. See, e g, Bradley, 1987. In: TERATOCARCINOMAS AND EMBRYONIC STEM CELLS: A PRACTICAL APPROACH. Robertson, ed. IRL, Oxford. pp. 113-152. A chimeric embryo can then be implanted into a suitable pseudopregnant female foster animal and the embryo brought to term. Progeny harboring the homologIously-recombined DNA in their germ cells can be used to breed animals in which all cells of the animal contain the homologously-recombined DNA by germline transmission of the transgene. Methods for constructing homologous recombination vectors and homologous recombinant animals are described further in Bradley, 1991. Curr. Opin. Biotechnol 2: 823-829; PCT International Publication Nos.: WO 90/11354; WO 91/01140; WO 92/0968; and WO 93/04169.

In another embodiment, transgenic non-humans animals can be produced that contain selected systems that allow for regulated expression of the transgene. One example of such a system is the cre/loxP recombinase system of bacteriophage P1. For a description of the cre/loxP recombinase system, See, e.g., Lakso, et al., 1992. Proc Natl Acad Sci. USA 89: 6232-6236. Another example of a recombinase system is the FLP recombinase system of Saccharocmyces cerevisiae See, O'Gorman, et al., 1991. Science 251:1351-1355. If a cre/loxP recombinase system is used to regulate expression of the transgene, animals containing transgenes encoding both the Cre recombinase and a selected protein are required. Such animals can be provided through the construction of “double” transgenic animals, e g., by mating two transgenic animals, one containing a transgene encoding a selected protein and the other containing a transgene encoding a recombinase.

Clones of the non-human transgenic animals described herein can also be produced according to the methods described in Wilmut, et al., 1997. Nature 385: 810-813. In brief, a cell (e g., a somatic cell) from the transgenic animal can be isolated and induced to exit the growth cycle and enter G₀ phase. The quiescent cell can then be fused, e.g., through the use of electrical pulses, to an enucleated oocyte from an animal of the same species from which the quiescent cell is isolated. The reconstructed oocyte is then cultured such that it develops to morula or blastocyte and then transferred to pseudopregnant female foster animal. The offspring borne of this female foster animal will be a clone of the animal from which the cell (e.g., the somatic cell) is isolated.

### Pharmaceutical Compositions

The MOLX nucleic acid molecules, MOLX proteins, and anti-MOLX antibodies (also referred to herein as “active compounds”) of the invention, and derivatives, fragments, analogs and homologs thereof, can be incorporated into pharmaceutical compositions suitable for administration. Such compositions typically comprise the nucleic acid molecule, protein, or antibody and a pharmaceutically acceptable carrier. As used herein, “pharmaceutically acceptable carrier” is intended to include any and all solvents, dispersion media, coatings, antibacterial and antifungal agents, isotonic and absorption delaying agents, and the like, compatible with pharmaceutical administration. Suitable carriers are described in the most recent edition of Remington's Pharmaceutical Sciences, a standard reference text in the field, which is incorporated herein by reference. Preferred examples of such carriers or diluents include, but are not limited to, water, saline, finger's solutions, dextrose solution, and 5% human serum albumin. Liposomes and non-aqueous vehicles such as fixed oils may also be used. The use of such media and agents for pharmaceutically active substances is well known in the art. Except insofar as any conventional media or agent is incompatible with the active compound, use thereof in the compositions is contemplated. Supplementary active compounds can also be incorporated into the compositions.

A pharmaceutical composition of the invention is formulated to be compatible with its intended route of administration. Examples of routes of administration include parenteral, e.g., intravenous, intradermal, subcutaneous, oral (e.g., inhalation), transdermal (i.e, topical), transmucosal, and rectal administration. Solutions or suspensions used for parenteral, intradermal, or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethyleniediaminetetraacetic acid (EDTA); buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents. for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound (e.g., an MOLX protein or anti-MOLX antibody) in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate. or orange flavoring.

For administration by inhalation. the compounds are delivered in the form of an aerosol spray from pressured container or dispenser which contains a suitable propellant, e g, a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

The compounds can also be prepared in the form of suppositories (e g, with conventional suppository bases such as cocoa butter and other glycericdes) or retention enemas for rectal delivery.

In one embodiment, the active compounds are prepared with carriers that will protect the compound against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art. The materials can also be obtained commercially from Alza Corporation and Nova Pharmaceuticals, Inc. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active compound calculated to produce the desired therapeutic effect in association with the required pharmaceutical carrier. The specification for the dosage unit forms of the invention are dictated by and directly dependent on the unique characteristics of the active compound and the particular therapeutic effect to be achieved, and the limitations inherent in the art of compounding such an active compound for the treatment of individuals.

The nucleic acid molecules of the invention can be inserted into vectors and used as gene therapy vectors. Gene therapy vectors can be delivered to a subject by, for example, intravenous injection, local administration (see, e g. U.S. Pat. No. 5,328,470) or by stereotactic injection (see, e.g., Chen, et al., 1994. Proc. Natl. Acad Sci. USA 91: 3054-3057). The pharmaceutical preparation of the gene therapy vector can include the gene therapy vector in an acceptable diluent, or can comprise a slow release matrix in which the gene delivery vehicle is imbedded. Alternatively, where the complete gene delivery vector can be produced intact from recombinant cells, e.g, retroviral vectors, the pharmaceutical preparation can include one or more cells that produce the gene delivery system.

The pharmaceutical compositions can be included in a containier, pack, or dispenser together with instructions for administration.

### Screening and Detection Methods

The isolated nucleic acid molecules of the invention can be used to express MOLX protein (e.g, via a recombinant expression vector in a host cell in gene therapy applications), to detect MOLX mRNA (e.g, in a biological sample) or a genetic lesion in an MOLX gene, and to modulate MOLX activity, as described further, below. In addition, the MOLX proteins can be used to screen drugs or compounds that modulate the MOLX protein activity or expression as well as to treat disorders characterized by insufficient or excessive production of MOLX protein or production of MOLX protein forms that have decreased or aberrant activity compared to MOLX wild-type protein (e g, diabetes (regulates insulin release); obesity (binds and transport lipids); metabolic disturbances associated with obesity, the metabolic syndrome X as well as anorexia and wasting disorders associated with chronic diseases and various cancers, and infectious disease (possesses anti-microbial activity) and the various dyslipidemias. In addition, the anti-MOLX antibodies of the invention can be used to detect and isolate MOLX proteins and modulate MOLX activity. In yet a further aspect, the invention can be used in methods to influence appetite, absorption of nutrients and the disposition of metabolic substrates in both a positive and negative fashion.

The invention further pertains to novel agents identified by the screening assays described herein and uses thereof for treatments as described, supra.

### Screening Assays

The invention provides a method (also referred to herein as a “screening assay”) for identifying modulators, i e, candidate or test compounds or agents (e g. peptides, peptidomimetics, small molecules or other drugs) that bind to MOLX proteins or have a stimulatory or inhibitory effect on, e.g., MOLX protein expression or MOLX protein activity. The invention also includes compounds identified in the screening assays described herein.

In one embodiment, the invention provides assays for screening candidate or test compounds which bind to or modulate the activity of the membrane-bound form of an MOLX protein or polypeptide or biologically-active portion thereof. The test compounds of the invention can be obtained using any of the numerous approaches in combinatorial library methods known in the art, includCing: biological libraries; spatially addressable parallel solid phase or solution phase libraries; synthetic library methods requiring deconvolution; the “one-bead one-compound” library method; and synthetic library methods using affinity chromatography selection. The biological library approach is limited to peptide libraries, while the other four approaches are applicable to peptide, non-peptide oligomer or small molecule libraries of compounds. See, e g., Lam, 1997. Anticancer Drug Design 12: 145.

A “small molecule” as used herein, is meant to refer to a composition that has a molecular weight of less than about 5 kD and most preferably less than about 4 kD. Small molecules can be, e.g., nucleic acids, peptides, polypeptides, peptidomimetics, carbohydrates, lipids or other organic or inorganic molecules. Libraries of chemical and/or biological mixtures, such as fungal, bacterial, or algal extracts, are known in the art and can be screened with any of the assays of the invention.

Examples of methods for the synthesis of molecular libraries can be found in the art, for example in: DeWitt, et al., 1993. Proc. Natl. Acad. Sci. U.S.A. 90: 6909; Erb, et al., 1994. Proc Natl. Acad. Sci. U.S.A. 91: 11422; Zuckermann, et al., 1994. J. Med Chem 37: 2678; Cho, et al., 1993. Science 261: 1303; Carrell, et al., 1994. Angew Chem. Int Ed Engl 33: 2059; Carell, et al., 1994. Angew. Chem Int Ed. Engl 33: 2061; and Gallop, et al., 1994. J. Med Chem 37: 1233.

Libraries of compounds may be presented in solution (e g., Houghten, 1992. Biotechniques 13: 412-421), or on beads (Lam, 1991. Nature 354: 82-84), on chips (Fodor, 1993. Nature 364: 555-556), bacteria (Ladner, U.S. Pat. No. 5,223,409), spores (Ladner, U.S. Pat. No. 5,233,409), plasmids (Cull, et al., 1992. Proc. Natl. Acad. Sci. USA 89: 1865-1869) or on phage (Scott and Smith, 1990. Science 249: 386-390; Devlin, 1990. Science 249: 404-406; Cwirla, et al., 1990. Proc. Natl Acad. Sci. U.SA. 87: 6378-6382; Felici, 1991. J Mol Biol 222: 301-310; Ladner, U.S. Pat. No. 5,233,409.).

In one embodiment, an assay is a cell-based assay in which a cell which expresses a membrane-bound form of MOLX protein, or a biologically-active portion thereof, on the cell surface is contacted with a test compound and the ability of the test compound to bind to an MOLX protein determined. The cell, for example, can of mammalian origin or a yeast cell. Determining the ability of the test compound to bind to the MOLX protein can be accomplished, for example, by coupling the test compound with a radioisotope or enzymatic label such that binding of the test compound to the MOLX protein or biologically-active portion thereof can be determined by detecting the labeled compound in a complex. For example, test compounds can be labeled with ¹²⁵I, ³⁵S, ¹⁴C, or ³H, either directly or indirectly, and the radioisotope detected by direct counting of radioemission or by scintillation counting. Alternatively, test compounds can be enzymatically-labeled with, for example, horseradish peroxidase, alkaline phosphatase, or luciferase, and the enzymatic label detected by determination of conversion of an appropriate substrate to product. In one embodiment, the assay comprises contacting a cell which expresses a membrane-bound form of MOLX protein, or a biologically-active portion thereof, on the cell surface with a known compound which binds MOLX to form an assay mixture, contacting the assay mixture with a test compound, and determining the ability of the test compound to interact with an MOLX protein, wherein determining the ability of the test compound to interact with an MOLX protein comprises determining the ability of the test compound to preferentially bind to MOLX protein or a biologically-active portion thereof as compared to the known compound.

In another embodiment, an assay is a cell-based assay comprising contacting a cell expressing a membrane-bound form of MOLX protein, or a biologically-active portion thereof, on the cell surface with a test compound and determining the ability of the test compound to modulate (e.g, stimulate or inhibit) the activity of the MOLX protein or biologically-active portion thereof. Determining the ability of the test compound to modulate the activity of MOLX or a biologically-active portion thereof can be accomplished, for example, by determining the ability of the MOLX protein to bind to or interact with an MOLX target molecule. As used herein, a “target molecule” is a molecule with which an MOLX protein binds or interacts in nature, for example, a molecule on the surface of a cell which expresses an MOLX interacting protein, a molecule on the surface of a second cell, a molecule in the extracellular milieu, a molecule associated with the internal surface of a cell membrane or a cytoplasmic molecule. An MOLX target molecule can be a non-MOLX molecule or an MOLX protein or polypeptide of the invention. In one embodiment, an MOLX target molecule is a component of a signal transduction pathway that facilitates transduction of an extracellular signal (e.g. a signal generated by binding of a compound to a membrane-bound MOLX molecule) through the cell membrane and into the cell. The target, for example, can be a second intercellular protein that has catalytic activity or a protein that facilitates the association of downstream signaling molecules with MOLX.

Determining the ability of the MOLX protein to bind to or interact with an MOLX target molecule can be accomplished by one of the methods described above for determining direct binding. In one embodiment, determining the ability of the MOLX protein to bind to or interact with an MOLX target molecule can be accomplished by determining the activity of the target molecule. For example, the activity of the target molecule can be determined by detecting induction of a cellular second messenger of the target (i.e intracellular Ca²⁺, diacylglycerol, IP₃, etc.), detecting catalytic/enzymatic activity of the target an appropriate substrate, detecting the induction of a reporter gene (comprising an MOLX-responsive regulatory element operatively linked to a nucleic acid encoding a detectable marker, e g., luciferase), or detecting a cellular response, for example, cell survival, cellular differentiation, or cell proliferation.

In yet another embodiment, an assay of the invention is a cell-free assay comprising contacting an MOLX protein or biologically-active portion thereof with a test compound and determining the ability of the test compound to bind to the MOLX protein or biologically-active portion thereof. Binding of the test compound to the MOLX protein can be determined either directly or indirectly as described above. In one such embodiment, the assay comprises contacting the MOLX protein or biologically-active portion thereof with a known compound which binds MOLX to form an assay mixture, contacting the assay mixture with a test compound, and determining the ability of the test compound to interact with an MOLX protein, wherein determining the ability of the test compound to interact with an MOLX protein comprises determining the ability of the test compound to preferentially bind to MOLX or biologically-active portion thereof as compared to the known compound.

In still another embodiment, an assay is a cell-free assay comprising contacting MOLX protein or biologically-active portion thereof with a test compound and determining the ability of the test compound to modulate (e.g. stimulate or inhibit) the activity of the MOLX protein or biologically-active portion thereof. Determining the ability of the test compound to modulate the activity of MOLX can be accomplished, for example, by determining the ability of the MOLX protein to bind to an MOLX target molecule by one of the methods described above for determining direct binding. In an alternative embodiment, determining the ability ot the test compound to modulate the activity of MOLX protein can be accomplished by determining the ability of the MOLX protein further modulate an MOLX target molecule. For example, the catalytic/enzymatic activity of the target molecule on an appropriate substrate can be determined as described, supra.

In yet another embodiment, the cell-free assay comprises contacting the MOLX protein or biologically-active portion thereof with a known compound which binds MOLX protein to form an assay mixture, contacting the assay mixture with a test compound, and determining the ability of the test compound to interact with an MOLX protein, wherein determining the ability of the test compound to interact with an MOLX protein comprises determining the ability of the MOLX protein to preferentially bind to or modulate the activity of an MOLX target molecule.

The cell-free assays of the invention are amenable to use of both the soluble form or the membrane-bound form of MOLX protein. In the case of cell-free assays comprising the membrane-bound form of MOLX protein, it may be desirable to utilize a solubilizing agent such that the membrane-bound form of MOLX protein is maintained in solution. Examples of such solubilizing agents include non-ionic detergents such as n-octylglucoside, n-dodecylglucoside, n-doclecylmaltoside, octanoyl-N-methylglucamide, decanoyl-N-methylglucamide, Triton® X-100, Triton® X-114, Thesit®, Isotridecypoly(ethylene glycol ether)ₙ, N-dodecyl--N,N-dimethyl-3-ammonio-1-propane sulfonate, 3-(3-cholamidopropyl) dimethylamminiol-1-propane sulfonate (CHAPS), or 3-(3-cholamidopropyl)dimethylamminiol-2-hydroxy-1-propane sulfonate (CHAPSO).

In more than one embodiment of the above assay methods of the invention, it may be desirable to immobilize either MOLX protein or its target molecule to facilitate separation of complexed from uncompilexed forms of one or both of the proteins, as well as to accommodate automation of the assay. Binding of a test compound to MOLX protein, or interaction of MOLX protein with a target molecule in the presence and absence of a candidate compound, can be accomplished in any vessel suitable for containing the reactants. Examples of such vessels include microtiter plates, test tubes, and micro-centrifuge tubes. In one embodiment, a fusion protein can be provided that adds a domain that allows one or both of the proteins to be bound to a matrix. For example, GST-MOLX fusion proteins or GST-target fusion proteins can be adsorbed onto glutathione sepharose beads (Sigma Chemical, St. Louis, Mo.) or glutathone derivatized microtiter plates, that are then combined with the test compound or the test compound and either the non-adsorbed target protein or MOLX protein, and the mixture is incubated under conditions conducive to complex formation (e g, at physiological conditions for salt and pH). Following incubation, the beads or microtiter plate wells are washed to remove any unbound components, the matrix immobilized in the case of beads, complex determined either directly or indirectly, for example, as described, supra. Alternatively, the complexes can be dissociated from the matrix, and the level of MOLX protein binding or activity determined using standard techniques.

Other techniques for immobilizing proteins on matrices can also be used in the screening assays of the invention. For example, either the MOLX protein or its target molecule can be immobilized utilizing conjugation of biotin and streptavidin. Biotinylated MOLX protein or target molecules can be prepared from biotin-NHS (N-hydroxy-succinimide) using techniques well-known within the art (e.g, biotinylation kit, Pierce Chemicals, Rockford, Ill.), and immobilized in the wells of streptavidin-coated 96 well plates (Pierce Chemical). Alternatively, antibodies reactive with MOLX protein or target molecules, but which do not interfere with binding of the MOLX protein to its target molecule, can be derivatized to the wells of the plate, and unbound target or MOLX protein trapped in the wells by antibody conjugation. Methods for detecting such complexes, in addition to those described above for the GST-immobilized complexes, include immunodetection of complexes using antibodies reactive with the MOLX protein or target molecule, as well as enzyme-linked assays that rely on detecting an enzymatic activity associated with the MOLX protein or target molecule.

In another embodiment, modulators of MOLX protein expression are identified in a method wherein a cell is contacted with a candidate compound and the expression of MOLX mRNA or protein in the cell is determined. The level of expression of MOLX mRNA or protein in the presence of the candidate compound is compared to the level of expression of MOLX mRNA or protein in the absence of the candidate compound. The candidate compound can then be identified as a modulator of MOLX mRNA or protein expression based upon this comparison. For example, when expression of MOLX mRNA or protein is greater (i.e., statistically significantly greater) in the presence of the candidate compound than in its absence, the candidate compound is identified as a stimulator of MOLX mRNA or protein expression. Alternatively, when expression of MOLX mRNA or protein is less (statistically significantly less) in the presence of the candidate compound than in its absence, the candidate compound is identified as an inhibitor of MOLX mRNA or protein expression. The level of MOLX mRNA or protein expression in the cells can be determined by methods described herein for detecting MOLX mRNA or protein.

In yet another aspect of the invention, the MOLX proteins can be used as “bait proteins” in a two-hybrid assay or three hybrid assay (see, e g, U.S. Pat. No. 5,283,317; Zervos, et al., 1993. Cell 72: 223-232; Madura, et al., 1993. J Biol. Chem 268: 12046-12054; Bartel, et al., 1993. Biotechniques 14: 920-924; Iwabuchi, et al., 1993. Oncogene 8: 1693-1696; and Brent WO 94/10300), to identify other proteins that bind to or interact with MOLX (“MOLX-binding proteins” or “MOLX-bp”) and modulate MOLX activity. Such MOLX-binding proteins are also likely to be involved in the propagation of signals by the MOLX proteins as, for example, upstream or downstream elements of the MOLX pathway.

The two-hybrid system is based on the modular nature of most transcription factors, which consist of separable DNA-binding and activation domains. Briefly, the assay utilizes two different DNA constructs. In one construct, the gene that codes for MOLX is fused to a gene encoding the DNA binding domain of a known transcription factor (e g., GAL-4). In the other construct, a DNA sequence, from a library of DNA sequences, that encodes an unidentified protein (“prey” or “sample”) is fused to a gene that codes for the activation domain of the known transcription factor. If the “bait” and the “prey” proteins are able to interact, in vivo, forming an MOLX-dependent complex, the DNA-binding and activation domains of the transcription factor are brought into close proximity. This proximity allows transcription of a reporter gene (e.g, LacZ) that is operably linked to a transcriptional regulatory site responsive to the transcription factor. Expression of the reporter gene can be detected and cell colonies containing the functional transcription factor can be isolated and used to obtain the cloned gene that encodes the protein which interacts with MOLX.

The invention further pertains to novel agents identified by the aforementioned screening assays and uses thereof for treatments as described herein.

### Detection Assays

Portions or fragments of the cDNA sequences identified herein (and the corresponding complete gene sequences) can be used in numerous ways as polynucleotide reagents. By way of example, and not of limitation, these sequences can be used to: (i) map their respective genes on a chromosome; and, thus, locate gene regions associated with genetic disease; (ii) identify an individual from a minute biological sample (tissue typing); and (iii) aid in forensic identification of a biological sample. Some of these applications are described in the subsections, below.

### Chromosome Mapping

Once the sequence (or a portion of the sequence) of a gene has been isolated, this sequence can be used to map the location of the gene on a chromosome. This process is called chromosome mapping. Accordingly, portions or fragments of the MOLX sequences, SEQ ID NOS:1, 3, 5, 7, 9, 11, 13, 15, 17, 19, 21, 23, 101, 103, 105, 107, 109, 112, 115, 117, 119, 121, and 124, or fragments or derivatives thereof, can be used to map the location of the MOLX genes, respectively, on a chromosome. The mapping of the MOLX sequences to chromosomes is an important first step in correlating these sequences with genes associated with disease.

Briefly, MOLX genes can be mapped to chromosomes by preparing PCR primers (preferably 15-25 bp in length) from the MOLX sequences. Computer analysis of the MOLX, sequences can be used to rapidly select primers that do not span more than one exon in the genomic DNA, thus complicating the amplification process. These primers can then be used for PCR screening of somatic cell hybrids containing individual human chromosomes. Only those hybrids containing the human gene corresponding to the MOLX sequences will yield an amplified fragment.

Somatic cell hybrids are prepared by fusing somatic cells from different mammals (e.g., human and mouse cells). As hybrids of human and mouse cells grow and divide, they gradually lose human chromosomes in random order, but retain the mouse chromosomes. By using media in which mouse cells cannot grow, because they lack a particular enzyme, but in which human cells can, the one human chromosome that contains the gene encoding the needed enzyme will be retained. By using various media, panels of hybrid cell lines can be established. Each cell line in a panel contains either a single human chromosome or a small number of human chromosomes, and a full set of mouse chromosomes, allowing easy mapping of individual genes to specific human chromosomes. See, e.g., D'Eustachio, et al., 1993. Science 220: 919-924. Somatic cell hybrids containing only fragments of human chromosomes can also be produced by using human chromosomes with translocations and deletions.

PCR mapping of somatic cell hybrids is a rapid procedure for assigning a particular sequence to a particular chromosome. Three or more sequences can be assigned per day using a single thermal cycler. Using the MOLX sequences to design oligonucleotide primers, sub-localization can be achieved with panels of fragments from specific chromosomes.

Fluorescence in situ hybridization (FISH) of a DNA sequence to a metaphase chromosomal spread can further be used to provide a precise chromosomal location in one step. Chromosome spreads can be made using cells whose division has been blocked in metaphase by a chemical like colcemid that disrupts the mitotic spindle. The chromosomes can be treated briefly with trypsin, and then stained with Giemsa. A pattern of light and dark bands develops on each chromosome, so that the chromosomes can be identified individually. The FISH technique can be used with a DNA sequence as short as 500 or 600 bases. However, clones larger than 1,000 bases have a higher likelihood of binding to a unique chromosomal location with sufficient signal intensity for simple detection. Preferably 1,000 bases, and more preferably 2,000 bases, will suffice to get good results at a reasonable amount of time. For a review of this technique, see, Verma, et al., HUMAN CHROMOSOMES: A MANUAL OF BASIC TECHNIQUES (Pergamon Press, New York 1988).

Reagents for chromosome mapping can be used individually to mark a single chromosome or a single site on that chromosome, or panels of reagents can be used for marking multiple sites and/or multiple chromosomes. Reagents corresponding to noncoding regions of the genes actually are preferred for mapping purposes. Coding sequences are more likely to be conserved within gene families, thus increasing the chance of cross hybridizations during chromosomal mapping.

Once a sequence has been mapped to a precise chromosomal location, the physical position of the sequence on the chromosome can be correlated with genetic map data. Such data are found, e g., in McKusick, MENDELIAN INHERITANCE IN MAN, available on-line through Johns Hopkins University Welch Medical Library). The relationship between genes and disease, mapped to the same chromosomal region, can then be identified through linkage analysis (co-inheritance of physically adjacent genes), described in, e.g., Egeland, et al., 1987. Nature, 325: 783-787.

Moreover, differences in the DNA sequences between individuals affected and unaffected with a disease associated with the MOLX gene, can be determined. If a mutation is observed in some or all of the affected individuals but not in any unaffected individuals, then the mutation is likely to be the causative agent of the particular disease. Comparison of affected and unaffected individuals generally involves first looking for structural alterations in the chromosomes, such as deletions or translocations that are visible from chromosome spreads or detectable using PCR based on that DNA sequence. Ultimately, complete sequencing of genes from several individuals can be performed to confirm the presence of a mutation and to distinguish mutations from polymorphisms.

### Tissue Typing

The MOLX sequences of the invention can also be used to identify individuals from minute biological samples. In this technique, an individual's genomic DNA is digested with one or more restriction enzymes, and probed on a Southern blot to yield unique bands for identification. The sequences of the invention are useful as additional DNA markers for RFLP (“restriction fragment length polymorphisms,” described in U.S. Pat. No. 5,272,057).

Furthermore, the sequences of the invention can be used to provide an alternative technique that determines the actual base-by-base DNA sequence of selected portions of an individual's genome. Thus, the MOLX sequences described herein can be used to prepare two PCR primers from the 5′- and 3′-termini of the sequences. These primers can then be used to amplify an individual's DNA and subsequently sequence it.

Panels of corresponding DNA sequences from individuals, prepared in this manner, can provide unique individual identifications, as each individual will have a unique set of such DNA sequences due to allelic differences. The sequences of the invention can be used to obtain such identification sequences from individuals and from tissue. The MOLX sequences of the invention uniquely represent portions of the human genome. Allelic variation occurs to some degree in the coding regions of these sequences, and to a greater degree in the noncoding regions. It is estimated that allelic variation between individual humans occurs with a frequency of about once per each 500 bases. Much of the allelic variation is due to single nucleotide polymorphisms (SNPs), which include restriction fragment length polymorphisms (RFLPs).

Each of the sequences described herein can, to some degree, be used as a standard against which DNA from an individual can be compared for identification purposes. Because greater numbers of polymorphisms occur in the noncoding regions, fewer sequences are necessary to differentiate individuals. The noncoding sequences can comfortably provide positive individual identification with a panel of perhaps 10 to 1,000 primers that each yield a noncoding amplified sequence of 100 bases. If predicted coding sequences, such as those in SEQ ID NOS:1, 3, 5, 7, 9, 11, 13, 15, 17, 19, 21, 23, 101, 103, 105, 107, 109, 112, 115, 117, 119, 121, and 124 are used, a more appropriate number of primers for positive individual identification would be 500-2,000.

### Predictive Medicine

The invention also pertains to the field of predictive medicine in which diagnostic assays, prognostic assays, pharmacogenomics, and monitoring clinical trials are used for prognostic (predictive) purposes to thereby treat an individual prophylactically. Accordingly one aspect of the invention relates to diagnostic assays for determining MOLX protein and/or nucleic acid expression as well as MOLX activity, in the context of a biological sample (e.g., blood, serum, cells, tissue) to thereby determine whether an individual is afflicted with a disease or disorder, or is at risk of developing a disorder, associated with aberrant MOLX expression or activity. The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers. The invention also provides for prognostic (or predictive) assays for determining whether an individual is at risk of developing a disorder associated with MOLX protein, nucleic acid expression or activity. For example, mutations in an MOLX gene can be assayed in a biological sample. Such assays can be used for prognostic or predictive purpose to thereby prophylactically treat all individual prior to the onset of a disorder characterized by or associated with MOLX protein, nucleic acid expression, or biological activity.

Another aspect of the invention provides methods for determining MOLX protein, nucleic acid expression or activity in an individual to thereby select appropriate therapeutic or prophylactic agents for that individual (referred to herein as “pharmacogenomics”). Pharmacogenomics allows for the selection of agents (e.g., drugs) for therapeutic or prophylactic treatment of an individual based on the genotype of the individual (e.g, the genotype of the individual examined to determine the ability of the individual to respond to a particular agent.)

Yet another aspect of the invention pertains to monitoring the influence of agents (e g., drugs, compounds) on the expression or activity of MOLX in clinical trials.

These and other agents are described in further detail in the following sections.

### Diagnostic Assays

An exemplary method for detecting the presence or absence of MOLX in a biological sample involves obtaining a biological sample from a test subject and contacting the biological sample with a compound or an agent capable of detecting MOLX protein or nucleic acid (e g, mRNA, genomic DNA) that encodes MOLX protein such that the presence of MOLX is detected in the biological sample. An agent for detecting MOLX mRNA or genomic DNA is a labeled nucleic acid probe capable of hybridizing to MOLX mRNA or genomic DNA. The nucleic acid probe can be, for example, a full-length MOLX nucleic acid, such as the nucleic acid of SEQ ID NOS:1, 3, 5, 7, 9, 11, 13, 15, 17, 19, 21, 23, 101, 103, 105, 107, 109, 112, 115, 117, 119, 121, and 124, or a portion thereof, such as an oligonucleotide of at least 15, 30, 50, 100, 250 or 500 nucleotides in length and sufficient to specifically hybridize under stringent conditions to MOLX mRNA or genomic DNA. Other suitable probes for use in the diagnostic assays of the invention are described herein.

An agent for detecting MOLX protein is an antibody capable of binding to MOLX protein, preferably an antibody with a detectable label. Antibodies can be polyclonal, or more preferably, monoclonal. An intact antibody, or a fragment thereof (e.g., Fab or F(ab′)₂) can be used. The term “labeled”, with regard to the probe or antibody, is intended to encompass direct labeling of the probe or antibody by coupling (i.e, physically linking) a detectable substance to the probe or antibody, as well as indirect labeling of the probe or antibody by reactivity with another reagent that is directly labeled. Examples of indirect labeling include detection of a primary antibody using a fluorescently-labeled secondary antibody and end-labeling of a DNA probe with biotin such that it can be detected with fluorescently-labeled streptavidin. The term “biological sample” is intended to include tissues, cells and biological fluids isolated from a subject, as well as tissues, cells and fluids present within a subject. That is, the detection method of the invention can be used to detect MOLX mRNA, protein, or genomic DNA in a biological sample in vitro as well as in vivo. For example, in vitro techniques for detection of MOLX mRNA include Northern hybridizations and in situ hybridizations. In vitro techniques for detection of MOLX protein include enzyme linked immunosorbent assays (ELISAs), Western blots, immunoprecipitations, and immunofluorescence. In vitro techniques for detection of MOLX genomic DNA include Southern hybridizations. Futhermore, in vivo techniques for detection of MOLX protein include introducing into a subject a labeled anti-MOLX antibody. For example, the antibody can be labeled with a radioactive marker whose presence and location in a subject can be detected by standard imaging techniques.

In one embodiment, the biological sample contains protein molecules from the test subject. Alternatively, the biological sample can contain mRNA molecules from the test subject or genomic DNA molecules from the test subject. A preferred biological sample is a peripheral blood leukocyte sample isolated by conventional means from a subject.

In another embodiment, the methods further involve obtaining a control biological sample from a control subject, contacting the control sample with a compound or agent capable of detecting MOLX protein, mRNA, or genomic DNA, such that the presence of MOLX protein, mRNA or genomic DNA is detected in the biological sample, and comparing the presence of MOLX protein, mRNA or genomic DNA in the control sample with the presence of MOLX protein, mRNA or genomic DNA in the test sample.

The invention also encompasses kits for detecting the presence of MOLX in a biological sample. For example, the kit can comprise: a labeled compound or agent capable of detecting MOLX protein or mRNA in a biological sample; means for determining the amount of MOLX in the sample; and means for comparing the amount of MOLX in the sample with a standard. The compound or agent can be packaged in a suitable container. The kit can further comprise instructions for using the kit to detect MOLX protein or nucleic acid.

### Prognostic Assays

The diagnostic methods described herein can furthermore be utilized to identify subjects having or at risk of developing a disease or disorder associated with aberrant MOLX expression or activity. For example, the assays described herein, such as the preceding diagnostic assays or the following assays, can be utilized to identify a subject having or at risk of developing a disorder associated with MOLX protein, nucleic acid expression or activity. Alternatively, the prognostic assays can be utilized to identify a subject having or at risk for developing a disease or disorder. Thus, the invention provides a method for identifying a disease or disorder associated with aberrant MOLX expression or activity in which a test sample is obtained from a subject and MOLX protein or nucleic acid (e.g., mRNA, genomic DNA) is detected, wherein the presence of MOLX protein or nucleic acid is diagnostic for a subject having or at risk of developing a disease or disorder associated with aberrant MOLX expression or activity. As used herein, a “test sample” refers to a biological sample obtained from a subject of interest. For example, a test sample can be a biological fluid (e.g., serum), cell sample, or tissue.

Furthermore, the prognostic assays described herein can be used to determine whether a subject can be administered an agent (e g., an agonist, antagonist, peptidomimetic, protein, peptide, nucleic acid, small molecule, or other drug candidate) to treat a disease or disorder associated with aberrant MOLX expression or activity. For example, such methods can be used to determine whether a subject can be effectively treated with an agent for a disorder. Thus, the invention provides methods for determining whether a subject can be effectively treated with an agent for a disorder associated with aberrant MOLX expression or activity in which a test sample is obtained and MOLX protein or nucleic acid is detected (e.g., wherein the presence of MOLX protein or nucleic acid is diagnostic for a subject that can be administered the agent to treat a disorder associated with aberrant MOLX expression or activity).

The methods of the invention can also be used to detect genetic lesions in an MOLX gene, thereby determining if a subject with the lesioned gene is at risk for a disorder characterized by aberrant cell proliferation and/or differentiation. In various embodiments, the methods include detecting, in a sample of cells from the subject, the presence or absence of a genetic lesion characterized by at least one of an alteration affecting the integrity of a gene encoding an MOLX-protein, or the misexpression of the MOLX gene. For example, such genetic lesions can be detected by ascertaining the existence of at least one of: (i) a deletion of one or more nucleotides from an MOLX gene; (ii) an addition of one or more nucleotides to an MOLX gene; (iii) a substitution of one or more nucleotides of an MOLX gene, (iv) a chromosomal rearrangement of an MOLX gene; (v) an alteration in the level of a messenger RNA transcript of an MOLX gene, (vi) aberrant modification of an MOLX gene, such as of the methylation pattern of the genomic DNA, (vii) the presence of a non-wild-type splicing pattern of a messenger RNA transcript of an MOLX gene, (viii) a non-wild-type level of an MOLX protein, (ix) allelic loss of an MOLX gene, and (x) inappropriate post-translational modification of an MOLX protein. As described herein, there are a large number of assay techniques known in the art which can be used for detecting lesions in an MOLX gene. A preferred biological sample is a peripheral blood leukocyte sample isolated by conventional means from a subject. However, any biological sample containing nucleated cells may be used, including, for example, buccal mucosal cells.

In certain embodiments, detection of the lesion involves the use of a probe/primer in a polymerase chain reaction (PCR) (see, e.g, U.S. Pat. Nos. 4,683,195 and 4,683,202), such as anchor PCR or RACE PCR, or, alternatively, in a ligation chain reaction (LCR) (see e g, Landegran, et al., 1988. Science 241: 1077-1080; and Nakazawa, et al., 1994. Proc Natl Acad. Sci. USA 91: 360-364), the latter of which can be particularly useful for detecting point mutations in the MOLX-gene (see, Abravaya, et al., 1995. Nucl. Acids Res. 23: 675-682). This method can include the steps of collecting a sample of cells from a patient, isolating nucleic acid (e g., genomic, mRNA or both) from the cells of the sample, contacting the nucleic acid sample with one or more primers that specifically hybridize to an MOLX gene under conditions such that hybridization and amplification of the MOLX gene (if present) occurs, and detecting the presence or absence of an amplification product, or detecting the size of the amplification product and comparing the length to a control sample. It is anticipated that PCR and/or LCR may be desirable to use as a preliminary amplification step in conjunction with any of the techniques used for detecting mutations described herein.

Alternative amplification methods include: self sustainied sequence replication (see, Guatelli, et al., 1990. Proc Natl. Acad. Sci. USA 87: 1874-1878), transcriptional amplification system (see, Kwoh, et al., 1989. Proc Natl. Acad. Sci USA 86: 1173-1177); Qβ Replicase (see, Lizardi, et al, 1988 BioTechnology 6: 1197), or any other nucleic acid amplification method, followed by the detection of the amplified molecules using techniques well known to those of skill in the art. These detection schemes are especially useful for the detection of nucleic acid molecules if such molecules are present in very low numbers.

In an alternative embodiment, mutations in an MOLX gene from a sample cell can be identified by alterations in restriction enzyme cleavage patterns. For example, sample and control DNA is isolated amplified (optionally), digested with one or more restriction endonucleases, and fragment length sizes are determined by gel electrophoresis and compared. Differences in fragment length sizes between sample and control DNA indicates mutations in the sample DNA. Moreover, the use of sequence specific ribozymes (see, e g., U.S. Pat. No. 5,493,531) can be used to score for the presence of specific mutations by development or loss of a ribozyme cleavage site.

In other embodiments, genetic mutations in MOLX can be identified by hybridizing a sample and control nucleic acids, e.g., DNA or RNA, to high-density arrays containing hundreds or thousands of oligonucleotides probes. See e.g, Cronin, et al., 1996. Human Mutation 7: 244-255; Kozal, et al., 1996. Nat. Med. 2: 753-759. For example, genetic mutations in MOLX can be identified in two dimensional arrays containing light-generated DNA probes as described in Cronin, et al., supra. Briefly, a first hybridization array of probes can be used to scan through long stretches of DNA in a sample and control to identify base changes between the sequences by making linear arrays of sequential overlapping probes. This step allows the identification of point mutations. This is followed by a second hybridization array that allows the characterization of specific mutations by using smaller, specialized probe arrays complementary to all variants or mutations detected. Each mutation array is composed of parallel probe sets, one complementary to the wild-type gene and the other complementary to the mutant gene.

In yet another embodiment, any of a variety of sequencing reactions known in the art can be used to directly sequence the MOLX gene and detect mutations by comparing the sequence of the sample MOLX with the corresponding wild-type (control) sequence. Examples of sequencing reactions include those based on techniques developed by Maxim and Gilbert, 1977. Proc. Natl. Acad. Sci USA 74: 560 or Sanger, 1977. Proc Natl Acad Sci. USA 74: 5463. It is also contemplated that any of a variety of automated sequencing procedures can be utilized when performing the diagnostic assays (see, e g, Naeve, et al., 1995. Biotechniques 19: 448), including sequencing by mass spectrometry (see, e.g., PCT International Publication No. WO 94/16101; Cohen, et al., 1996. Adv. Chromatography 36: 127-162; and Griffin, et al., 1993. Appl. Biochem. Biotechnol 38: 147-159).

Other methods for detecting mutations in the MOLX gene include methods in which protection from cleavage agents is used to detect mismatched bases in RNA/RNA or RNA/DNA heteroduplexes. See, e g, Myers, et al., 1985. Science 230: 1242. In general, the art technique of “mismatch cleavage” starts by providing heteroduplexes of formed by hybridizing (labeled) RNA or DNA containing the wild-type MOLX sequence with potentially mutant RNA or DNA obtained from a tissue sample. The double-stranded duplexes are treated with an agent that cleaves single-stranded regions of the duplex such as which will exist due to basepair mismatches between the control and sample strands. For instance, RNA/DNA duplexes can be treated with RNase and DNA/DNA hybrids treated with S₁ nuclease to enzymatically digesting the mismatched regions. In other embodiments, either DNA/DNA or RNA/DNA duplexes can be treated with hydroxylamine or osmium tetroxide and with piperidine in order to digest mismatched regions. After digestion of the mismatched regions, the resulting material is then separated by size on denaturing polyacrylamide gels to determine the site of mutation. See, e.g., Cotton, et al., 1988. Proc Natl Acad Sci USA 85: 4397; Saleeba, et al., 1992. Methods Enzymol. 217: 286-295. In an embodiment, the control DNA or RNA can be labeled for detection.

In still another embodiment, the mismatch cleavage reaction employs one or more proteins that recognize mismatched base pairs in double-stranded DNA (so called “DNA mismatch repair” enzymes) in defined systems for detecting and mapping point mutations in MOLX cDNAs obtained from samples of cells. For example, the mutY enzyme of E. coli cleaves A at G/A mismatches and the thymidine DNA glycosylase from HeLa cells cleaves T at G/T mismatches. See, e g, Hsu et al., 1994. Carcinogenesis 15: 1657-1662. According to an exemplary embodiment, a probe based on an MOLX sequence, e g., a wild-type MOLX sequence, is hybridized to a cDNA or other DNA product from a test cell(s). The duplex is treated with a DNA mismatch repair enzyme, and the cleavage products, if any, can be detected from electrophoresis protocols or the like. See, e g, U.S. Pat. No. 5,459,039.

In other embodiments, alterations in electrophoretic mobility will be used to identify mutations in MOLX genes. For example, single strand conformation polymorphism (SSCP) may be used to detect differences in electrophoretic mobility between mutant and wild type nucleic acids. See, e.g., Orita, et al., 1989. Proc. Natl. Acad. Sci USA: 86: 2766; Cotton, 1993. Mutat. Res. 285: 125-144; Hayashi, 1992. Genet. Anal Tech Appl 9: 73-79. Single-stranded DNA fragments of sample and control MOLX nucleic acids will be denatured and allowed to renature. The secondary structure of single-stranded nucleic acids varies according to sequence, the resulting alteration in electrophoretic mobility enables the detection of even a single base change. The DNA fragments may be labeled or detected with labeled probes. The sensitivity of the assay may be enhanced by using RNA (rather than DNA), in which the secondary structure is more sensitive to a change in sequence. In one embodiment, the subject method utilizes heteroduplex analysis to separate double stranded heteroduplex molecules on the basis of changes in electrophoretic mobility. See, e.g, Keen, et al., 1991. Trends Genet 7: 5.

In yet another embodiment, the movement of mutant or wild-type fragments in polyacrylamide gels containing a gradient of denaturant is assayed using denaturing gradient gel electrophoresis (DGGE). See, e.g., Myers, et al., 1985. Nature 313: 495. When DGGE is used as the method of analysis, DNA will be modified to insure that it does not completely denature, for example by adding a GC clamp of approximately 40 bp of high-melting GC-rich DNA by PCR. In a further embodiment, a temperature gradient is used in place of a denaturing gradient to identify differences in the mobility of control and sample DNA. See, e.g, Rosenbaum and Reissner, 1987. Biophys. Chem. 265: 12753.

Examples of other techniques for detecting point mutations include, but are not limited to, selective oligonucleotide hybridization, selective amplification, or selective primer extension. For example, oligonucleotide primers may be prepared in which the known mutation is placed centrally and then hybridized to target DNA under conditions that permit hybridization only if a perfect match is found. See, e.g., Saiki, et al., 1986. Nature 324: 163; Saiki, et al., 1989. Proc Natl Acad Sci. 86: 6230. Such allele specific oligonucleotides are hybridized to PCR amplified target DNA or a number of different mutations when the oligonucleotides are attached to the hybridizing membrane and hybridized with labeled target DNA.

Alternatively, allele specific amplification technology that depends on selective PCR amplification may be used in conjunction with the instant invention. Oligonucleotides used as primers for specific amplification may carry the mutation of interest in the center of the molecule (so that amplification depends on differential hybridization; see, e.g., Gibbs, et al., 1989. Nucl. Acids Res. 17: 2437-2448) or at the extreme 3′-terminus of one primer where, under appropriate conditions, mismatch can prevent, or reduce polymerase extension (see, e.g., Prossner, 1993. Tibtech. 11: 238). In addition it may be desirable to introduce a novel restriction site in the region of the mutation to create cleavage-based detection. See, e g, Gasparini, et al., 1992. Mol. Cell Probes 6: 1. It is anticipated that in certain embodiments amplification may also be performed using Taq ligase for amplification. See, e.g, Barany, 1991. Proc Natl. Acad. Sci. USA 88: 189. In such cases, ligation will occur only if there is a perfect match at the 3-terminus of the 5′ sequence, making it possible to detect the presence of a known mutation at a specific site by looking for the presence or absence of amplification.

The methods described herein may be performed, for example, by utilizing pre-packaged diagnostic kits comprising at least one probe nucleic acid or antibody reagent described herein, which may be conveniently used, e.g., in clinical settings to diagnose patients exhibiting symptoms or family history of a disease or illness involving an MOLX gene.

Furthermore, any cell type or tissue, preferably peripheral blood leukocytes, in which MOLX is expressed may be utilized in the prognostic assays described herein. However, any biological sample contailing nucleated cells may be used, including, for example, buccal mucosal cells.

### Pharmacogenomics

Agents, or modulators that have a stimulatory or inhibitory effect on MOLX activity (e g., MOLX gene expression), as identified by a screening assay described herein can be administered to individuals to treat (prophylactically or therapeutically) disorders (The disorders include metabolic disorders, diabetes, obesity, infectious disease, anorexia, cancer-associated cachexia, cancer, neurodegenerative disorders, Alzheimer's Disease, Parkinson's Disorder, immune disorders, and hematopoietic disorders, and the various dyslipidemias, metabolic disturbances associated with obesity, the metabolic syndrome X and wasting disorders associated with chronic diseases and various cancers.) In conjunction with such treatment, the pharmacogenomics (i.e., the study of the relationship between an individual's genotype and that individual's response to a foreign compound or drug) of the individual may be considered. Differences in metabolism of therapeutics can lead to severe toxicity or therapeutic failure by altering the relation between dose and blood concentration of the pharmacologically active drug. Thus, the pharmacogenomics of the individual permits the selection of effective agents (e.g, drugs) for prophylactic or therapeutic treatments based on a consideration of the individual's genotype. Such pharmacogenomics can further be used to determine appropriate dosages and therapeutic regimens. Accordingly, the activity of MOLX protein, expression of MOLX nucleic acid, or mutation content of MOLX genes in an individual can be determined to thereby select appropriate agent(s) for therapeutic or prophylactic treatment of the individual.

Pharmacogenomics deals with clinically significant hereditary variations in the response to drugs due to altered drug disposition and abnormal action in affected persons. See e g., Eichelbaum, 1996. Clin. Exp. Pharmacol. Physiol, 23: 983-985; Linder, 1997. Clin. Chem., 43: 254-266. In general, two types of pharmacogenetic conditions can be differentiated. Genetic conditions transmitted as a single factor altering the way drugs act on the body (altered drug action) or genetic conditions transmitted as single factors altering the way the body acts on drugs (altered drug metabolism). These pharmacogenetic conditions can occur either as rare defects or as polymorphisms. For example, glucose-6-phosphate dehydrogenase (G6PD) deficiency is a common inherited enzymopathy in which the main clinical complication is hemolysis after ingestion of oxidant drugs (anti-malarials, sulfonamides, analgesics, nitrofuranis) and consumption of fava beans.

As an illustrative embodiment, the activity of drug metabolizing enzymes is a major determinant of both the intensity and duration of drug action. The discovery of genetic polymorphisms of drug metabolizing enzymes (e g, N-acetyltransferase 2 (NAT 2) and cytochrome P450 enzymes CYP2D6 and CYP2C19) has provided an explanation as to why some patients do not obtain the expected drug effects or show exaggerated drug response and serious toxicity after taking the standard and safe dose of a drug. These polymorphisms are expressed in two phenotypes in the populations, the extensive metabolizer (EM) and poor metabolizer (PM). The prevalence of PM is different among different populations. For example, the gene coding for CYP2D6 is highly polymorphic and several mutations have been identified in PM, which all lead to the absence of functional CYP2D6. Poor metabolizers of CYP2D6 and CYP2C19 quite frequently experience exaggerated drug response and side effects when they receive standard doses. If a metabolite is the active therapeutic moiety, PM show no therapeutic response, as demonstrated for the analgesic effect of codeine mediated by its CYP2D6-formed metabolite morphine. At the other extreme are the so called ultra-rapid metabolizers who do not respond to standard doses. Recently, the molecular basis of ultra-rapid metabolism has been identified to be due to CYP2D6 gene amplification.

Thus, the activity of MOLX protein, expression of MOLX nucleic acid, or mutation content of MOLX genes in an individual can be determined to thereby select appropriate agent(s) for therapeutic or prophylactic treatment of the individual. In addition, pharmacogenetic studies can be used to apply genotyping of polymorphic alleles encoding drug-metabolizing enzymes to the identification of an individual's drug responsiveness phenotype. This knowledge, when applied to dosing or drug selection, can avoid adverse reactions or therapeutic failure and thus enhance therapeutic or prophylactic efficiency when treating a subject with an MOLX modulator, such as a modulator identified by one of the exemplary screening assays described herein.

### Monitoring of Effects During Clinical Trials

Monitoring the influence of agents (e.g., drugs, compounds) on the expression or activity of MOLX (e g., the ability to modulate aberrant cell proliferation and/or differentiation) can be applied not only in basic drug screening, but also in clinical trials. For example, the effectiveness of an agent determined by a screening assay as described herein to increase MOLX gene expression, protein levels, or upregulate MOLX activity, can be monitored in clinical trails of subjects exhibiting decreased MOLX gene expression, protein levels, or downregulated MOLX activity. Alternatively, the effectiveness of an agent determined by a screening assay to decrease MOLX gene expression, protein levels, or downregulate MOLX activity, can be monitored in clinical trails of subjects exhibiting increased MOLX gene expression, protein levels, or upregulated MOLX activity. In such clinical trials, the expression or activity of MOLX and, preferably, other genes that have been implicated in, for example, a cellular proliferation or immune disorder can be used as a “read out” or markers of the immune responsiveness of a particular cell.

By way of example, and not of limitation, genes, including MOLX, that are modulated in cells by treatment with an agent (e.g., compound, drug or small molecule) that modulates MOLX activity (e g, identified in a screening assay as described herein) can be identified. Thus, to study the effect of agents on cellular proliferation disorders, for example, in a clinical trial, cells can be isolated and RNA prepared and analyzed for the levels of expression of MOLX and other genes implicated in the disorder. The levels of gene expression (i.e, a gene expression pattern) can be quantified by Northern blot analysis or RT-PCR, as described herein, or alternatively by measuring the amount of protein produced, by one of the methods as described herein, or by measuring the levels of activity of MOLX or other genes. In this manner, the gene expression pattern can serve as a marker, indicative of the physiological response of the cells to the agent. Accordingly, this response state may be determined before, and at various points during, treatment of the individual with the agent.

In one embodiment, the invention provides a method for monitoring the effectiveness of treatment of a subject with an agent (e.g, an agonist, antagonist, protein, peptide, peptidomimetic, nucleic acid, small molecule, or other drug candidate identified by the screening assays described herein) comprising the steps of (i) obtaining a pre-administration sample from a subject prior to administration of the agent; (ii) detecting the level of expression of an MOLX protein, mRNA, or genomic DNA in the preadministration sample; (iii) obtaining one or more post-administration samples from the subject; (iv) detecting the level of expression or activity of the MOLX protein, mRNA, or genomic DNA in the post-administration samples; (v) comparing the level of expression or activity of the MOLX protein, mRNA, or genomic DNA in the pre-administration sample with the MOLX protein, mRNA, or genomic DNA in the post administration sample or samples; and (vi) altering the administration of the agent to the subject accordingly. For example, increased administration of the agent may be desirable to increase the expression or activity of MOLX to higher levels than detected, i.e., to increase the effectiveness of the agent. Alternatively, decreased administration of the agent may be desirable to decrease expression or activity of MOLX to lower levels than detected, i e, to decrease the effectiveness of the agent.

### Methods of Treatment

The invention provides for both prophylactic and therapeutic methods of treating a subject at risk of (or susceptible to) a disorder or having a disorder associated with aberrant MOLX expression or activity. The disorders include cardiomyopathy, atherosclerosis, hypertension, congenital heart defects, aortic stenosis, atrial septal defect (ASD), atrioventricular (A-V) canal defect, ductus arteriosus, pulmonary stenosis, subaortic stenosis, ventricular septal defect (VSD), valve diseases, tuberous sclerosis, scleroderma, obesity, transplantation, adrenoleukodystrophy, congenital adrenal hyperplasia, prostate cancer, neoplasm; adenocarcinoma, lymphoma, uterus cancer, fertility, hemophilia, hypercoagulation, idiopathic thrombocytopenic pupura, immunodeficiencies, graft versus host disease, AIDS, bronchial asthma, Crohn's disease; multiple sclerosis, treatment of Albright Hereditary Ostoeodystrophy, and othel diseases, disorders and conditions of the like.

These methods of treatment will be discussed more fully, below.

### Disease and Disorders

Diseases and disorders that are characterized by increased (relative to a subject not suffering from the disease or disorder) levels or biological activity may be treated with Therapeutics that antagonize (i e, reduce or inhibit) activity. Therapeutics that antagonize activity may be administered in a therapeutic or prophylactic manner. Therapeutics that may be utilized include, but are not limited to: (i) an aforementioned peptide, or analogs, derivatives, fragments or homologs thereof; (ii) antibodies to an aforementioned peptide; (iii) nucleic acids encoding an aforementioned peptide; (iv) administration of antisense nucleic acid and nucleic acids that are “dysfunctional” (i e, due to a heterologous insertion within the coding sequences of coding sequences to an aforementioned peptide) that are utilized to “knockout” endoggenous function of an aforementioned peptide by homologous recombination (see, e g, Capecchi, 1989. Science 244: 1288-1292); or (v) modulators (i.e, inhibitors, agonists and antagonists, including additional peptide mimetic of the invention or antibodies specific to a peptide of the invention) that alter the interaction between an aforementioned peptide and its binding partner.

Diseases and disorders that are characterized by decreased (relative to a subject not suffering from the disease or disorder) levels or biological activity may be treated with Therapeutics that increase (i.e., are agonists to) activity. Therapeutics that upregulate activity may be administered in a therapeutic or prophylactic manner. Therapeutics that may be utilized include, but are not limited to, an aforementioned peptide, or analogs, derivatives, fragments or homologs thereof; or an agonist that increases bioavailability.

Increased or decreased levels can be readily detected by quantifying peptide and/or RNA, by obtaining a patient tissue sample (e g., from biopsy tissue) and assaying it in vitro for RNA or peptide levels, structure and/or activity of the expressed peptides (or mRNAs of an aforementioned peptide). Methods that are well-known within the art include, but are not limited to, immunoassays (e g, by Western blot analysis, immunoprecipitation followed by sodium docdecyl sulfate (SDS) polyacrylamide gel electrophoresis, immunocytochemistry, etc.) and/or hybridization assays to detect expression of mRNAs (e g, Northern assays, dot blots, in situ hybridization, and the like).

### Prophylactic Methods

In one aspect, the invention provides a method for preventing, in a subject, a disease or condition associated with an aberrant MOLX expression or activity, by administering to the subject an agent that modulates MOLX expression or at least one MOLX activity. Subjects at risk for a disease that is caused or contributed to by aberrant MOLX expression or activity can be identified by, for example, any or a combination of diagnostic or prognostic assays as described herein. Administration of a prophylactic agent can occur prior to the manifestation of symptoms characteristic of the MOLX aberrancy, such that a disease or disorder is prevented or, alternatively, delayed in its progression. Depending upon the type of MOLX aberrancy, for example, an MOLX agonist or MOLX antagonist agent can be used for treating the subject. The appropriate agent can be determined based on screening assays described herein. The prophylactic methods of the invention are further discussed in the following subsections.

### Therapeutic Methods

Another aspect of the invention pertains to methods of modulating MOLX expression or activity for therapeutic purposes. The modulatory method of the invention involves contacting a cell with an agent that modulates one or more of the activities of MOLX protein activity associated with the cell. An agent that modulates MOLX protein activity can be an agent as described herein, such as a nucleic acid or a protein, a naturally-occurring cognate ligand of an MOLX protein, a peptide, an MOLX peptidomimetic, or other small molecule. In one embodiment, the agent stimulates one or more MOLX protein activity. Examples of such stimulatory agents include active MOLX protein and a nucleic acid molecule encoding MOLX that has been introduced into the cell. In another embodiment, the agent inhibits one or more MOLX protein activity. Examples of such inhibitory agents include antisense MOLX nucleic acid molecules and anti-MOLX antibodies. These modulatory methods can be performed in vitro (e g, by culturing the cell with the agent) or, alternatively, in vivo (e.g., by administering the agent to a subject). As such, the invention provides methods of treating an individual afflicted with a disease or disorder characterized by aberrant expression or activity of an MOLX protein or nucleic acid molecule. In one embodiment, the method involves administering an agent (e.g., an agent identified by a screening assay described herein), or combination of agents that modulates (e g, up-regulates or down-regulates) MOLX expression or activity. In another embodiment, the method involves administering an MOLX protein or nucleic acid molecule as therapy to compensate for reduced or aberrant MOLX expression or activity.

Stimulation of MOLX activity is desirable in situations in which MOLX is abnormally downregulated and/or in which increased MOLX activity is likely to have a beneficial effect. One example of such a situation is where a subject has a disorder characterized by aberrant cell proliferation and/or differentiation (e g, cancer or immune associated disorders). Another example of such a situation is where the subject has a gestational disease (e.g., preclampsia).

### Determination of the Biological Effect of the Therapeutic

In various embodiments of the inventions, suitable in vitro or in vivo assays are performed to determine the effect of a specific Therapeutic and whether its administration is indicated for treatment of the affected tissue.

In various specific embodiments, in vitro assays may be performed with representative cells of the type(s) involved in the patient's disorder, to determine if a given Therapeutic exerts the desired effect upon the cell type(s). Compounds for use in therapy may be tested in suitable animal model systems including, but not limited to rats, mice, chicken, cows, monkeys, rabbits, and the like, prior to testing in human subjects. Similarly, for in vivo testing, any of the animal model system known in the art may be used prior to administration to human subjects.

### Prophylactic and Therapeutic Uses of the Compositions of the Invention

The MOLX nucleic acids and proteins of the invention are useful in potential prophylactic and therapeutic applications implicated in a variety of disorders including, but not limited to: metabolic disorders, diabetes, obesity, infectious disease, anorexia, cancer-associated cancer, neurodegenerative disorders, Alzheimer's Disease. Parkinson's Disorder, immune disorders, hematopoietic disorders, and the various dyslipidemias, metabolic disturbances associated with obesity, the metabolic syndrome X and wasting disorders associated with chronic diseases and various cancers.

As an example, a cDNA encoding the MOLX protein of the invention may be useful in gene therapy, and the protein may be useful when adiniistered to a subject in need thereof. By way of non-limiting example, the compositions of the invention will have efficacy for treatment of patients suffering from: metabolic disorders, diabetes, obesity, infectious disease, anorexia, cancer-associated cachexia, cancer, neurodegenerative disorders, Alzheimer's Disease, Parkinson's Disorder, immune disorders, hematopoietic disorders, and the various dyslipidemias.

Both the novel nucleic acid encoding the MOLX protein, and the MOLX protein of the invention, or fragments thereof, may also be useful in diagnostic applications, wherein the presence or amount of the nucleic acid or the protein are to be assessed. A further use could be as an anti-bacterial molecule (i e, some peptides have been found to possess anti-bacterial properties). These materials are further useful in the generation of antibodies which immunospecifically-bind to the novel substances of the invention for use in therapeutic or diagnostic methods.

### EXAMPLES

### Example 1

### Quantitative Expression Analysis of Clones in Various Cells and Tissues

The quantitative expression of various clones was assessed using microtiter plates containing RNA samples from a variety of normal and pathology-derived cells, cell lines and tissues using real time quantitative PCR (RTQ PCR; TAQMAN®). RTQ PCR was performed on a Perkin-Elmer Biosystems ABI PRISM® 7700 Sequence Detection System. Various collections of samples are assembled on the plates, and referred to as Panel 1 (containing cells and cell lines from normal and cancer sources), Panel 2 (containing samples derived from tissues, in particular from surgical samples, from normal and cancer sources), Panel 3 (containing samples derived from a wide variety of cancer sources), Panel 4 (containing cells and cell lines from normal cells and cells related to inflammatory conditions) and Panel CNSD.01 (containing samples from normal and diseased brains).

First, the RNA samples were normalized to constitutively expressed genes such as β-actin and GAPDH. RNA (˜50 ng total or a ˜1 ng polyA+) was converted to cDNA using the TAQMAN® Reverse Transcription Reagents Kit (PE Biosystems, Foster City, Calif.; Catalog No. N808-0234) and random hexamers according to the manufacturer's protocol. Reactions were performed in 20 ul and incubated for 30 min. at 48° C. cDNA (5 ul) was then transferred to a separate plate for the TAQMAN® reaction using β-actin and GAPDH TAQMAN® Assay Reagents (PE Biosystems; Catalog Nos. 4310881E and 4310884E, respectively) and TAQMAN® universal PCR Master Mix (PE Biosystems; Catalog No. 4304447) according to the manufacturer's protocol. Reactions were performed in 25 ul using the following parameters: 2 min. at 50° C.; 10 min. at 95° C.; 15 sec. at 95° C./1 min. at 60° C. (40 cycles). Results were recorded as CT values (cycle at which a given sample crosses a threshold level of fluorescence) using a log scale, with the difference in RNA concentration between a given sample and the sample with the lowest CT value being represented as 2 to the power of delta CT. The percent relative expression is then obtained by taking the reciprocal of this RNA difference and multiplying by 100. The average CT values obtained for β-actin and GAPDH were used to normalize RNA samples. The RNA sample generating the highest CT value required no further diluting, while all other samples were diluted relative to this sample according to their β-actin/GAPDH average CT values.

Normalized RNA (5 ul) was converted to cDNA and analyzed via TAQMAN® using One Step RT-PCR Master Mix Reagents (PE Biosystems; Catalog No. 4309169) and gene-specific primers according to the manufacturer's instructions. Probes and primers were designed for each assay according to Perkin Elmer Biosystem's Primer Express Software package (version 1 for Apple Computer's Macintosh Power PC) or a similar algorithm using the target sequence as input. Default settings were used for reaction conditions and the following parameters were set before selecting primers: primer concentration=250 nM, primer melting temperature (Tₘ) range=58°-60° C., primer optimal Tm=59° C., maximum primer difference=2° C., probe does not have 5′ G, probe Tₘ must be 10° C. greater than primer Tₘ, amplicon size 75 bp to 100 bp. The probes and primers selected (see below) were synthesized by Synthegen (Houston, Tex., USA). Probes were double purified by HPLC to remove uncoupled dye and evaluated by mass spectroscopy to verify coupling of reporter and quencher dyes to the 5′ and 3′ ends of the probe respectively. Their final concentrations were: forward and reverse primers, 900 nM each, and probe, 200 nM.

### PCR conditions:

Normalized RNA from each tissue and each cell line was spotted in each well of a 96 well PCR plate (Perkin Elmer Biosystems). PCR cocktails including two probes (a probe specific for the target clone and another gene-specific probe multiplexed with the target probe) were set up using 1× TaqMan™ PCR Master Mix for the PE Biosystems 7700, with 5 mM MgCl2, dNTPs (dA, G, C, U at 1:1:1:2 ratios), 0.25 U/ml AmpliTaq Gold™ (PE Biosystems), and 0.4 U/□l RNase inhibitor, and 0.25 U/μl reverse transcriptase. Reverse transcription was performed at 48° C. for 30 minutes followed by amplification/PCR cycles as follows: 95° C. 10 min, then 40 cycles of 95° C. for 15 seconds, 60° C. for 1 minute.

In the results for Panel 1, the following abbreviations are used:

ca. = carcinoma,
* = established from metastasis,
met = metastasis,
s cell var = small cell variant,
non-s = non-sm = non-small,
squam = squamous,
pl. eff = pl effusion = pleural effusion,
glio = glioma,
astro = astrocytoma, and
neuro = neuroblastoma.

### Panel 2

The plates for Panel 2 generally include 2 control wells and 94 test samples composed of RNA or cDNA isolated from human tissue procured by surgeons working in close cooperation with the National Cancer Institute's Cooperative Human Tissue Network (CHTN) or the National Disease Research Initiative (NDRI). The tissues are derived from human malignancies and in cases where indicated many malignant tissues have “matched margins ” obtained from noncancerous tissue just adjacent to the tumor. These are termed normal adjacent tissues and are denoted “NAT” in the results below. The tumor tissue and the “matched margins” are evaluated by two independent pathologists (the surgical pathologists and again by a pathologists at NDRI or CHTN). This analysis provides a gross histopathological assessment of tumor differentiation grade. Moreover, most samples include the original surgical pathology report that provides information regarding the clinical stage of the patient. These matched margins are taken from the tissue surrounding (i.e. immediately proximal) to the zone of surgery (designated “NAT”, for normal adjacent tissue, in Table RR). In addition, RNA and cDNA samples were obtained from various human tissues derived from autopsies performed on elderly people or sudden death victims (accidents, etc.). These tissues were ascertained to be free of disease and were purchased from various commercial sources such as Clontech (Palo Alto, Calif.), Research Genetics, and Invitrogen.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

### Panel 3D

The plates of Panel 3D are comprised of 94 cDNA samples and two control samples. Specifically, 92 of these samples are derived from cultured human cancer cell lines, 2 samples of human primary cerebellar tissue and 2 controls. The human cell lines are generally obtained from ATCC (American Type Culture Collection, Manassas, Va.), National Cancer Institute or the German tumor cell bank and fall into the following tissue groups: Squamous cell carcinoma of the tongue, breast cancer, prostate cancer, melanoma, epidermoid carcinoma, sarcomas, bladder carcinomas, pancreatic cancers, kidney cancers, leukemias/lymphomas, ovarian/uterine/cervical, gastric, colon, lung and CNS cancer cell lines. In addition, there are two independent samples of cerebellum. These cells are all cultured under standard recommended conditions and RNA extracted using the standard procedures. The cell lines in panel 3D and 1.3D are of the most common cell lines used in the scientific literature.

RNA integrity from all samples is controlled for quality by visual assessment of agarose gel electropherograms using 28S and 18S ribosomal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

### Panel 4

Panel 4 includes samples on a 96 well plate (2 control wells, 94 test samples) composed of RNA (Panel 4r) or cDNA (Panel 4d) isolated from various human cell lines or tissues related to inflammatory conditions. Total RNA from control normal tissues such as colon and lung (Stratagene, La Jolla, Calif.) and thymus and kidney (Clontech) were employed. Total RNA from liver tissue from cirrhosis patients and kidney from lupus patients was obtained from BioChain (Biochain Institute, Inc., Hayward, Calif.). Intestinal tissue for RNA preparation from patients diagnosed as having Crohn's disease and ulcerative colitis was obtained from the National Disease Research Interchange (NDRI) (Philadelphia, Pa.).

Astrocytes, lung fibroblasts, dermal fibroblasts, coronary artery smooth muscle cells, small airway epithelium, bronchial epithelium, microvascular dermal endothielial cells, microvascular lung endothelial cells, human pulmonary aortic endothelial cells, human umbilical vein endothelial cells were all purchased from Clonetics (Walkersville, Md.) and grown in the media supplied for these cell types by Clonetics. These primary cell types were activated with various cytokines or combinations of cytokines for 6 and/or 12-14 hours, as indicated. The following cytokines were used; IL-1 beta at approximately 1-5 ng/ml, TNF alpha at approximately 5-10 ng/ml, IFN gamma at approximately 20-50 ng/ml, IL-4 at approximately 5-10 ng/ml, IL-9 at approximately 5-10 ng/ml, IL-13 at approximately 5-10 ng/ml. Endothelial cells were sometimes starved for various times by culture in the basal media from Clonetics with 0.1% serum.

MononIuclear cells were prepared from blood of employees at CuraGen Corporation, using Ficoll. LAK cells were prepared from these cells by culture in DMEM 5% FCS (Hyclone), 100 μM non essential amino acids (Gibco/Life Technologies, Rockville, Md.), 1 mM sodium pyruvate (Gibco), mercaptoethanol 5.5×10⁻⁵ M (Gibco), and 10 mM Hepes (Gibco) and Interleukin 2 for 4-6 days. Cells were then either activated with 10-20 ng/ml PMA and 1-2 μg/ml ionomycin, IL-12 at 5-10 ng/ml, IFN gamma at 20-50 ng/ml and IL-18 at 5-10 ng/ml for 6 hours. In some cases, mononuclear cells were cultured for 4-5 days in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) with PHA (phytohemagglutinin) or PWM (pokeweed mitogen) at approximately 5 μg/ml. Samples were taken at 24, 48 and 72 hours for RNA preparation. MLR (mixed lymphocyte reaction) samples were obtained by taking blood from two donors, isolating the mononuclear cells using Ficoll and mixing the isolated mononuclear cells 1:1 at a final concentration of approximately 2×10⁶ cells/ml in DMEM 5% FCS (Hyclone), 100 μM non essential amino acids (Gibco), 1 mM sodium pyruvate (Gibco), mercaptoethanol (5.5×10⁻⁵ M) (Gibco), and 10 mM Hepes (Gibco). The MLR was cultured and samples taken at various time points ranging from 1-7 days for RNA preparation.

Monocytes were isolated from mononuclear cells using CD14 Miltenyi Beads, +ve VS selection columns and a Vario Magnet according to the manufacturer's instructions. Monocytes were differentiated into dendritic cells by culture in DMEM 5% fetal calf serum (FCS) (Hyclone, Logan, Utah), 100 μM non essential amino acids (Gibco), 1 mM sodium pyruvate (Gibco), mercaptoethanol 5.5×10⁻⁵ M (Gibco), and 10 mM Hepes (Gibco), 50 ng/ml GMCSF and 5 ng/ml IL-4 for 5-7 days. Macrophages were prepared by culture of monocytes for 5-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and 10% AB Human Serum or MCSF at approximately 50 ng/ml. Monocytes, macrophages and dendritic cells were stimulated for 6 and 12-14 hours with lipopolysaccharide (LPS) at 100 ng/ml. Dendritic cells were also stimulated with anti-CD40 monoclonal antibody (Pharmingen) at 10 μg/ml for 6 and 12-14 hours.

CD4 lymphocytes, CD8 lymphocytes and NK cells were also isolated from mononuclear cells using CD4, CD8 and CD56 Miltenyi beads, positive VS selection columns and a Vario Magnet according to the manufacturer's instructions. CD45RA and CD45RO CD4 lymphocytes were isolated by depleting mononuclear cells of CD8, CD56, CD14 and CD19 cells using CD8, CD56, CD14 and CD19 Miltenyi beads and positive selection. Then CD45RO beads were used to isolate the CD45RO CD4 lymphocytes with the remaining cells being CD45RA CD4 lymphocytes. CD45RA CD4, CD45RO CD4 and CD8 lymphocytes were placed in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and plated at 10⁶ cells/ml onto Falcon 6 well tissue culture plates that had been coated overnight with 0.5 μg/ml anti-CD28 (Pharmingen) and 3 ug/ml anti-CD3 (OKT3, ATCC) in PBS. After 6 and 24 hours, the cells were harvested for RNA preparation. To prepare chronically activated CD8 lymphocytes, we activated the isolated CD8 lymphocytes for 4 days on anti-CD28 and anti-CD3 coated plates and then harvested the cells and expanded them in DMEM 5% FCS (Hyclone), 100 μM non essential amino acids (Gibco). 1 mM sodium pyruvate (Gibco), mercaptoethanol 5.5×10⁻⁵ M (Gibco), and 10 mM Hepes (Gibco) and IL-2. The expanded CD8 cells were then activated again with plate bound anti-CD3 and anti-CD28 for 4 days and expanded as before. RNA was isolated 6 and 24 hours after the second activation and after 4 days of the second expansion culture. The isolated NK cells were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco) and IL-2 for 4-6 days before RNA was prepared.

To obtain B cells, tonsils were procured from NDRI. The tonsil was cut up with sterile dissecting scissors and then passed through a sieve. Tonsil cells were then spun down and resupended at 10⁶ Cells/ml in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). To activate the cells, we used PWM at 5 μg/ml or anti-CD40 (Pharmingen) at approximately 10 μg/ml and IL-4 at 5-10 ng/ml. Cells were harvested for RNA preparation at 24, 48 and 72 hours.

To prepare the primary and secondary Th1l/Th2 and Tr1 cells, six-well Falcon plates were coated overnight with 10 μg/ml anti-CD28 (Pharmingen) and 2 μg/ml OKT3 (ATCC), and then washed twice with PBS. Umbilical cord blood CD4 lymphocytes (Poietic Systems, German Town, Md.) were cultured at 10⁵-10⁶ cells/ml in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (4 ng/ml). IL-12 (5 ng/ml) and anti-IL4 (1 □g/ml) were used to direct to Th1, while IL-4 (5 ng/ml) and anti-IFN gamma (1 □g/ml) were used to direct to Th2 and IL-10 at 5 ng/ml was used to direct to Tr1. After 4-5 days, the activated Th1, Th2 and Tr1 lymphocytes were washed once in DMEM and expanded for 4-7 days in DMEM 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco) and IL-2 (1 ng/ml). Following this, the activated Th1, Th2 and Tr1 lymphocytes were re-stimulated for 5 days with anti-CD28/OKT3 and cytokines as described above, but with the addition of anti-CD95L (1 □g/ml) to prevent apoptosis. After 4-5 days, the Th1, Th2 and Tr1 lymphocytes were washed and then expanded again with IL-2 for 4-7 days. Activated Th1 and Th2 lymphocytes were maintained in this way for a maximum of three cycles. RNA was prepared from primary and secondary Th1, Th2 and Tr1 after 6 and 24 hours following the second and third activations with plate bound anti-CD3 and anti-CD28 mAbs and 4 days into the second and third expansion cultures in Interleukin 2.

The following leukocyte cells lines were obtained from the ATCC: Ramos, EOL-1, KU-812. EOL cells were further differentiated by culture in 0.1 mM dbcAMP at 5 ×10⁵ cells/ml for 8 days, changing the media every 3 days and adjusting the cell concentration to 5×10⁵ cells/ml. For the culture of these cells, we used DMEM or RPMI (as recommended by the ATCC), with the addition of 5% FCS (Hyclone), 100 μM non essential amino acids (Gibco), 1 mM sodium pyruvate (Gibco), mercaptoethanol 5.5×10⁻⁵ M (Gibco), 10 mM Hepes (Gibco). RNA was either prepared from resting cells or cells activated with PMA at 10 ng/ml and ionomycin at 1 μg/ml for 6 and 14 hours. Keratinocyte line CCD106 and an airway epithelial tumor line NCI-H292 were also obtained from the ATCC. Both were cultured in DMEM 5% FCS (Hyclone), 100 μM non essential amino acids (Gibco), 1 mM sodium pyruvate (Gibco), mercaptoethanol 5.5×10⁻⁵ M (Gibco), and 10 mM Hepes (Gibco). CCDI106 cells were activated for 6 and 14 hours with approximately 5 ng/ml TNF alpha and 1 ng/ml IL-1 beta, while NCI-H292 cells were activated for 6 and 14 hours with the followling cytokines: 5 ng/ml IL-4, 5 ng/ml IL-9, 5 ng/ml IL-13 and 25 ng/ml IFN gamma.

For these cell lines and blood cells, RNA was prepared by lysing approximately 10⁷ cells/ml using Trizol (Gibco BRL). Briefly, 1/10 volume of bromochloropropane (Molecular Research Corporation) was added to the RNA sample, vortexed and after 10 minutes at room temperature, the tubes were spun at 14,000 rpm in a Sorvall SS34 rotor. The aqueous phase was removed and placed in a 15 ml Falcon Tube. An equal volume of isopropanol was added and left at −20 degrees C. overnight. The precipitated RNA was spun down at 9,000 rpm for 15 min in a Sorvall SS34 rotor and washed in 70% ethanol. The pellet was redissolved in 300 μl of RNAse-free water and 35 μl buffer (Promega) 5 μl DTT, 7 μl RNAsin and 8 μl DNAse were added. The tube was incubated at 37 degrees C. for 30 minutes to remove contaminating genomic DNA, extracted once with phenol chloroform and re-precipitated with 1/10 volume of 3 M sodium acetate and 2 volumes of 100% ethanol. The RNA was spun down and placed in RNAse free water. RNA was stored at −80 degrees C.

### Panel CNSD.01

The plates for Panel CNSD.01 include two control wells and 94 test samples comprised of cDNA isolated from postmortem human brain tissue obtained from the Harvard Brain Tissue Resource Center. Brains are removed from calvaria of donors between 4 and 24 hours after death, sectioned by neuroanatomists, and frozen at −80° C. in liquid nitrogen vapor. All brains are sectioned and examined by neuropathologists to confirm diagnoses with clear associated neuropathology.

Disease diagnoses are taken from patient records. The panel contains two brains from each of the following diagnoses: Alzheimer's disease, Parkinson's disease, Huntington's disease, Progressive Supernuclear Palsy, Depression, and “Normal controls”. Within each of these brains, the following regions are represented: cingulate gyrus, temporal pole, globus palladus, substantia nigra, Brodman Area 4 (primary motor strip), Brodman Area 7 (parietal cortex), Brodman Area 9 (prefrontal cortex), and Brodman area 17 (occipital cortex). Not all brain regions are represented in all cases; e.g., Huntington's disease is characterized in part by neurodegeneration in the globus palladus, thus this region is impossible to obtain from confirmed Huntington's cases. Likewise Parkinson's disease is characterized by degeneration of the substantia nigra making this region more difficult to obtain. Normal control brains were examined for neuropathology and found to be free of any pathology consistent with neurodegeneration.

RNA integrity from all samples is controlled for quality by visual assessment of agarose gel electropherograms using 28S and 18S ribosomnal RNA staining intensity ratio as a guide (2:1 to 2.5:1 28s:18s) and the absence of low molecular weight RNAs that would be indicative of degradation products. Samples are controlled against genomic DNA contamination by RTQ PCR reactions run in the absence of reverse transcriptase using probe and primer sets designed to amplify across the span of a single exon.

In the labels employed to identify tissues in the CNS panel, the following abbreviations are used:

PSP = Progressive supranuclear palsy
Sub Nigra = Substantia nigra
Glob Palladus = Globus palladus
Temp Pole = Temporal pole
Cing Gyr = Cingulate gyrus
BA 4 = Brodman Area 4

cl A. MOL1a

Expression of gene SC29674552_EXT was assessed using the primer-probe sets Ag267 and Ag1308, described in Tables 10 and 11. Results of the RTQ-PCR runs are shown in Tables 12, 13, 14, 15, and 16

TABLE 10
Probe Name Ag267

| | | | | Start | SEQ ID |
|---|---|---|---|---|---|
| Primers | Sequences | TM | Length | Position | NO: |
| Forward | 5′-TGCAGCGACCATCGTTCA-3′ | | 18 | 50 | 65 |
| Probe | TET-5′-CTGCTGTAACATTCATCAATCTGGTCACTGCA-3′-TAMRA | | 32 | 76 | 66 |
| Reverse | 5′-GGGTACATGGGCGCCAT-3′ | | 17 | 109 | 67 |

TABLE 11
Probe Name: Ag1308

| | | | | Start | SEQ ID |
|---|---|---|---|---|---|
| Primers | Sequences | TM | Length | Position | NO: |
| Forward | 5′-GAGTGTGACATTCCAGGACACT-3′ | 59.1 | 22 | 139 | 68 |
| Probe | FAM-5′-ATGGTGGCACCTGCCTCAACCTG-3′-TAMRA | 71.6 | 23 | 167 | 69 |
| Reverse | 5′-GCACTGGCACTGGTAGGAA-3′ | 59.8 | 19 | 195 | 70 |

TABLE 12
Panel 1

| | Relative | | Relative |
|---|---|---|---|
| | Expression(%) | | Expression(%) |
| Tissue Name | 1tm420t_ag267 | Tissue Name | 1tm420t_ag267 |
| Endothelial cells | 4.4 | Kidney (fetal) | 19.8 |
| Endothelial cells (treated) | 5.3 | Renal ca. 786-0 | 17.4 |
| Pancreas | 11.5 | Renal ca. A498 | 20.6 |
| Pancreatic ca. CAPAN 2 | 7.0 | Renal ca. RXF 393 | 18.4 |
| Adipose | 40.3 | Renal ca. ACHN | 20.4 |
| Adrenal gland | 5.8 | Renal ca. UO-31 | 20.3 |
| Thyroid | 11.5 | Renal ca. TK-10 | 100.0 |
| Salivary gland | 7.5 | Liver | 7.3 |
| Pituitary gland | 4.9 | Liver (fetal) | 2.3 |
| Brain (fetal) | 1.2 | Liver ca. (hepatoblast) HepG2 | 0.0 |
| Brain (whole) | 7.9 | Lung | 5.4 |
| Brain (amygdala) | 5.3 | Lung (fetal) | 4.2 |
| Brain (cerebellum) | 16.6 | Lung ca. (small cell) LX-1 | 2.8 |
| Brain (hippocampus) | 1.8 | Lung ca. (small cell) NCl-H69 | 5.5 |
| Brain (substantia nigra) | 2.0 | Lung ca. (s.cell var.) SHP-77 | 3.2 |
| Brain (thalamus) | 1.6 | Lung ca. (large cell) NCl-H460 | 6.7 |
| Brain (hypothalamus) | 0.9 | Lung ca. (non-sm. cell) A549 | 6.5 |
| Spinal cord | 3.0 | Lung ca. (non-s.cell) NCl-H23 | 9.0 |
| CNS ca. (glio/astro) U87-MG | 70.7 | Lung ca. (non-s.cell) HOP-62 | 14.4 |
| CNS ca. (glio/astro) U-118-MG | 21.6 | Lung ca. (non-s.cl) NCl-H522 | 56.6 |
| CNS ca. (astro) SW1783 | 20.7 | Lung ca. (squam.) SW 900 | 28.1 |
| CNS ca.* (neuro; met ) SK-N- | 0.0 | Lung ca. (squam.) NCl-H596 | 2.9 |
| AS | | | |
| CNS ca. (astro) SF-539 | 14.6 | Mammary gland | 35.1 |
| CNS ca. (astro) SNB-75 | 15.8 | Breast ca.* (pl. effusion) MCF-7 | 32.3 |
| CNS ca. (glio) SNB-19 | 62.8 | Breast ca.* (pl.ef) MDA-MB- | 19.6 |
| | | 231 | |
| CNS ca. (glio) U251 | 5.9 | Breast ca.* (pl. effusion) T47D | 19.1 |
| CNS ca. (glio) SF-295 | 23.0 | Breast ca. BT-549 | 11.0 |
| Heart | 5.7 | Breast ca. MDA-N | 7.9 |
| Skeletal muscle | 1.3 | Ovary | 44.4 |
| Bone marrow | 4.1 | Ovarian ca. OVCAR-3 | 15.9 |
| Thymus | 32.3 | Ovarian ca. OVCAR-4 | 44.4 |
| Spleen | 3.4 | Ovarian ca. OVCAR-5 | 30.1 |
| Lymph node | 5.0 | Ovarian ca. OVCAR-8 | 50.7 |
| Colon (ascending) | 16.8 | Ovarian ca. IGROV-1 | 10.4 |
| Stomach | 11.1 | Ovarian ca.* (ascites) SK-OV-3 | 11.9 |
| Small intestine | 2.6 | Uterus | 8.3 |
| Colon ca. SW480 | 6.2 | Placenta | 21.0 |
| Colon ca.* (SW480 met) SW620 | 0.9 | Prostate | 11.3 |
| Colon ca. HT29 | 6.2 | Prostate ca.* (bone met)PC-3 | 22.2 |
| Colon ca. HCT-116 | 19.2 | Testis | 92.0 |
| Colon ca. CaCo-2 | 15.0 | Melanoma Hs688(A).T | 13.5 |
| Colon ca. HCT-15 | 20.2 | Melanoma* (met) Hs688(B).T | 20.3 |
| Colon ca. HCC-2998 | 3.9 | Melanoma UACC-62 | 8.5 |
| Gastric ca.* (liver met) NCl- | 22.8 | Melanoma M14 | 15.2 |
| N87 | | | |
| Bladder | 13.3 | Melanoma LOX IMVI | 54.7 |
| Trachea | 10.3 | Melanoma* (met) SK-MEL-5 | 6.8 |
| Kidney | 6.4 | Melanoma SK-MEL-28 | 38.4 |

TABLE 13
Panel 1.2

| | Relative | | Relative |
|---|---|---|---|
| | Expression(%) | | Expression(%) |
| Tissue Name | 1.2tm1287t_ag267 | Tissue Name | 1.2tm1287t_ag267 |
| Endothelial cells | 8.2 | Renal ca. 786-0 | 16.8 |
| Endothelial cells (treated) | 15.0 | Renal ca. A498 | 32.5 |
| Pancreas | 1.4 | Renal ca. RXF 393 | 15.6 |
| Pancreatic ca. CAPAN 2 | 2.9 | Renal ca. ACHN | 15.3 |
| Adrenal Gland (new lot*) | 14.7 | Renal ca. UO-31 | 15.9 |
| Thyroid | 2.1 | Renal ca. TK-10 | 53.2 |
| Salivary gland | 14.8 | Liver | 7.7 |
| Pituitary gland | 3.2 | Liver (fetal) | 3.8 |
| Brain (fetal) | 1.9 | Liver ca. (hepatoblast) HepG2 | 0.0 |
| Brain (whole) | 7.1 | Lung | 6.0 |
| Brain (amygdala) | 7.7 | Lung (fetal) | 3.6 |
| Brain (cerebellum) | 5.0 | Lung ca. (small cell) LX-1 | 2.0 |
| Brain (hippocampus) | 8.7 | Lung ca. (small cell) NCl-H69 | 4.5 |
| Brain (thalamus) | 1.5 | Lung ca. (s. cell var.) SHP-77 | 0.3 |
| Cerebral Cortex | 52.8 | Lung ca. (large cell) NCl-H460 | 14.8 |
| Spinal cord | 6.4 | Lung ca. (non-sm. cell) A549 | 8.3 |
| CNS ca. (glio/astro) U87-MG | 100.0 | Lung ca. (non-s. cell) NCl-H23 | 13.4 |
| CNS ca. (glio/astro) U-118-MG | 37.1 | Lung ca. (non-s. cell) HOP-62 | 30.4 |
| CNS ca. (astro) SW 1783 | 14.2 | Lung ca. (non-s. cl) NCl-H522 | 57.4 |
| CNS ca.* (neuro; met) SK-N- | 0.1 | Lung ca. (squam.) SW 900 | 20.9 |
| AS | | | |
| CNS ca. (astro) SF-539 | 11.9 | Lung ca. (squam.) NCl-H596 | 2.7 |
| CNS ca. (astro) SNB-75 | 9.6 | Mammary gland | 13.3 |
| CNS ca. (glio) SNB-19 | 80.7 | Breast ca.* (pl. effusion) MCF-7 | 31.9 |
| CNS ca. (glio) U251 | 26.6 | Breast ca.* (pl. ef) MDA-MB-231 | 23.7 |
| CNS ca. (glio) SF-295 | 6.6 | Breast ca.* (pl. effusion) T47D | 12.2 |
| Heart | 21.0 | Breast ca. BT-549 | 11.7 |
| Skeletal Muscle (new lot*) | 4.9 | Breast ca MDA-N | 12.2 |
| Bone marrow | 2.2 | Ovary | 34.2 |
| Thymus | 2.1 | Ovarian ca. OVCAR-3 | 35.8 |
| Spleen | 2.7 | Ovarian ca. OVCAR-4 | 32.3 |
| Lymph node | 9.9 | Ovarian ca. OVCAR-5 | 21.0 |
| Colorectal | 8.8 | Ovarian ca. OVCAR-8 | 32.5 |
| Stomach | 8.7 | Ovarian ca. IGROV-1 | 20.0 |
| Small intestine | 4.2 | Ovarian ca.* (ascites) SK-OV-3 | 22.2 |
| Colon ca. SW480 | 2.5 | Uterus | 5.2 |
| Colon ca.* (SW480 met) SW620 | 0.5 | Placenta | 24.3 |
| Colon ca. HT29 | 1.4 | Prostate | 9.2 |
| Colon ca. HCT-116 | 13.4 | Prostate ca.* (bone met) PC-3 | 30.6 |
| Colon ca. CaCo-2 | 7.5 | Testis | 8.7 |
| 83219 CC Well to Mod Diff | 6.9 | Melanoma Hs688(A).T | 10.4 |
| (ODO3866) | | | |
| Colon ca. HCC-2998 | 14.7 | Melanoma* (met) Hs688(B).T | 15.8 |
| Gastric ca.* (liver met) NCl- | 38.2 | Melanoma UACC-62 | 27.0 |
| N87 | | | |
| Bladder | 37.1 | Melanoma M14 | 13.1 |
| Trachea | 3.5 | Melanoma LOX IMVI | 8.4 |
| Kidney | 7.3 | Melanoma* (met) SK-MEL-5 | 12.9 |
| Kidney (fetal) | 29.5 | Adipose | 27.5 |

TABLE 14
Panel 2D

| | Relative | | Relative |
|---|---|---|---|
| | Expression(%) | | Expression(%) |
| Tissue Name | 2Dtm2336t_ag267 | Tissue Name | 2Dtm2336t_ag267 |
| Normal Colon GENPAK | 23.7 | Kidney NAT Clontech | 7.7 |
| 061003 | | 8120608 | |
| 83219 CC Well to Mod Diff | 6.1 | Kidney Cancer Clontech | 0.7 |
| (ODO3866) | | 8120613 | |
| 83220 CC NAT (ODO3866) | 8.2 | Kidney NAT Clontech | 10.2 |
| | | 8120614 | |
| 83221 CC Gr.2 rectosigmoid | 3.8 | Kidney Cancer Clontech | 24.0 |
| ODO3868 | | 9010320 | |
| 83222 CC NAT (ODO3868) | 3.5 | Kidney NAT Clontech | 24.3 |
| | | 9010321 | |
| 83235 CC Mod Diff | 3.7 | Normal Uterus GENPAK | 8.4 |
| (ODO3920) | | 061018 | |
| 83236 CC NAT (ODO3920) | 6.4 | Uterus Cancer GENPAK | 15.5 |
| | | 064011 | |
| 83237 CC Gr.2 ascend colon | 7.5 | Normal Thyroid Clontech A+ | 11.0 |
| (ODO3921) | | 6570-1 | |
| 83238 CC NAT (ODO3921) | 4.0 | Thyroid Cancer GENPAK | 27.9 |
| | | 064010 | |
| 83241 CC from Partial | 7.6 | Thyroid Cancer INVITROGEN | 14.0 |
| Hepatectomy (ODO4309) | | A302152 | |
| 83242 Liver NAT (ODO4309) | 7.5 | Thyroid NAT INVITROGEN | 21.6 |
| | | A302153 | |
| 87472 Colon mets to lung | 6.0 | Normal Breast GENPAK | 33.7 |
| (OD04451-01) | | 061019 | |
| 87473 Lung NAT (OD04451- | 8.4 | 84877 Breast Cancer | 11.9 |
| 02) | | (OD04566) | |
| Normal Prostate Clontech A+ | 7.7 | 85975 Breast Cancer | 18.3 |
| 6546-1 | | (OD04590-01) | |
| 84140 Prostate Cancer | 22.1 | 85976 Breast Cancer Mets | 37.1 |
| (OD04410) | | (OD04590-03) | |
| 84141 Prostate NAT | 19.8 | 87070 Breast Cancer | 31.4 |
| (OD04410) | | Metastasis (OD04655-05) | |
| 87073 Prostate Cancer | 12.5 | GENPAK Breast Cancer | 15.2 |
| (OD04720-01) | | 064006 | |
| 87074 Prostate NAT | 27.5 | Breast Cancer Res. Gen. 1024 | 30.8 |
| (OD04720-02) | | | |
| Normal Lung GENPAK | 21.5 | Breast Cancer Clontech | 100.0 |
| 061010 | | 9100266 | |
| 83239 Lung Met to Muscle | 10.2 | Breast NAT Clontech 9100265 | 45.4 |
| (ODO4286) | | | |
| 83240 Muscle NAT | 10.2 | Breast Cancer INVITROGEN | 29.3 |
| (ODO4286) | | A209073 | |
| 84136 Lung Malignant Cancer | 16.0 | Breast NAT INVITROGEN | 26.1 |
| (OD03126) | | A2090734 | |
| 84137 Lung NAT (OD03126) | 15.9 | Normal Liver GENPAK | 6.1 |
| | | 061009 | |
| 84871 Lung Cancer (OD04404) | 8.0 | Liver Cancer GENPAK 064003 | 11.7 |
| 84872 Lung NAT (OD04404) | 19.2 | Liver Cancer Research | 6.4 |
| | | Genetics RNA 1025 | |
| 84875 Lung Cancer (OD04565) | 3.4 | Liver Cancer Research | 10.6 |
| | | Genetics RNA 1026 | |
| 84876 Lung NAT (OD04565) | 8.4 | Paired Liver Cancer Tissue | 14.7 |
| | | Research Genetics RNA 6004- | |
| | | T | |
| 85950 Lung Cancer (OD04237- | 27.4 | Paired Liver Tissue Research | 4.4 |
| 01) | | Genetics RNA 6004-N | |
| 85970 Lung NAT (OD04237- | 15.8 | Paired Liver Cancer Tissue | 11.5 |
| 02) | | Research Genetics RNA 6005- | |
| | | T | |
| 83255 Ocular Mel Met to Liver | 11.0 | Paired Liver Tissue Research | 5.4 |
| (ODO4310) | | Genetics RNA 6005-N | |
| 83256 Liver NAT (OD04310) | 7.0 | Normal Bladder GENPAK | 19.5 |
| | | 061001 | |
| 84139 Melanoma Mets to Lung | 12.1 | Bladder Cancer Research | 9.3 |
| (OD04321) | | Genetics RNA 1023 | |
| 84138 Lung NAT (OD04321) | 23.5 | Bladder Cancer INVITROGEN | 14.7 |
| | | A302173 | |
| Normal Kidney GENPAK | 25.2 | 87071 Bladder Cancer | 13.3 |
| 061008 | | (OD04718-01) | |
| 83786 Kidney Ca, Nuclear | 24.1 | 87072 Bladder Normal | 17.3 |
| grade 2 (OD04338) | | Adjacent (OD04718-03) | |
| 83787 Kidney NAT (OD04338) | 7.7 | Normal Ovary Res. Gen. | 14.5 |
| 83788 Kidney Ca Nuclear | 14.5 | Ovarian Cancer GENPAK | 26.8 |
| grade 1/2 (OD04339) | | 064008 | |
| 83789 Kidney NAT (OD04339) | 9.2 | 87492 Ovary Cancer | 15.5 |
| | | (OD04768-07) | |
| 83790 Kidney Ca, Clear cell | 19.9 | 87493 Ovary NAT (OD04768- | 9.9 |
| type (OD04340) | | 08) | |
| 83791 Kidney NAT (OD04340) | 15.3 | Normal Stomach GENPAK | 6.3 |
| | | 061017 | |
| 83792 Kidney Ca, Nuclear | 14.6 | Gastric Cancer Clontech | 3.7 |
| grade 3 (OD04348) | | 9060358 | |
| 83793 Kidney NAT (OD04348) | 14.3 | NAT Stomach Clontech | 5.0 |
| | | 9060359 | |
| 87474 Kidney Cancer | 19.3 | Gastric Cancer Clontech | 11.8 |
| (OD04622-01) | | 9060395 | |
| 87475 Kidney NAT (OD04622- | 5.3 | NAT Stomach Clontech | 5.9 |
| 03) | | 9060394 | |
| 85973 Kidney Cancer | 17.2 | Gastric Cancer Clontech | 14.5 |
| (OD04450-01) | | 9060397 | |
| 85974 Kidney NAT (OD04450- | 10.4 | NAT Stomach Clontech | 2.8 |
| 03) | | 9060396 | |
| Kidney Cancer Clontech | 17.9 | Gastric Cancer GENPAK | 5.7 |
| 8120607 | | 064005 | |

TABLE 15
Panel 2.2

| | Relative | | Relative |
|---|---|---|---|
| | Expression | | Expression |
| Tissue Name | 2.2x4tm6515f_ag1308_b2 | Tissue Name | 2.2x4tm6515f_ag1308_b2 |
| Normal Colon GENPAK | 26.3 | 83793 Kidney NAT (OD04348) | 83.7 |
| 061003 | | | |
| 97759 Colon cancer (OD06064) | 20.5 | 98938 Kidney malignant cancer | 9.7 |
| | | (OD06204B) | |
| 97760 Colon cancer NAT | 6.5 | 98939 Kidney normal adjacent | 15.3 |
| (OD06064) | | tissue (OD06204E) | |
| 97778 Colon cancer (OD06159) | 2.6 | 85973 Kidney Cancer | 55.1 |
| | | (OD04450-01) | |
| 97779 Colon cancer NAT | 17.2 | 85974 Kidney NAT (OD04450- | 23.4 |
| (OD06159) | | 03) | |
| 98861 Colon cancer (OD06297- | 2.7 | Kidney Cancer Clontech | 2.1 |
| 04) | | 8120613 | |
| 98862 Colon cancer NAT | 25.9 | Kidney NAT Clontech 8120614 | 12.5 |
| (OD06297-015) | | | |
| 83237 CC Gr.2 ascend colon | 9.2 | Kidney Cancer Clontech | 12.6 |
| (OD03921) | | 9010320 | |
| 83238 CC NAT (ODO3921) | 11.3 | Kidney NAT Clontech 9010321 | 7.0 |
| 97766 Colon cancer metastasis | 5.4 | Kidney Cancer Clontech | 19.0 |
| (OD06104) | | 8120607 | |
| 97767 Lung NAT (OD06104) | 4.6 | Kidney NAT Clontech 8120608 | 6.5 |
| 87472 Colon mets to lund | 22.6 | Normal Uterus GENPAK | 31.5 |
| (OD04451-01) | | 061018 | |
| 87473 Lung NAT (OD04451- | 18.3 | Uterus Cancer GENPAK | 21.4 |
| 02) | | 064011 | |
| Normal Prostate Clontech A+ | 7.2 | Normal Thyroid Clontech A+ | |
| 6546-1 (8090438) | | 6570-1 (7080817) | |
| 84140 Prostate Cancer | 11.0 | Thyroid Cancer GENPAK | 16.7 |
| (OD04410) | | 064010 | |
| 84141 Prostate NAT | 20.3 | Thyroid Cancer INVITROGEN | 44.5 |
| (OD04410) | | A302152 | |
| Normal Ovary Res. Gen. | 29.1 | Thyroid NAT INVITROGEN | 14.0 |
| | | A302153 | |
| 98863 Ovarian cancer | 31.5 | Normal Breast GENPAK | 39.9 |
| (OD06283-03) | | 061019 | |
| 98865 Ovarian cancer | 19.3 | 84877 Breast Cancer | 11.0 |
| NAT/fallopian tube (OD06283- | | (OD04566) | |
| 07) | | | |
| Ovarian Cancer GENPAK | 22.2 | Breast Cancer Res. Gen. 1024 | 49.2 |
| 064008 | | | |
| 97773 Ovarian cancer | 12.4 | 85975 Beast Cance | 50.8 |
| (OD06145) | | OD04590-01) | |
| 97775 Ovarian cancer NAT | 36.2 | 85976 Breast Cancer Mets | 42.9 |
| (OD06145) | | (OD04590-03) | |
| 98853 Ovarian cancer | 9.3 | 87070 Breast Cancer Metastasis | 72.1 |
| (OD06455-03) | | (OD04655-05) | |
| 98854 Ovarian NAT | 7.6 | GENPAK Breast Cancer | 29.0 |
| (OD06455-07) Fallopian tube | | 064006 | |
| Normal Lung GENPAK 061010 | 25.4 | Breast Cancer Clontech | 41.1 |
| | | 9100266 | |
| 92337 Invasive poor diff. lung | 10.9 | Breast NAT Clontech 9100265 | 15.6 |
| adeno(ODO4945-01 | | | |
| 92338 Lung NAT (ODO4945- | 20.2 | Breast Cancer INVITROGEN | 13.5 |
| 03) | | A209073 | |
| 84136 Lung Malignant Cancer | 10.5 | Breast NAT INVITROGEN | 43.8 |
| (OD03126) | | A2090734 | |
| 84137 Lung NAT (OD03126) | 24.5 | 97763 Breast cancer | 49.6 |
| | | (0D06083) | |
| 90372 Lung Cancer | 25.9 | 97764 Breast cancer node | 44.3 |
| (OD05014A) | | metastasis (OD06083) | |
| 90373 Lung NAT (OD05014B) | 26.1 | Normal Liver GENPAK | 38.6 |
| | | 061009 | |
| 97761 Lung cancer (OD06081) | 8.3 | Liver Cancer Research Genetics | 13.2 |
| | | RNA 1026 | |
| 97762 Lung cancer NAT | 19.4 | Liver Cancer Research Genetics | 36.4 |
| (OD06081) | | RNA 1025 | |
| 85950 Lung Cancer (OD04237- | 13.3 | Paired Liver Cancer Tissue | 23.8 |
| 01) | | Research Genetics RNA 6004- | |
| | | T | |
| 85970 Lung NAT (OD04237- | 41.6 | Paired Liver Tissue Research | 5.9 |
| 02) | | Genetics RNA 6004-N | |
| 83255 Ocular Mel Met to Liver | 12.9 | Paired Liver Cancer Tissue | 25.1 |
| (OD04310) | | Research Genetics RNA 6005- | |
| | | T | |
| 83256 Liver NAT (ODO4310) | 11.5 | Paired Liver Tissue Research | 47.4 |
| | | Genetics RNA 6005-N | |
| 84139 Melanoma Mets to Lung | 22.9 | Liver Cancer GENPAK 064003 | 36.9 |
| (OD04321) | | | |
| 84138 Lung NAT (OD04321) | 13.6 | Normal Bladder GENPAK | 18.9 |
| | | 061001 | |
| Normal Kidney GENPAK | 19.9 | Bladder Cancer Research | 9.6 |
| 061008 | | Genetics RNA 1023 | |
| 83786 Kidney Ca, Nuclear | 59.8 | Bladder Cancer INVITROGEN | 24.5 |
| grade 2 (OD04338) | | A302173 | |
| 83787 Kidney NAT (OD04338) | 23.8 | Normal Stomach GENPAK | 43.1 |
| | | 061017 | |
| 83788 Kidney Ca Nuclear grade | 100.0 | Gastric Cancer Clontech | 6.2 |
| 1/2 (OD04339) | | 9060397 | |
| 83789 Kidney NAT (OD04339) | 7.3 | NAT Stomach Clontech | 7.3 |
| | | 9060396 | |
| 83790 Kidney Ca. Clear cell | 15.0 | Gastric Cancer Clontech | 9.3 |
| type (OD04340) | | 9060395 | |
| 83791 Kidney NAT (OD04340) | 22.5 | NAT Stomach Clontech | 13.3 |
| | | 9060394 | |
| 83792 Kidney Ca, Nuclear | 14.3 | Gastric Cancer GENPAK | 9.4 |
| grade 3 (OD04348) | | 064005 | |

TABLE 16
Panel 4D

| | Relative | Relative |
|---|---|---|
| | Expression (%) | Expression (%) |
| Tissue Name | 4Dtm1935t_ag267 | 4Dtm1888f_ag1308 |
| 93768_Secondary Th1_anti-CD28/anti-CD3 | 51.0 | 23.2 |
| 93769_Secondary Th2_anti-CD28/anti-CD3 | 43.5 | 24.1 |
| 93770_Secondary Tr1_anti-CD28/anti-CD3 | 42.6 | 23.8 |
| 93573_Secondary Th1_resting day 4-6 in IL-2 | 11.7 | 7.4 |
| 93572_Secondary Th2_resting day 4-6 in IL-2 | 21.9 | 12.4 |
| 93571_Secondary Tr1_resting day 4-6 in IL-2 | 12.1 | 8.0 |
| 93568_primary Th1_anti-CD28/anti-CD3 | 47.0 | 29.5 |
| 93569_primary Th2_anti-CD28/anti-CD3 | 28.1 | 22.8 |
| 93570_primary Tr1_anti-CD28/anti-CD3 | 45.1 | 37.9 |
| 93565_primary Th1_resting dy 4-6 in IL-2 | 51.8 | 49.3 |
| 93566_primary Th2_resting dy 4-6 in IL-2 | 23.0 | 27.9 |
| 93567_primary Tr1_resting dy 4-6 in IL-2 | 34.2 | 27.4 |
| 93351_CD45RA CD4 lymphocyte_anti-CD28/anti-CD3 | 32.3 | 16.5 |
| 93352_CD45RO CD4 lymphocyte_anti-CD28/anti-CD3 | 52.5 | 29.3 |
| 93251_CD8 Lymphocytes_anti-CD28/anti-CD3 | 22.8 | 14.9 |
| 93353_chronic CD8 Lymphocytes 2ry_resting | 20.2 | 12.6 |
| dy 4-6 in IL-2 | | |
| 93574_chronic CD8 Lymphocytes 2ry_activated CD3/CD28 | 22.1 | 14.2 |
| 93354_CD4_none | 6.8 | 8.0 |
| 93252_Secondary Th1/Th2/Tr1_anti-CD95 CH11 | 31.0 | 23.0 |
| 93103_LAK cells_resting | 62.0 | 30.8 |
| 93788_LAK cells_IL-2 | 47.3 | 27.4 |
| 93787_LAK cells_IL-2 + IL-12 | 41.5 | 23.3 |
| 93789_LAK cells_IL-2 + IFN gamma | 36.1 | 27.4 |
| 93790_LAK cells_IL-2 + IL-18 | 27.0 | 19.8 |
| 93104_LAK cells_PMA/ionomycin and IL-18 | 38.2 | 21.2 |
| 93578_NK Cells IL-2_resting | 17.0 | 14.7 |
| 93109_Mixed Lymphocyte Reaction_Two Way MLR | 30.8 | 27.7 |
| 93110_Mixed Lymphocyte Reaction_Two Way MLR | 19.6 | 12.2 |
| 93111_Mixed Lymphocyte Reaction_Two Way MLR | 17.3 | 10.4 |
| 93112_Mononuclear Cells (PBMCs)_resting | 23.0 | 17.2 |
| 93113_Mononuclear Cells (PBMCs)_PWM | 91.4 | 56.6 |
| 93114_Mononuclear Cells (PBMCs)_PHA-L | 52.5 | 31.9 |
| 93249_Ramos (B cell)_none | 14.6 | 14.7 |
| 93250_Ramos (B cell)_ionomycin | 18.2 | 21.9 |
| 93349_B lymphocytes_PWM | 43.2 | 30.6 |
| 93350_B lymphoytes_CD40L and IL-4 | 12.7 | 11.3 |
| 92665_EOL-1 (Eosinophil)_dbcAMP differentiated | 5.4 | 5.7 |
| 93248_EOL-1 (Eosinophil)_dbcAMP/PMAionomycin | 14.2 | 12.9 |
| 93356_Dendritic Cells_none | 28.5 | 16.2 |
| 93355_Dendritic Cells_LPS 100 ng/ml | 23.0 | 14.7 |
| 93775_Dendritic Cells_anti-CD40 | 21.5 | 12.3 |
| 93774_Monocytes_resting | 81.8 | 58.2 |
| 93776_Monocytes_LPS 50 ng/ml | 100.0 | 100.0 |
| 93581_Macrophages_resting | 75.3 | 33.4 |
| 93582_Macrophages_LPS 100 ng/ml | 54.3 | 30.8 |
| 93098_HUVEC (Endothelial)_none | 12.7 | 6.3 |
| 93099_HUVEC (Endothelial)_starved | 18.3 | 12.7 |
| 93100_HUVEC (Endothelial)_IL-1b | 6.9 | 4.9 |
| 93779_HUVEC (Endothelial)_IFN gamma | 11.7 | 6.9 |
| 93102_HUVEC (Endothelial)_TNF alpha + IFN gamma | 12.9 | 6.4 |
| 93101_HUVEC (Endothelial)_TNF alpha + IL4 | 20.7 | 10.7 |
| 93781_HUVEC (Endothelial)_IL-11 | 4.8 | 2.8 |
| 93583_Lung Microvascular Endothelial Cells_none | 14.8 | 7.4 |
| 93584_Lung Microvascular Endothelial Cells_TNFa | 25.3 | 9.8 |
| (4 ng/ml) and IL1b (1 ng/ml) | | |
| 92662_Microvascular Dermal endothelium_none | 27.4 | 16.4 |
| 92663_Microsvasular Dermal endothelium_TNFa | 40.3 | 17.8 |
| (4 ng/ml) and IL1b (1 ng/ml) | | |
| 93773_Bronchial epithelium_TNFa (4 ng/ml) | 37.6 | 24.1 |
| and IL1b (1 ng/ml)** | | |
| 93347_Small Airway Epithelium_none | 11.6 | 7.3 |
| 93348_Small Airway Epithelium_TNFa (4 ng/ml) | 81.8 | 53.2 |
| and IL1b (1 ng/ml) | | |
| 92668_Coronery Artery SMC_resting | 38.2 | 24.5 |
| 92669_Coronery Artery SMC_TNFa (4 ng/ml) | 32.3 | 24.0 |
| and IL1b (1 ng/ml) | | |
| 93107_astrocytes_resting | 14.7 | 8.6 |
| 93108_astrocytes_TNFa (4 ng/ml) and IL1b (1 ng/ml) | 24.7 | 14.2 |
| 92666_KU-812 (Basophil)_resting | 12.4 | 9.9 |
| 92667_KU-812 (Basophil)_PMA/ionoycin | 46.7 | 29.3 |
| 93579_CCD1106 (Keratinocytes)_none | 13.7 | 8.9 |
| 93580_CCD1106 (Keratinocytes)_TNFa and IFNg** | 81.8 | 57.4 |
| 93791_Liver Cirrhosis | 5.4 | 4.3 |
| 93792_Lupus Kidney | 6.6 | 6.4 |
| 93577_NCl-H292 | 32.8 | 27.4 |
| 93358_NCl-H292_IL-4 | 41.8 | 34.9 |
| 93360_NCl-H292_IL-9 | 33.4 | 29.7 |
| 93359_NCl-H292_IL-13 | 27.0 | 21.9 |
| 93357_NCl-H292_IFN gamma | 18.6 | 15.1 |
| 93777_HPAEC_- | 7.5 | 4.6 |
| 93778_HPAEC_IL-1 beta/TNA alpha | 26.6 | 14.5 |
| 93254_Normal Human Lung Fibroblast_none | 27.0 | 12.9 |
| 93253_Normal Human Lung Fibroblast_TNFa (4 ng/ml) | 11.7 | 7.5 |
| and IL-1b (1 ng/ml) | | |
| 93257_Normal Human Lung Fibroblast_IL-4 | 31.9 | 15.9 |
| 93256_Normal Human Lung Fibroblast_IL-9 | 0.0 | 12.8 |
| 93255_Normal Human Lung Fibroblast_IL-13 | 38.7 | 26.6 |
| 93258_Normal Human Lung Fibroblast_{—IFN gamma} | 52.5 | 26.1 |
| 93106_Dermal Fibroblasts CCD1070_resting | 57.0 | 34.9 |
| 93361_Dermal Fibroblasts CCD1070_TNF alpha 4 ng/ml | 87.1 | 60.7 |
| 93105_Dermal Fibroblasts CCD1070_IL-1 beta 1 ng/ml | 46.7 | 37.4 |
| 93772_dermal fibroblast_IFN gamma | 28.9 | 14.5 |
| 93771_dermal fibroblast_IL-4 | 51.0 | 27.7 |
| 93259_IBD Colitis 1** | 4.9 | 4.8 |
| 93260_IBD Colitis 2 | 1.1 | 0.8 |
| 93261_IBD Crohns | 1.0 | 1.0 |
| 735010_Colon_normal | 12.4 | 6.7 |
| 735019_Lung_none | 24.7 | 11.7 |
| 64028-1_Thymus_{—none} | 33.4 | 27.5 |
| 64030-1_Kidney_{—none} | 22.5 | 20.6 |

Panel 1 Summary: Ag267 Among the normal tissues on this panel, highest expression of the MOL1a gene is detected in testis (CT value=25) and adipose. High expression in adipose might suggest that the MOL1a gene plays a role in the development of metabolic diseases, such as obesity or diabetes. In addition, expression of this gene is high in a renal cancer cell line (CT value=25). Moderate expression of the MOL1a gene is also seen in most regions of normal brain. Strikingly, the MOL1a transcript appears at much higher levels in a number of CNS cancer cell lines. Therefore, inhibition of the MOL1a gene product using a monoclonal antibody and/or small molecule therapeutic may be useful for the treatment of some renal cell and CNS carcinomas.

Panel 1.2 Summary: Ag267 Expression of the MOL1a gene is highest in the cerebral cortex (CT value=25) with more moderate expression detected in most other regions of normal brain, suggesting a role for this gene in neurological function. Consistent with the results seen in Panel 1, this gene is strikingly overexpressed in a number of CNS cancer cell lines (specifically glioma and astrocytoma). Moderate overexpression of the MOL1a gene is also detected in renal cell cancer and lung cancer cell lines relative to the normal controls. The MOL1a gene product displays moderate similarity to the Notch protein that has been shown to be involved in cell signalling and has been implicated in oncogenesis. Therefore, the MOL1a gene may be a good marker for CNS or other cancers and would potentially serve as a good drug target for the treatment of certain cancers. This gene is also well expressed in several metabolic tissues (specifically adipose, liver and pancreas) and may thus have application for the treatment of metabolic diseases such as diabetes and obesity. Of particular interest is the good expression (CT value=30.6) in pancreas. The human pancreas-specific gene SEL-11 is thought to be a negative regulator of the notch receptor (Harada, Y. et al. J Hum Genet 44(5):330-6, 1999). Thus, the notch-like MOL1a gene and notch receptor may have potential therapeutic use in diseases involving the pancreas.

Panel 1.3D Summary: Ag267 Among normal tissues, highest MOL1a transcript levels are found in adipose (CT value=30). As was seen for Panels1 and 1.2, moderate expression of this gene is detected in most regions of normal brain and the gene is strikingly over expressed in a number of CNS cancer cell lines. In general, expression of the MOL1a gene appears to be higher in cell lines when compared to tissue samples. A cluster of expression associated with brain, breast and renal cancer cell lines is evident. Thus, the expression of this gene could be associated with cancer cells when compared to normal, since these cell lines are derived from cancers. Alternatively, the expression of this gene could be associated with cell division, since a high percentage of cells in culture are actively dividing when compared to cells in tissue.

Panel 2D Summary: Ag267 Expression of the MOL1a gene in panel 2D appears to be widespread across most of the samples. However, there seems to be significant dysregulation in breast cancers when compared to normal adjacent tissues. Thus, therapeutic modulation of this gene might show utility in the treatment of breast cancers.

Panel 2.2 Summary: Ag1308 The expression of this gene appears to be widespread across most of the samples in panel 2.2. In a couple of instances of renal cell cancer, there seems to be significant dysregulation of the expression of this gene when compared to normal adjacent tissue. Thus, therapeutic modulation of this gene might be useful in the treatment of a sub-set of renal cancers.

Panel 4D Summary: Ag267/Ag1308 The MOL1a transcript is broadly expressed in fibroblasts, keratinocytes, B cells, and T cells, although at a moderate level. High expression of the transcript is also foud in monocytes, whether activated or not. In addition, the transcript is up-regulated (7 fold) in keratinocytes and small airway epithelium by treatment with TNFa and IL-1. The Notch-like protein encoded by the MOL1a gene may regulate cell survival based on its homology to other Notch proteins. Therefore, protein therapeutics (agonist or antagonists) against the MOL1a gene product may be beneficial in the treatment of lung diseases, such as asthma and emphysema, or in the treatment of skin diseases, such as psoriasis and contact sensitivity. cl B. MOL2

Expression of gene MOL2 was assessed using the primer-probe set Ag2120, described in Table 17. Results of the RTQ-PCR runs are shown in Tables 18, 19, 20, and 21

TABLE 17
Probe Name Ag2120

| | | | | Start | SEQ ID |
|---|---|---|---|---|---|
| Primers | Sequences | TM | Length | Position | NO: |
| Forward | 5′-GCTGATTGCAAGAAGATGTTTC-3′ | 59 | 22 | 103 | 71 |
| Probe | TET-5′-TTTTGTCAGCCCTGATTTCTTCGACA-3′-TAMRA | 68.8 | 26 | 140 | 72 |
| Reverse | 5′-CCGATATGTCAGAATCTGCATT-3′ | 59.1 | 22 | 166 | 73 |

TABLE 18
Panel 1.3D

| | Relative | Relative |
|---|---|---|
| | Expression (%) | Expression (%) |
| Tissue Name | 1.3Dtm3025t_ag2120 | 1.3dtm3058t_ag2120 |
| Liver adenocarcinoma | 0.5 | 0.1 |
| Pancreas | 0.3 | 0.2 |
| Pancreatic ca. CAPAN 2 | 1.0 | 1.1 |
| Adrenal gland | 12.8 | 10.8 |
| Thyroid | 2.1 | 1.3 |
| Salivary gland | 0.6 | 1.1 |
| Pituitary gland | 0.6 | 0.3 |
| Brain (fetal) | 7.9 | 8.8 |
| Brain (whole) | 26.8 | 23.8 |
| Brain (amygdala) | 27.5 | 17.8 |
| Brain (cerebellum) | 17.4 | 19.2 |
| Brain (hippocampus) | 64.6 | 58.6 |
| Brain (substantia nigra) | 2.2 | 3.5 |
| Brain (thalamus) | 3.6 | 3.0 |
| Cerebral Cortex | 100.0 | 100.0 |
| Spinal cord | 0.5 | 1.2 |
| CNS ca. (glio/astro) | 0.8 | 0.5 |
| U87-MG | | |
| CNS ca. (glio/astro) | 3.2 | 4.7 |
| U-118-MG | | |
| CNS ca. (astro) SW1783 | 0.7 | 0.8 |
| CNS ca.* (neuro; met) | 0.4 | 0.2 |
| SK-N-AS | | |
| CNS ca. (astro) SF-539 | 0.4 | 0.3 |
| CNS ca. (astro) SNB-75 | 0.5 | 0.2 |
| CNS ca. (glio) SNB-19 | 0.4 | 1.2 |
| CNS ca. (glio) U251 | 1.6 | 1.3 |
| CNS ca. (glio) SF-295 | 1.9 | 0.8 |
| Heart (fetal) | 3.3 | 2.2 |
| Heart | 0.3 | 0.6 |
| Fetal Skeletal | 6.8 | 9.3 |
| Skeletal muscle | 3.4 | 4.0 |
| Bone marrow | 0.9 | 1.6 |
| Thymus | 0.9 | 0.5 |
| Spleen | 0.4 | 0.0 |
| Lymph node | 1.2 | 0.6 |
| Colorectal | 1.6 | 1.1 |
| Stomach | 1.0 | 0.2 |
| Small intestine | 0.2 | 2.0 |
| Colon ca. SW480 | 0.8 | 1.6 |
| Colon ca.* (SW480 met) | 0.0 | 1.4 |
| SW620 | | |
| Colon ca. HT29 | 0.2 | 0.5 |
| Colon ca. HCT-116 | 0.3 | 0.2 |
| Colon ca. CaCo-2 | 0.6 | 0.0 |
| 83219 CC Well to Mod | 1.5 | 0.5 |
| Diff ODO3866 | | |
| Colon ca. HCC-2998 | 0.6 | 0.3 |
| Gastric ca.* (liver met) | 0.5 | 1.2 |
| NCI-N87 | | |
| Bladder | 1.8 | 1.0 |
| Trachea | 0.6 | 1.3 |
| Kidney | 1.3 | 2.5 |
| Kidney (fetal) | 1.7 | 4.0 |
| Renal ca. 786-0 | 3.8 | 3.7 |
| Renal ca. A498 | 2.6 | 3.0 |
| Renal ca. RXF 393 | 2.2 | 2.3 |
| Renal ca. ACHN | 1.1 | 0.8 |
| Renal ca. UO-31 | 0.5 | 1.2 |
| Renal ca. TK-10 | 3.7 | 3.6 |
| Liver | 0.8 | 0.0 |
| Liver (fetal) | 0.2 | 0.0 |
| Liver ca. (hepatoblast) | 0.7 | 0.8 |
| HepG2 | | |
| Lung | 0.2 | 0.7 |
| Lung (fetal) | 0.4 | 0.6 |
| Lung ca. (small cell) | 1.2 | 0.1 |
| LX-1 | | |
| Lung ca. (small cell) | 44.8 | 40.9 |
| NCI-H69 | | |
| Lung ca. (s.cell var.) | 5.6 | 5.2 |
| SHP-77 | | |
| Lung ca. (large cell) | 54.3 | 74.2 |
| NCI-H460 | | |
| Lung ca. (non-sm. cell) | 0.5 | 0.0 |
| A549 | | |
| Lung ca. (non-s.cell) | 1.5 | 2.0 |
| NCI-H23 | | |
| Lung ca (non-s.cell) | 7.0 | 5.3 |
| HOP-62 | | |
| Lung ca. (non-s.cl) | 1.1 | 0.0 |
| NCI-H522 | | |
| Lung ca. (squam.) | 0.3 | 0.8 |
| SW 900 | | |
| Lung ca. (squam.) | 8.4 | 11.1 |
| NCI-H596 | | |
| Mammary gland | 2.4 | 2.0 |
| Breast ca.* (p1. effusion) | 1.0 | 0.7 |
| MCF-7 | | |
| Breast ca.* (p1.ef) | 0.0 | 0.0 |
| MDA-MB-231 | | |
| Breast ca.* (p1. effusion) | 17.3 | 18.7 |
| T47D | | |
| Breast ca. BT-549 | 4.2 | 4.7 |
| Breast ca. MDA-N | 9.3 | 9.9 |
| Ovary | 12.4 | 9.9 |
| Ovarian ca. OVCAR-3 | 0.1 | 0.0 |
| Ovarian ca. OVCAR-4 | 0.5 | 2.7 |
| Ovarian ca. OVCAR-5 | 0.9 | 0.9 |
| Ovarian ca. OVCAR-8 | 2.1 | 4.0 |
| Ovarian ca. IGROV-1 | 0.4 | 0.3 |
| Ovarian ca.* (ascites) | 0.8 | 0.0 |
| SK-OV-3 | | |
| Uterus | 1.2 | 0.7 |
| Placenta | 1.8 | 0.1 |
| Prostate | 0.5 | 0.6 |
| Prostate ca.* | 3.1 | 2.2 |
| (bone met) PC-3 | | |
| Testis | 1.2 | 1.3 |
| Melanoma Hs688(A).T | 0.8 | 0.9 |
| Melanoma* (met) | 1.2 | 2.7 |
| Hs688(B).T | | |
| Melanoma UACC-62 | 0.7 | 0.7 |
| Melanoma M14 | 4.0 | 5.5 |
| Melanoma LOX IMVI | 1.6 | 0.3 |
| Melanoma* (met) | 4.2 | 3.7 |
| SK-MEL-5 | | |
| Adipose | 3.1 | 1.9 |

TABLE 19
Panel 2D

| | Relative Expression (%) | |
|---|---|---|
| Tissue Name | 2Dtm3026t_ag2120 | 2Dtm3035t_ag2120 |
| Normal Colon GENPAK | 7.3 | 7.4 |
| 061003 | | |
| 83219 CC Well to Mod Diff | 4.3 | 7.9 |
| (ODO3866) | | |
| 83220 CC NAT (ODO3866) | 1.7 | 2.9 |
| 83221 CC Gr.2 rectosigmoid | 2.3 | 1.5 |
| (ODO3868) | | |
| 83222 CC NAT (ODO3868) | 1.7 | 3.8 |
| 83235 CC Mod Diff | 0.2 | 0.9 |
| (ODO3920) | | |
| 83236 CC NAT (ODO3920) | 0.1 | 2.5 |
| 83237 CC Gr.2 ascend colon | 0.5 | 0.4 |
| (ODO3921) | | |
| 83238 CC NAT ODO3921) | 5.8 | 6.4 |
| 83241 CC from Partial | 6.1 | 8.7 |
| Hepatectomy (ODO4309) | | |
| 83242 Liver NAT (ODO4309) | 4.0 | 2.2 |
| 87472 Colon mets to lung | 0.0 | 0.3 |
| (ODO4451-01) | | |
| 87473 Lung NAT | 2.9 | 3.9 |
| (ODO4451-02) | | |
| Normal Prostate Clontech | 3.0 | 7.8 |
| A + 6546-1 | | |
| 84140 Prostate Cancer | 8.5 | 9.0 |
| (ODO4410) | | |
| 84141 Prostate NAT | 23.5 | 21.5 |
| (ODO4410) | | |
| 87073 Prostate Cancer | 7.3 | 7.1 |
| (ODO4720-01) | | |
| 87074 Prostate NAT | 1.7 | 11.3 |
| (ODO4720-02) | | |
| Normal Lung GENPAK | 9.2 | 7.2 |
| 061010 | | |
| 83239 Lung Met to Muscle | 0.7 | 0.7 |
| (ODO4286) | | |
| 83240 Muscle NAT | 11.3 | 12.5 |
| (ODO4286) | | |
| 84136 Lung | 7.9 | 4.7 |
| Malignant_Cancer | | |
| (ODO3126) | | |
| 84137 Lung NAT (ODO3126) | 6.4 | 7.5 |
| 84871 Lung Cancer | 3.0 | 3.8 |
| (ODO4404) | | |
| 84872 Lung NAT (ODO4404) | 2.6 | 2.8 |
| 84875 Lung Cancer | 1.8 | 3.4 |
| (ODO4565) | | |
| 84876 Lung NAT (ODO4565) | 3.6 | 2.8 |
| 85950 Lung Cancer | 22.5 | 17.3 |
| (ODO4237-01) | | |
| 85970 Lung NAT | 3.5 | 4.5 |
| (ODO4237-02) | | |
| 83255 Ocular Mel Met to | 2.1 | 4.1 |
| Liver (ODO4310) | | |
| 83256 Liver NAT (ODO4310) | 1.2 | 1.2 |
| 84139 Melanoma Mets to | 2.5 | 1.7 |
| Lung (ODO4321) | | |
| 84138 Lung NAT (ODO4321) | 5.3 | 3.3 |
| Normal Kidney GENPAK | 93.3 | 100.0 |
| 061008 | | |
| 83786 Kidney Ca, Nuclear | 55.9 | 92.7 |
| grade 2 (ODO4338) | | |
| 83787 Kidney NAT | 37.9 | 36.6 |
| (ODO4338) | | |
| 83788 Kidney Ca Nuclear | 67.4 | 76.3 |
| grade 1/2 (ODO4339) | | |
| 83789 Kidney NAT | 25.3 | 33.2 |
| (ODO4339) | | |
| 83790 Kidney Ca, Clear cell | 52.5 | 43.5 |
| type (ODO4340) | | |
| 83791 Kidney NAT | 0.0 | 35.6 |
| (ODO4340) | | |
| 83792 Kidney Ca, Nuclear | 5.0 | 7.6 |
| grade 3 (ODO4348) | | |
| 83793 Kidney NAT | 20.7 | 26.6 |
| (ODO4348) | | |
| 87474 Kidney Cancer | 9.3 | 7.3 |
| (ODO4622-01) | | |
| 87475 Kidney NAT | 7.1 | 10.2 |
| (ODO4622-03) | | |
| 85973 Kidney Cancer | 27.0 | 29.3 |
| (ODO4450-01) | | |
| 85974 Kidney NAT | 34.2 | 33.7 |
| (ODO4450-03) | | |
| Kidney Cancer Clontech | 4.1 | 3.3 |
| 8120607 | | |
| Kidney NAT Clontech | 9.2 | 12.8 |
| 8120608 | | |
| Kidney Cancer Clontech | 2.2 | 3.3 |
| 8120613 | | |
| Kidney NAT Clontech | 7.3 | 14.9 |
| 8120614 | | |
| Kidney Cancer Clontech | 22.4 | 26.4 |
| 9010320 | | |
| Kidney NAT Clontech | 18.3 | 26.8 |
| 9010321 | | |
| Normal Uterus GENPAK | 9.6 | 8.6 |
| 061018 | | |
| Uterus Cancer GENPAK | 2.7 | 2.6 |
| 064011 | | |
| Normal Thyroid Clontech | 5.1 | 5.3 |
| A + 6570-1 | | |
| Thyroid Cancer GENPAK | 39.2 | 44.1 |
| 064010 | | |
| Thyroid Cancer | 30.8 | 24.5 |
| INVITROGEN A302152 | | |
| Thyroid NAT | 3.3 | 3.6 |
| INVITROGEN A302153 | | |
| Normal Breast | 5.0 | 4.5 |
| GENPAK 061019 | | |
| 84877 Breast Cancer | 0.8 | 1.7 |
| (ODO4566) | | |
| 85975 Breast Cancer | 9.7 | 7.6 |
| (ODO4590-01) | | |
| 85976 Breast Cancer | 26.1 | 33.4 |
| Mets (ODO4590-03) | | |
| 87070 Breast Cancer | 3.4 | 4.1 |
| Metastasis (ODO4655-05) | | |
| GENPAK Breast Cancer | 3.3 | 4.4 |
| 064006 | | |
| Breast Cancer Res. Gen. 1024 | 7.2 | 8.3 |
| Breast Cancer Clontech | 3.1 | 3.7 |
| 9100266 | | |
| Breast NAT Clontech | 3.3 | 3.9 |
| 9100265 | | |
| Breast Cancer INVITROGEN | 8.2 | 8.9 |
| A209073 | | |
| Breast NAT INVITROGEN | 16.2 | 11.6 |
| A2090734 | | |
| Normal Liver GENPAK | 1.4 | 0.9 |
| 061009 | | |
| Liver Cancer GENPAK | 1.8 | 5.1 |
| 064003 | | |
| Liver Cancer Research | 1.3 | 2.0 |
| Genetics RNA 1025 | | |
| Liver Cancer Research | 3.4 | 2.4 |
| Genetics RNA 1026 | | |
| Paired Liver Cancer Tissue | 1.6 | 0.4 |
| Research Genetics RNA | | |
| 6004-Γ | | |
| Paired Liver Tissue Research | 1.3 | 1.0 |
| Genetics RNA 6004-N | | |
| Paired Liver Cancer Tissue | 0.0 | 6.5 |
| Research Genetics RNA | | |
| 6005-T | | |
| Paired Liver Tissue Research | 1.6 | 0.3 |
| Genetics RNA 6005-N | | |
| Normal Bladder GENPAK | 3.4 | 9.5 |
| 061001 | | |
| Bladder Cancer Research | 10.7 | 6.5 |
| Genetics RNA 1023 | | |
| Bladder Cancer | 1.8 | 2.1 |
| INVITROGEN A302173 | | |
| 87071 Bladder Cancer | 1.7 | 5.2 |
| (ODO4718-01) | | |
| 87072 Bladder Normal | 4.2 | 6.3 |
| Adjacent (ODO4718-03) | | |
| Normal Ovary Res. Gen | 9.5 | 7.4 |
| Ovarian Cancer GENPAK | 100.0 | 95.3 |
| 064008 | | |
| 87492 Ovary Cancer | 4.6 | 7.6 |
| (ODO4768-07) | | |
| 87493 Ovary NAT | 7.3 | 5.7 |
| (ODO4768-08) | | |
| Normal Stomach GENPAK | 1.7 | 3.8 |
| 061017 | | |
| Gastric Cancer Clontech | 2.1 | 0.5 |
| 9060358 | | |
| NAT Stomach Clontech | 1.6 | 3.1 |
| 9060359 | | |
| Gastric Cancer Clontech | 3.3 | 3.1 |
| 9060395 | | |
| NAT Stomach Clontech | 2.2 | 3.3 |
| 9060394 | | |
| Gastric Cancer Clontech | 11.0 | 14.5 |
| 9060397 | | |
| NAT Stomach Clontech | 2.8 | 4.3 |
| 9060396 | | |
| Gastric Cancer GENPACK | 2.2 | 6.5 |
| 064005 | | |

TABLE 20
Panel 4D

| | Relative | | Relative |
|---|---|---|---|
| | Expression (%) | | Expression (%) |
| Tissue Name | 4Dtm3027t_ag2120 | Tissue Name | 4Dtm3027t_ag2120 |
| 93768_Secondary | 0.4 | 93100_HUVEC (Endothelial)_IL-1b | 11.2 |
| Th1_anti-CD28/anti-CD3 | | | |
| 93769_Secondary | 0.8 | 93779_HUVEC | 10.1 |
| Th2_anti-CD28/anti-CD3 | | (Endothelial)_IFN gamma | |
| 93770_Secondary | 3.1 | 93102_HUVEC (Endothelial)_TNF | 4.1 |
| Tr1_anti-CD28/anti-CD3 | | alpha + IFN gamma | |
| 93573_Secondary | 3.4 | 93101_HUVEC (Endothelial)_TNF | 13.2 |
| Th1_resting day 4-6 in IL-2 | | alpha + IL4 | |
| 93572_Secondary | 1.5 | 93781_HUVEC (Endothelial)_IL-11 | 8.1 |
| Th2_resting day 4-6 in IL-2 | | | |
| 93571_Secondary | 1.5 | 93583_Lung Microvascular Endothelial | 3.2 |
| Tr1_resting day 4-6 in IL-2 | | Cells_none | |
| 93568_primary | 0.3 | 93584_Lung Microvascular Endothelial | 2.4 |
| Th1_anti-CD28/anti-CD3 | | Cells_TNFa (4 ng/ml) and IL1b (1 ng/ml) | |
| 93569_primary | 0.8 | 92662_Microvascular Dermal | 0.0 |
| Th2_anti-CD28/anti-CD3 | | endothelium_none | |
| 93570_primary | 0.4 | 92663_Microvasular Dermal | 0.0 |
| Tr1_anti-CD28/anti-CD3 | | endothelium_TNFa (4 ng/ml) | |
| | | and IL1b (1 ng/ml) | |
| 93565_primary | 3.1 | 93773_Bronchial | 3.1 |
| Th1_resting dy 4-6 in IL-2 | | epithelium_TNFa (4 ng/ml) | |
| | | and IL1b (1 ng/ml)** | |
| 93566_primary | 5.5 | 93347_Small Airway | 5.4 |
| Th2_resting dy 4-6 in IL-2 | | Epithelium_none | |
| 93567_primary | 0.6 | 93348_Small Airway | 3.0 |
| Tr1_resting dy 4-6 in IL-2 | | Epithelium_TNFa (4 ng/ml) | |
| | | and IL1b (1 ng/ml) | |
| 93351_CD45RA CD4 | 1.3 | 92668_Coronery Artery SMC_resting | 35.6 |
| lymphocyte_anti-CD28/anti-CD3 | | | |
| 93352_CD45RO CD4 | 2.4 | 92669_Coronery Artery | 39.0 |
| lymphocyte_anti-CD28/anti-CD3 | | SMC_TNFa (4 ng/ml) and | |
| | | IL1b (1 ng/ml) | |
| 93251_CD8 | 2.2 | 93107_astrocytes_resting | 1.1 |
| Lymphocytes_anti-CD28/anti-CD3 | | | |
| 93353_chronic CD8 | 4.8 | 93108_astrocytes_TNFa (4 ng/ml) and | 0.9 |
| Lymphocytes 2ry_resting dy | | IL1b (1 ng/ml) | |
| 4-6 in IL-2 | | | |
| 93574_chronic | 2.4 | 92666_KU-812 (Basophil)_resting | 0.5 |
| CD8 Lymphocytes 2ry_activated | | | |
| CD3/CD28 | | | |
| 93354_CD4_none | 0.9 | 92667_KU-812 | 2.0 |
| | | (Basophil)_PMA/ionoycin | |
| 93252_Secondary | 0.7 | 93579_CCD1106 (Keratinocytes)_none | 0.9 |
| Th1/Th2/Tr1_anti-CD95 CH11 | | | |
| 93103_LAK cells_resting | 7.2 | 93580_CCD1106 | 2.6 |
| | | (Keratinocytes)_TNFa and IFNg** | |
| 93788_LAK cells_IL-2 | 0.4 | 93791_Liver Cirrhosis | 5.5 |
| 93787_LAK cells_IL-2 + IL-12 | 2.8 | 93792_Lupus Kidney | 9.3 |
| 93789_LAK cells_IL-2 + IFN gamma | 7.7 | 93577_NCI-H292 | 2.0 |
| 93790_LAK cells_IL-2 + IL-18 | 4.1 | 93358_NCI-H292_IL-4 | 5.3 |
| 93104_LAK cells_PMA/ionomycin | 3.2 | 93360_NCI-H292_IL-9 | 3.8 |
| and IL-18 | | | |
| 93578_NK Cells IL-2_resting | 2.3 | 93359_NCI-H292_IL-13 | 1.3 |
| 93109_Mixed Lymphocyte | 2.7 | 93357_NCI-H292_IFN gamma | 3.4 |
| Reaction_Two Way MLR | | | |
| 93110_Mixed Lymphocyte | 4.0 | 93777_HPAEC_- | 9.0 |
| Reaction_Two Way MLR | | | |
| 93111_Mixed Lymphocyte | 5.2 | 93778_HPAEC_IL-1 beta/TNA alpha | 15.4 |
| Reaction_Two Way MLR | | | |
| 93112_Mononuclear Cells | 6.4 | 93254_Normal Human Lung | 1.7 |
| (PBMCs)_resting | | Fibroblast_none | |
| 93113_Mononuclear Cells | 5.2 | 93253_Normal Human Lung | 0.0 |
| (PBMCs)_PWM | | Fibroblast_TNFa (4 ng/ml) | |
| | | and IL-1b (1 ng/ml) | |
| 93114_Mononuclear Cells | 1.8 | 93257_Normal Human Lung | 2.8 |
| (PBMCs)_PHA-L | | Fibroblast_IL-4 | |
| 93249_Ramos (B cell)_none | 3.9 | 93256_Normal Human Lung | 1.6 |
| | | Fibroblast_IL-9 | |
| 93250_Ramos (B cell)_ionomycin | 1.6 | 93255_Normal Human Lung | 1.0 |
| | | Fibroblast_IL-13 | |
| 93349_B lymphocytes_PWM | 0.0 | 93258_Normal Human Lung | 1.4 |
| | | Fibroblast_IFN gamma | |
| 93350_B lymphoytes_CD40L and IL-4 | 3.2 | 93106_Dermal Fibroblasts | 0.6 |
| | | CCD1070_resting | |
| 92665_EOL-1 (Eosinophil)_dbcAMP | 67.4 | 93361_Dermal Fibroblasts | 2.3 |
| differentiated | | CCD1070_TNF alpha 4 ng/ml | |
| 93248_EOL-1 (Eosinophil)_dbcAMP/ | 56.6 | 93105_Dermal Fibroblasts | 5.4 |
| PMAionomycin | | CCD1070_IL-1 beta 1 ng/ml | |
| 93356_Dendritic Cells_none | 6.0 | 93772_dermal fibroblasts_IFN gamma | 0.5 |
| 93355_Dendritic Cells_LPS 100 ng/ml | 15.5 | 93771_dermal fibroblast_IL-4 | 3.0 |
| 93775_Dendritic Cells_anti-CD40 | 13.5 | 93259_IBD Colitis 1** | 1.3 |
| 93774_Monocytes_resting | 46.0 | 93260_IBD Colitis 2 | 5.8 |
| 93776_Monocytes_LPS 50 ng/ml | 4.9 | 93261_IBD Crohns | 4.2 |
| 93581_Macrophages_resting | 47.3 | 735010_Colon_normal | 5.8 |
| 93582_Macrophages_LPS 100 ng/ml | 24.8 | 735019_Lung_none | 7.0 |
| 93098_HUVEC (Endothelial)_none | 10.2 | 64028-1_Thymus_none | 100.0 |
| 93099_HUVEC (Endothelial)_starved | 23.8 | 64030-1_Kidney_none | 18.2 |

TABLE 21
Panel CNSD.01

| | Relative | | Relative |
|---|---|---|---|
| | Expression (%) | | Expression (%) |
| Tissue Name | CNS1 × 4tm6184t_ag2120_a2 | Tissue Name | CNS1 × 4tm6184t_ag2120_a2 |
| 102633_BA4 Control | 46.5 | 102605_BA17 PSP | 20.1 |
| 102641_BA4 Control 2 | 42.9 | 102612_BA17 PSP2 | 10.5 |
| 102625_BA4 Alzheimer's2 | 5.2 | 102637_Sub Nigra Control | 10.6 |
| 102649_BA4 Parkinson's | 38.5 | 102645_Sub Nigra Control2 | 29.1 |
| 102656_BA4 Parkinson's2 | 100.0 | 102629_Sub Nigra Alzheimer's2 | 5.8 |
| 102664_BA4 Huntington's | 53.8 | 102660_Sub Nigra Parkinson's2 | 20.6 |
| 102671_BA4 Huntington's | 4.3 | 102667_Sub Nigra Huntington's | 21.5 |
| 102603_BA4 PSP | 4.5 | 102674_Sub Nigra Huntington's2 | 14.7 |
| 102610_BA4 PSP2 | 14.1 | 102614_Sub Nigra PSP2 | 3.6 |
| 102588_BA4 Depression | 11.8 | 102592_Sub Nigra Depression | 3.2 |
| 102596_BA4 Depression2 | 3.5 | 102599_Sub Nigra Depression2 | 2.4 |
| 102634_BA7 Control | 45.6 | 102636_Glob Palladus Control | 1.8 |
| 102642_BA7 Control2 | 37.9 | 102644_Glob Palladus Control2 | 7.8 |
| 102626_BA7 Alzheimer's2 | 8.5 | 102620_Glob Palladus Alzheimer's | 6.4 |
| 102650_BA7 Parkinson's | 11.7 | 102628_Glob Palladus Alzheimer's | 2.5 |
| 102657_BA7 Parkinson's2 | 54.6 | 102652_Glob Palladus Parkinson's | 34.4 |
| 102665_BA7 Huntington's | 41.1 | 102659_Glob Palladus Parkinson's2 | 3.1 |
| 102672_BA7 Huntington's | 34.3 | 102606_Glob Palladus PSP | 5.2 |
| 102604_BA7 PSP | 36.2 | 102613_Glob Palladus PSP2 | 0.0 |
| 102611_BA7 PSP2 | 21.2 | 102591_Glob Palladus Depression | 3.7 |
| 102589_BA7 Depression | 4.5 | 102638_Temp Pole Control | 11.0 |
| 102632_BA9 Control | 18.1 | 102622_Temp Pole Control2 | 40.1 |
| 102640_BA9 Control2 | 76.7 | 102622_Temp Pole Alzheimer's | 2.7 |
| 102617_BA9 Alzheimer's | 8.0 | 102630_Temp Pole Alzheimer's2 | 3.4 |
| 102624_BA9 Alzheimer's | 4.6 | 102653_Temp Pole Parkinson's | 13.9 |
| 102648_BA9 Parkinson's | 28.7 | 102661_Temp Pole Parkinson's2 | 20.8 |
| 102655_BA9 Parkinson's2 | 55.5 | 102668_Temp Pole Huntington's | 27.6 |
| 102663_BA9 Huntington's | 34.2 | 102607_Temp Pole PSP | 2.5 |
| 102670_BA9 Huntington's2 | 4.7 | 102615_Temp Pole PSP2 | 3.7 |
| 102602_BA9 PSP | 11.6 | 102600_Temp Pole Depression2 | 1.8 |
| 102609_BA9 PSP2 | 3.8 | 102639_Cing Gyr Control | 70.7 |
| 102587_BA9 Depression | 5.0 | 102647_Cing Gyr Control2 | 23.9 |
| 102595_BA9 Depression2 | 3.2 | 102623_Cing Gyr Alzheimer's | 13.8 |
| 102635_BA17 Control | 37.0 | 102631_Cing Gyr Alzheimer'2 | 7.8 |
| 102643_BA17 Control2 | 62.0 | 102654_Cing Gyr Parkinson's | 13.2 |
| 102627_BA17 Alzheimer's2 | 2.9 | 102662_Cing Gyr Parkinson's2 | 23.0 |
| 102651_BA17 Parkinson's | 13.4 | 102669_Cing Gyr Huntington's | 40.3 |
| 102658_BA17 Parkinson's2 | 61.3 | 102676_Cing Gyr Huntington'2 | 12.9 |
| 102666_BA17 Huntington's | 28.0 | 102608_Cing Gyr PSP | 7.6 |
| 102673_BA17 Huntington's2 | 8.4 | 102616_Cing GYr PSP2 | 5.6 |
| 102590_BA17 Depression | 2.3 | 102601_Cing Gyr Depression | 4.1 |
| 102597_BA17 Depression2 | 10.1 | 102601_Cing Gyr Depression2 | 4.6 |

Panel 1.3D Summary: Ag2120 Two replicate experiments using the same probe and primer set show very comparable results. Expression of the MOL2 gene is highest in the cerebral cortex (CT value=29). Moderate expression is detected in all other regions of the brain except thalamus and substantia nigra; this observation sug(lgests that the MOL2 gene may be associated with normal brain homeostasis. Thus, this protein shows a brain-preferential expression; see write-up on Panel CNS.01 for discussion of utility. In addition, expression of the MOL2 gene appears to be down-regulated in CNS cancer cell lines. Overexpression of the MOL2 gene is also detected in several lung cancer cell lines relative to normal control. Therefore, this gene might be a good target for the detection or treatment of CNS and lung cancers.

Panel 2D Summary: Ag2120 Two replicate experiments using the same probe and primer set show very comparable results. Expression of the MOL2 gene in panel 2D reveals an association of expression in thyroid, breast and kidney cancers when compared to their respective normal adjacent tissues. Thus, therapeutic modulation of this gene with inhibitory monoclonal antibodies and/or small moleculte therapeutics may show utility in treatment of these diseases. In addition, the MOL2 gene might be useful as a marker for thyroid, breast and kidney cancers.

Panel 4D Summary: Ag2120 The MOL2 gene is expressed at highest levels in the thymus (CT value=31), In addition, the transcript is also expressed in eosinophils, monocytes, macrophages and coronary artery. Interestingly, it is down regulated in LPS-treated monocytes and to a lesser degree in LPS treated macrophages. Therefore, protein therapeutics (agonists or antagonists) designed against the protein encoded for by this transcript could reduce inflammatory process observed in asthma, emphysema, osteoarthritis and sepsis.

Panel CNSD.01 Summary: Ag2120 The insulin and insulin-like growth factors belong to a family of polypeptides essential for proper regulation of physiologic processes such as energy metabolism, cell proliferation, development, and differentiation. The insulin-like growth factors bind to IGF with high affinity and compete with the IGF receptor for IGF binding. Transgenic mice overexpressing insulin-like growth factor binding proteins (IGFBPs) tend to show brain developmental abnormalities, suggesting a role for these proteins in neurodevelopment. Furthermore, treatment with glycosaminoglycans (which increases muscle re-innervation after motor neuron death) upregulates serum levels of both IGF and IGFBP. Thus, the novel IGFBP encoded by the MOL2 gene may be useful in the treatment of diseases such as ALS, multiple sclerosis, and peripheral nerve injury on the basis of its homology to other established IGFBPs. The expression profile of this gene suggests that it is expressed preferentially in the brain, with highest levels in the cerebral cortex and hippocampus, two regions that are known to degenerate in Alzheimer's disease. Examination of the expression profile on Panel CNS.01 shows that most regions of both control and diseased brains express this protein; however the levels are decreased in the motor cortex in progressive supranuclear palsy and depression. Thus, this protein may additionally be of use in the treatment of Alzheimer's disease, progressive supranuclear palsy, and depression. cl C. MOL3a

Expression of gene MOL3a was assessed using the primer-probe set Ag1493, described in Table 22. Results of the RTQ-PCR runs are shown in Tables 23, 24, 25, and 26.

TABLE 22
Probe Name Ag1493

| | | | | Start | SEQ ID |
|---|---|---|---|---|---|
| Primers | Sequences | TM | Length | Position | NO: |
| Forward | 5′-GTGAAATCTGGCGTGGAGTATA-3′ | 59.1 | 22 | 1224 | 74 |
| Probe | FAM-5′-CCTTGATGGGCACAGCCATCTTG-3′-TAMRA | 70 | 23 | 1274 | 75 |
| Reverse | 5′-GTACTGGTTCCCAGGTACATGA-3′ | 58.8 | 22 | 1318 | 76 |

TABLE 23
Panel 1.2

| | Relative | | Relative |
|---|---|---|---|
| | Expression (%) | | Expression (%) |
| Tissue Name | 1.2tm2058f_ag1493 | Tissue Name | 1.2tm2058f_ag1493 |
| Endothelial cells | 0.2 | Renal ca. 786-0 | 0.0 |
| Endothelial cells (treated) | 2.4 | Renal ca. A498 | 0.5 |
| Pancreas | 0.1 | Renal ca. RXΓ 393 | 0.9 |
| Pancreatic ca. CAPAN2 | 0.1 | Renal ca. ACHN | 0.2 |
| Adrenal Gland (new lot*) | 2.6 | Renal ca. UO-31 | 1.3 |
| Thyroid | 1.0 | Renal ca. TK-10 | 0.8 |
| Salivary gland | 21.8 | Liver | 1.4 |
| Pituitary gland | 0.4 | Liver (fetal) | 2.3 |
| Brain (fetal) | 3.3 | Liver ca. | 0.7 |
| | | (hepatoblast) HepG2 | |
| Brain (whole) | 2.1 | Lung | 0.8 |
| Brain (amygdala) | 8.0 | Lung (fetal) | 1.0 |
| Brain cerebellum | 0.3 | Lung ca. (small cell) | 0.4 |
| | | LX-1 | |
| Brain (hippocampus) | 15.0 | Lung ca. (small cell) | 1.9 |
| | | NCI-H69 | |
| Brain (thalamus) | 3.5 | Lung ca. (s.cell var.) | 0.0 |
| | | SHP-77 | |
| Cerebral Cortex | 30.1 | Lung ca. (large cell) | 0.7 |
| | | NCI-H460 | |
| Spinal cord | 0.2 | Lung ca. (non-sm cell) | 1.0 |
| | | A549 | |
| CNS ca. (glio/astro) | 0.2 | Lung ca. (non-s cell) | 0.2 |
| U87-MG | | NCI-H23 | |
| CNS ca. (glio/astro) | 0.3 | Lung ca. (non-s. cell) | 1.8 |
| U-118-MG | | HOP-62 | |
| CNS ca. (astro) SW1783 | 0.0 | Lung ca. (non-s. cl) | 11.9 |
| | | NCI-H522 | |
| CNS ca.* (neuro; met) | 0.5 | Lung ca. (squam.) SW 900 | 0.8 |
| SK-N-AS | | | |
| CNS ca. (astro) SF-539 | 0.4 | Lung ca. (squam.) | 1.0 |
| | | NCI-H596 | |
| CNS ca. (astro) SNB-75 | 0.1 | Mammary gland | 2.5 |
| CNS ca. (glio) SNB-19 | 0.4 | Breast ca.* (pl.ef) | 0.0 |
| | | MDA-MB-231 | |
| CNS ca. (glio) SF-295 | 1.3 | Breast ca.* (pl effusion) | 17.6 |
| | | T47D | |
| Heart | 5.1 | Breast ca. BT-549 | 0.2 |
| Skeletal Muscle (new lot*) | 8.0 | Breast ca. MDA-N | 0.4 |
| Bone marrow | 15.6 | Ovary | 2.7 |
| Thymus | 1.2 | Ovarian ca. OVCAR-3 | 8.3 |
| Spleen | 10.4 | Ovarian ca. OVCAR-4 | 16.6 |
| Lymph node | 0.7 | Ovarian ca. OVCAR-5 | 5.1 |
| Colorectal | 0.9 | Ovarian ca. OVCAR-8 | 0.8 |
| Stomach | 2.9 | Ovarian ca. IGROV-1 | 70.7 |
| Small intestine | 4.7 | Ovarian ca.* (ascites) | 0.7 |
| | | SK-OV-3 | |
| Colon ca. SW480 | 0.2 | Uterus | 0.6 |
| Colon ca.* (SW480 met) | 0.0 | Placenta | 0.6 |
| SW620 | | | |
| Colon ca. HT29 | 2.3 | Prostate | 27.9 |
| Colon ca. HCT-116 | 0.0 | Prostate ca.* | 0.3 |
| | | (bone met) PC-3 | |
| Colon ca. CaCo-2 | 0.0 | Testis | 0.6 |
| 83219 CC Well to | 1.4 | Melanoma Hs688 (A).T | 0.2 |
| Mod Diff (ODO3866) | | | |
| Colon ca. HCC-2998 | 2.8 | Melanoma* (met) Hs688 | 0.4 |
| | | (B).T | |
| Gastric ca.* (liver met) | 1.6 | Melanoma UACC-62 | 0.5 |
| NCI-N87 | | | |
| Bladder | 6.9 | Melanoma M14 | 0.1 |
| Trachea | 0.5 | Melanoma LOX IMVI | 0.1 |
| Kidney | 73.7 | Melanoma* (met) | 0.0 |
| | | SK-MEL-5 | |
| Kidney (fetal) | 4.7 | Adipose | 100.0 |

TABLE 24
Panel 1.3D

| | Relative | | Relative |
|---|---|---|---|
| | Expression (%) | | Expression (%) |
| Tissue Name | 1.3dx4tm5350f_ag1493_b1 | Tissue Name | 1.3dx4tm5350f_ag1493_b1 |
| Liver adenocarcinoma | 1.2 | Kidney (fetal) | 18.7 |
| Pancreas | 0.0 | Renal ca. 786-0 | 0.0 |
| Pancreatic ca. CAPAN 2 | 1.9 | Renal ca. A498 | 0.0 |
| Adrenal gland | 1.7 | Renal ca. RXF 393 | 2.9 |
| Thyroid | 38.0 | Renal ca. ACHN | 1.0 |
| Salivary gland | 30.8 | Renal ca. UO-31 | 0.8 |
| Pituitary gland | 4.5 | Renal ca. TK-10 | 0.8 |
| Brain (fetal) | 48.3 | Liver | 0.4 |
| Brain (whole) | 60.7 | Liver (fetal) | 11.2 |
| Brain (amygdala) | 100.0 | Liver ca. (hepatoblast) | 0.6 |
| | | HepG2 | |
| Brain (cerebellum) | 9.2 | Lung | 29.6 |
| Brain (hippocampus) | 68.2 | Lung (fetal) | 15.6 |
| Brain | 11.4 | Lung ca. (small cell) | 0.2 |
| (substantia nigra) | | LX-1 | |
| Brain (thalamus) | 20.8 | Lung ca. (small cell) | 0.0 |
| | | NCI-H69 | |
| Cerebral Cortex | 41.1 | Lung ca. (s.cell var.) | 0.0 |
| | | SHP-77 | |
| Spinal cord | 10.0 | Lung ca. (large cell) | 0.5 |
| | | NCI-H460 | |
| CNS ca. (glio/astro) | 0.0 | Lung ca. (non-sm. cell) | 0.4 |
| U87-MG | | A549 | |
| CNS ca. (glio/astro) | 1.3 | Lung ca. (non-s. cell) | 0.0 |
| U-118-MG | | NCI-H23 | |
| CNS ca. (astro) SW1783 | 0.7 | Lung ca. (non-s.cell) | 2.6 |
| | | HOP-62 | |
| CNS ca.* (neuro; met) | 1.9 | Lung ca. (non-s.cl) | 3.0 |
| SK-N-AS | | NCI-H522 | |
| CNS ca. (astro) SF-539 | 0.5 | Lung ca. (squam.) SW 900 | 0.2 |
| CNS ca. (astro) SNB-75 | 0.4 | Lung ca. (squam.) | 0.0 |
| | | NCI-H596 | |
| CNS ca. (glio) SNB-19 | 0.4 | Mammary gland | 22.3 |
| CNS ca. (glio) U251 | 1.2 | Breast ca.* (p1. effusion) | 14.1 |
| | | MCF-7 | |
| CNS ca. (glio) SF-295 | 0.6 | Breast ca.* (p1. ef) MDA- | 0.0 |
| | | MB-231 | |
| Heart (fetal) | 1.3 | Breast ca.* (p1. effusion) | 53.3 |
| | | T47D | |
| Heart | 1.4 | Breast ca. BT-549 | 0.5 |
| Fetal Skeletal | 4.2 | Breast ca. MDA-N | 0.0 |
| Skeletal muscle | 8.8 | Ovary | 2.1 |
| Bone marrow | 78.4 | Ovarian ca. OVCAR-3 | 8.7 |
| Thymus | 3.9 | Ovarian ca. OVCAR-4 | 25.1 |
| Spleen | 53.3 | Ovarian ca. OVCAR-5 | 3.8 |
| Lymph node | 37.3 | Ovarian ca. OVCAR-8 | 2.6 |
| Colorectal | 6.6 | Ovarian ca. IGROV-1 | 29.2 |
| Stomach | 23.1 | Ovarian ca.* (ascites) | 0.5 |
| | | SK-OV-3 | |
| Small intestine | 12.5 | Uterus | 8.9 |
| Colon ca. SW480 | 0.0 | Placenta | 10.9 |
| Colon ca.* (SW480 met) | 0.0 | Prostate | 50.9 |
| SW620 | | | |
| Colon ca. HT29 | 2.9 | Prostate ca.* (bone met) | 0.0 |
| | | PC-3 | |
| Colon ca. HCT-116 | 0.0 | Testis | 18.7 |
| Colon ca. CaCo-2 | 0.8 | Melanoma Hs688 (A).T | 0.5 |
| 83219 CC Well to Mod | 8.2 | Melanoma* (met) | 0.0 |
| Diff (ODO3866) | | Hs688 (B).T | |
| Colon ca. HCC-2998 | 0.8 | Melanoma UACC-62 | 0.0 |
| Gastric ca.* (liver met) | 2.8 | Melanoma M14 | 0.5 |
| NCI-N87 | | | |
| Bladder | 5.3 | Melanoma LOX IMVI | 0.0 |
| Trachea | 35.6 | Melanoma* (met) | 0.0 |
| | | SK-MEL-5 | |
| Kidney | 15.0 | Adipose | 60.2 |

TABLE 25
Panel 2D

| | Relative | | Relative |
|---|---|---|---|
| | Expression (%) | | Expression (%) |
| Tissue Name | 2Dtm2527f_ag1493 | Tissue Name | 2Dtm2527f_ag1493 |
| Normal Colon GENPAK | 41.2 | Kidney NAT Clontech | 28.3 |
| 061003 | | 8120608 | |
| 83219 CC Well to Mod | 8.0 | Kidney Cancer Clontech | 10.7 |
| Diff (ODO3866) | | 8120613 | |
| 83220 CC NAT | 9.3 | Kidney NAT Clontech | 46.0 |
| (ODO3866) | | 8120614 | |
| 83221 CC Gr.2 | 5.1 | Kidney Cancer Clontech | 48.6 |
| rectosigmoid (ODO3868) | | 9010320 | |
| 83222 CC NAT | 2.0 | Kidney NAT Clontech | 36.6 |
| (ODO3868) | | 9010321 | |
| 83235 CC Mod Diff | 6.3 | Normal Uterus GENPAK | 8.4 |
| (ODO3920) | | 061018 | |
| 83236 CC NAT | 4.4 | Uterus Cancer GENPAK | 17.4 |
| (ODO3920) | | 064011 | |
| 83237 CC Gr.2 | 12.5 | Normal Thyroid Clontech | 100.0 |
| ascend colon (ODO3921) | | A + 6570-1 | |
| 83238 CC NAT | 6.1 | Thyroid Cancer GENPACK | 39.8 |
| (ODO3921) | | 064010 | |
| 83241 CC from | 17.0 | Thyroid Cancer | 28.3 |
| Partial Hepatectomy | | INVITROGEN A302152 | |
| (ODO4309) | | | |
| 83242 Liver NAT | 7.6 | Thyroid NAT INVITROGEN | 44.4 |
| (ODO4309) | | A302153 | |
| Colon mets to lung | 16.6 | Normal Breast GENPAK | 36.3 |
| (ODO4451-01) | | 061019 | |
| Lung NAT | 25.5 | 84877 Breast Cancer | 24.7 |
| (ODO4451-02) | | (ODO4566) | |
| Normal Prostate | 82.4 | 85975 Breast Cancer | 29.7 |
| Clontech | | | |
| A + 6546-1 | | (ODO4590-01) | |
| 84140 Prostate Cancer | 36.3 | 85976 Breast Cancer | 17.8 |
| (ODO4410) | | Mets (ODO4590-03) | |
| 84141 Prostate NAT | 47.0 | 87070 Breast Cancer | 79.6 |
| (ODO4410) | | Metastasis (ODO4655-05) | |
| 87073 Prostate Cancer | 36.1 | GENPACK Breast Cancer | 25.9 |
| (ODO4720) | | 064006 | |
| 87074 Prostate NAT | 51.4 | Breast Cancer Res. Gen. 1024 | 55.5 |
| (ODO4720-02) | | | |
| Normal Lung | 41.8 | Breast Cancer | 24.0 |
| GENPACK 061010 | | Clontech 9100266 | |
| Lung Met to Muscle | 14.4 | Breast NAT Clontech | 13.7 |
| (ODO4286) | | 9100265 | |
| 83240 Muscle NAT | 4.5 | Breast Cancer INVITROGEN | 29.1 |
| (ODO4286) | | | |
| 84136 Lung Malignant | 36.1 | Breast NAT INVITROGEN | 29.9 |
| Cancer (ODO3126) | | A2090734 | |
| 84137 Lung NAT | 71.2 | Normal Liver GENPACK | 1.0 |
| (ODO3126) | | 061009 | |
| 84871 Lung Cancer | 68.3 | Liver Cancer GENPAK | 1.6 |
| (ODO4404) | | 064003 | |
| 84872 Lung NAT | 33.7 | Liver Cancer Research | 7.4 |
| (ODO4404) | | Genetics RNA 1025 | |
| 84875 Lung Cancer | 25.5 | Liver Cancer Research | 4.3 |
| (ODO4565) | | Genetics RNA 1026 | |
| 84876 Lung NAT | 18.4 | Paired Liver Cancer Tissue | 8.5 |
| (ODO4565) | | Research Genetrics RNA | |
| | | 6004-T | |
| 85976 Lung Cancer | 45.1 | Paired Liver Tissue Research | 10.1 |
| (ODO4237-01) | | Genetics RNA 6004-N | |
| 85970 Lung NAT | 55.1 | Paired Liver Cancer Tissue | 7.5 |
| (ODO4237-02) | | Research Genetics RNA | |
| | | 6005-T | |
| 83255 Ocular Mel Met | 8.7 | Paired Liver Tissue Research | 2.8 |
| to Liver (ODO4310) | | Genetics RNA 6005-N | |
| 83256 Liver NAT | 4.7 | Normal Bladder GENPACK | 11.7 |
| (ODO4310) | | 061001 | |
| 84139 Melanoma Mets | 4.0 | Bladder Cancer Research | 6.4 |
| to Lung (ODO4321) | Genetics RNA 1023 | | |
| 84138 Lung NAT | 50.3 | Bladder Cancer | 50.3 |
| (ODO4321) | | INVITROGEN A302173 | |
| Normal Kidney | 53.2 | 87071 Bladder Cancer | 35.4 |
| GENPACK 061008 | | (ODO4718-01) | |
| 83786 Kidney Ca, | 13.6 | 87072 Bladder Normal | 13.6 |
| Nuclear grade 2 | | Adjacent (ODO4718-03) | |
| (ODO4338) | | | |
| 83787 Kidney NAT | 54.0 | Normal Ovary Res. Gen. | 6.4 |
| (ODO4338) | | | |
| 83788 Kidney Ca | 23.0 | Ovarian Cancer GENPACK | 22.1 |
| Nuclear grade 1/2 | | 064008 | |
| (ODO4339) | | | |
| 83789 Kidney NAT | 26.6 | 87492 Ovary Cancer | 29.3 |
| (ODO4339) | | (ODO4768-07) | |
| 83790 Kidney Ca. | 31.0 | 87493 Ovary NAT | 20.7 |
| Clear cell type | | (ODO4768-08) | |
| (ODO4340) | | | |
| 83791 Kidney NAT | 49.7 | Normal Stomach GENPAK | 21.9 |
| (ODO4340) | | 061017 | |
| 83792 Kidney Ca, | 7.8 | Gastric Cancer Clontech | 12.6 |
| Nuclear grade 3 | | 9060358 | |
| (ODO4348) | | | |
| 83793 Kidney NAT | 35.8 | NAT Stomach Clontech | 33.7 |
| (ODO4348) | | 9060359 | |
| 87474 Kidney Cancer | 17.8 | Gastric Cancer Clontech | 15.6 |
| (ODO4622-01) | | 906395 | |
| 87475 Kidney NAT | 15.4 | NAT Stomach Clontech | 51.8 |
| (ODO4622-03) | | 9060394 | |
| 85973 Kidney Cancer | 1.7 | Gastric Cancer Clontech | 16.8 |
| (ODO4450-01) | | 9060397 | |
| 85974 Kidney NAT | 43.2 | NAT Stomach Clontech | 12.4 |
| (ODO4450-03) | | 9060396 | |
| Kidney Cancer Clontech | 4.3 | Gastric Cancer GENPAK | 12.9 |
| 8120607 | | 064005 | |

TABLE 26
Panel 4.1D

| | Relative | | Relative |
|---|---|---|---|
| | Expression (%) | | Expression (%) |
| Tissue Name | 4.1dx4tm6520f_ag1493_a1 | Tissue Name | 4.1dx4tm6520f_ag1493_a1 |
| 93768_Secondary Th1_anti- | 3.8 | 93100_HUVEC | 0.0 |
| CD28/anti-CD3 | | (Endothelial)_IL-1b | |
| 93769_Secondary Th2_anti- | 27.0 | 93779_HUVEC | 0.6 |
| CD28/anti-CD3 | | (Endothelial)_IFN gamma | |
| 93770_Secondary Tr1_anti- | 11.9 | 93102_HUVEC | 1.0 |
| CD28/anti-CD3 | | (Endothelial)_TNF | |
| | | alpha + IFN gamma | |
| 93573_Secondary | 1.1 | 93101_HUVEC | 0.9 |
| Th1_resting | | (Endothelial)_TNF | |
| day 4-6 in IL-2 | | alpha + IL-4 | |
| 93572_Secondary | 3.2 | 93781_HUVEC | 0.6 |
| Th2_resting | | (Endothelial)_IL-11 | |
| day 4-6 in IL-2 | | | |
| 93571_Secondary | 1.6 | 93583_Lung Microvascular | 1.6 |
| Tr1_resting | | Endothelial Cells_none | |
| day 4-6 in IL-2 | | | |
| 93568_primary Th1_anti- | 6.7 | 93584_Lung Microvascular | |
| CD28/anti-CD3 | | Endothelial Cells_TNFa | |
| | | (4 ng/ml) and IL1b (1 ng/ml) | |
| 93569_primary Th2_anti- | 8.4 | 92662_Microvascular | 0.2 |
| CD28/anti-CD3 | | Dermal endothelium_none | |
| 93570_primary Tr1_anti- | 1.9 | 92663_Microvasular | 0.3 |
| CD28/anti-CD3 | | Dermal endothelium_TNFa | |
| | | (4 ng/ml) and IL1b (1 ng/ml) | |
| 93565_primary Th1_resting | 0.4 | 93773_Bronchial | 2.1 |
| dy 4-6 in IL-2 | | epithelium_TNFa | |
| | | (4 ng/ml) and IL1b | |
| | | (1 ng/ml)** | |
| 93566_primary Th2_resting | 0.7 | 93347_Small Airway | 1.0 |
| dy 4-6 in IL-2 | | Epithelium_none | |
| 93567_primary Tr1_resting | 0.2 | 93348_Small Airway | 4.2 |
| dy 4-6 in IL-2 | | Epithelium_TNFa | |
| | | (4 ng/ml) and IL1b (1 ng/ml) | |
| 93351_CD45RA CD4 | 3.8 | 92668_Coronery Artery | 0.1 |
| lymphocyte_anti- | | SMC_resting | |
| CD/28/anti-CD3 | | | |
| 93352_CD45RO CD4 | 7.1 | 92669_Coronery Artery | 0.4 |
| lymphocyte_anti- | | SMC_TNFa (4 ng/ml) | |
| CD3 | | and IL1b (1 ng/ml) | |
| 93251_CD8 | | | |
| Lymphocytes_anti- | 6.1 | 93107_astrocytes_resting | 0.1 |
| CD28/anti-CD3 | | | |
| 93353_chronic CD8 | 25.6 | 93108_astrocytes_TNFa | 0.0 |
| Lymphocytes 2ry_resting | | (4 ng/ml) and IL1b (1 ng/ml) | |
| dy 4-6 in IL-2 | | | |
| 93574_chronic CD8 | 9.9 | 92666_KU-812 | 0.3 |
| Lymphocytes 2ry_activated | | (Basophil)_resting | |
| CD3/CD28 | | | |
| 93354_CD4_none | 0.5 | 92667_KU-812 | 0.5 |
| | | (Basophil)_PMA/ionoycin | |
| 93252_Secondary | 1.2 | 93579_CCD1106 | 4.2 |
| Th1/Th2/Tr1_anti- | | (Keratinocytes)_none | |
| CD95 CH11 | | | |
| 93103_LAK | 25.6 | 93580_CCD1106 | 4.0 |
| cells_resting | | (Keratinocytes)_TNFa | |
| | | and IFNg** | |
| 93788_LAK | | | |
| cells_IL-2 | 3.2 | 93791_Liver Cirrhosis | 1.7 |
| 93787_LAK | 6.4 | 93577_NCI-H292 | 3.3 |
| cells_IL-2 + IL-12 | | | |
| 93789_IL-2 + IFN | 5.3 | 93358_NCI-H292_IL-4 | 5.5 |
| gamma | | | |
| 93790_LAK | | | |
| cells_IL-2 + IL-18 | 4.6 | 93360_NCI-H292_IL-9 | 4.6 |
| 93104_LAK | 11.6 | 93359_NCI-H292_IL-13 | 6.3 |
| cells_PMA/ionomycin | | | |
| and IL-18 | | | |
| 93578_NK Cells | 3.0 | 93357_NCI-H292_IFN | 3.4 |
| IL-2_resting | | gamma | |
| 93109_Mixed Lymphocyte | 17.2 | 93777_HPAEC_- | 0.2 |
| Reaction_Two Way MLR | | | |
| 93110_Mixed Lymphocyte | 13.5 | 93778_HPAEC_{—IL-1} | 0.5 |
| Reaction_Two Way MLR | | beta/TNA alpha | |
| 93111_Mixed Lymphocyte | 5.4 | 93254_Normal Human Lung | 0.5 |
| Reaction_Two Way MLR | | Fibroblast_none | |
| 93112_Mononuclear Cells | 9.1 | 93253_Normal Human Lung | 0.3 |
| (PBMCs)_resting | | Fibroblast_TNFa | |
| | | (4 ng/ml) and IL-1b (1 ng/ml) | |
| 93113_Mononuclear Cells | 11.5 | 93257_Normal Human Lung | 0.2 |
| (PBMCs)_PWM | | Fibroblast_IL-4 | |
| 93114_Mononuclear Cells | 20.5 | 93256_Normal Human Lung | 0.3 |
| (PBMCs)_PHA-L | | Fibroblast_IL-9 | |
| 93249_Ramos | 70.5 | 93255_Normal Human Lung | 0.6 |
| (B cell)_none | | Fibroblast_IL-13 | |
| 93250_Ramos | 100.0 | 93258_Normal Human Lung | 0.5 |
| (B cell)_ionomycin | | Fibroblast_IFN gamma | |
| 93349_B | 12.4 | 93106_Dermal Fibroblasts | 0.5 |
| lymphocytes_PWM | | CCD1070_resting | |
| 93350_B | 64.9 | 93361_Dermal Fibroblasts | 4.1 |
| lymphocytes_CD40L | | CCD1070_TNF alpha | |
| and IL-4 | | 4 ng/ml | |
| 92665_EOL-1 | 10.0 | 93105_Dermal Fibroblasts | 0.1 |
| (Eosinophil)_dbcAMP | | CCD1070_IL-1 beta 1 ng/ml | |
| differentiated | | | |
| 93248_EOL-1 | 6.9 | 93772_dermal | 1.0 |
| (Eosinophil)_dbcAMP/ | | fibroblast_IFN gamma | |
| PMAionomycin | | | |
| 93356_Dendritic | 10.1 | 93771_dermal | 2.2 |
| Cells_none | | fibroblast_IL-4 | |
| 93355_Dendritic | 21.1 | 93892_Dermal | 1.0 |
| Cells_LPS 100 ng/ml | | fibroblasts_none | |
| 93775_Dendritic | 9.9 | 99202_Neutrophils_TNFa + | 9.2 |
| Cells_anti-CD40 | | LPS | |
| 93774_Monocytes_resting | 38.2 | 99203_Neutrophils_none | 38.1 |
| 93776_Monocytes_LPS | 51.1 | 735010_Colon_normal | 1.2 |
| 50 ng/ml | | | |
| 93581_Macrophages_resting | 18.4 | 735019_Lung_none | 5.5 |
| 93582_Macrophages_LPS | 19.7 | 64028-1_Thymus_none | 8.2 |
| 100 ng/ml | | | |
| 93098_HUVEC | 0.0 | 64030-1_Kidney_none | 9.8 |
| (Endothelial)_none | | | |
| 93099_HUVEC | 0.3 | | |
| (Endothelial)_starved | | | |

TABLE 27
Panel CNSD.01

| | Relative | | Relative |
|---|---|---|---|
| | Expression (%) | | Expression (%) |
| Tissue Name | cns1 × 4tm6179f_ag1493_b1 | Tissue Name | cns1 × 4tm6179f_ag1493_b1 |
| 102633_BA4 Control | 14.8 | 102605_BA17 PSP | 15.9 |
| 102641_BA4 Control2 | 51.1 | 102612 BA17 PSP2 | 8.3 |
| 102625_BA4 Alzheimer's2 | 3.6 | 102637_Sub Nigra | 28.5 |
| | | Control | |
| 102649_BA4 Parkinson's | 36.3 | 102645_Sub Nigra | 47.1 |
| | | Control2 | |
| 102656_BA4 Parkinson's | 54.3 | 102629_Sub Nigra | 3.3 |
| | | Alzheimer's | |
| 102664_BA4 Huntington's | 17.2 | 102660_Sub Nigra | 61.6 |
| | | Parkinson's | |
| 102671_BA4 Huntington's | 4.3 | 102667_Sub Nigra | 37.6 |
| | | Huntington's | |
| 102603_BA4 PSP | 0.0 | 102674_Sub Nigra | 8.2 |
| | | Huntington's | |
| 102610_BA4 PSP2 | 14.0 | 102614_Sub Nigra | 4.6 |
| | | PSP2 | |
| 102588_BA4 Depression | 14.9 | 102592_Sub Nigra | 6.8 |
| | | Depression | |
| 102596_BA4 Depression2 | 2.6 | 102599_Sub Nigra | 6.6 |
| | | Depression2 | |
| 102634_BA7 Control | 16.5 | 102636_Glob | 10.2 |
| | | Palladus Control | |
| 102642_BA7 Control2 | 13.7 | 102644_Glob | 9.0 |
| | | Palladus Control2 | |
| 102626_BA7 Alzheimer's | 3.9 | 102620_Glob | 4.4 |
| | | Palladus Alzheimer's | |
| 102650_BA7 Parkinson's | 18.2 | 102628_Glob | 2.6 |
| | | Palladus Alzheimer's2 | |
| 102657_BA7 Parkinson's | 45.6 | 102652_Glob | 80.4 |
| | | Palladus Parkinson's | |
| 102665_BA7 Huntington's | 35.9 | 102659_Glob | 5.2 |
| | | Palladus Parkinson's2 | |
| 102672_BA7 Huntington's2 | 18.7 | 102606_Glob | 6.4 |
| | | Palladus PSP | |
| 102604_BA7 PSP | 54.8 | 102613_Glob | 2.7 |
| | | Palladus PSP2 | |
| 102611_BA7 PSP2 | 17.8 | 102591_Glob | 4.0 |
| | | Palladus Depression | |
| 102589_BA7 Depression | 11.2 | 102638_Temp | 13.8 |
| | | Pole Control | |
| 102632_BA9 Control | 16.1 | 102646_Temp | 100.0 |
| | | Pole Control2 | |
| 102640_BA9 Control2 | 96.5 | 102622_Temp | 0.8 |
| | | Pole Alzheimer's | |
| 102617_BA9 Alzheimer's | 0.0 | 102630_Temp | 3.0 |
| | | Pole Alzheimer's2 | |
| 102624_BA9 Alzheimer's2 | 7.0 | 102653_Temp | 51.3 |
| | | Pole Parkinson's | |
| 102648_BA9 Parkinson's | 29.5 | 102661_Temp | 34.2 |
| | | Pole Parkinson's | |
| 102655_BA9 Parkinson's2 | 47.8 | 102668_Temp | 50.4 |
| | | Pole Huntington's | |
| 102663_BA9 Huntington's | 27.6 | 102607_Temp | 1.2 |
| | | Pole PSP | |
| 102670_BA9 Huntington's2 | 14.9 | 102615_Temp | 1.7 |
| | Pole PSP2 | | |
| 102602_BA9 PSP | 9.0 | 102600_Temp | 8.9 |
| | | Pole Depression2 | |
| 102609_BA9 PSP2 | 3.1 | 102639_Cing | 50.1 |
| | | Gyr Control | |
| 102587_BA9 Depression | 3.2 | 102647_Cing | 55.7 |
| | | Gyr Control2 | |
| 102595_BA9 Depression2 | 5.3 | 102623 Cing | 18.9 |
| | | Gyr Alzheimer's | |
| 102635_BA17 Control | 17.9 | 102631_Cing | 0.9 |
| | | Gyr Alzheimer's2 | |
| 102643_BA17 Control2 | 37.2 | 102654_Cing | 35.5 |
| | | Gyr Parkinson's | |
| 102627_BA17 Alzheimer's2 | 5.7 | 102662_Cing | 84.5 |
| | | Gyr Parkinson's | |
| 102651_BA17 Parkinson's | 45.7 | 102669_Cing | 67.5 |
| | | Gyr Huntington's | |
| 102658_BA17 Parkinson's2 | 18.1 | 102676_Cing | 23.3 |
| | | Gyr Huntington's2 | |
| 102666_BA17 Huntington's | 15.1 | 102608_Cing | 14.0 |
| | | Gyr PSP | |
| 102673_BA17 Huntington's2 | 13.0 | 102616_Cing | 7.9 |
| | | Gyr PSP2 | |
| 102590_BA17 Depression | 9.4 | 102594_Cing | 3.0 |
| | | Gyr Depression | |
| 102597_BA17 Depression2 | 31.9 | 102601_Cing | 11.8 |
| | | Gyr Depression2 | |

Panel 1.2 Summary: Ag1493 The high expression of the MOL3a gene seen in adipose (CT value=25) is most likely skewed due to genomic DNA contamination in this sample. Otherwise, the gene is expressed mainly in normal tissues, including brain (particularly cerebral cortex), kidney, and prostate. Expression of the MOL3a gene in skeletal muscle and liver may suggest function in metabolic diseases, including obesity and diabetes. Furthermore, MOL3a expression is down regulated in a number of tumor cell lines relative to the normal controls suggesting a potential utility of this gene in the treatment of cancer.

Panel 1.3D Summary: Ag1493 In this panel, highest expression of the MOL3a gene is detected in the amygdala of the brain (CT value=29.6). This may suggest that the MOL3a gene plays a role in normal brain function, including fear and anxiety response. In addition, high expression is also observed in adipose and bone marrow suggesting potential roles in metabolic and immune function. Overall, expression of the MOL3a gene in panel 1.3D reveals that it is associated mostly with normal tissues. In a couple of instances, the expression of this gene is seen in clusters of cell lines, specifically in breast and ovarian cancer cell lines. Thus, therapeutic modulation of expression of this gene may be of utility in the treatment breast and ovarian cancers. Alternatively, replacement of the MOL3a protein that is missing from some cancer cells using recombinant protein might provide a useful treatment for these types of cancers.

Panel 2D Summary: Ag1493 Expression of the MOL3a gene is highest in thyroid and appears to be widespread across many samples on Panel 2D. However, overall there appears to be generally higher expression in normal tissues when compared to cancerous counterparts. Thus, therapeutic modulation of this gene or gene product might show utility for a range of oncology indications. Semaphorins and their receptors are known signals for axon guidance; they are also suspected to regulate developmental processes involving cell migration and morphogenesis, and have been implicated in immune function and tumor progression.

Panel 4.1D Summary: Ag1493 The MOL3a transcript is highly expressed in a B cell line as well as in B cells stimulated with CD40L and IL4. Expression of this transcript is also found to a lesser degree in monocytes and macrophages independently of their activation status. Of interest, CD100, which is an activation molecule on T cells, is a member of the semaphorin protein family. The semaphiorin B-like protein encoded by the MOL3a transcript could therefore also serve as a B cell activation marker. The semaphorin family has additionally been reported to play a role in chemotaxis. Thus, protein therapeutics or monoclonal antibodies raised against the MOL3a protein, could inhibit spontaneous and chemokine induced migration of B cells and monocytes and potentially regulate B cell differentiation and B cell isotype switching. Regulation of this molecule by protein therapeutics or monoclonal antibodies could also function to regulate immunity and be important for the treatment of autoimmune diseases, allergic diseases, and immune rejection in transplantation. In support of this hypothesis, recent studies indicate that semaphorins bind with high affinity to at least two different receptor families and are biologically active on immune cells as well as neuronal cells (Curr Opin Immunol 1999 August;11(4):387-91).

Panel CNSD.01 Summary: Ag1493 Semaphorins can act as axon guidance proteins, specifically through their ability to act as chemorepellents that inhibit CNS regenerative capacity. Although there is considerable variance between individuals in MOL3a gene expression levels in this panel, levels of this protein are reduced to less than ⅓ of that seen in controls in the temporal cortex of Alzheimer's patients (which shows marked synaptogenic loss in mid to late phases of the disease) as well as in diseases not associated with neurodegeneration of the temporal cortex. Therefore, manipulation of levels of this protein may be of use in inducing a compensatory synaptogenic response to neuronal death in Alzheimer's disease. cl D. MOL4a

Expression of gene MOL4a was assessed using the primer-probe set Ag1216, described in Table 28. Results of the RTQ-PCR runs are shown in Tables 29, 30, 31, and 32.

TABLE 28
Probe Name: Ag1216

| | | | | Start | SEQ ID |
|---|---|---|---|---|---|
| Primers | Sequences | TM | Length | Position | NO: |
| Forward | 5′-CCCGAAGAATGAAAAGTACACA-3′ | 59.1 | 22 | 5263 | 77 |
| Probe | FAM-5′-CCCATGGAATTCAAGACCCTGAACAA-3′-TAMRA | 59.7 | 26 | 5285 | 78 |
| Reverse | 5′-AATGGGTAGAAGTTGGCTCTGT-3′ | 59.2 | 22 | 5331 | 79 |

TABLE 29
Panel 1.2

| | Relative | | Relative |
|---|---|---|---|
| | Expression(%) | | Expression(%) |
| | 1.2tm1404f_{—} | | 1.2tm1404f_{—} |
| Tissue Name | ag1216 | Tissue Name | ag1216 |
| Endothelial cells | 0.0 | Renal ca. 786-0 | 100.0 |
| Endothelial cells (treated) | 76.8 | Renal ca. A498 | 3.0 |
| Pancreas | 11.3 | Renal ca. RXF 393 | 45.4 |
| Pancreatic ca. CAPAN 2 | 0.0 | Renal ca. ACHN | 0.2 |
| Adrenal Gland (new lot*) | 12.2 | Renal ca. UO-31 | 0.0 |
| Thyroid | 11.6 | Renal ca. TK-10 | 0.0 |
| Salivary gland | 2.7 | Liver | 7.5 |
| Pituitary gland | 12.6 | Liver (fetal) | 7.7 |
| Brain (fetal) | 77.9 | Liver ca. (hepatoblast) HepG2 | 0.0 |
| Brain (whole) | 73.2 | Lung | 6.1 |
| Brain (amygdala) | 33.7 | Lung (fetal) | 13.1 |
| Brain (cerebellum) | 6.8 | Lung ca. (small cell) LX-1 | 0.0 |
| Brain (hippocampus) | 75.3 | Lung ca. (small cell) NCI-H69 | 0.0 |
| Brain (thalamus) | 9.9 | Lung ca. (s. cell var.) SHP-77 | 0.0 |
| Cerebral Cortex | 87.7 | Lung ca. (large cell)NCI-H460 | 0.0 |
| Spinal cord | 8.3 | Lung ca. (non-sm. cell) A549 | 0.0 |
| CNS ca. (glio/astro) U87-MG | 0.0 | Lung ca. (non-s. cell) NCI-H23 | 0.0 |
| CNS ca. (glio/astro) U-118-MG | 1.5 | Lung ca (non-s. cell) HOP-62 | 0.0 |
| CNS ca. (astro) SW1783 | 0.0 | Lung ca. (non-s. cl) NCI-H522 | 0.0 |
| CNS ca.* (neuro; met) SK-N- | 11.1 | Lung ca. (squam.) SW 900 | 0.0 |
| AS | | | |
| CNS ca. (astro) SF-539 | 0.0 | Lung ca. (squam.) NCI-H596 | 0.0 |
| CNS ca. (astro) SNB-75 | 6.4 | Mammary gland | 13.0 |
| CNS ca. (glio) SNB-19 | 4.4 | Breast ca.* (pl. effusion) MCF- | 0.0 |
| | | 7 | |
| CNS ca. (glio) U251 | 4.2 | Breast ca.* (pl. ef) MDA-MB- | 0.0 |
| | | 231 | |
| CNS ca. (glio) SF-295 | 0.0 | Breast ca.* (pl. effusion) T47D | 0.0 |
| Heart | 61.1 | Breast ca. BT-549 | 1.8 |
| Skeletal Muscle (new lot*) | 8.2 | Breast ca. MDA-N | 0.0 |
| Bone marrow | 0.1 | Ovary | 10.2 |
| Thymus | 0.2 | Ovarian ca. OVCAR-3 | 66.4 |
| Spleen | 0.0 | Ovarian ca. OVCAR-4 | 1.3 |
| Lymph node | 5.1 | Ovarian ca. OVCAR-5 | 3.5 |
| Colorectal | 0.2 | Ovarian ca. OVCAR-8 | 0.0 |
| Stomach | 5.9 | Ovarian ca. IGROV-1 | 0.3 |
| Small intestine | 11.8 | Ovarian ca.* (ascites) SK-OV-3 | 0.0 |
| Colon ca. SW480 | 0.0 | Uterus | 7.7 |
| Colon ca.* (SW480 met)SW620 | 2.7 | Placenta | 8.1 |
| Colon ca. HT29 | 0.0 | Prostate | 4.6 |
| Colon ca. HCT-116 | 0.0 | Prostate ca.* (bone met)PC-3 | 0.0 |
| Colon ca. CaCo-2 | 0.5 | Testis | 4.6 |
| 83219 CC Well to Mod Diff | 0.5 | Melanoma Hs688(A).T | 0.0 |
| (ODO3866) | | | |
| Colon ca. HCC-2998 | 0.0 | Melanoma* (met) Hs688(B).T | 0.0 |
| Gastric ca.* (liver met) NCI- | 0.0 | Melanoma UACC-62 | 0.0 |
| N87 | | | |
| Bladder | 6.7 | Melanoma M14 | 0.0 |
| Trachea | 3.1 | Melanoma LOX IMVI | 0.0 |
| Kidney | 32.3 | Melanoma* (met) SK-MEL-5 | 0.0 |
| Kidney (fetal) | 51.8 | Adipose | 1.5 |

TABLE 30
Panel 2.2

| | Relative | | Relative |
|---|---|---|---|
| | Expression(%) | | Expression(%) |
| | 2.2x4tm6515f_{—} | | 2.2x4tm6515f_{—} |
| Tissue Name | ag1216_b1 | Tissue Name | ag1216_b1 |
| Normal Colon GENPAK | 0.1 | 83793 Kidney NAT (OD04348) | 11.6 |
| 061003 | | | |
| 97759 Colon cancer (OD06064) | 0.0 | 98938 Kidney malignant cancer | 0.0 |
| | | (OD06204B) | |
| 97760 Colon cancer NAT | 0.4 | 98939 Kidney normal adjacent | 1.4 |
| (OD06064) | | tissue (OD06204E) | |
| 97778 Colon cancer (OD06159) | 0.0 | 85973 Kidney Cancer | 1.3 |
| | | (OD04450-01) | |
| 97779 Colon cancer NAT | 0.8 | 85974 Kidney NAT (OD04450- | 3.2 |
| (OD06159) | | 03) | |
| 98861 Colon cancer (OD06297- | 0.0 | Kidney Cancer Clontech | 0.0 |
| 04) | | 8120613 | |
| 98862 Colon cancer NAT | 0.6 | Kidney NAT Clontech 8120614 | 3.5 |
| (OD06297-015) | | | |
| 83237 CC Gr.2 ascend colon | 0.2 | Kidney Cancer Clontech | 3.5 |
| (ODO3921) | | 9010320 | |
| 83238 CC NAT (ODO3921) | 0.0 | Kidney NAT Clontech 9010321 | 0.9 |
| 97766 Colon cancer metastasis | 0.2 | Kidney Cancer Clontech | 8.3 |
| (OD06104) | | 8120607 | |
| 97767 Lung NAT (OD06104) | 1.1 | Kidney NAT Clontech 8120608 | 0.7 |
| 87472 Colon mets to lung | 0.3 | Normal Uterus GENPAK | 2.9 |
| (OD04451-01) | | 061018 | |
| 87473 Lung NAT (OD04451- | 0.4 | Uterus Cancer GENPAK | 0.2 |
| 02) | | 064011 | |
| Normal Prostate Clontech A+ | 0.5 | Normal Thyroid Clontech A+ | 0.4 |
| 6546-1 (8090438) | | 6570-1 (7080817) | |
| 84140 Prostate Cancer | 0.2 | Thyroid Cancer GENPAK | 0.2 |
| (OD04410) | | 064010 | |
| 84141 Prostate NAT | 0.0 | Thyroid Cancer INVITROGEN | 0.3 |
| (OD04410) | | A302152 | |
| Normal Ovary Res. Gen. | 2.3 | Thyroid NAT INVITROGEN | 0.3 |
| | | A302153 | |
| 98863 Ovarian cancer | 4.6 | Normal Breast GENPAK | 1.3 |
| (OD06283-03) | | 061019 | |
| 98865 Ovarian cancer | 1.3 | 84877 Breast Cancer | 0.4 |
| NAT/fallopian tube (OD06283- | | (OD04566) | |
| 07) | | | |
| Ovarian Cancer GENPAK | 9.7 | Breast Cancer Res. Gen. 1024 | 0.4 |
| 064008 | | | |
| 97773 Ovarian cancer | 0.0 | 85975 Breast Cancer | 1.6 |
| (OD06145) | | (OD04590-01) | |
| 97775 Ovarian cancer NAT | 0.0 | 85976 Breast Cancer Mets | 0.8 |
| (OD06145) | | (OD04590-03) | |
| 98853 Ovarian cancer | 0.2 | 87070 Breast Cancer Metastasis | 0.6 |
| (OD06455-03) | | (OD04655-05) | |
| 98854 Ovarian NAT | 1.9 | GENPAK Breast Cancer | 0.9 |
| (OD06455-07) Fallopian tube | | 064006 | |
| Normal Lung GENPAK 061010 | 0.6 | Breast Cancer Clontech | 1.4 |
| | | 9100266 | |
| 92337 Invasive poor diff. lung | 0.4 | Breast NAT Clontech 9100265 | 0.6 |
| adeno (ODO4945-01 | | | |
| 92338 Lung NAT (ODO4945- | 0.2 | Breast Cancer INVITROGEN | 0.0 |
| 03) | | A209073 | |
| 84136 Lung Malignant Cancer | 0.0 | Breast NAT INVITROGEN | 1.4 |
| (OD03126) | | A2090734 | |
| 84137 Lung NAT (OD03126) | 0.4 | 97763 Breast cancer | 0.8 |
| | | (OD06083) | |
| 90372 Lung Cancer | 1.0 | 97764 Breast cancer node | 0.9 |
| (OD05014A) | | metastasis (OD06083) | |
| 90373 Lung NAT (OD05014B) | 2.1 | Normal Liver GENPAK | 0.3 |
| | | 061009 | |
| 97761 Lung cancer (OD06081) | 0.8 | Liver Cancer Research Genetics | 0.7 |
| | | RNA 1026 | |
| 97762 Lung cancer NAT | 0.4 | Liver Cancer Research Genetics | 1.6 |
| (OD06081) | | RNA 1025 | |
| 85950 Lung Cancer (OD04237- | 0.4 | Paired Liver Cancer Tissue | 0.0 |
| 01) | | Research Genetics RNA 6004- | |
| | | T | |
| 85970 Lung NAT (OD04237- | 0.0 | Paired Liver Tissue Research | 0.0 |
| 02) | | Genetics RNA 6004-N | |
| 83255 Ocular Mel Met to Liver | 0.2 | Paired Liver Cancer Tissue | 1.3 |
| (ODO4310) | | Research Genetics RNA 6005- | |
| | | T | |
| 83256 Liver NAT (ODO4310) | 0.0 | Paired Liver Tissue Research | 0.4 |
| | | Genetics RNA 6005-N | |
| 84139 Melanoma Mets to Lung | 0.7 | Liver Cancer GENPAK 064003 | 0.5 |
| (OD04321) | | | |
| 84138 Lung NAT (OD04321) | 0.0 | Normal Bladder GENPAK | 0.8 |
| | | 061001 | |
| Normal Kidney GENPAK | 1.7 | Bladder Cancer Research | 0.7 |
| 061008 | | Genetics RNA 1023 | |
| 83786 Kidney Ca, Nuclear | 5.3 | Bladder Cancer INVITROGEN | 0.2 |
| grade 2 (OD04338) | | A302173 | |
| 83787 Kidney NAT (OD04338) | 6.2 | Normal Stomach GENPAK | 1.0 |
| | | 061017 | |
| 83788 Kidney Ca Nuclear grade | 100.0 | Gastric Cancer Clontech | 0.2 |
| ½ (OD04339) | | 9060397 | |
| 83789 Kidney NAT (OD04339) | 3.0 | NAT Stomach Clontech | 0.4 |
| | | 9060396 | |
| 83790 Kidney Ca, Clear cell | 26.3 | Gastric Cancer Clontech | 0.4 |
| type (OD04340) | | 9060395 | |
| 83791 Kidney NAT (OD04340) | 0.9 | NAT Stomach Clontech | 0.7 |
| | | 9060394 | |
| 83792 Kidney Ca, Nuclear | 5.1 | Gastric Cancer GENPAK | 1.1 |
| grade 3 (OD04348) | | 064005 | |

TABLE 31
Panel 4D

| | Relative Expression(%) | |
|---|---|---|
| | 4Dtm2072f_{—} | 4Dtm2246f_{—} |
| Tissue Name | ag1216 | ag1216 |
| 93768_Secondary Th1_anti-CD28/anti-CD3 | 0.0 | 0.0 |
| 93769_Secondary Th2_anti-CD28/anti-CD3 | 0.0 | 0.0 |
| 93770_Secondary Tr1_anti-CD28/anti-CD3 | 0.0 | 0.0 |
| 93573_Secondary Th1_resting day 4-6 in IL-2 | 0.0 | 0.0 |
| 93572_Secondary Th2_resting day 4-6 in IL-2 | 0.0 | 0.0 |
| 93571_Secondary Tr1_resting day 4-6 in IL-2 | 0.0 | 0.0 |
| 93568_primary Th1_anti-CD28/anti-CD3 | 0.0 | 0.0 |
| 93569_primary Th2_anti-CD28/anti-CD3 | 0.0 | 0.0 |
| 93570_primary Tr1_anti-CD28/anti-CD3 | 0.0 | 0.0 |
| 93565_primary Th1_resting dy 4-6 in IL-2 | 0.0 | 0.0 |
| 93566_primary Th2_resting dy 4-6 in IL-2 | 1.1 | 0.0 |
| 93567_primary Tr1_resting dy 4-6 in IL-2 | 0.0 | 0.9 |
| 93351_CD45RA CD4 lymphocyte_anti-CD28/anti-CD3 | 0.0 | 0.0 |
| 93352_CD45RO CD4 lymphocyte_anti-CD28/anti-CD3 | 0.8 | 0.0 |
| 93251_CD8 Lymphocytes_anti-CD28/anti-CD3 | 0.0 | 0.0 |
| 93353_chronic CD8 Lymphocytes 2ry_resting dy 4-6 in IL-2 | 0.0 | 1.3 |
| 93574_chronic CD8 Lymphocytes 2ry_activated CD3/CD28 | 0.0 | 0.0 |
| 93354_CD4_none | 0.0 | 0.9 |
| 93252_Secondary Th1/Th2/Tr1_anti-CD95 CH 11 | 0.0 | 0.0 |
| 93103_LAK cells_resting | 0.0 | 0.0 |
| 93788_LAK cells_IL-2 | 0.0 | 0.0 |
| 93787_LAK cells_IL-2 + IL-12 | 0.0 | 1.9 |
| 93789_LAK cells_IL-2 + IFN gamma | 1.1 | 0.0 |
| 93790_LAK cells_IL-2 + IL-18 | 0.0 | 0.0 |
| 93104_LAK cells_PMA/ionomycin and IL-18 | 0.0 | 0.0 |
| 93578_NK Cells IL-2_resting | 0.0 | 0.0 |
| 93109_Mixed Lymphocyte Reaction_Two Way MLR | 0.0 | 0.0 |
| 93110_Mixed Lymphocyte Reaction_Two Way MLR | 0.0 | 0.0 |
| 93111_Mixed Lymphocyte Reaction_Two Way MLR | 0.0 | 0.0 |
| 93112_Mononuclear Cells (PBMCs)_resting | 0.0 | 0.0 |
| 93113_Mononuclear Cells (PBMCs)_PWM | 2.8 | 2.5 |
| 93114_Mononuclear Cells (PBMCs)_PHA-L | 0.0 | 3.1 |
| 93249_Ramos (B cell)_none | 0.0 | 0.0 |
| 93250_Ramos (B cell)_ionomycin | 0.0 | 0.0 |
| 93349_B lymphocytes_PWM | 0.0 | 0.0 |
| 93350_B lymphoytes_CD40L and IL-4 | 1.1 | 0.0 |
| 92665_EOL-1 (Eosinophil)_dbcAMP differentiated | 0.0 | 0.0 |
| 93248_EOL-1 (Eosinophil)_dbcAMP/PMA ionomycin | 0.0 | 0.0 |
| 93356_Dendritic Cells_none | 0.0 | 0.0 |
| 93355_Dendritic Cells_LPS 100 ng/ml | 0.0 | 0.0 |
| 93775_Dendritic Cells_anti-CD40 | 0.0 | 0.0 |
| 93774_Monocytes_resting | 0.0 | 0.0 |
| 93776_Monocytes_LPS 50 ng/ml | 0.0 | 0.0 |
| 93581_Macrophages_resting | 0.0 | 0.0 |
| 93582_Macrophages_LPS 100 ng/ml | 0.8 | 0.0 |
| 93098_HUVEC (Endothelial)_none | 0.0 | 0.0 |
| 93099_HUVEC (Endothelial)_starved | 0.0 | 0.0 |
| 93100_HUVEC (Endothelial)_IL-1b | 0.0 | 1.3 |
| 93779_HUVEC (Endothelial)_IFN gamma | 0.0 | 0.0 |
| 93102_HUVEC (Endothelial)_TNF alpha + IFN gamma | 0.0 | 0.0 |
| 93101_HUVEC (Endothelial)_TNF alpha + IL4 | 0.0 | 0.0 |
| 93781_HUVEC (Endothelial)_IL-11 | 0.9 | 0.0 |
| 93583_Lung Microvascular Endothelial Cells_none | 0.0 | 0.0 |
| 93584_Lung Microvascular Endothelial Cells_TNFa (4 ng/ml) | 0.0 | 0.0 |
| and IL1b (1 ng/ml) | | |
| 92662_Microvascular Dermal endothelium_none | 0.0 | 0.0 |
| 92663_Microsvasular Dermal endothelium_TNFa (4 ng/ml) and | 0.0 | 0.0 |
| IL1b (1 ng/ml) | | |
| 93773_Bronchial epithelium_TNFa (4 ng/ml) and IL1b (1 | 20.3 | 31.9 |
| ng/ml)** | | |
| 93347_Small Airway Epithelium_none | 1.8 | 2.0 |
| 93348_Small Airway Epithelium_TNFa (4 ng/ml) and IL1b (1 | 2.7 | 2.5 |
| ng/ml) | | |
| 92668_Coronery Artery SMC_resting | 0.0 | 0.0 |
| 92669_Coronery Artery SMC_TNFa (4 ng/ml) and IL1b (1 | 0.0 | 0.0 |
| ng/ml) | | |
| 93107_astrocytes_resting | 12.8 | 16.3 |
| 93108_astrocytes_TNFa (4 ng/ml) and IL1b (1 ng/ml) | 5.3 | 13.5 |
| 92666_KU-812 (Basophil)_resting | 0.0 | 0.0 |
| 92667_KU-812 (Basophil)_PMA/ionoycin | 0.0 | 0.0 |
| 93579_CCD1106 (Keratinocytes)_none | 1.4 | 0.0 |
| 93580_CCD1106 (Keratinocytes)_TNFa and IFNg** | 0.0 | 4.1 |
| 93791_Liver Cirrhosis | 1.9 | 2.3 |
| 93792_Lupus Kidney | 5.9 | 10.3 |
| 93577_NCI-H292 | 1.6 | 0.0 |
| 93358_NCI-H292_IL-4 | 0.7 | 0.0 |
| 93360_NC1-H292_IL-9 | 0.0 | 2.8 |
| 93359_NCI-H292_IL-13 | 0.0 | 0.0 |
| 93357_NCI-H292_IFN gamma | 0.0 | 0.0 |
| 93777_HPAEC_- | 0.0 | 0.0 |
| 93778_HPAEC_IL-1 beta/TNA alpha | 0.0 | 0.0 |
| 93254_Normal Human Lung Fibroblast_none | 0.0 | 0.0 |
| 93253_Normal Human Lung Fibroblast_TNFa (4 ng/ml) and IL- | 0.0 | 0.0 |
| 1b (1 ng/ml) | | |
| 93257_Normal Human Lung Fibroblast_IL-4 | 0.0 | 0.0 |
| 93256_Normal Human Lung Fibroblast_IL-9 | 0.0 | 0.0 |
| 93255_Normal Human Lung Fibroblast_IL-13 | 0.0 | 0.0 |
| 93258_Normal Human Lung Fibroblast_IFN gamma | 0.0 | 0.0 |
| 93106_Dermal Fibroblasts CCD1070_resting | 0.0 | 0.0 |
| 93361_Dermal Fibroblasts CCD1070_TNF alpha 4 ng/ml | 0.0 | 0.0 |
| 93105_Dermal Fibroblasts CCD1070_IL-1 beta 1 ng/ml | 0.0 | 0.0 |
| 93772_dermal fibroblast_IFN gamma | 0.0 | 0.0 |
| 93771_dermal fibroblast_IL-4 | 0.0 | 1.5 |
| 93259_IBD Colitis 1** | 1.5 | 2.8 |
| 93260_IBD Colitis 2 | 0.0 | 1.2 |
| 93261_IBD Crohns | 1.4 | 4.0 |
| 735010_Colon_normal | 3.4 | 2.7 |
| 735019_Lung_none | 52.1 | 42.3 |
| 64028-1_Thymus_none | 100.0 | 100.0 |
| 64030-1_Kidney_none | 1.6 | 0.0 |

Panel 32. Panel CNSD.01

| | Relative | | Relative |
|---|---|---|---|
| | Expression(%) | | Expression(%) |
| | cns1x4tm6177f_{—} | | cns1x4tm6177f_{—} |
| Tissue Name | ag1216_b2 | Tissue Name | ag1216_b2 |
| 102633_BA4 Control | 18.7 | 102605_BA17 PSP | 38.3 |
| 102641_BA4 Control2 | 62.0 | 102612_BA17 PSP2 | 19.2 |
| 102625_BA4 Alzheimer's2 | 17.2 | 102637_Sub Nigra Control | 6.8 |
| 102649_BA4 Parkinson's | 79.4 | 102645_Sub Nigra Control2 | 12.2 |
| 102656_BA4 Parkinson's2 | 54.7 | 102629_Sub Nigra | 10.8 |
| | | Alzheimer's2 | |
| 102664_BA4 Huntington's | 37.5 | 102660_Sub Nigra Parkinson's2 | 17.4 |
| 102671_BA4 Huntington's2 | 17.0 | 102667_Sub Nigra | 21.1 |
| | | Huntington's | |
| 102603_BA4 PSP | 16.5 | 102674_Sub Nigra | 8.6 |
| | | Huntington's2 | |
| 102610_BA4 PSP2 | 22.9 | 102614_Sub Nigra PSP2 | 1.9 |
| 102588_BA4 Depression | 45.0 | 102592_Sub Nigra Depression | 1.9 |
| 102596_BA4 Depression2 | 27.0 | 102599_Sub Nigra Depression2 | 3.6 |
| 102634_BA7 Control | 45.2 | 102636_Glob Palladus Control | 21.2 |
| 102642_BA7 Control2 | 25.5 | 102644_Glob Palladus Control2 | 12.8 |
| 102626_BA7 Alzheimer's2 | 20.0 | 102620_Glob Palladus | 3.0 |
| | | Alzheimer's | |
| 102650_BA7 Parkinson's | 48.1 | 102628_Glob Palladus | 4.2 |
| | | Alzheimer's2 | |
| 102657_BA7 Parkinson's2 | 27.3 | 102652_Glob Palladus | 100.0 |
| | | Parkinson's | |
| 102665_BA7 Huntington's | 58.5 | 102659_Glob Palladus | 10.6 |
| | | Parkinson's2 | |
| 102672_BA7 Huntington's2 | 65.1 | 102606_Glob Palladus PSP | 4.3 |
| 102604_BA7 PSP | 93.8 | 102613_Glob Palladus PSP2 | 6.8 |
| 102611_BA7 PSP2 | 34.8 | 102591_Glob Palladus | 9.1 |
| | | Depression | |
| 102589_BA7 Depression | 20.9 | 102638_Temp Pole Control | 29.7 |
| 102632_BA9 Control | 35.4 | 102646_Temp Pole Control2 | 63.9 |
| 102640_BA9 Control | 58.7 | 102622_Temp Pole Alzheimer's | 7.3 |
| 102617_BA9 Alzheimer's | 9.2 | 102630_Temp Pole | 16.4 |
| | | Alzheimer's2 | |
| 102624_BA9 Alzheimer's2 | 36.1 | 102653_Temp Pole Parkinson's | 88.4 |
| 102648_BA9 Parkinson's | 33.0 | 102661_Temp Pole | 50.2 |
| | | Parkinson's2 | |
| 102655_BA9 Parkinson's2 | 32.2 | 102668_Temp Pole | 63.5 |
| | | Huntington's | |
| 102663_BA9 Huntington's | 50.4 | 102607_Temp Pole PSP | 22.1 |
| 102670_BA9 Huntington's2 | 18.6 | 102615_Temp Pole PSP2 | 11.4 |
| 102602_BA9 PSP | 14.3 | 102600_Temp Pole | 36.9 |
| | | Depression2 | |
| 102609_BA9 PSP2 | 7.6 | 102639_Cing Gyr Control | 60.1 |
| 102587_BA9 Depression | 14.9 | 102647_Cing Gyr Control2 | 48.0 |
| 102595_BA9 Depression2 | 18.3 | 102623_Cing Gyr Alzheimer's | 19.4 |
| 102635_BA17 Control | 87.9 | 102631_Cing Gyr Alzheimer's2 | 24.8 |
| 102643_BA17 Control2 | 73.0 | 102654_Cing Gyr Parkinson's | 29.7 |
| 102627_BA17 Alzheimer's2 | 36.7 | 102662_Cing Gyr Parkinson's2 | 33.2 |
| 102651_BA17 Parkinson's | 81.0 | 102669_Cing Gyr Huntington's | 36.0 |
| 102658_BA17 Parkinson's2 | 95.4 | 102676_Cing Gyr | 27.7 |
| | | Huntington's2 | |
| 102666_BA17 Huntington's | 78.7 | 102608_Cing Gyr PSP | 20.7 |
| 102673_BA17 Huntington's2 | 37.6 | 102616_Cing Gyr PSP2 | 3.9 |
| 102590_BA17 Depression | 33.7 | 102594_Cing Gyr Depression | 34.6 |
| 102597_BA17 Depression2 | 73.7 | 102601_Cing Gyr Depression2 | 21.0 |

Panel 1.2 Summary: Ag1216 The MOL4a gene is well expressed in a variety of normal tissues including kidney, heart, brain, thymus and lung. Of interest is the robust expression in activated endothelial cells, which may indicate that this gene is important for angiogenesis or lymphocyte trafficking. Inflammatory lymphocytes preferentially traffic into tissues by crossing activated endothelium. Expression of the MOL4a gene appears to be up regulated in renal cell carcinomas. In contrast, expression of the MOL4a gene is down regulated in a number of cancer cell lines (including pancreatic, CNS, breast, and lung) relative to the normal controls. No expression of this gene is detected in a variety of melanoma cell lines. Therefore, modulation of MOL4a gene function may provide an effective treatment for a variety of cancers.

Panel 2.2 Summary: Ag1216 Expression of the MOL4a gene appears to be associated with kidney cancers. This is in good agreement with the data obtained in Panel 1.2 and suggests that therapeutic modulation of this gene using inhibitory monoclonal antibodies or small molecules may prove useful in the treatment of kidney cancers. In addition, the MOL4a gene may be a useful marker for the detection of renal cell carcinomas.

Panel 4D Summary: Ag1216 Two replicate experiments using the same probe and primer set were in good agreement. The MOL4a transcript is highly expressed in thymus. To a much lesser degree, the transcript is also expressed in the lung as well as in small airway epithelium treated with TNF-a and IL-1b. Therefore, protein therapeutics designed against the protein encoded for by this transcript could reduce inflammation in asthma or other lung disease such as emphysema.

Panel CNSD.01 Summary: Ag1216 Semaphorins can act as axon guidance proteins, specifically through their ability to act as chemorepellents that inhibit CNS regenerative capacity. Manipulation of levels of the MOL4a semaphorin-like protein may therefore be of use in inducing a compensatory synaptogenic response to neuronal death in Alzheimer's disease, Parkinson's disease, Huntington's disease, spinocerebellar ataxia, progressive supranuclear palsy, multiple sclerosis, ALS, head trauma, stroke, or any other disease/condition associated with neuronal loss. cl MOL4b

TABLE A
Ag1216

| | | | | | |
|---|---|---|---|---|---|
| | | | | Start | SEQ ID |
| Primers | Sequences | TM | Length | Position | NO: |

| | | | | | |
|---|---|---|---|---|---|
| Forward 5′-CCCGAAGAATGAAAAGTACACA-3′ | 59.1 | 22 | 5263 | 126 | |
| Probe | FAM-5′-CCCATGGAATTCAAGACCCTGAACAA-3′-TAMRA | 69.7 | 26 | 5285 | 127 |
| Reverse | 5′-AATGGGTAGAAGTTGGCTCTGT-3′ | 59.2 | 22 | 5331 | 128 |

TABLE AA
Panel 1.2

| | Rel. Expr., % |
|---|---|
| Tissue Name | 1.2tm1404f_ag1216 |
| Endothelial cells | 0 |
| Endothelial cells (treated) | 76.8 |
| Pancreas | 11.3 |
| Pancreatic ca. CAPAN 2 | 0 |
| Adrenal Gland (new lot*) | 12.2 |
| Thyroid | 11.6 |
| Salavary gland | 2.7 |
| Pituitary gland | 12.6 |
| Brain (fetal) | 77.9 |
| Brain (whole) | 73.2 |
| Brain (amygdala) | 33.7 |
| Brain (cerebellum) | 6.8 |
| Brain (hippocampus) | 75.3 |
| Brain (thalamus) | 9.9 |
| Cerebral Cortex | 87.7 |
| Spinal cord | 8.3 |
| CNS ca. (glio/astro) U87-MG | 0 |
| CNS ca. (glio/astro) U-118-MG | 1.5 |
| CNS ca. (astro) SW1783 | 0 |
| CNS ca.* (neuro; met) SK-N-AS | 11.1 |
| CNS ca. (astro) SF-539 | 0 |
| CNS ca. (astro) SNB-75 | 6.4 |
| CNS ca. (glio) SNB-19 | 4.4 |
| CNS ca. (glio) U251 | 4.2 |
| CNS ca. (glio) SF-295 | 0 |
| Heart | 61.1 |
| Skeletal Muscle (new lot*) | 8.2 |
| Bone marrow | 0.1 |
| Thymus | 0.2 |
| Spleen | 0 |
| Lymph node | 5.1 |
| Colorectal | 0.2 |
| Stomach | 5.9 |
| Small intestine | 11.8 |
| Colon ca. SW480 | 0 |
| Colon ca.* (SW480 met)SW620 | 2.7 |
| Colon ca. HT29 | 0 |
| Colon ca. HCT-116 | 0 |
| Colon ca. CaCo-2 | 0.5 |
| 83219 CC Well to Mod Diff (ODO3866) | 0.5 |
| Colon ca. HCC-2998 | 0 |
| Gastric ca.* (liver met) NCI-N87 | 0 |
| Bladder | 6.7 |
| Trachea | 3.1 |
| Kidney | 32.3 |
| Kidney (fetal) | 51.8 |
| Renal ca. 786-0 | 100 |
| Renal ca. A498 | 3 |
| Renal ca. RXF 393 | 45.4 |
| Renal ca. ACHN | 0.2 |
| Renal ca. UO-31 | 0 |
| Renal ca. TK-10 | 0 |
| Liver | 7.5 |
| Liver (fetal) | 7.7 |
| Liver ca. (hepatoblast) HepG2 | 0 |
| Lung | 6.1 |
| Lung (fetal) | 13.1 |
| Lung ca. (small cell) LX-1 | 0 |
| Lung ca. (small cell) NCI-H69 | 0 |
| Lung ca. (s. cell var.) SHP-77 | 0 |
| Lung ca. (large cell)NCI-H460 | 0 |
| Lung ca. (non-sm. cell) A549 | 0 |
| Lung ca. (non-s. cell) NCI-H23 | 0 |
| Lung ca (non-s. cell) HOP-62 | 0 |
| Lung ca. (non-s. cl) NCI-H522 | 0 |
| Lung ca. (squam.) SW 900 | 0 |
| Lung ca. (squam.) NCI-H596 | 0 |
| Mammary gland | 13 |
| Breast ca.* (pl. effusion) MCF-7 | 0 |
| Breast ca.* (pl. ef) MDA-MB-231 | 0 |
| Breast ca.* (pl. effusion) T47D | 0 |
| Breast ca. BT-549 | 1.8 |
| Breast ca. MDA-N | 0 |
| Ovary | 10.2 |
| Ovarian ca. OVCAR-3 | 66.4 |
| Ovarian ca. OVCAR-4 | 1.3 |
| Ovarian ca. OVCAR-5 | 3.5 |
| Ovarian ca. OVCAR-8 | 0 |
| Ovarian ca. IGROV-1 | 0.3 |
| Ovarian ca.* (ascites) SK-OV-3 | 0 |
| Uterus | 7.7 |
| Placenta | 8.1 |
| Prostate | 4.6 |
| Prostate ca.* (bone met)PC-3 | 0 |
| Testis | 4.6 |
| Melanoma Hs688(A).T | 0 |
| Melanoma* (met) Hs688(B).T | 0 |
| Melanoma UACC-62 | 0 |
| Melanoma M14 | 0 |
| Melanoma LOX IMVI | 0 |
| Melanoma* (met) SK-MEL-5 | 0 |
| Adipose | 1.5 |

TABLE AB
Panel 2.2

| | Rel. Expr., % |
|---|---|
| Tissue Name | 2.2x4tm6515f_ag1216_b1 |
| Normal Colon GENPAK 061003 | 0.1 |
| 97759 Colon cancer (OD06064) | 0 |
| 97760 Colon cancer NAT (OD06064) | 0.4 |
| 97778 Colon cancer (OD06159) | 0 |
| 97779 Colon cancer NAT (OD06159) | 0.8 |
| 98861 Colon cancer (OD06297-04) | 0 |
| 98862 Colon cancer NAT (OD06297-015) | 0.6 |
| 83237 CC Gr.2 ascend colon (ODO3921) | 0.2 |
| 83238 CC NAT (ODO3921) | 0 |
| 97766 Colon cancer metastasis (OD06104) | 0.2 |
| 97767 Lung NAT (OD06104) | 1.1 |
| 87472 Colon mets to lung (OD04451-01) | 0.3 |
| 87473 Lung NAT (OD04451-02) | 0.4 |
| Normal Prostate Clontech A+ 6546-1 (8090438) | 0.5 |
| 84140 Prostate Cancer (OD04410) | 0.2 |
| 84141 Prostate NAT (OD04410) | 0 |
| Normal Ovary Res. Gen. | 2.3 |
| 98863 Ovarian cancer (OD06283-03) | 4.6 |
| 98865 Ovarian cancer NAT/fallopian tube (OD06283-07) | 1.3 |
| Ovarian Cancer GENPAK 064008 | 9.7 |
| 97773 Ovarian cancer (OD06145) | 0 |
| 97775 Ovarian cancer NAT (OD06145) | 0 |
| 98853 Ovarian cancer (OD06455-03) | 0.2 |
| 98854 Ovarian NAT (OD06455-07) Fallopian tube | 1.9 |
| Normal Lung GENPAK 061010 | 0.6 |
| 92337 Invasive poor diff. lung adeno (ODO4945-01 | 0.4 |
| 92338 Lung NAT (ODO4945-03) | 0.2 |
| 84136 Lung Malignant Cancer (OD03126) | 0 |
| 84137 Lung NAT (OD03126) | 0.4 |
| 90372 Lung Cancer (OD05014A) | 1 |
| 90373 Lung NAT (OD05014B) | 2.1 |
| 97761 Lung cancer (OD06081) | 0.8 |
| 97762 Lung cancer NAT (OD06081) | 0.4 |
| 85950 Lung Cancer (OD04237-01) | 0.4 |
| 85970 Lung NAT (OD04237-02) | 0 |
| 83255 Ocular Mel Met to Liver (ODO4310) | 0.2 |
| 83256 Liver NAT (ODO4310) | 0 |
| 84139 Melanoma Mets to Lung (OD04321) | 0.7 |
| 84138 Lung NAT(OD04321) | 0 |
| Normal Kidney GENPAK 061008 | 1.7 |
| 83786 Kidney Ca, Nuclear grade 2 (OD04338) | 5.3 |
| 83787 Kidney NAT (OD04338) | 6.2 |
| 83788 Kidney Ca Nuclear grade ½ (OD04339) | 100 |
| 83789 Kidney NAT (OD04339) | 3 |
| 83790 Kidney Ca, Clear cell type (OD04340) | 26.3 |
| 83791 Kidney NAT (OD04340) | 0.9 |
| 83792 Kidney Ca, Nuclear grade 3 (OD04348) | 5.1 |
| 83793 Kidney NAT (OD04348) | 11.6 |
| 98938 Kidney malignant cancer (OD06204B) | 0 |
| 98939 Kidney normal adjacent tissue (OD06204E) | 1.4 |
| 85973 Kidney Cancer (OD04450-01) | 1.3 |
| 85974 Kidney NAT (OD04450-03) | 3.2 |
| Kidney Cancer Clontech 8120613 | 0 |
| Kidney NAT Clontech 8120614 | 3.5 |
| Kidney Cancer Clontech 9010320 | 3.5 |
| Kidney NAT Clontech 9010321 | 0.9 |
| Kidney Cancer Clontech 8120607 | 8.3 |
| Kidney NAT Clontech 8120608 | 0.7 |
| Normal Uterus GENPAK 061018 | 2.9 |
| Uterus Cancer GENPAK 064011 | 0.2 |
| Normal Thyroid Clontech A+ 6570-1 (7080817) | 0.4 |
| Thyroid Cancer GENPAK 064010 | 0.2 |
| Thyroid Cancer INVITROGEN A302152 | 0.3 |
| Thyroid NAT INVITROGEN A302153 | 0.3 |
| Normal Breast GENPAK 061019 | 1.3 |
| 84877 Breast Cancer (OD04566) | 0.4 |
| Breast Cancer Res. Gen. 1024 | 0.4 |
| 85975 Breast Cancer (OD04590-01) | 1.6 |
| 85976 Breast Cancer Mets (OD04590-03) | 0.8 |
| 87070 Breast Cancer Metastasis (OD04655-05) | 0.6 |
| GENPAK Breast Cancer 064006 | 0.9 |
| Breast Cancer Clontech 9100266 | 1.4 |
| Breast NAT Clontech 9100265 | 0.6 |
| Breast Cancer INVITROGEN A209073 | 0 |
| Breast NAT INVITROGEN A2090734 | 1.4 |
| 97763 Breast cancer (OD06083) | 0.8 |
| 97764 Breast cancer node metastasis (OD06083) | 0.9 |
| Normal Liver GENPAK 061009 | 0.3 |
| Liver Cancer Research Genetics RNA 1026 | 0.7 |
| Liver Cancer Research Genetics RNA 1025 | 1.6 |
| Paired Liver Cancer Tissue Research Genetics RNA 6004-T | 0 |
| Paired Liver Tissue Research Genetics RNA 6004-N | 0 |
| Paired Liver Cancer Tissue Research Genetics RNA 6005-T | 1.3 |
| Paired Liver Tissue Research Genetics RNA 6005-N | 0.4 |
| Liver Cancer GENPAK 064003 | 0.5 |
| Normal Bladder GENPAK 061001 | 0.8 |
| Bladder Cancer Research Genetics RNA 1023 | 0.7 |
| Bladder Cancer INVITROGEN A302173 | 0.2 |
| Normal Stomach GENPAK 061017 | 1 |
| Gastric Cancer Clontech 9060397 | 0.2 |
| NAT Stomach Clontech 9060396 | 0.4 |
| Gastric Cancer Clontech 9060395 | 0.4 |
| NAT Stomach Clontech 9060394 | 0.7 |
| Gastric Cancer GENPAK 064005 | 1.1 |

TABLE AC
Panel 4D

| | Rel. Expr., % | Rel. Expr., % |
|---|---|---|
| | 4Dtm2072f_ag1 | 4Dtm2246f_ag |
| Tissue Name | 216 | 1216 |
| 93768_Secondary Th1_anti-CD28/anti-CD3 | 0 | 0 |
| 93769_Secondary Th2_anti-CD28/anti-CD3 | 0 | 0 |
| 93770_Secondary Tr1_anti-CD28/anti-CD3 | 0 | 0 |
| 93573_Secondary Th1_resting day 4-6 in IL-2 | 0 | 0 |
| 93572_Secondary Th2_resting day 4-6 in IL-2 | 0 | 0 |
| 93571_Secondary Tr1_resting day 4-6 in IL-2 | 0 | 0 |
| 93568_primary Th1_anti-CD28/anti-CD3 | 0 | 0 |
| 93569_primary Th2_anti-CD28/anti-CD3 | 0 | 0 |
| 93570_primary Tr1_anti-CD28/anti-CD3 | 0 | 0 |
| 93565_primary Th1_resting dy 4-6 in IL-2 | 0 | 0 |
| 93566_primary Th2_resting dy 4-6 in IL-2 | 1.1 | 0 |
| 93567_primary Tr1_resting dy 4-6 in IL-2 | 0 | 0.9 |
| 93351_CD45RA CD4 lymphocyte_anti-CD28/anti-CD3 | 0 | 0 |
| 93352_CD45RO CD4 lymphocyte_anti-CD28/anti-CD3 | 0.8 | 0 |
| 93251_CD8 Lymphocytes_anti-CD28/anti-CD3 | 0 | 0 |
| 93353_chronic CD8 Lymphocytes 2ry_resting dy 4-6 in IL-2 | 0 | 1.3 |
| 93574_chronic CD8 Lymphocytes 2ry_activated CD3/CD28 | 0 | 0 |
| 93354_CD4_none | 0 | 0.9 |
| 93252_Secondary Th1/Th2/Tr1_anti-CD95 CH11 | 0 | 0 |
| 93103_LAK cells_resting | 0 | 0 |
| 93788_LAK cells_IL-2 | 0 | 0 |
| 93787_LAK cells_IL-2 + IL-12 | 0 | 1.9 |
| 93789_LAK cells_IL-2 + IFN gamma | 1.1 | 0 |
| 93790_LAK cells_IL-2 + IL-18 | 0 | 0 |
| 93104_LAK cells_PMA/ionomycin and IL-18 | 0 | 0 |
| 93578_NK Cells IL-2_resting | 0 | 0 |
| 93109_Mixed Lymphocyte Reaction_Two Way MLR | 0 | 0 |
| 93110_Mixed Lymphocyte Reaction_Two Way MLR | 0 | 0 |
| 93111_Mixed Lymphocyte Reaction_Two Way MLR | 0 | 0 |
| 93112_Mononuclear Cells (PBMCs)_resting | 0 | 0 |
| 93113_Mononuclear Cells (PBMCs)_PWM | 2.8 | 2.5 |
| 93114_Mononuclear Cells (PBMCs)_PHA-L | 0 | 3.1 |
| 93249_Ramos (B cell)_none | 0 | 0 |
| 93250_Ramos (B cell)_ionomycin | 0 | 0 |
| 93349_B lymphocytes_PWM | 0 | 0 |
| 93350_B lymphoytes_CD40L and IL-4 | 1.1 | 0 |
| 92665_EOL-1 (Eosinophil)_dbcAMP differentiated | 0 | 0 |
| 93248_EOL-1 (Eosinophil)_dbcAMP/PMAionomycin | 0 | 0 |
| 93356_Dendritic Cells_none | 0 | 0 |
| 93355_Dendritic Cells_LPS 100 ng/ml | 0 | 0 |
| 93775_Dendritic Cells_anti-CD40 | 0 | 0 |
| 93774_Monocytes_resting | 0 | 0 |
| 93776_Monocytes_LPS 50 ng/ml | 0 | 0 |
| 93581_Macrophages_resting | 0 | 0 |
| 93582_Macrophages_LPS 100 ng/ml | 0.8 | 0 |
| 93098_HUVEC (Endothelial)_none | 0 | 0 |
| 93099_HUVEC (Endothelial)_starved | 0 | 0 |
| 93100_HUVEC (Endothelial)_IL-1b | 0 | 1.3 |
| 93779_HUVEC (Endothelial)_IFN gamma | 0 | 0 |
| 93102_HUVEC (Endothelial)_TNF alpha + IFN gamma | 0 | 0 |
| 93101_HUVEC (Endothelial)_TNF alpha + IL4 | 0 | 0 |
| 93781_HUVEC (Endothelial)_IL-11 | 0.9 | 0 |
| 93583_Lung Microvascular Endothelial Cells_none | 0 | 0 |
| 93584_Lung Microvascular Endothelial Cells_TNFa (4 ng/ml) | 0 | 0 |
| and IL1b (1 ng/ml) | | |
| 92662_Microvascular Dermal endothelium_none | 0 | 0 |
| 92663_Microsvasular Dermal endothelium_TNFa (4 ng/ml) and | 0 | 0 |
| IL1b (1 ng/ml) | | |
| 93773_Bronchial epithelium_TNFa (4 ng/ml) and IL1b (1 | 20.3 | 31.9 |
| ng/ml)** | | |
| 93347_Small Airway Epithelium_none | 1.8 | 2 |
| 93348_Small Airway Epithelium_TNFa (4 ng/ml) and IL1b (1 | 2.7 | 2.5 |
| ng/ml) | | |
| 92668_Coronery Artery SMC_resting | 0 | 0 |
| 92669_Coronery Artery SMC_TNFa (4 ng/ml) and IL1b (1 | 0 | 0 |
| ng/ml) | | |
| 93107_astrocytes_resting | 12.8 | 16.3 |
| 93108_astrocytes_TNFa (4 ng/ml) and IL1b (1 ng/ml) | 5.3 | 13.5 |
| 92666_KU-812 (Basophil)_resting | 0 | 0 |
| 92667_KU-812 (Basophil)_PMA/ionoycin | 0 | 0 |
| 93579_CCD1106 (Keratinocytes)_none | 1.4 | 0 |
| 93580_CCD1106 (Keratinocytes)_TNFa and IFNg** | 0 | 4.1 |
| 93791_Liver Cirrhosis | 1.9 | 2.3 |
| 93792_Lupus Kidney | 5.9 | 10.3 |
| 93577_NCI-H292 | 1.6 | 0 |
| 93358_NCI-H292_IL-4 | 0.7 | 0 |
| 93360_NCI-H292_IL-9 | 0 | 2.8 |
| 93359_NCI-H292_IL-13 | 0 | 0 |
| 93357_NCI-H292_IFN gamma | 0 | 0 |
| 93777_HPAEC_- | 0 | 0 |
| 93778_HPAEC_IL-1 beta/TNA alpha | 0 | 0 |
| 93254_Normal Human Lung Fibroblast_none | 0 | 0 |
| 93253_Normal Human Lung Fibroblast_TNFa (4 ng/ml) and IL- | 0 | 0 |
| 1b (1 ng/ml) | | |
| 93257_Normal Human Lung Fibroblast_IL-4 | 0 | 0 |
| 93256_Normal Human Lung Fibroblast_IL-9 | 0 | 0 |
| 93255_Normal Human Lung Fibroblast_IL-13 | 0 | 0 |
| 93258_Normal Human Lung Fibroblast_IFN gamma | 0 | 0 |
| 93106_Dermal Fibroblasts CCD1070_resting | 0 | 0 |
| 93361_Dermal Fibroblasts CCD1070_TNF alpha 4 ng/ml | 0 | 0 |
| 93105_Dermal Fibroblasts CCD1070_IL-1 beta 1 ng/ml | 0 | 0 |
| 93772_dermal fibroblast_IFN gamma | 0 | 0 |
| 93771_dermal fibroblast_IL-4 | 0 | 1.5 |
| 93259_IBD Colitis 1** | 1.5 | 2.8 |
| 93260_IBD Colitis 2 | 0 | 1.2 |
| 93261_IBD Crohns | 1.4 | 4 |
| 735010_Colon_normal | 3.4 | 2.7 |
| 735019_Lung_none | 52.1 | 42.3 |
| 64028-1_Thymus_none | 100 | 100 |
| 64030-1_Kidney_none | 1.6 | 0 |

Potential Role(s) of MOL4b in Tumorgenesis: Semaphorins are cell surface receptors involved in axon guidance molecules with chemorepulsive activity, and are suggested to play a major role in navigating axonal networks throughout development into their correct destinations. They have been found to act as receptor for neurophilin both in neuronal and non-neuronal cell, specifically endothelial cells. Panel 1 indicate that MOL4b is induced in activated endothelial cells and it is expressed by tumor cell derived from Kidney and ovarian tumors. It therefore likely that in thecse cell types, MOL4b expression contributes to migration and survival activities

Impact of therapeutic targeting of MOL4b: Therapeutic targeting with a human monoclonal antibody of MOL4b might block the migration of cancer cells, and/or supporting stromal elements, specifically endothelial cells, and promote cell death rather than cell survival especially in those cancer types, like kidney and ovarian tumors where the gene is overexpressed in the tumor compared to the normal adjacent tissue cl E. MOL5a

Expression of gene MOL5a was assessed using the primer-probe sets Ag1215 and Ag1382 (identical sequences), described in Tables 33 and 34. Results of the RTQ-PCR runs are shown in Tables 35, 36, and 37.

TABLE 33
Probe Name Ag1215/Ag1382

| | | | | Start | SEQ ID |
|---|---|---|---|---|---|
| Primers | Sequences | TM | Length | Position | NO: |
| Forward | 5′-AACCCATTATCCTGCGTAACAT-3′ | 59.6 | 22 | 619 | 80 |
| Probe | FAM-5′-CCCCACCACTCCATGAAGACAGAGTA-3′-TAMRA | 68.5 | 26 | 645 | 81 |
| Reverse | 5′-CCTACAAAGTGAGGTTCGTTGA-3′ | 59.3 | 22 | 685 | 82 |

TABLE 34
Panel 1.2

| | Relative | Relative |
|---|---|---|
| | Expression | Expression |
| | (%) | (%) |
| | 1.2tm1403f_{—} | 1.2tm1581t_{—} |
| Tissue Name | ag1215 | ag1382 |
| Endothelial cells | 21.9 | 17.3 |
| Endothelial cells (treated) | 100.0 | 40.9 |
| Pancreas | 14.7 | 0.4 |
| Pancreatic ca. CAPAN 2 | 3.4 | 0.7 |
| Adrenal Gland (new lot*) | 26.4 | 10.4 |
| Thyroid | 18.8 | 0.3 |
| Salivary gland | 21.5 | 5.8 |
| Pituitary gland | 23.8 | 1.4 |
| Brain (fetal) | 33.9 | 1.4 |
| Brain (whole) | 69.3 | 3.8 |
| Brain (amygdala) | 13.9 | 3.6 |
| Brain (cerebellum) | 67.4 | 3.0 |
| Brain (hippocampus) | 45.1 | 12.6 |
| Brain (thalamus) | 19.1 | 26.4 |
| Cerebral Cortex | 53.2 | 30.1 |
| Spinal cord | 45.4 | 7.2 |
| CNS ca. (glio/astro) U87-MG | 7.8 | 21.3 |
| CNS ca. (glio/astro) U-118-MG | 6.8 | 13.5 |
| CNS ca. (astro) SW1783 | 2.0 | 1.8 |
| CNS ca.* (neuro; met) SK-N-AS | 50.3 | 27.5 |
| CNS ca. (astro) SF-539 | 20.3 | 21.2 |
| CNS ca. (astro) SNB-75 | 13.7 | 2.8 |
| CNS ca. (glio) SNB-19 | 19.5 | 28.9 |
| CNS ca. (glio) U251 | 15.2 | 0.0 |
| CNS ca. (glio) SF-295 | 15.0 | 5.1 |
| Heart | 63.3 | 12.2 |
| Skeletal Muscle (new lot*) | 27.7 | 0.8 |
| Bone marrow | 2.9 | 0.9 |
| Thymus | 4.3 | 2.8 |
| Spleen | 33.4 | 15.6 |
| Lymph node | 23.7 | 5.4 |
| Colorectal | 8.4 | 2.4 |
| Stomach | 36.3 | 9.6 |
| Small intestine | 27.9 | 11.7 |
| Colon ca. SW480 | 6.1 | 1.8 |
| Colon ca.* (SW480 met)SW620 | 10.4 | 40.3 |
| Colon ca. HT29 | 3.0 | 2.5 |
| Colon ca. HCT-116 | 6.9 | 11.0 |
| Colon ca. CaCo-2 | 13.1 | 28.9 |
| 83219 CC Well to Mod Diff (ODO3866) | 3.1 | 1.8 |
| Colon ca. HCC-2998 | 12.6 | 9.5 |
| Gastric ca.* (liver met) NCI-N87 | 18.4 | 17.6 |
| Bladder | 40.1 | 15.5 |
| Trachea | 13.8 | 6.0 |
| Kidney | 19.5 | 42.9 |
| Kidney (fetal) | 30.4 | 61.6 |
| Renal ca. 786-0 | 6.1 | 6.7 |
| Renal ca. A498 | 13.0 | 9.3 |
| Renal ca. RXF 393 | 8.4 | 6.0 |
| Renal ca. ACHN | 10.7 | 4.9 |
| Renal ca. UO-31 | 8.8 | 3.6 |
| Renal ca. TK-10 | 15.4 | 6.2 |
| Liver | 15.2 | 3.2 |
| Liver (fetal) | 13.3 | 5.9 |
| Liver ca. (hepatoblast) HepG2 | 21.8 | 12.1 |
| Lung | 19.3 | 0.7 |
| Lung (fetal) | 28.1 | 4.5 |
| Lung ca. (small cell) LX-1 | 24.5 | 24.0 |
| Lung ca. (small cell) NCI-H69 | 8.1 | 12.7 |
| Lung ca. (s. cell var.) SHP-77 | 3.8 | 1.8 |
| Lung ca. (large cell)NCI-H460 | 40.3 | 19.5 |
| Lung ca. (non-sm. cell) A549 | 13.1 | 13.9 |
| Lung ca. (non-s. cell) NCI-H23 | 28.1 | 24.5 |
| Lung ca (non-s. cell) HOP-62 | 42.3 | 9.7 |
| Lung ca. (non-s. cl) NCI-H522 | 90.1 | 44.1 |
| Lung ca. (squam.) SW 900 | 37.4 | 57.8 |
| Lung ca. (squam.) NCI-H596 | 9.8 | 4.2 |
| Mammary gland | 42.6 | 9.0 |
| Breast ca.* (pl. effusion) MCF-7 | 85.3 | 56.6 |
| Breast ca.* (pl. ef) MDA-MB-231 | 5.3 | 1.4 |
| Breast ca.* (pl. effusion) T47D | 5.6 | 16.6 |
| Breast ca. BT-549 | 5.0 | 2.6 |
| Breast ca. MDA-N | 16.7 | 3.7 |
| Ovary | 49.0 | 23.0 |
| Ovarian ca. OVCAR-3 | 46.3 | 50.7 |
| Ovarian ca. OVCAR-4 | 11.3 | 10.6 |
| Ovarian ca. OVCAR-5 | 28.5 | 66.9 |
| Ovarian ca. OVCAR-8 | 19.8 | 100.0 |
| Ovarian ca. IGROV-1 | 18.2 | 13.3 |
| Ovarian ca.* (ascites) SK-OV-3 | 25.7 | 10.3 |
| Uterus | 24.5 | 3.5 |
| Placenta | 90.1 | 82.9 |
| Prostate | 28.5 | 15.0 |
| Prostate ca.* (bone met)PC-3 | 39.0 | 15.4 |
| Testis | 8.4 | 0.6 |
| Melanoma Hs688(A).T | 3.8 | 1.2 |
| Melanoma* (met) Hs688(B).T | 2.9 | 0.8 |
| Melanoma UACC-62 | 23.2 | 11.4 |
| Melanoma M14 | 13.7 | 6.7 |
| Melanoma LOX IMVI | 9.6 | 1.8 |
| Melanoma* (met) SK-MEL-5 | 27.4 | 8.9 |
| Adipose | 6.2 | 12.8 |

TABLE 35
Panel 2.2

| | Relative | | Relative |
|---|---|---|---|
| | Expression(%) | | Expression(%) |
| | 2.2x4tm6515f_{—} | | 2.2x4tm6515f_{—} |
| Tissue Name | ag1215_a2 | Tissue Name | ag1215_a2 |
| Normal Colon GENPAK | 31.4 | 83793 Kidney NAT (OD04348) | 39.7 |
| 061003 | | | |
| 97759 Colon cancer (OD06064) | 34.3 | 98938 Kidney malignant cancer | 15.4 |
| | | (OD06204B) | |
| 97760 Colon cancer NAT | 18.6 | 98939 Kidney normal adjacent | 10.4 |
| (OD06064) | | tissue (OD06204E) | |
| 97778 Colon cancer (OD06159) | 2.7 | 85973 Kidney Cancer | 15.3 |
| | | (OD04450-01) | |
| 97779 Colon cancer NAT | 22.5 | 85974 Kidney NAT (OD04450- | 20.3 |
| (OD06159) | | 03) | |
| 98861 Colon cancer (OD06297- | 3.2 | Kidney Cancer Clontech | 1.4 |
| 04) | | 8120613 | |
| 98862 Colon cancer NAT | 30.6 | Kidney NAT Clontech 8120614 | 20.3 |
| (OD06297-015) | | | |
| 83237 CC Gr.2 ascend colon | 11.6 | Kidney Cancer Clontech | 10.5 |
| (ODO3921) | | 9010320 | |
| 83238 CC NAT (ODO3921) | 8.4 | Kidney NAT Clontech 9010321 | 9.0 |
| 97766 Colon cancer metastasis | 5.1 | Kidney Cancer Clontech | 32.2 |
| (OD06104) | | 8120607 | |
| 97767 Lung NAT (OD06104) | 8.3 | Kidney NAT Clontech 8120608 | 12.1 |
| 87472 Colon mets to lung | 15.3 | Normal Uterus GENPAK | 31.8 |
| (OD04451-01) | | 061018 | |
| 87473 Lung NAT (OD04451- | 4.3 | Uterus Cancer GENPAK | 31.4 |
| 02) | | 064011 | |
| Normal Prostate Clontech A+ | 11.2 | Normal Thyroid Clontech A+ | 3.0 |
| 6546-1 (8090438) | | 6570-1 (7080817) | |
| 84140 Prostate Cancer | 10.0 | Thyroid Cancer GENPAK | 14.8 |
| (OD04410) | | 064010 | |
| 84141 Prostate NAT | 14.3 | Thyroid Cancer INVITROGEN | 37.7 |
| (OD04410) | | A302152 | |
| Normal Ovary Res. Gen. | 74.7 | Thyroid NAT INVITROGEN | 7.0 |
| | | A302153 | |
| 98863 Ovarian cancer | 27.6 | Normal Breast GENPAK | 35.4 |
| (OD06283-03) | | 061019 | |
| 98865 Ovarian cancer | 6.4 | 84877 Breast Cancel | 15.6 |
| NAT/fallopian tube (OD06283- | | (OD04566) | |
| 07) | | | |
| Ovarian Cancer GENPAK | 16.2 | Breast Cancer Res. Gen. 1024 | 51.4 |
| 064008 | | | |
| 97773 Ovarian cancer | 8.8 | 85975 Breast Cancer | 3.68 |
| (OD06145) | | (OD04590-01) | |
| 97775 Ovarian cancer NAT | 24.6 | 85976 Breast Cancer Mets | 21.0 |
| (OD06145) | | (OD04590-03) | |
| 98853 Ovarian cancer | 9.9 | 87070 Breast Cancer Metastasis | 66.6 |
| (OD06455-03) | | (OD04655-05) | |
| 98854 Ovarian NAT | 12.9 | GENPAK Breast Cancer | 19.0 |
| (OD06455-07) Fallopian tube | | 064006 | |
| Normal Lung GENPAK 061010 | 18.0 | Breast Cancer Clontech | 27.7 |
| | | 9100266 | |
| 92337 Invasive poor diff. lung | 11.8 | Breast NAT Clontech 9100265 | 21.6 |
| adeno (ODO4945-01 | | | |
| 92338 Lung NAT (ODO4945- | 13.8 | Breast Cancer INVITROGEN | 15.1 |
| 03) | | A209073 | |
| 84136 Lung Malignant Cancer | 31.7 | Breast NAT INVITROGEN | 28.5 |
| (OD03126) | | A2090734 | |
| 84137 Lung NAT (OD03126) | 5.6 | 97763 Breast cancer | 100.0 |
| | | (OD06083) | |
| 90372 Lung Cancer | 17.7 | 97764 Breast cancer node | 64.9 |
| (OD05014A) | | metastasis (OD06083) | |
| 90373 Lung NAT (OD05014B) | 13.7 | Normal Liver GENPAK | 17.1 |
| | | 061009 | |
| 97761 Lung cancer (OD06081) | 10.2 | Liver Cancer Research Genetics | 15.0 |
| | | RNA 1026 | |
| 97762 Lung cancer NAT | 8.2 | Liver Cancer Research Genetics | 36.3 |
| (OD06081) | | RNA 1025 | |
| 85950 Lung Cancer (OD04237- | 15.0 | Paired Liver Cancer Tissue | 18.1 |
| 01) | | Research Genetics RNA 6004- | |
| | | T | |
| 85970 Lung NAT (OD04237- | 24.0 | Paired Liver Tissue Research | 6.5 |
| 02) | | Genetics RNA 6004-N | |
| 83255 Ocular Mel Met to Liver | 25.5 | Paired Liver Cancer Tissue | 33.3 |
| (ODO4310) | | Research Genetics RNA 6005- | |
| | | T | |
| 83256 Liver NAT (ODO4310) | 18.1 | Paired Liver Tissue Research | 31.6 |
| | | Genetics RNA 6005-N | |
| 84139 Melanoma Mets to Lung | 41.3 | Liver Cancer GENPAK 064003 | 8.9 |
| (OD04321) | | | |
| 84138 Lung NAT (OD04321) | 9.1 | Normal Bladder GENPAK | 14.7 |
| | | 061001 | |
| Normal Kidney GENPAK | 7.5 | Bladder Cancer Research | 6.0 |
| 061008 | | Genetics RNA 1023 | |
| 83786 Kidney Ca, Nuclear | 34.7 | Bladder Cancer INVITROGEN | 28.9 |
| grade 2 (OD04338) | | A302173 | |
| 83787 Kidney NAT (OD04338) | 7.6 | Normal Stomach GENPAK | 33.6 |
| | | 061017 | |
| 83788 Kidney Ca Nuclear grade | 38.3 | Gastric Cancer Clontech | 3.6 |
| ½ (OD04339) | | 9060397 | |
| 83789 Kidney NAT (OD04339) | 6.8 | NAT Stomach Clontech | 12.2 |
| | | 9060396 | |
| 83790 Kidney Ca, Clear cell | 19.2 | Gastric Cancer Clontech | 15.1 |
| type (OD04340) | | 9060395 | |
| 83791 Kidney NAT (OD04340) | 18.7 | NAT Stomach Clontech | 21.2 |
| | | 9060394 | |
| 83792 Kidney Ca, Nuclear | 10.4 | Gastric Cancer GENPAK | 17.4 |
| grade 3 (OD04348) | | 064005 | |

Panel 36. Panel 4D

| | Relative | Relative |
|---|---|---|
| | Expression(%) | Expression(%) |
| | 4Dtm2070f_{—} | 4Dtm2425t_{—} |
| Tissue Name | ag1215 | ag1382 |
| 93768_Secondary Th1_anti-CD28/anti-CD3 | 27.9 | 19.6 |
| 93769_Secondary Th2_anti-CD28/anti-CD3 | 35.4 | 25.5 |
| 93770_Secondary Tr1_anti-CD28/anti-CD3 | 42.0 | 37.6 |
| 93573_Secondary Th1_resting day 4-6 in IL-2 | 29.5 | 18.8 |
| 93572_Secondary Th2_resting day 4-6 in IL-2 | 27.5 | 21.9 |
| 93571_Secondary Tr1_resting day 4-6 in IL-2 | 33.7 | 23.2 |
| 93568_primary Th1_anti-CD28/anti-CD3 | 35.1 | 28.1 |
| 93569_primary Th2_anti-CD28/anti-CD3 | 31.4 | 25.7 |
| 93570_primary Tr1_anti-CD28/anti-CD3 | 55.9 | 42.6 |
| 93565_primary Th1_resting dy 4-6 in IL-2 | 91.4 | 100.0 |
| 93566_primary Th2_resting dy 4-6 in IL-2 | 68.8 | 64.6 |
| 93567_primary Tr1_resting dy 4-6 in IL-2 | 55.5 | 52.1 |
| 93351_CD45RA CD4 lymphocyte_anti-CD28/anti-CD3 | 21.6 | 17.8 |
| 93352_CD45RO CD4 lymphocyte_anti-CD28/anti-CD3 | 24.5 | 17.2 |
| 93251_CD8 Lymphocytes_anti-CD28/anti-CD3 | 22.1 | 15.5 |
| 93353_chronic CD8 Lymphocytes 2ry_resting dy 4-6 in IL-2 | 17.1 | 12.4 |
| 93574_chronic CD8 Lymphocytes 2ry_activated CD3/CD28 | 29.7 | 18.2 |
| 93354_CD4_none | 17.9 | 15.4 |
| 93252_Secondary Th1/Th2/Tr1_anti-CD95 CH11 | 71.7 | 67.4 |
| 93103_LAK cells_resting | 17.1 | 10.3 |
| 93788_LAK cells_IL-2 | 21.5 | 15.7 |
| 93787_LAK cells_IL-2 + IL-12 | 18.4 | 17.1 |
| 93789_LAK cells_IL-2 + IFN gamma | 29.5 | 22.2 |
| 93790_LAK cells_IL-2 + IL-18 | 18.0 | 22.1 |
| 93104_LAK cells_PMA/ionomycin and IL-18 | 10.7 | 7.3 |
| 93578_NK Cells IL-2_resting | 31.9 | 17.2 |
| 93109_Mixed Lymphocyte Reaction_Two Way MLR | 9.5 | 7.6 |
| 93110_Mixed Lymphocyte Reaction_Two Way MLR | 6.2 | 6.8 |
| 93111_Mixed Lymphocyte Reaction_Two Way MLR | 10.5 | 8.5 |
| 93112_Mononuclear Cells (PBMCs)_resting | 10.7 | 10.3 |
| 93113_Mononuclear Cells (PBMCs)_PWM | 50.7 | 38.4 |
| 93114_Mononuclear Cells (PBMCs)_PHA-L | 32.5 | 31.2 |
| 93249_Ramos (B cell)_none | 0.0 | 0.0 |
| 93250_Ramos (B cell)_ionomycin | 0.0 | 0.0 |
| 93349_B lymphocytes_PWM | 36.1 | 34.2 |
| 93350_B lymphoytes_CD40L and IL-4 | 18.3 | 17.2 |
| 92665_EOL-1 (Eosinophil)_dbcAMP differentiated | 43.8 | 30.1 |
| 93248_EOL-1 (Eosinophil)_dbcAMP/PMAionomycin | 73.2 | 63.7 |
| 93356_Dendritic Cells_none | 1.6 | 0.6 |
| 93355_Dendritic Cells_LPS 100 ng/ml | 1.2 | 5.0 |
| 93775_Dendritic Cells_anti-CD40 | 0.9 | 0.9 |
| 93774_Monocytes_resting | 2.9 | 1.8 |
| 93776_Monocytes_LPS 50 ng/ml | 29.7 | 19.2 |
| 93581_Macrophages_resting | 4.9 | 2.3 |
| 93582_Macrophages_LPS 100 ng/ml | 7.2 | 4.4 |
| 93098_HUVEC (Endothelial)_none | 9.2 | 6.5 |
| 93099_HUVEC (Endothelial)_starved | 18.6 | 14.7 |
| 93100_HUVEC (Endothelial)_IL-1b | 3.9 | 2.6 |
| 93779_HUVEC (Endothelial)_IFN gamma | 19.2 | 17.1 |
| 93102_HUVEC (Endothelial)_TNF alpha + IFN gamma | 2.1 | 2.4 |
| 93101_HUVEC (Endothelial)_TNF alpha + IL4 | 15.3 | 12.2 |
| 93781_HUVEC (Endothelial)_IL-11 | 13.6 | 15.3 |
| 93583_Lung Microvascular Endothelial Cells_none | 19.9 | 20.4 |
| 93584_Lung Microvascular Endothelial Cells_TNFa (4 ng/ml) | 18.0 | 14.9 |
| and IL1b (1 ng/ml) | | |
| 92662_Microvascular Dermal endothelium_none | 29.7 | 26.8 |
| 92663_Microsvasular Dermal endothelium_TNFa (4 ng/ml) and | 36.9 | 33.4 |
| IL1b (1 ng/ml) | | |
| 93773_Bronchial epithelium_TNFa (4 ng/ml) and IL1b (1 | 41.5 | 25.7 |
| ng/ml)** | | |
| 93347_Small Airway Epithelium_none | 13.3 | 8.8 |
| 93348_Small Airway Epithelium_TNFa (4 ng/ml) and IL1b (1 | 56.6 | 45.4 |
| ng/ml) | | |
| 92668_Coronery Artery SMC_resting | 22.2 | 19.8 |
| 92669_Coronery Artery SMC_TNFa (4 ng/ml) and IL1b (1 | 28.1 | 19.9 |
| ng/ml) | | |
| 93107_astrocytes_resting | 13.0 | 20.2 |
| 93108_astrocytes_TNFa (4 ng/ml) and IL1b (1 ng/ml) | 23.7 | 18.6 |
| 92666_KU-812 (Basophil)_resting | 97.3 | 75.3 |
| 92667_KU-812 (Basophil)_PMA/ionoycin | 100.0 | 90.1 |
| 93579_CCD1106 (Keratinocytes)_none | 16.6 | 13.8 |
| 93580_CCD1106 (Keratinocytes)_TNFa and IFNg** | 67.8 | 11.3 |
| 93791_Liver Cirrhosis | 14.3 | 12.3 |
| 93792_Lupus Kidney | 28.7 | 18.8 |
| 93577_NCI-H292 | 27.4 | 28.3 |
| 93358_NCI-H292_IL-4 | 61.1 | 55.1 |
| 93360_NCI-H292_IL-9 | 37.6 | 31.6 |
| 93359_NCI-H292_IL-13 | 44.4 | 42.9 |
| 93357_NCI-H292_IFN gamma | 21.0 | 20.3 |
| 93777_HPAEC_- | 21.5 | 17.2 |
| 93778_HPAEC_IL-1 beta/TNA alpha | 11.0 | 9.5 |
| 93254_Normal Human Lung Fibroblast_none | 31.0 | 20.6 |
| 93253_Normal Human Lung Fibroblast_TNFa (4 ng/ml) and IL- | 26.1 | 23.5 |
| 1b (1 ng/ml) | | |
| 93257_Normal Human Lung Fibroblast_IL-4 | 40.6 | 37.1 |
| 93256_Normal Human Lung Fibroblast_IL-9 | 21.3 | 17.7 |
| 93255_Normal Human Lung Fibroblast_IL-13 | 56.3 | 53.2 |
| 93258_Normal Human Lung Fibroblast_IFN gamma | 59.5 | 45.4 |
| 93106_Dermal Fibroblasts CCD1070_resting | 47.0 | 33.4 |
| 93361_Dermal Fibroblasts CCD1070_TNF alpha 4 ng/ml | 73.7 | 55.1 |
| 93105_Dermal Fibroblasts CCD1070_IL-1 beta 1 ng/ml | 36.6 | 37.4 |
| 93772_dermal fibroblast_IFN gamma | 8.8 | 9.7 |
| 93771_dermal fibroblast_IL-4 | 20.2 | 19.6 |
| 93259_IBD Colitis 1** | 13.2 | 10.7 |
| 93260_IBD Colitis 2 | 4.1 | 2.0 |
| 93261_IBD Crohns | 3.1 | 3.1 |
| 735010_Colon_normal | 29.9 | 24.3 |
| 735019_Lung_none | 46.0 | 38.4 |
| 64028-1_Thymus_none | 60.3 | 54.0 |
| 64030-1_Kidney_none | 32.1 | 26.4 |

TABLE 37
Panel CNSD.01

| | Relative | | Relative |
|---|---|---|---|
| | Expression (%) | | Expression (%) |
| | cns1x4tm6177f | | cns1x4tm6177f |
| Tissue Name | _ag1215_b1 | Tissue Name | _ag1215_b1 |
| 102633_BA4 Control | 20.2 | 102605_BA17 PSP | 31.1 |
| 102641_BA4 Control2 | 34.5 | 102612_BA17 PSP2 | 9.9 |
| 102625_BA4 Alzheimer's2 | 9.0 | 102637_Sub Nigra Control | 54.5 |
| 102649_BA4 Parkinson's | 32.9 | 102645_Sub Nigra Control2 | 31.7 |
| 102656_BA4 Parkinson's2 | 46.8 | 102629_Sub Nigra | 24.2 |
| | | Alzheimer's2 | |
| 102664_BA4 Huntington's | 36.2 | 102660_Sub Nigra Parkinson's2 | 91.5 |
| 102671_BA4 Huntington's2 | 11.1 | 102667_SubNigra | 80.6 |
| | | Huntington's | |
| 102603_BA4 PSP | 15.8 | 102674_Sub Nigra | 43.0 |
| | | Huntington's2 | |
| 102610_BA4 PSP2 | 45.9 | 102614_Sub Nigra PSP2 | 24.8 |
| 102588_BA4 Depression | 19.6 | 102592_Sub Nigra Depression | 18.5 |
| 102596_BA4 Depression2 | 7.8 | 102599_Sub Nigra Depression2 | 12.0 |
| 102634_BA7 Control | 26.1 | 102636_Glob Palladus Control | 20.2 |
| 102642_BA7 Control2 | 32.8 | 102644_Glob Palladus Control2 | 10.7 |
| 102626_BA7 Alzheimer's2 | 4.5 | 102620_Glob Palladus | 21.6 |
| | | Alzheimer's | |
| 102650_BA7 Parkinson's | 22.9 | 102628_Glob Palladus | 4.9 |
| | | Alzheimer's2 | |
| 102657_BA7 Parkinson's2 | 35.5 | 102652_Glob Palladus | 100.0 |
| | | Parkinson's | |
| 102665_BA7 Huntington's | 39.1 | 102659_Glob Palladus | 23.0 |
| | | Parkinson's2 | |
| 102672_BA7 Huntington's2 | 40.4 | 102606_Glob Palladus PSP | 10.1 |
| 102604_BA7 PSP | 31.2 | 102613_Glob Palladus PSP2 | 11.8 |
| 102611_BA7 PSP2 | 32.2 | 102591_Glob Palladus | 23.4 |
| | | Depression | |
| 102589_BA7 Depression | 6.2 | 102638_Temp Pole Control | 7.7 |
| 102632_BA9 Control | 12.5 | 102646_Temp Pole Control2 | 29.2 |
| 102640_BA9 Control2 | 48.5 | 102622_Temp Pole Alzheimer's | 4.0 |
| 102617_BA9 Alzheimer's | 6.9 | 102630_Temp Pole | 3.6 |
| | | Alzheimer's2 | |
| 102624_BA9 Alzheimer's2 | 5.4 | 102653_Temp Pole Parkinson's | 22.5 |
| 102648_BA9 Parkinson's | 25.2 | 102661_Temp Pole | 22.1 |
| | | Parkinson's2 | |
| 102655_BA9 Parkinson's2 | 33.4 | 102668_Temp Pole | 30.6 |
| | | Huntington's | |
| 102663_BA9 Huntington's | 44.8 | 102607_Temp Pole PSP | 3.8 |
| 102670_BA9 Huntington's2 | 13.7 | 102615_Temp Pole PSP2 | 2.2 |
| 102602_BA9 PSP | 23.4 | 102600_Temp Pole | 7.1 |
| | | Depression2 | |
| 102609_BA9 PSP2 | 5.0 | 102639_Cing Gyr Control | 58.9 |
| 102587_BA9 Depression | 10.8 | 102647_Cing Gyr Control2 | 40.2 |
| 102595_BA9 Depression2 | 11.8 | 102623_Cing Gyr Alzheimer's | 19.3 |
| 102635_BA17 Control | 39.6 | 102631_Cing Gyr Alzheimer's2 | 11.8 |
| 102643_BA17 Control2 | 40.0 | 102654_Cing Gyr Parkinson's | 42.1 |
| 102627_BA17 Alzheimer's2 | 7.6 | 102662_Cing Gyr Parkinson's2 | 35.7 |
| 102651_BA17 Parkinson's | 31.2 | 102669_Cing Gyr Huntington's | 62.0 |
| 102658_BA17 Parkinson's2 | 35.2 | 102676_Cing Gyr | 28.7 |
| | | Huntington's2 | |
| 102666_BA17 Huntington's | 47.2 | 102608_Cing Gyr PSP | 66.8 |
| 102673_BA17 Huntington's2 | 25.0 | 102616_Cing Gyr PSP2 | 5.2 |
| 102590_BA17 Depression | 22.2 | 102594_Cing Gyr Depression | 14.7 |
| 102597_BA17 Depression2 | 41.7 | 102601_Cing Gyr Depression2 | 32.0 |

Panel 1.2 Summary: Ag1215/Ag1382 Two replicate experiments were performed using probe and primer sets of identical sequences; however, relatively disparate results were obtained on this panel. For Ag1215, the MOL5a gene is expressed at high levels across most of the tissues on this panel with highest expression in treated endothelial cells (CT value=23). For Ag1382, the MOL5 a gene is expressed at high levels across most of the tissues on this panel with highest expression in an ovarian cancer cell line (CT value=22). To summarize the expression profile, there appears to be widespread expression of the MOL5a gene in a number of tissues and cell lines. Furthermore, the expression of this gene seems to be associated with reproductive tissues and cancer cell lines whose origins are such. For instance, there is significant expression in ovarian cell lines, breast cell lines and placenta tissue. There is also moderate expression in kidney tissues and lung cell lines.

Panel 2.2 Summary: Ag1215 There appears to be widespread expression of the MOL5a gene in the samples of panel 2.2. Specifically. there seems to be an association of expression in breast cancer and normal ovarian tissue. This is reasonably consistent with the results obtained from Panel 1.2. In addition, there is also some correlation with expression in normal kidney tissue when compared to kidney cancers, also consistent with the observations in Panel 1.2. Thus, therapeutic modulation of this gene or gene product might show utility in the treatment of breast cancer, ovarian cancer or kidney cancer.

Panel 4D Summary: Ag1215/Ag1382 Results from two replicate experiments performed using probe and primer sets of identical sequences are in reasonable agreement. The MOL5a transcript is widely expressed in cell lines from this panel (CT values=25-30), including thymus, lung, muco-epidermoid cell lines, fibroblasts from diverse origin, and activated T cells. In addition, the MOL5a gene is expressed in normal colon but not in colons from patients with Crohn's disease or colitis. Thus, protein therapeutics designed with the putative semaphorin encoded for by this protein could reduce or eliminate inflammation and tissue destruction due to IBD. Ifigh expression of this transcript was found on primary resting Th1 T cells, and also primary resting Th2 and Tr1 T cells. The high expression of this transcript in secondary T cells treated with CD95 suggests that this transcript encodes for a protein involved in activation of cell death. Furthermore, high expression of the MOL5A transcript is also found in activated basophils and eosinophils, suggesting a role for this protein in allergic disorder such as asthma, contact hypersensitivity, and hypersensitive immediate reactions. Antibody or protein therapeutics designed against the protein encoded for by this transcript could therefore reduce or inhibit inflammation in allergy, asthma, emphysema, psoriasis and/or autoimmunity.

Panel CNSD.01 Summary: Ag1215 Semaphorins can act as axon guidance proteins. specifically through the ability to act as chemorepellents that inhibit CNS regenerative capacity. Manipulation of levels of the MOL4 semaphorin-like protein may therefore be of use in inducing a compensatory synaptogenic response to neuronal death in Alzheimer's disease, Parkinson's disease, Huntington's disease, spinocerebellar ataxia, progressive supranuclear palsy, multiple sclerosis, ALS, head trauma, stroke, or any other disease/condition associated with neuronal loss. cl F. MOL5d

TABLE BA
Probe Name: Ag1215

| | | | | Start | SEQ ID |
|---|---|---|---|---|---|
| Primers | Sequences | TM | Length | Position | NO: |
| Forward | 5′-AACCCATTATCCTGCGTAACAT-3′ | 59.6 | 22 | 619 | 129 |
| Probe | FAM-5′-CCCCACCACTCCATGAAGACAGAGTA-3′-TAMRA | 68.5 | 26 | 645 | 130 |
| Reverse | 5′-CCTACAAAGTCAGGTTCGTTGA-3′ | 59.3 | 22 | 685 | 131 |

TABLE BB
Probe Name: Ag1382

| | | | | Start | SEQ ID |
|---|---|---|---|---|---|
| Primers | Sequences | TM | Length | Position | NO: |
| Forward | 5′-AACCCATTATCCTGCGTAACAT-3′ | 59.6 | 22 | 373 | 132 |
| Probe | TET-5′-CCCCACCACTCCATGAAGACAGAGTA-3′-TAMRA | 68 5 | 26 | 399 | 133 |
| Reverse | 5′-CCTACAAAGTGAGGTTCGTTGA-3′ | 59.3 | 22 | 439 | 134 |

TABLE BC
Panel 1.2

| | Rel. Expr., % |
|---|---|
| Tissue Name | tm7170f_ag1215_{—b1} |
| D6005-01_Human adipose | 4.1 |
| 112193_Metastatic melanoma | 8.1 |
| 112192_Metastatic melanoma | 6.1 |
| 95280_Epidermis (metastatic melanoma) | 40.2 |
| 95279_Epidermis (metastatic melanoma) | 12 |
| Melanoma (met)_SK-MEL-5 | 22.4 |
| 112196_Tongue (oncology) | 6 |
| 113461_Testis Pool | 3 |
| Prostate ca.(bone met)_PC-3 | 12.6 |
| 113455_Prostate Pool | 4.6 |
| 103396_Placenta | 26.2 |
| 113463_Uterus Pool | 3.8 |
| Ovarian carcinoma_OVCAR-3 | 20 |
| Ovarian carcinoma(ascites)_SK-OV-3 | 8.8 |
| 95297_Adenocarcinoma (ovary) | 3.9 |
| Ovarian carcinoma_OVCAR-5 | 21.3 |
| Ovarian carcinoma_IGROV-1 | 9.6 |
| Ovarian carcinoma_OVCAR-8 | 8.2 |
| 103368_Ovary | 7.2 |
| MCF7_breast carcinoma(pleural effusion) | 100 |
| Breast ca. (pleural effusion)_MDA-MB-231 | 10.9 |
| 112189_ductal cell carcinoma(breast) | 17.1 |
| Breast ca. (pleural effusion)_T47D | 33.1 |
| Breast carcinoma_MDA-N | 7.9 |
| 113452_Breast Pool | 13.2 |
| 103398_Trachea | 9 |
| 112354_lung | 1.4 |
| 103374_Fetal Lung | 20.9 |
| 94921_Small cell carcinoma of the lung | 0.9 |
| Lung ca.(small cell)_LX-1 | 12.8 |
| 94919_Small cell carcinoma of the lung | 4.3 |
| Lung ca.(s.cell var.)_SHP-77 | 8.1 |
| 95268_Lung (Large cell carcinoma) | 9.4 |
| 94920_Small cell carcinoma of the lung | 3 |
| Lung ca.(non-s.cell)_NCI-H23 | 33.8 |
| Lung ca.(large cell)_NCI-H460 | 8.8 |
| Lung ca.(non-s.cell)_HOP-62 | 11.2 |
| Lung ca.(non-s.cl)_NCI-H522 | 18.3 |
| 103392_Liver | 0.5 |
| 103393_Fetal Liver | 6.8 |
| Liver ca.(hepatoblast)_HepG2 | 9.4 |
| 113465_Kidney Pool | 18.7 |
| 103373_Fetal Kidney | 5 |
| Renal ca._786-0 | 7.9 |
| 112188_renal cell carcinoma | 6.8 |
| Renal ca._ACHN | 7.8 |
| 112190_Renal cell carcinoma | 8.7 |
| Renal ca._TK-10 | 10 |
| Bladder | 9.5 |
| Gastric ca.(liver met)_NCI-N87 | 11.6 |
| 112197_Stomach | 0 |
| 94938_Colon Adenocarcinoma | 2.4 |
| Colon ca._SW480 | 13.6 |
| Colon ca.(SW480 met)_SW620 | 7.9 |
| Colon ca._HT29 | 1.6 |
| Colon ca._HCT-116 | 11.6 |
| Colon ca._CaCo-2 | 10.6 |
| 83219_CC Well to Mod Diff (ODO3866) | 7.1 |
| 94936_Colon Adenocarcinoma | 2.5 |
| 94930_Colon | 0 |
| 94935_Colon Adenocarcinoma | 0.4 |
| 113468_Colon Pool | 9.9 |
| 113457_Small Intestine Pool | 20.1 |
| 113460_Stomach Pool | 7.6 |
| 113467_Bone Marrow Pool | 5.2 |
| 103371_Fetal Heart | 6.7 |
| 113451_Heart Pool | 4.2 |
| 113466_Lymph Node Pool | 14.2 |
| 103372_Fetal Skeletal Muscle | 5.6 |
| 113456_Skeletal Muscle Pool | 3.4 |
| 113459_Spleen Pool | 10.3 |
| 113462_Thymus Pool | 6.4 |
| CNS ca. (glio/astro)_U87-MG | 9.2 |
| CNS ca. (glio/astro)_U-118-MG | 12.4 |
| CNS ca. (neuro;met)_SK-N-AS | 18.7 |
| 95264_Brain astrocytoma | 17.8 |
| CNS ca. (astro)_SNB-75 | 42.8 |
| CNS ca. (glio)_SNB-19 | 7.1 |
| CNS ca. (glio)_SF-295 | 14.8 |
| 113447_Brain (Amygdala) Pool | 10.9 |
| 103382_Brain (cerebellum) | 46.7 |
| 64019-1_brain(fetal) | 33 |
| 113448_Brain (Hippocampus) Pool | 9.4 |
| 113464_Cerebral Cortex Pool | 7.4 |
| 113449_Brain (Substantia nigra) Pool | 9.4 |
| 113450_Brain (Thalamus) Pool | 11.7 |
| 103384_Brain (whole) | 16.1 |
| 113458_Spinal Cord Pool | 12.6 |
| 103375_Adrenal Gland | 8.6 |
| 113454_Pituitary gland Pool | 1 |
| 103397_Salivary Gland | 5.2 |
| 103369_Thyroid (female) | 3.5 |
| Pancreatic ca._CAPAN2 | 5 |
| 113453_Pancreas Pool | 18.1 |

TABLE BD
Panel 2.2

| | Rel. Expr., % |
|---|---|
| Tissue Name | 2.2x4tm6515f_ag1215_a2 |
| Normal Colon GENPAK 061003 | 31.4 |
| 97759 Colon cancer (OD06064) | 34.3 |
| 97760 Colon cancer NAT (OD06064) | 18.6 |
| 97778 Colon cancer (OD06159) | 2.7 |
| 97779 Colon cancer NAT (OD06159) | 22.5 |
| 98861 Colon cancer (OD06297-04) | 3.2 |
| 98862 Colon cancer NAT (OD06297-015) | 30.6 |
| 83237 CC Gr.2 ascend colon (ODO3921) | 11.6 |
| 83238 CC NAT (OD03921) | 8.4 |
| 97766 Colon cancer metastasis (OD06104) | 5.1 |
| 97767 Lung NAT (OD06104) | 8.3 |
| 87472 Colon mets to lung (OD04451-01) | 15.3 |
| 87473 Lung NAT (OD04451-02) | 4.3 |
| Normal Prostate Clontech A + 6546-1 (8090438) | 11.2 |
| 84140 Prostate Cancer (OD04410) | 10 |
| 84141 Prostate NAT (OD04410) | 14.3 |
| Normal Ovary Res. Gen. | 74.7 |
| 98863 Ovarian cancer (OD06283-03) | 27.6 |
| 98865 Ovarian cancer NAT/fallopian tube (OD06283-07) | 6.4 |
| Ovarian Cancer GENPAK 064008 | 16.2 |
| 97773 Ovarian cancer (OD06145) | 8.8 |
| 97775 Ovarian cancer NAT (OD06145) | 24.6 |
| 98853 Ovarian cancer (OD06455-03) | 9.9 |
| 98854 Ovarian NAT (OD06455-07) Fallopian tube | 12.9 |
| Normal Lung GENPAK 061010 | 18 |
| 92337 Invasive poor diff. lung adeno (ODO4945-01 | 11.8 |
| 92338 Lung NAT (ODO4945-03) | 13.8 |
| 84136 Lung Malignant Cancer (OD03126) | 31.7 |
| 84137 Lung NAT (OD03126) | 5.6 |
| 90372 Lung Cancer (OD05014A) | 17.7 |
| 90373 Lung NAT (OD05014B) | 13.7 |
| 97761 Lung cancer (OD06081) | 10.2 |
| 97762 Lung cancer NAT (OD06081) | 8.2 |
| 85950 Lung Cancer (OD04237-01) | 15 |
| 85970 Lung NAT (OD04237-02) | 24 |
| 83255 Ocular Mel Met to Liver (ODO4310) | 25.5 |
| 83256 Liver NAT (ODO4310) | 18.1 |
| 84139 Melanoma Mets to Lung (OD4321) | 41.3 |
| 84138 Lung NAT (OD04321) | 9.1 |
| Normal Kidney GENPAK 061008 | 7.5 |
| 83786 Kidney Ca, Nuclear grade 2 (OD04338) | 34.7 |
| 83787 Kidney NAT (OD04338) | 7.6 |
| 83788 Kidney Ca Nuclear grade 1/2 (OD04339) | 38.3 |
| 83789 Kidney NAT (OD04339) | 6.8 |
| 83790 Kidney Ca, Clear cell type (OD04340) | 19.2 |
| 83791 Kidney NAT (OD04340) | 18.7 |
| 83792 Kidney Ca, Nuclear grade 3 (OD04348) | 10.4 |
| 83793 Kidney NAT (OD04348) | 39.7 |
| 98938 Kidney malignant cancer (OD06204B) | 15.4 |
| 98939 Kidney normal adjacent tissue (OD06204E) | 10.4 |
| 85973 Kidney Cancer (OD04450-01) | 15.3 |
| 85974 Kidney NAT (OD04450-03) | 20.3 |
| Kidney Cancer Clontech 8120613 | 1.4 |
| Kidney NAT Clontech 8120614 | 20.3 |
| Kidney Cancer Clontech 9010320 | 10.5 |
| Kidney NAT Clontech 9010321 | 9 |
| Kidney Cancer Clontech 8120607 | 32.2 |
| Kidney NAT Clontech 8120608 | 12.1 |
| Normal Uterus GENPAK 061018 | 31.8 |
| Uterus Cancer GENPAK 064011 | 31.4 |
| Normal Thyroid Clontech A + 6570-1 (7080817) | 3 |
| Thyroid Cancer GENPAK 064010 | 14.8 |
| Thyroid Cancer INVITROGEN A302152 | 37.7 |
| Thyroid NAT INVITROGEN A302153 | 7 |
| Normal Breast GENPAK 061019 | 35.4 |
| 84877 Breast Cancer (OD04566) | 15.6 |
| Breast Cancer Res. Gen. 1024 | 51.4 |
| 85975 Breast Cancer (OD04590-01) | 36.8 |
| 85976 Breast Cancer Mets (OD04590-03) | 21 |
| 87070 Breast Cancer Metastasis (OD04655-05) | 66.6 |
| GENPAK Breast Cancer 064006 | 19 |
| Breast Cancer Clontech 9100266 | 27.7 |
| Breast NAT Clontech 9100265 | 21.6 |
| Breast Cancer INVITROGEN A209073 | 15.1 |
| Breast NAT INVITROGEN A2090734 | 28.5 |
| 97763 Breast cancer (OD06083) | 100 |
| 97764 Breast cancer node metastasis (OD06083) | 64.9 |
| Normal Liver GENPAK 061009 | 17.1 |
| Liver Cancer Research Genetics RNA 1026 | 15 |
| Liver Cancer Research Genetics RNA 1025 | 36.3 |
| Paired Liver Cancer Tissue Research Genetics RNA 6004-T | 18.1 |
| Paired Liver Tissue Research Genetics RNA 6004-N | 6.5 |
| Paired Liver Cancer Tissue Research Genetics RNA 6005-T | 33.3 |
| Paired Liver Tissue Research Genetics RNA 6005-N | 31.6 |
| Liver Cancer GENPAK 064003 | 8.9 |
| Normal Bladder GENPAK 061001 | 14.7 |
| Bladder Cancer Research Genetics RNA 1023 | 6 |
| Bladder Cancer INVITROGEN A302173 | 28.9 |
| Normal Stomach GENPAK 061017 | 33.6 |
| Gastric Cancer Clontech 9060397 | 3.6 |
| NAT Stomach Clontech 9060396 | 12.2 |
| Gastric Cancer Clontech 9060395 | 15.1 |
| NAT Stomach Clontech 9060394 | 21.2 |
| Gastric Cancer GENPAK 064005 | 17.4 |

TABLE BE
Panel 4D

| | Rel. Expr., % |
|---|---|
| Tissue Name | 4Dtm2070f_ag1215 |
| 93768_Secondary Th1_anti-CD28/anti-CD3 | 27.9 |
| 93769_Secondary Th2_anti-CD28/anti-CD3 | 35.4 |
| 93770_Secondary Tr1_anti-CD28/anti-CD3 | 42 |
| 93573_Secondary Th1_resting day 4-6 in IL-2 | 29.5 |
| 93572_Secondary Th2_resting day 4-6 in IL-2 | 27.5 |
| 93571_Secondary Tr1_resting day 4-6 in IL-2 | 33.7 |
| 93568_primary Th1_anti-CD28/anti-CD3 | 35.1 |
| 93569_primary Th2_anti-CD28/anti-CD3 | 31.4 |
| 93570_primary Tr1_anti-CD28/anti-CD3 | 55.9 |
| 93565_primary Th1_resting dy 4-6 in IL-2 | 91.4 |
| 93566_primary Th2_resting dy 4-6 in IL-2 | 68.8 |
| 93567_primary Tr1_resting dy 4-6 in IL-2 | 55.5 |
| 93351_CD45RA CD4 lymphocyte_anti-CD28/anti-CD3 | 21.6 |
| 93352_CD45RO CD4 lymphocyte_anti-CD28/anti-CD3 | 24.5 |
| 93251_CD8 Lymphocytes_anti-CD28/anti-CD3 | 22.1 |
| 93353_chronic CD8 Lymphocytes 2ry_resting dy 4-6 in IL-2 | 17.1 |
| 93574_chronic CD8 Lymphocytes 2ry_activated CD3/CD28 | 29.7 |
| 93354_CD4_none | 17.9 |
| 93252_Secondary Th1/Th2/Tr1_anti-CD95 CH11 | 71.7 |
| 93103_LAK cells_resting | 17.1 |
| 93788_LAK cells_IL-2 | 21.5 |
| 93787_LAK cells_IL-2 + IL-12 | 18.4 |
| 93789_LAK cells_IL-2 + IFN gamma | 29.5 |
| 93790_LAK cells_IL-2 + IL-l8 | 18 |
| 93104_LAK cells_PMA/ionomycin and IL-18 | 10.7 |
| 93578_NK Cells IL-2_resting | 31.9 |
| 93109_Mixed Lymphocyte Reaction_Two Way MLR | 9.5 |
| 93110_Mixed Lymphocyte Reaction_Two Way MLR | 6.2 |
| 93111_Mixed Lymphocyte Reaction_Two Way MLR | 10.5 |
| 93112_Mononuclear Cells (PBMCs)_resting | 10.7 |
| 93113_Mononuclear Cells (PBMCs)_PWM | 50.7 |
| 93114_Mononuclear Cells (PBMCs)_PHA-L | 32.5 |
| 93249_Ramos (B cell)_none | 0 |
| 93250_Ramos (B cell)_ionomycin | 0 |
| 93349_B lymphocytes_PWM | 36.1 |
| 93350_B lymphoytes_CD40L and IL-4 | 18.3 |
| 92665_EOL-1 (Eosinophil)_dbcAMP differentiated | 43.8 |
| 93248_EOL-1 (Eosinophil)_dbcAMP/PMAionomycin | 73.2 |
| 93356_Dendritic Cells_none | 1.6 |
| 93355_Dendritic Cells_LPS 100 ng/ml | 1.2 |
| 93775_Dendritic Cells_anti-CD40 | 0.9 |
| 93774_Monocytes_resting | 2.9 |
| 93776_Monocytes LPS 50 ng/ml | 29.7 |
| 93581_Macrophages_resting | 4.9 |
| 93582_Macrophages_LPS 100 ng/ml | 7.2 |
| 93098_HUVEC (Endothelial)_none | 9.2 |
| 93099_HUVEC (Endothelial)_starved | 18.6 |
| 93100_HUVEC (Endothelial)_IL-1b | 3.9 |
| 93779_HUVEC (Endothelial)_IFN gamma | 19.2 |
| 93102_HUVEC (Endothelial)_TNF alpha + IFN gamma | 2.1 |
| 93101_HUVEC (Endothelial)_TNF alpha + IL4 | 15.3 |
| 93781_HUVEC (Endothelial)_IL-11 | 13.6 |
| 93583_Lung Microvascular Endothelial Cells_none | 19.9 |
| 93584_Lung Microvascular Endothelial Cells_TNFa (4 ng/ml) and IL1b (1 ng/ml) | 18 |
| 92662_Microvascular Dermal endothelium_none | 29.7 |
| 92663_Microsvasular Dermal endothelium_TNFa (4 ng/ml) and IL1b (1 ng/ml) | 36.9 |
| 93773_Bronchial epithelium_TNFa (4 ng/ml) and IL1b (1 ng/ml)** | 41.5 |
| 93347_Small Airway Epithelium_none | 13.3 |
| 93348_Small Airway Epithelium_TNFa (4 ng/ml) and IL1b (1 ng/ml) | 56.6 |
| 92668_Coronery Artery SMC_resting | 22.2 |
| 92669_Coronery Artery SMC_TNFa (4 ng/ml) and IL1b (1 ng/ml) | 28.1 |
| 93107_astrocytes_resting | 13 |
| 93108_astrocytes_TNFa (4 ng/ml) and IL1b (1 ng/ml) | 23.7 |
| 92666_KU-812 (Basophil)_resting | 97.3 |
| 92667_KU-812 (Basophil)_PMA/ionoycin | 100 |
| 93579_CCD1106 (Keratinocytes)_none | 16.6 |
| 93580_CCD1106 (Keratinocytes)_TNFa and IFNg** | 67.8 |
| 93791_Liver Cirrhosis | 14.3 |
| 93792_Lupus Kidney | 28.7 |
| 93577_NCI-H292 | 27.4 |
| 93358_NCI-H292_IL-4 | 61.1 |
| 93360_NCI-H292_IL-9 | 37.6 |
| 93359_NCI-H292_IL-13 | 44.4 |
| 93357_NCI-H292_IFN gamma | 21 |
| 93777_HPAEC_- | 21.5 |
| 93778_HPAEC_IL-1 beta/TNA alpha | 11 |
| 93254_Normal Human Lung Fibroblast_none | 31 |
| 93253_Normal Human Lung Fibroblast_TNFa (4 ng/ml) and IL-1b (1 ng/ml) | 26.1 |
| 93237_Normal Human Lung Fibroblast_IL-4 | 40.6 |
| 93256_Normal Human Lung Fibroblast_IL-9 | 21.3 |
| 93255_Normal Human Lung Fibroblast_IL-13 | 56.3 |
| 93258_Normal Human Lung Fibroblast_IFN gamma | 59.5 |
| 93106_Dermal Fibroblasts CCD1070_resting | 47 |
| 93361_Dermal Fibroblasts CCD1070_TNF alpha 4 ng/ml | 73.7 |
| 93105_Dermal Fibroblasts CCD1070_IL-1 beta 1 ng/ml | 36.6 |
| 93772_dermal fibroblast_IFN gamma | 8.8 |
| 93771_dermal Fibroblast_IL-4 | 20.2 |
| 93259_IBD Colitis 1** | 13.2 |
| 93260_IBD Colitis 2 | 4.1 |
| 93261_IBD Crohns | 3.1 |
| 735010_Colon_normal | 29.9 |
| 735019_Lung_none | 46 |
| 64028-1_Thymus_none | 60.3 |
| 64030-1_Kidney_none | 32.1 |

TABLE BF
Panel 1.2

| | Rel. Expr., % |
|---|---|
| Tissue Name | 1 2tm1581t_ag1382 |
| Endothelial cells | 17.3 |
| Heart (fetal) | 40.9 |
| Pancreas | 0.4 |
| Pancreatic ca. CAPAN 2 | 0.7 |
| Adrenal Gland (new lot*) | 10.4 |
| Thyroid | 0.3 |
| Salavary gland | 5.8 |
| Pituitary gland | 1.4 |
| Brain (fetal) | 1.4 |
| Brain (whole) | 3.8 |
| Brain (amygdala) | 3.6 |
| Brain (cerebellum) | 3 |
| Brain (hippocampus) | 12.6 |
| Brain (thalamus) | 26.4 |
| Cerebral Cortex | 30.1 |
| Spinal cord | 7.2 |
| CNS ca (glio/astro) U87-MG | 21.3 |
| CNS ca (glio/astro) U-118-MG | 13.5 |
| CNS ca. (astro) SW1783 | 1.8 |
| CNS ca.* (neuro, met) SK-N-AS | 27.5 |
| CNS ca (astro) SF-539 | 21.2 |
| CNS ca. (astro) SNB-75 | 2.8 |
| CNS ca (glio) SNB-19 | 28.9 |
| CNS ca. (glio) U251 | 0.1 |
| CNS ca (glio) SF-295 | 5.1 |
| Heart | 12.2 |
| Skeletal Muscle (new lot*) | 0.8 |
| Bone marrow | 0.9 |
| Thymus | 2.8 |
| Spleen | 15.6 |
| Lymph node | 5.4 |
| Colorectal | 2.4 |
| Stomach | 9.6 |
| Small intestine | 11.7 |
| Colon ca SW480 | 1.8 |
| Colon ca * (SW480 met)SW620 | 40.3 |
| Colon ca HT29 | 2.5 |
| Colon ca HCT-116 | 11 |
| Colon ca CaCo-2 | 28.9 |
| 83219 CC Well to Mod Diff (ODO3866) | 1.8 |
| Colon ca HCC-2998 | 9.5 |
| Gastric ca * (liver met) NCI-N87 | 17.6 |
| Bladder | 15.5 |
| Trachea | 6 |
| Kidney | 42.9 |
| Kidney (fetal) | 61.6 |
| Renal ca 786-0 | 6.7 |
| Renal ca. A498 | 9.3 |
| Renal ca RXF 393 | 6 |
| Renal ca ACHN | 4.9 |
| Renal ca. UO-31 | 3.6 |
| Renal ca. TK-10 | 6.2 |
| Liver | 3.2 |
| Liver (fetal) | 5.9 |
| Liver ca (hepatoblast) HepG2 | 12.1 |
| Lung | 0.7 |
| Lung (fetal) | 4.5 |
| Lung ca (small cell) LX-1 | 24 |
| Lung ca. (small cell) NCI-H69 | 12.7 |
| Lung ca (s.cell var) SHP-77 | 1.8 |
| Lung ca (large cell)NCI-H460 | 19.5 |
| Lung ca (non-sm cell) A549 | 13.9 |
| Lung ca (non-s.cell) NCI-H23 | 24.5 |
| Lung ca (non-s.cell) HOP-62 | 9.7 |
| Lung ca. (non-s.cl) NCI-H522 | 44.1 |
| Lung ca (squam.) SW 900 | 57.8 |
| Lung ca. (squam.) NCI-H596 | 4.2 |
| Mammary gland | 9 |
| Breast ca * (pl. effusion) MCF-7 | 56.6 |
| Breast ca.* (pl.ef) MDA-MB-231 | 1.4 |
| Breast ca * (pl effusion) T47D | 16.6 |
| Breast ca BT-549 | 2.6 |
| Breast ca. MDA-N | 3.7 |
| Ovary | 23 |
| Ovarian ca. OVCAR-3 | 50.7 |
| Ovarian ca. OVCAR-4 | 10.6 |
| Ovarian ca OVCAR-5 | 66.9 |
| Ovarian ca. OVCAR-8 | 100 |
| Ovarian ca IGROV-1 | 13.3 |
| Ovarian ca * (ascites) SK-OV-3 | 10.3 |
| Uterus | 3.5 |
| Placenta | 82.9 |
| Prostate | 15 |
| Prostate ca * (bone met)PC-3 | 15.4 |
| Testis | 0.6 |
| Melanoma Hs688(A) T | 1.2 |
| Melanoma* (met) Hs688(B) T | 0.8 |
| Melanoma UACC-62 | 11.4 |
| Melanoma M14 | 6.7 |
| Melanoma LOX IMVI | 1.8 |
| Melanoma* (met) SK-MEL-5 | 8.9 |
| Adipose | 12.8 |

TABLE BG
Panel 4D

| | Rel Expr, % |
|---|---|
| Tissue Name | 4Dtm2425t_ag1382 |
| 93768_Secondary Th1_anti-CD28/anti-CD3 | 19.6 |
| 93769_Secondary Th2_anti-CD28/anti-CD3 | 25.5 |
| 93770_Secondary Tr1_anti-CD28/anti-CD3 | 37.6 |
| 93573_Secondary Th1_resting day 4-6 in IL-2 | 18.8 |
| 93572_Secondary Th2_resting day 4-6 in IL-2 | 21.9 |
| 93571_Secondary Tr1_resting day 4-6 in IL-2 | 23.2 |
| 93568_primary Th1_anti-CD28/anti-CD3 | 28.1 |
| 93569_primary Th2_anti-CD28/anti-CD3 | 25.7 |
| 93570_primary Tr1_anti-CD28/anti-CD3 | 42.6 |
| 93565_primary Th1_resting dy 4-6 in IL-2 | 100 |
| 93566_primary Th2_resting dy 4-6 in IL-2 | 64.6 |
| 93567_primary Tr1_resting dy 4-6 in IL-2 | 52.1 |
| 93351_CD45RA CD4 lymphocyte_anti-CD28/anti-CD3 | 17.8 |
| 93352_CD45RO CD4 lymphocyte_anti-CD28/anti-CD3 | 17.2 |
| 93251_CD8 Lymphocytes_anti-CD28/anti-CD3 | 15.5 |
| 93353_chronic CD8 Lymphocytes 2ry_resting dy 4-6 in IL-2 | 12.4 |
| 93574_chronic CD8 Lymphocytes 2ry_activated CD3/CD28 | 18.2 |
| 93354_CD4_none | 15.4 |
| 93252_Secondary Th1/Th2/Tr1_anti-CD95 CH11 | 67.4 |
| 93103_LAK cells_resting | 10.3 |
| 93788_LAK cells_IL-2 | 15.7 |
| 93787_LAK cells_IL-2 + IL-12 | 17.1 |
| 93789_LAK cells_IL-2 + IFN gamma | 22.2 |
| 93790_LAK cells_IL-2 + IL-18 | 22.1 |
| 93104_LAK cells_PMA/ionomycin and IL-18 | 7.3 |
| 93578_NK Cells IL-2_resting | 17.2 |
| 93109_Mixed Lymphocyte Reaction_Two Way MLR | 7.6 |
| 93110_Mixed Lymphocyte Reaction_Two Way MLR | 6.8 |
| 93111_Mixed Lymphocyte Reaction_Two Way MLR | 8.5 |
| 93112_Mononuclear Cells (PBMCs)_resting | 10.3 |
| 93113_Mononuclear Cells (PBMCs)_PWM | 38.4 |
| 93114_Mononuclear Cells (PBMCs)_PHA-L | 31.2 |
| 93249_Ramos (B cell)_none | 0 |
| 93250_Ramos (B cell)_ionomycin | 0 |
| 93349_B lymphocytes_PWM | 34.2 |
| 93350_B lymphoytes_CD40L and IL-4 | 17.2 |
| 92665_EOL-1 (Eosinophil)_dbcAMP differentiated | 30.1 |
| 93248_EOL-1 (Eosinophil)_dbcAMP/PMAionomycin | 63.7 |
| 93356_Dendritic Cells_none | 0.6 |
| 93355_Dendritic Cells_LPS 100 ng/ml | 5 |
| 93775_Dendritic Cells_anti-CD40 | 0.9 |
| 93774_Monocytes_resting | 1.8 |
| 93776_Monocytes_LPS 50 ng/ml | 19.2 |
| 93581_Macrophages_resting | 2.3 |
| 93582_Macrophages_LPS 100 ng/ml | 4.4 |
| 93098_HUVEC (Endothelial)_none | 6.5 |
| 93099_HUVEC (Endothelial)_starved | 14.7 |
| 93100_HUVEC (Endothelial)_IL-1b | 2.6 |
| 93779_HUVEC (Endothelial)_IFN gamma | 17.1 |
| 93102_HUVEC (Endothelial)_TNF alpha + IFN gamma | 2.4 |
| 93101_HUVEC (Endothelial)_TNF alpha + IL4 | 12.2 |
| 93781_HUVEC (Endothelial)_IL-11 | 15.3 |
| 93583_Lung Microvascular Endothelial Cells_none | 20.4 |
| 93584_Lung Microvascular Endothelial Cells_TNFa (4 ng/ml) and IL1b (1 ng/ml) | 14.9 |
| 92662_Microvascular Dermal endothelium_none | 26.8 |
| 92663_Microsvasular Dermal endothelium_TNFa (4 ng/ml) and IL1b (1 ng/ml) | 33.4 |
| 93773_Bronchial epithelium_TNFa (4 ng/ml) and IL1b (1 ng/ml)** | 25.7 |
| 93347_Small Airway Epithelium_none | 8.8 |
| 93348_Small Airway Epithelium_TNFa (4 ng/ml) and IL1b (1 ng/ml) | 45.4 |
| 92668_Coronery Artery SMC_resting | 19.8 |
| 92669_Coronery Artery SMC_TNFa (4 ng/ml) and IL1b (1 ng/ml) | 19.9 |
| 93107_astrocytes_resting | 20.2 |
| 93108_astrocytes_TNFa (4 ng/ml) and IL1b (1 ng/ml) | 18.6 |
| 92666_KU-812 (Basophil)_resting | 75.3 |
| 92667_KU-812 (Basophil)_PMA/ionoycin | 90.1 |
| 93579_CCD1106 (Keratinocytes)_none | 13.8 |
| 93580_CCD1106 (Keratinocytes)_TNFa and IFNg** | 11.3 |
| 93791_Liver Cirrhosis | 12.3 |
| 93792_Lupus Kidney | 18.8 |
| 93577_NCI-H292 | 28.3 |
| 93358_NCI-H292_IL-4 | 55.1 |
| 93360_NCI-H292_IL-9 | 31.6 |
| 93359_NCI-H292_IL-13 | 42.9 |
| 93357_NCI-H292_IFN gamma | 20.3 |
| 93777_HPAEC_- | 17.2 |
| 93778_HPAEC_IL-1 beta/TNA alpha | 9.5 |
| 93254_Normal Human Lung Fibroblast_none | 20.6 |
| 93253_Normal Human Lung Fibroblast_TNFa (4 ng/ml) and IL-1b (1 ng/ml) | 23.5 |
| 93257_Normal Human Lung Fibroblast_IL-4 | 37.1 |
| 93256_Normal Human Lung Fibroblast_IL-9 | 17.7 |
| 93255_Normal Human Lung Fibroblast_IL-13 | 53.2 |
| 93258_Normal Human Lung Fibroblast_IFN gamma | 45.4 |
| 93106_Dermal Fibroblasts CCD1070_resting | 33.4 |
| 93361_Dermal Fibroblasts CCD1070_TNF alpha 4 ng/ml | 55.1 |
| 93105_Dermal Fibroblasts CCD1070_IL-1 beta 1 ng/ml | 37.4 |
| 93772_dermal fibroblast_IFN gamma | 9.7 |
| 93771_dermal fibroblast_IL-4 | 19.6 |
| 93259_IBD Colitis 1** | 10.7 |
| 93260_IBD Colitis 2 | 2 |
| 93261_IBD Crohns | 3.1 |
| 735010_Colon_normal | 24.3 |
| 735019_Lung_none | 38.4 |
| 64028-1_Thymus_none | 54 |
| 64030-1_Kidney_none | 26.4 |

TABLE BH
Panel 1.4

| | Rel. Expr, % |
|---|---|
| Tissue Name | 1.4x4tm7306t_ag37_b2 |
| D6005-01_Human adipose | 15.2 |
| 112193_Metastatic melanoma | 13.6 |
| 112192_Metastatic melanoma | 14.6 |
| 95280_Epidermis (metastatic melanoma) | 52.1 |
| 95279_Epidermis (metastatic melanoma) | 25.2 |
| Melanoma (met)_SK-MEL-5 | 41.3 |
| 112196_Tongue (oncology) | 30.5 |
| 113461_Testis Pool | 3.5 |
| Prostate ca.(bone met)_PC-3 | 21.3 |
| 113455_Prostate Pool | 11.8 |
| 103396_Placenta | 19.7 |
| 113463_Uterus Pool | 13.2 |
| Ovarian carcinoma_OVCAR-3 | 27.5 |
| Ovarian carcinoma(ascites)_SK-OV-3 | 23.8 |
| 95297_Adenocarcinoma (ovary) | 11.5 |
| Ovarian carcinoma_OVCAR-5 | 37.5 |
| Ovarian carcinoma_IGROV-1 | 30.2 |
| Ovarian carcinoma_OVCAR-8 | 23.3 |
| 103368_Ovary | 2.1 |
| MCF7_breast carcinoma(pleural effusion) | 100 |
| Breast ca. (pleural effusion)_MDA- | 17.5 |
| MB-231 | |
| 112189_ductal cell carcinoma(breast) | 23.1 |
| Breast ca. (pleural effusion)_T47D | 62.8 |
| Breast carcinoma_MDA-N | 10.8 |
| 113452_Breast Pool | 43.3 |
| 103398_Trachea | 18.3 |
| 112354_lung | 2.2 |
| 103374_Fetal Lung | 34.7 |
| 94921_Small cell carcinoma of the lung | 2.6 |
| Lung ca.(small cell)_LX-1 | 21.7 |
| 94919_Small cell carcinoma of the lung | 6.1 |
| Lung ca (s.cell var.)_SHP-77 | 11.8 |
| 95268_Lung (Large cell carcinoma) | 11.3 |
| 94920_Small cell carcinoma of the lung | 6.5 |
| Lung ca (non-s.cell)_NCI-H23 | 50 |
| Lung ca.(large cell)_NCI-H460 | 16.6 |
| Lung ca.(non-s.cell)_HOP-62 | 21.6 |
| Lung ca.(non-s.cl)_NCI-H522 | 30.6 |
| 103392_Liver | 1.7 |
| 103393_Fetal Liver | 5.1 |
| Liver ca (hepatoblast)_HepG2 | 12.7 |
| 113465_Kidney Pool | 61.7 |
| 103373_Fetal Kidney | 12.7 |
| Renal ca._786-0 | 15.8 |
| 112188_renal cell carcinoma | 8.2 |
| Renal ca_ACHN | 14.1 |
| 112190_Renal cell carcinoma | 14.8 |
| Renal ca_TK-10 | 20 |
| Bladder | 19.9 |
| Gastric ca.(liver met)_NCI-N87 | 19.7 |
| 112197_Stomach | 0.3 |
| 94938_Colon Adenocarcinoma | 8.7 |
| Colon ca _SW480 | 25.9 |
| Colon ca (SW480 met)_SW620 | 17.1 |
| Colon ca_HT29 | 3.6 |
| Colon ca._HCT-116 | 20.1 |
| Colon ca._CaCo-2 | 29.2 |
| 83219_CC Well to Mod Diff(ODO3866) | 31.5 |
| 94936_Colon Aclenocarcinoma | 5.4 |
| 94930_Colon | 0.1 |
| 94935_Colon Adenocarcinoma | 0.9 |
| 113468_Colon Pool | 36.6 |
| 113457_Small Intestine Pool | 33.9 |
| 113460_Stomach Pool | 13.1 |
| 113467_Bone Marrow Pool | 12.9 |
| 103371_Fetal Heart | 12.7 |
| 113451_Heart Pool | 11.4 |
| 113466_Lymph Node Pool | 32.3 |
| 103372_Fetal Skeletal Muscle | 6.4 |
| 113456_Skeletal Muscle Pool | 5.3 |
| 113459_Spleen Pool | 20.5 |
| 113462_Thymus Pool | 15.8 |
| CNS ca (glio/astro)_U87-MG | 14.5 |
| CNS ca (glio/astro)_U-118-MG | 21.3 |
| CNS ca (neuro,met)_SK-N-AS | 33.5 |
| 95264_Brain astrocytoma | 35.3 |
| CNS ca (astro)_SNB-75 | 74.1 |
| CNS ca (glio)_SNB-19 | 16.5 |
| CNS ca. (glio)_SF-295 | 33.7 |
| 113447_Brain (Amygdala) Pool | 21.2 |
| 103382_Brain (cerebellum) | 64.8 |
| 64019-1_brain(fetal) | 43.8 |
| 113448_Brain (Hippocampus) Pool | 14.1 |
| 113464_Cerebral Cortex Pool | 23.8 |
| 113449_Brain (Substantia nigra) Pool | 26.3 |
| 113450_Brain (Thalamus) Pool | 18.5 |
| 103384_Brain (whole) | 27.9 |
| 113458_Spinal Cord Pool | 20.8 |
| 103375_Adrenal Gland | 14.4 |
| 113454_Pituitary gland Pool | 3.1 |
| 103397_Salivary Gland | 4.7 |
| 103369_Thyroid (female) | 6.7 |
| Pancreatic ca._CAPAN2 | 14.2 |
| 113453_Pancreas Pool | 26.4 |

TABLE BI
Panel 3.2

| | Rel. Expr., % |
|---|---|
| Tissue Name | tm9863f_ag1215_al |
| 94905_Daoy_Medulloblastoma/Cerebellum_sscDNA | 2.5 |
| 94906_TE671_Medulloblastom/Cerebellum_sscDNA | 7.2 |
| 94907_D283 Med_Medulloblastoma/Cerebellum_sscDNA | 8.7 |
| 94908_PFSK-1_Primitive Neuroectodermal/Cerebellum_sscDNA | 9.3 |
| 94909_XF-498_CNS_sscDNA | 18.7 |
| 94910_SNB-78_CNS/glioma_sscDNA | 7.4 |
| 94911_SF-268_CNS/glioblasroma_sscDNA | 7.4 |
| 94912_T98G_Glioblastoma_sscDNA | 11.7 |
| 96776_SK-N-SH_Neuroblastoma (metastasis)_sscDNA | 8.4 |
| 94913_SF-295_CNS/glioblastoma_sscDNA | 6.4 |
| 132565_NT2 pool_sscDNA | 22.8 |
| 94914_Cerebellum_sscDNA | 20.9 |
| 96777_Cerebellum_sscDNA | 20.2 |
| 94916_NCI-H292_Mucoepidermoid lung carcinoma_sscDNA | 7.2 |
| 94917_DMS-114_Small cell lung cancer_sscDNA | 13.6 |
| 94918_DMS-79_Small cell lung cancer/neuroendocrine_sscDNA | 100 |
| 94919_NCI-H146_Small cell lung cancer/neuroendocrine_sscDNA | 11 |
| 94920_NCI-H526_Small cell lung cancer/neuroendocrine_sscDNA | 14.7 |
| 94921_NCI-N417_Small cell lung cancer/neuroendocrine_sscDNA | 2.5 |
| 94923_NCI-H82_Small cell lung cancer/neuroendocrine_sscDNA | 6.2 |
| 94924_NCI-H157_Squamous cell lung cancer (metastasis)_sscDNA | 8.9 |
| 94925_NCI-H1155_Large cell lung cancer/neuroendocrine_sscDNA | 10.1 |
| 94926_NCI-H1299_Large cell lung cancer/neuroendocrine_sscDNA | 14.8 |
| 94927_NCI-H1727_Lung carcinoid_sscDNA | 22.5 |
| 94928_NCI-UMC-11_Lung carcinoid_sscDNA | 11.3 |
| 94929_LX-1_Small cell lung cancer_sscDNA | 7.1 |
| 94930_Colo-205_Colon cancer_sscDNA | 0 |
| 94931_KM12_Colon cancer_sscDNA | 3 |
| 94932_KM20L2_Colon cancer_sscDNA | 1 |
| 94933_NCI-H716_Colon cancer_sscDNA | 4 |
| 94935_SW-48_Colon adenocarcinoma_sscDNA | 0.6 |
| 94936_SW1116_Colon adenocarcinoma_sscDNA | 3.7 |
| 94937_LS 174T_Colon adenocarcinoma_sscDNA | 5.4 |
| 94938_SW-948_Colon adenocarcinoma_sscDNA | 0.8 |
| 94939_SW-480_Colon adenocarcinoma_sscDNA | 5.9 |
| 94940_NCI-SNU-5_Gastric carcinoma_sscDNA | 7.3 |
| 112197_KATO III_Stomach_sscDNA | 0 |
| 94943_NCI-SNU-16_Gastric carcinoma_sscDNA | 2.9 |
| 94944_NCI-SNU-1_Gastric carcinoma_sscDNA | 2.4 |
| 94946_RF-1_Gastric adenocarcinoma_sscDNA | 3.3 |
| 94947_RF-48_Gastric adenocarcinoma_sscDNA | 3.6 |
| 96778_MKN-45_Gastric carcinoma_sscDNA | 10.4 |
| 94949_NCI-N87_Gastric carcinoma_sscDNA | 7.7 |
| 94951_OVCAR-5_Ovarian carcinoma_sscDNA | 2.7 |
| 94952_RL95-2_Uterine carcinoma_sscDNA | 3.9 |
| 94953_HelaS3_Cervical adenocarcinoma_sscDNA | 4.3 |
| 94954_Ca Ski_Cervical epidermoid carcinoma (metastasis)_sscDNA | 14.8 |
| 94955_ES-2_Ovarian clear cell carcinoma_sscDNA | 6.6 |
| 94957_Ramos/6h stim_Stimulated with PMA/ionomycin 6h_sscDNA | 0 |
| 94958_Ramos/14h stim_Stimulated with PMA/ionomycin 14h_sscDNA | 0 |
| 94962_MEG-01_Chronic myclogenous leukemia (megokaryoblast)_sscDNA | 12.4 |
| 94963_Raji_Burkitt's lymphonia_sscDNA | 0 |
| 94964_Daudi_Burkitt's lymphoma_sscDNA | 0 |
| 94965_U266_B-cell plasmacytoma/myeloma_sscDNA | 0.3 |
| 94968_CA46_Burkitt's lymphoma_sscDNA | 0 |
| 94970_RL_non-Hodgkin's B-cell lymphoma_sscDNA | 0 |
| 94972_JM1_pre-B-cell lymphoma/leukemia_sscDNA | 0 |
| 94973_Jurkat_T cell leukemia_sscDNA | 1.2 |
| 94974_TF-1_Erythroleukemia_sscDNA | 16.5 |
| 94975_HUT 78T-cell lymphoma_sscDNA | 4.3 |
| 94977_U937_Histiocytic lymphoma_sscDNA | 10.8 |
| 94980_KU-812_Myelogenous leukemia_sscDNA | 13.9 |
| 94981_769-P_Clear cell renal carcinoma_sscDNA | 6.3 |
| 94983_Caki-2_Clear cell renal carcinoma_sscDNA | 6.3 |
| 94984_SW 839_Clear cell renal carcinoma_sscDNA | 5.7 |
| 94986_G401_Wilms' tumor_sscDNA | 3.4 |
| 126768_293 cells_sscDNA | 2.9 |
| 94987_Hs766T_Pancreatic carcinoma (LN metastasis)_sscDNA | 7.1 |
| 94988_CAPAN-1_Pancreatic adenocarcinoma (liver metastasis)_sscDNA | 5.4 |
| 94989_SU86.86_Pancreatic carcinoma (liver metastasis)_sscDNA | 13.5 |
| 94990_BxPC-3_Pancreatic adenocarcinoma_sscDNA | 6.3 |
| 94991_HPAC_Pancreatic adenocarcinoma_sscDNA | 18.4 |
| 94992_MIA PaCa-2 Pancreatic carcinoma_sscDNA | 2.1 |
| 94993_CFPAC-1_Pancreatic ductal adenocarcinoma_sscDNA | 16.6 |
| 94994_PANC-1_Pancreatic epithelioid ductal carcinoma_sscDNA | 9.5 |
| 94996_T24_Bladder carcinma (transitional cell)_sscDNA | 5 |
| 94997_5637_Bladder carcinoma_sscDNA | 8.9 |
| 94998_HT-1197_Bladder carcinoma_sscDNA | 11 |
| 94999_UM-UC-3_Bladder carcinma (transitional cell)_sscDNA | 3.5 |
| 95000_A204_Rhabdomyosarcoma_sscDNA | 6.3 |
| 95001_HT-1080_Fibrosarcoma_sscDNA | 7.7 |
| 95002_MG-63_Osreosarcoma (bone)_sscDNA | 5.6 |
| 95003_SK-LMS-1_Leiomyosarcoma (vulva)_sscDNA | 9.7 |
| 95004_SJRH30_Rhabdomyosarcoma (met to bone marrow)_sscDNA | 11 |
| 95005_A431_Epidermoid carcinoma_sscDNA | 3.1 |
| 95007_WM266-4_Melanoma_sscDNA | 5.3 |
| 112195_DU 145_Prostate_sscDNA | 6.8 |
| 95012_MDA-MB-468_Breast adenocarcinoma_sscDNA | 1.9 |
| 112196_SSC-4_Tongue_sscDNA | 5.1 |
| 112194_SSC-9_Tongue_sscDNA | 2.6 |
| 112191_SSC-15_Tongue_sscDNA | 4.1 |
| 95017_CAL 27_Squamous cell carcinoma of tongue_sscDNA | 7.8 |

TABLE BJ
Panel Hass

| | Rel. Expr., % |
|---|---|
| Tissue Name | tm9864f_ag1215_al |
| 118807 - MCF-7 C1 | 50.2 |
| 118808 - MCF-7 C2 | 58.1 |
| 118809 - MCF-7 C3 | 41.9 |
| 118810 - MCF-7 C4 | 59.5 |
| 118811 - MCF-7 C5 | 56.9 |
| 118812 - MCF-7 C6 | 72.9 |
| 118813 - MCF-7 C7 | 29.8 |
| 118815 - MCF-7 C9 | 32.2 |
| 118816 - MCF-7 C10 | 73.6 |
| 118817 - MCF-7 C11 | 12.7 |
| 118818 - MCF-7 C12 | 46.6 |
| 118819 - MCF-7 C13 | 35.6 |
| 118821 - MCF-7 C15 | 18.4 |
| 118822 - MCF-7 C16 | 100 |
| 118823 - MCF-7 C17 | 60.5 |
| 118824 - T24 D1 | 10.4 |
| 118825 - T24 D2 | 5.8 |
| 118826 - T24 D3 | 9.4 |
| 118827 - T24 D4 | 11.9 |
| 118828 - T24 D5 | 8.5 |
| 118829 - T24 D6 | 11.8 |
| 118830 - T24 D7 | 3.1 |
| 118832 - T24 D9 | 3.2 |
| 118833 - T24 D10 | 4.8 |
| 118834 - T24 D11 | 4.8 |
| 118835 - T24 D12 | 6.6 |
| 118836 - T24 D13 | 2.5 |
| 118838 - T24 D15 | 3.2 |
| 118839 - T24 D16 | 3.2 |
| 118840 - T24 D17 | 4.6 |
| 118841 - CAPaN B1 | 6.3 |
| 118842 - CAPaN B2 | 4 |
| 118843 - CAPaN B3 | 1.4 |
| 118844 - CAPaN B4 | 6.9 |
| 118845 - CAPaN B5 | 4.7 |
| 118846 - CAPaN B6 | 6.1 |
| 118847 - CAPaN B7 | 3.7 |
| 118848 - CAPaN B8 | 5.3 |
| 118849 - CAPaN B9 | 5 |
| 118850 - CAPaN B10 | 8.1 |
| 118851 - CAPaN B11 | 5.6 |
| 118852 - CAPaN B12 | 4.3 |
| 118853 - CAPaN B13 | 6.6 |
| 118854 - CAPaN B14 | 5.6 |
| 118855 - CAPaN B15 | 3.3 |
| 118856 - CAPaN B16 | 7 |
| 118857 - CAPaN B17 | 7.4 |
| 133019 - U87-MG F1 (B) | 3.9 |
| 118876 - U87-MG F2 | 1.8 |
| 118877 - U87-MG F3 | 2.9 |
| 118878 - U87-MG F4 | 4.2 |
| 118879 - U87-MG F5 | 7.8 |
| 118880 - U87-MG F6 | 8.5 |
| 118881 - U87-MG F7 | 3.5 |
| 118882 - U87-MG F8 | 5.1 |
| 118883 - U87-MG F9 | 2.6 |
| 118884 - U87-MG F10 | 5.2 |
| 118885 - U87-MG F11 | 5.6 |
| 118886 - U87-MG F12 | 5.2 |
| 118887 - U87-MG F13 | 3.5 |
| 118888 - U87-MG F14 | 5.9 |
| 118889 - U87-MG F15 | 3.8 |
| 118890 - U87-MG F16 | 6.6 |
| 118891 - U87-MG F17 | 5.7 |
| 118893 - LnCAP A1 | 3.6 |
| 118894 - LnCAP A2 | 3.1 |
| 118895 - LnCAP A3 | 3.5 |
| 118896 - LnCAP A4 | 3.4 |
| 118897 - LnCAP A5 | 2.7 |
| 118898 - LnCAP A6 | 2.6 |
| 118899 - LnCAP A7 | 4.3 |
| 118900 - LnCAP A8 | 3.5 |
| 118901 - LnCAP A9 | 3.4 |
| 118902 - LnCAP A10 | 3.5 |
| 118903 - LnCAP A11 | 7.5 |
| 118904 - LnCAP A12 | 0.7 |
| 118905 - LnCAP A13 | 0.8 |
| 118906 - LnCAP A14 | 0.5 |
| 118907 - LnCAP A15 | 0.3 |
| 118908 - LnCAP A16 | 5.9 |
| 118909 - LnCAP A17 | 3.9 |
| 118892 - Primary Astrocytes | 6 |
| 121824 - Primary Renal Proximal Tubule | 3.2 |
| Epithelial cell A2 | |
| 121827 - Primary melanocytes A5 | 4.8 |
| 126443 - 341 medullo | 0.8 |
| 126444 - 487 medullo | 19.4 |
| 126445 - 425 medullo | 1.8 |
| 126446 - 690 medullo | 9 |
| 126447 - 54 adult glioma | 3.8 |
| 126448 - 245 adult glioma | 7.7 |
| 126449 - 317 adult glioma | 8.3 |
| 126450 - 212 glioma | 7.4 |
| 126451 - 456 glioma | 8.4 |

### Example 2

### TaqMan Data for MOL7

TaqMan data was acquired for MOL7 as described in Eaxample 1 using the primers specified. The relative expression of MOL7 in the described tissues is represented in the graphs below.

[image]

[image]

### Example 3

### SeqCallingTM Technology

cDNA was derived from various human samples representing multiple tissue types, normal and diseased states, physiological states, and developmental states from different donors. Samples were obtained as whole tissue, cell lines, primary cells or tissue cultured primary cells and cell lines. Cells and cell lines may have been treated with biological or chemical agents that regulate gene expression for example, growth factors, chemokines, steroids. The cDNA thus derived was then sequenced using CuraGen's proprietary SeqCalling technology. Sequence traces were evaluated manually and edited for corrections if appropriate. cDNA sequences from all samples were assembled with themselves and with public ESTs using bioinformatics programs to generate CuraGen's human SeqCalling database of SeqCalling assemblies. Each assembly contains one or more overlapping cDNA sequences derived from one or more human samples. Fragments and ESTs were included as components for an assembly when the extent of identity with another component of the assembly was at least 95% over 50 bp. Each assembly can represent a gene and/or its variants such as splice forms and/or single nucleotide polymorphisms (SNPs) and their combinations.

Variant sequences are included in this application. A variant sequence can include a single nucleotide polymorphism (SNP). A SNP can, in some instances, be referred to as a “cSNP” to denote that the nucleotide sequence containing the SNP originates as a cDNA. A SNP can arise in several ways. For example, a SNP may be due to a substitution of one nucleotide for another at the polymorphic site. Such a substitution can be either a transition or a transversion. A SNP can also arise from a deletion of a nucleotide or an insertion of a nucleotide, relative to a reference allele. In this case, the polymorphic site is a site at which one allele bears a gap with respect to a particular nucleotide in another allele. SNPs occurring within genes may result in an alteration of the amino acid encoded by the gene at the position of the SNP. Intragenic SNPs may also be silent, however, in the case that a codon including a SNP encodes the same amino acid as a result of the redundancy of the genetic code. SNPs occurring outside the region of a gene, or in an intron within a gene, do not result in changes in any amino acid sequence of a protein but may result in altered regulation of the expression pattern for example, alteration in temporal expression, physiological response regulation, cell type expression regulation, intensity of expression, stability of transcribed message.

Method of novel SNP Identification: SNPs are identified by analyzing sequence assemblies using CuraGen's proprietary SNPTool algorithm. SNPTool identifies variation in assemblies with the following criteria: SNPs are not analyzed within 10 base pairs on both ends of an alignment; Window size (number of bases in a view) is 10; The allowed number of mismatches in a window is 2; Minimum SNP base quality (PHRED score) is 23; Minimum number of changes to score an SNP is 2/assembly position. SNPTool analyzes the assembly and displays SNP positions, associated individual variant sequences in the assembly, the depth of the assembly at that given position, the putative assembly allele frequency, and the SNP sequence variation. Sequence traces are then selected and brought into view for manual validation. The consensus assembly sequence is imported into CuraTools along with variant sequence changes to identify potential amino acid changes resulting from the SNP sequence variation. Comprehensive SNP data analysis is then exported into the SNPCalling database.

Method of novel SNP Confirmation: SNPs are confirmed employing a validated method know as Pyrosequencing (Pyrosequencing, Westborough, Mass.). Detailed protocols for Pyrosequencing can be found in:

Alderborn et al. Determination of Single Nucleotide Polymorphisms by Real-time Pyrophosphate DNA Sequencing. (2000). Genome Research. 10, Issue 8, August. 1249-1265.

In brief, Pyrosequencing is a real time primer extension process of genotyping. This protocol takes double-stranded, biotinylated PCR products from genomic DNA samples and binds them to streptavidin beads. These beads are then denatured producing single stranded bound DNA. SNPs are characterized utilizing a technique based on all indirect bioluminometric assay of pyrophosphate (PPi) that is released from each dNTP upon DNA chain elongation. Following Klenow polymerase-mediated base incorporation, PPi is released and used as a substrate, together with adenosine 5′-phosphosulfate (APS), for ATP sulfurylase, which results in the formation of ATP. Subsequently, the ATP accomplishes the conversion of luciferin to its oxi-derivative by the action of luciferase. The ensuing light output becomes proportional to the number of added bases, up to about four bases. To allow processivity of the method dNTP excess is degraded by apyrase, which is also present in the starting reaction mixture, so that only dNTPs are added to the template during the sequencing. The process has been fully automated and adapted to a 96-well format, which allows rapid screening of large SNP panels.

### Example 4

### SAGE data

Serial Analysis of Gene Expression, or SAGE, is an experimental technique designed to gain a quantitative measure of gene expression. The SAGE technique itself includes several steps utilizing molecular biological, DNA sequencing and bioinformatics techniques. These steps (reviewed in Adams M D, “Serial analysis of gene expression: ESTs get smaller.” Bioessays. 18(4):261-2 (1996)) have been used to produce 9 or 10 base “tags”, which are then, in some manner, assigned gene descriptions. For experimental reasons, these tags are immediately adjacent to the 3′ end of the 3′-most NlaIII restriction site in cDNA sequences. The Cancer Genome Anatomy Project, or CGAP, is an NCI-initiated and sponsored project, which hopes to delineate the molecular fingerprint of the cancer cell. It has created a database of those cancer-related projects that used SAGE analysis in order to gain insight into the initiation and development of cancer in the human body. The SAGE expression profiles reported in this invention are generated by first identifying the Unigene accession ID associated with the given MTC gene by querying the Unigene database at http://www.ncbi.nlm.nih.gov/UniGene/. This page has then a link to the SAGE: Gene to Tag mapping

http://www.ncbi.nlm.nih.gov/SAGE/SAGEcid.cgi?cid=“unigeneID”).

This generated the reports that are included in this application, which list the number of tags found for the given gene in a given sample along with the relative expression. This information is then used to understand whether the gene has a more general role in tumorogenesis and/or tumor progression. A list of the SAGE libraries (venerated by CGAP and used in the analysis can be found at

http:www.ncbi.nlm.nih.gov/SAGE/sagelb.cgi. cl MOL4b

### NOV5d SAGE Data

SAGE data

| [symbol] | [symbol] | [symbol] | [symbol] | [symbol] |
|---|---|---|---|---|
| [symbol] | [symbol] | [symbol] | [symbol] | [symbol] |

### NOV5d SAGE Data

SAGE data
Hs 7188: same domain, immunoglobin domain
(Ig), was membrane domain TM) and short
SAGE library data and reliable by primary
Reliable tags found in SAGE libraries.

| [symbol] | [symbol] | [symbol] | [symbol] | [symbol] | [symbol] |
|---|---|---|---|---|---|
| | SAGE NC1 | 19 | [symbol] | 1 | 50115 |
| | SAGE NC2 | 20 | [symbol] | 1 | 49552 |
| | SAGE Duke H1043 | 13 | [symbol] | 1 | 76673 |
| | SAGE Duke 1273 | 51 | [symbol] | 2 | 38836 |
| | SAGE TSU | 88 | [symbol] | 1 | 11356 |
| | SAGE Duke H1020 | 19 | [symbol] | 1 | 52371 |
| | SAGE 293-CTRL | 46 | [symbol] | 2 | 43442 |
| | SAGE HCT116 | 49 | [symbol] | 3 | 60322 |
| | SAGE Cage 2 | 81 | [symbol] | 5 | 61601 |
| | SAGE Chem INCuF | 16 | [symbol] | 1 | 62267 |
| | SAGE Chem Normal Pr | 15 | [symbol] | 1 | 66193 |
| | SAGE Chem Tumor Pr | 14 | [symbol] | 1 | 68384 |
| | SAGE CAPAN1 | 290 | [symbol] | 11 | 37926 |
| | SAGE CAPaN2 | 104 | [symbol] | 4 | 38240 |
| | SAGE HS766T | 95 | [symbol] | 3 | 31506 |
| | SAGE Panel | 120 | [symbol] | 3 | 24879 |
| | SAGE HK | 62 | [symbol] | 2 | 32157 |
| | SAGE H126 | 30 | [symbol] | 1 | 32420 |
| | SAGE Duke H54 last | 29 | [symbol] | 2 | 67101 |
| | SAGE Duke H54 EGFR III | 17 | [symbol] | 1 | 57164 |
| | SAGE Duke H392 | 52 | [symbol] | 3 | 57529 |
| | SAGE SW837 | 32 | [symbol] | 2 | 60986 |
| | SAGE REQ | 19 | [symbol] | 1 | 52064 |
| | SAGE CPDR LNCaP-C | 24 | [symbol] | 1 | 41590 |
| | SAGE CPDR LNCaP-T | 22 | [symbol] | 1 | 44122 |
| | SAGE 293-IND | 40 | [symbol] | 1 | 24481 |
| | SAGE PR317 normal prostate | 33 | [symbol] | 2 | 54919 |
| | SAGE PR317 prostate tumor | 15 | [symbol] | 1 | 65109 |
| | SAGE pooled GBM | 64 | [symbol] | 4 | 61841 |
| | SAGE EB542 whitematter | 31 | [symbol] | 3 | 94806 |
| | SAGE normal pool(6th) | 31 | [symbol] | 2 | 63064 |
| | SAGE NC1 | 19 | [symbol] | 1 | 50115 |
| | SAGE Pane 91-16113 | 58 | [symbol] | 2 | 33941 |
| | SAGE Pane 96-6252 | 83 | [symbol] | 3 | 35745 |
| | SAGE OVCA432-2 | 699 | [symbol] | 2 | 2861 |
| | SAGE OV1063-3 | 128 | [symbol] | 5 | 38938 |
| | SAGE Tu102 | 34 | [symbol] | 2 | 57636 |
| | SAGE Tu96 | 61 | [symbol] | 3 | 49005 |
| | SAGE Duke mhh-1 | 288 | [symbol] | 14 | 48488 |
| | SAGE Inc2 | 53 | [symbol] | 1 | 18528 |
| | SAGE PTEN | 106 | [symbol] | 1 | 9380 |
| | SAGE 95-347 | 44 | [symbol] | 3 | 67240 |
| | SAGE 95-259 | 76 | [symbol] | 3 | 39473 |
| | SAGE 95-260 | 22 | [symbol] | 1 | 45179 |
| | SAGE 95-348 | 16 | [symbol] | 1 | 60484 |
| | SAGE HOSE a | 61 | [symbol] | 3 | 48413 |
| | SAGE ES7-1 | 63 | [symbol] | 2 | 31502 |
| | SAGE OVT-5 | 185 | [symbol] | 2 | 10802 |
| | SAGE Medullo 3871 | 23 | [symbol] | 1 | 43274 |
| | SAGE PERITO-13 | 74 | [symbol] | 4 | 53728 |
| | SAGE Meso-13 | 57 | [symbol] | 2 | 35032 |
| | SAGE DukeH1126 | 71 | [symbol] | 2 | 27820 |
| | SAGE gastric cancer | 42 | [symbol] | 3 | 70155 |
| | repograft X101 | | | | |
| | SAGE gastric cancer-Q234 | 30 | [symbol] | 2 | 65291 |
| | SAGE Duke Indocyte | 41 | [symbol] | 2 | 48169 |
| | SAGE normal lung | 78 | [symbol] | 7 | 88901 |
| | SAGE breast epithelium | 173 | [symbol] | 10 | 57785 |
| | SAGE breast hyperplasma | 95 | [symbol] | 6 | 62585 |
| | SAGE QC14 | 57 | [symbol] | 1 | 17476 |
| | SAGE gliom 1150 | 15 | [symbol] | 1 | 62523 |
| | SAGE normal heart | 11 | [symbol] | 1 | 84072 |
| | SAGE hemadorpa 146 | 65 | [symbol] | 5 | 75850 |
| | SAGE OV1-6 | 307 | [symbol] | 13 | 42336 |
| | SAGE OVT-7 | 91 | [symbol] | 5 | 54914 |
| | SAGE Duke HMVEC | 114 | [symbol] | 6 | 52532 |
| | SAGE Duke HMVEC + VEGP | 103 | [symbol] | 6 | 57928 |
| | SAGE mammary epithelium | 20 | [symbol] | 1 | 48167 |
| | SAGE DCIS | 24 | [symbol] | 1 | 41230 |
| | SAGE Duke 757 | 51 | [symbol] | 1 | 19503 |
| | SAGE normal cerebellum | 38 | [symbol] | 3 | 51135 |
| | SAGE OVT-8 | 119 | [symbol] | 4 | 33575 |
| | SAGE DUKE 48NT | 248 | [symbol] | 3 | 12091 |
| | SAGE A2780-9 | 269 | [symbol] | 6 | 22256 |
| | SAGE ML10-10 | 122 | [symbol] | 7 | 36943 |
| | SAGE Duke H247 normal | 99 | [symbol] | 6 | 60543 |
| | SAGE Duke H247 Hyposis | 55 | [symbol] | 4 | 71937 |
| | SAGE JOSE23-11 | 20 | [symbol] | 1 | 48498 |
| | SAGE Duke E1043 | 39 | [symbol] | 3 | 76673 |

What is claimed is:

1. An isolated polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 102, 104, 106, 108, 111, 114, 116, 118, 120, 123, and 125;
(b) a variant of a mature form of an amino acid sequence selected from the group consisting of SEQ ID NOS:2, 4, 6, 8, 10, 12, 14, 16, 18, 20, 22, 24, 102, 104, 106, 108, 111, 114, 116, 118, 120, 123, and 125, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 102, 104, 106, 108, 111, 114, 116, 118, 120, 123, and 125; and
(d) a variant of an amino acid sequence selected from the group consisting of SEQ ID NOS:2, 4, 6, 8, 10, 12, 14, 16, 18, 20, 22, 24, 102, 104, 106, 108, 111, 114, 116, 118, 120, 123, and 125, wherein one or more amino acid residues in said variant differs from the amino acid sequence of said mature form, provided that said variant differs in no more than 15% of amino acid residues from said amino acid sequence.

2 The polypeptide of claim 1, wherein said polypeptide comprises the amino acid sequence of a naturally-occurring allelic variant of an amino acid sequence selected from the group consisting of SEQ ID NOS:2, 4, 6, 8, 10, 12, 14, 16, 18, 20, 22, 24, 102, 104, 106, 108, 111, 114, 116, 118, 120, 123,and 125.

3. The polypeptide of claim 2, wherein said allelic variant comprises an amino acid sequence that is the translation of a nucleic acid sequence differing by a single nucleotide from a nucleic acid sequence selected from the group consisting of SEQ ID NOS:1, 3, 5, 7, 9, 11, 13, 15, 17, 19, 21, 23, 101, 103, 105, 107, 109, 112, 115, 117, 119, 121, and 124.

4. The polypeptide of claim 1, wherein the amino acid sequence of said variant comprises a conservative amino acid substitution.

5. An isolated nucleic acid molecule comprising a nucleic acid sequence encoding a polypeptide comprising an amino acid sequence selected from the group consisting of:
(a) a mature form of an amino acid sequence selected from the group consisting of SEQ ID NOS:2, 4, 6, 8, 10, 12, 14, 16, 18, 20, 22, 24, 102, 104, 106, 108, 111, 114, 116, 118, 120, 123, and 125;
(b) a variant of a mature form of an amino acid sequence selected from the group consisting of SEQ ID NOS:2, 4, 6, 8, 10, 12, 14, 16, 18, 20, 22, 24, 102, 104, 106, 108, 111, 114, 116, 118, 120, 123, and 125, wherein one or more amino acid residues in said variant differs from the amino acid sequence of said mature form, provided that said variant differs in no more than 15% of the amino acid residues from the amino acid sequence of said mature form;
(c) an amino acid sequence selected from the group consisting of SEQ ID NOS:2, 4, 6, 8, 10, 12, 14, 16, 18, 20, 22, 24, 102, 104, 106, 108, 111, 114, 116, 118, 120, 123, and 125;
(d) a variant of an amino acid sequence selected from the group consisting SEQ ID NOS:2, 4, 6, 8, 10, 12, 14, 16, 18, 20, 22, 24, 102, 104, 106, 108, 111, 114, 116, 118, 120, 123, and 125, wherein one or more amino acid residues in said variant differs from the amino acid sequence of said mature form, provided that said variant differs in no more than 15% of amino acid residues from said amino acid sequence;
(e) a nucleic acid fragment encoding at least a portion of a polypeptide comprising an amino acid sequence chosen from the group consisting of SEQ ID NOS:2, 4, 6, 8, 10, 12, 14, 16, 18, 20, 22, 24, 102, 104, 106, 108, 111, 114, 116, 118, 120, 123, and 125, or a variant of said polypeptide, wherein one or more amino acid residues in said variant differs from the amino acid sequence of said mature form, provided that said variant differs in no more than 15% of amino acid residues from said amino acid sequence; and
(f) a nucleic acid molecule comprising the complement of (a), (b), (c), (d) or (c).

6. The nucleic acid molecule of claim 5, wherein the nucleic acid molecule comprises the nucleotide sequence of a naturally-occurring allelic nucleic acid variant.

7. The nucleic acid molecule of claim 5, wherein the nucleic acid molecule encodes a polypeptide comprising the amino acid sequence of a naturally-occurring polypeptide variant.

8. The nucleic acid molecule of claim 5, wherein the nucleic acid molecule differs by a single nucleotide from a nucleic acid sequence selected from the group consisting of SEQ ID NOS:1, 3, 5, 7, 9, 11, 13, 15, 17, 19, 21, 23, 101, 103, 105, 107, 109, 112, 115, 117, 119, 121, and 124.

9. The nucleic acid molecule of claim 5, wherein said nucleic acid molecule comprises a nucleotide sequence selected from the group consisting of:
(a) a nucleotide sequence selected from the group consisting of SEQ ID NOS:1, 3, 5, 7, 9, 11, 13, 15, 17, 19, 21, 23, 101, 103, 105, 107, 109, 112, 115, 117, 119, 121, and 124;
(b) a nucleotide sequence differing by one or more nucleotides from a nucleotide sequence selected from the group consisting of SEQ ID NOS:1, 3, 5, 7, 9, 11, 13, 15, 17, 19, 21, 23, 101, 103, 105, 107, 109, 112, 115, 117, 119, 121, and 124, provided that no more than 20% of the nucleotides differ from said nucleotide sequence;
(c) a nucleic acid fragment of (a); and
(d) a nucleic acid fragment of (b).

10. The nucleic acid molecule of claim 5, wherein said nucleic acid molecule hybridizes under stringent conditions to a nucleotide sequence chosen from the group consisting of SEQ ID NOS:3, 5, 7, 9, 11, 13, 15, 17, 19, 21, 23, 101, 103, 105, 107, 109, 112, 115, 117, 119, 121, and 124, or a complement of said nucleotide sequence.

11. The nucleic acid molecule of claim 5, wherein the nucleic acid molecule comprises a nucleotide sequence selected from the group consisting of:
(a) a first nucleotide sequence comprising a coding sequence differing by one or more nucleotide sequences from a coding sequence encoding said amino acid sequence, provided that no more than 20% of the nucleotides in the coding sequence in said first nucleotide sequence differ from said coding sequence;
(b) an isolated second polynucleotide that is a complement of the first polynucleotide; and
(c) a nucleic acid fragment of (a) or (b).

12. A vector comprising the nucleic acid molecule of claim 11.

13. The vector of claim 12, further comprising a promoter operably-linked to said nucleic acid molecule.

14. A cell comprising the vector of claim 12.

15. An antibody that binds immunospecifically to the polypeptide of claim 1.

16. The antibody of claim 15, wherein said antibody is a monoclonal antibody.

17. The antibody of claim 15, wherein the antibody is a humanized antibody.

18. A method for determining the presence or amount of the polypeptide of claim 1 in a sample, the method comprising:
(a) providing the sample;
(b) contacting the sample with an antibody that binds immunospecifically to the polypeptide; and
(c) determining the presence or amount of antibody bound to said polypeptide, thereby determining the presence or amount of polypeptide in said sample.

19. A method for determining the presence or amount of the nucleic acid molecule of claim 5 in a sample, the method comprising:
(a) providing the sample;
(b) contacting the sample with a probe that binds to said nucleic acid molecule; and
(c) determining the presence or amount of the probe bound to said nucleic acid molecule, cl thereby determining the presence or amount of the nucleic acid molecule in said sample.

20. The method of claim 19 wherein presence or amount of the nucleic acid molecule is used as a marker for cell or tissue type.

21. The method of claim 20 wherein the cell or tissue type is cancerous.

22. A method of identifying an agent that binds to a polypeptide of claim 1, the method comprising:
(a) contacting said polypeptide with said agent; and
(b) determining whether said agent binds to said polypeptide.

23. The method of claim 22 wherein the agent is a cellular receptor or a downstream effector.

24. A method for identifying an agent that modulates the expression or activity of the polypeptide of claim 1, the method comprising:
(a) providing a cell expressing said polypeptide;
(b) contacting the cell with said agent, and
(c) determining whether the agent modulates expression or activity of said polypeptide, cl whereby an alteration in expression or activity of said peptide indicates said agent modulates expression or activity of said polypeptide.

25. A method for modulating the activity of the polypeptide of claim 1, the method comprising contacting a cell sample expressing the polypeptide of said claim with a compound that binds to said polypeptide in an amount sufficient to modulate the activity of the polypeptide.

26. A method of treating or preventing a MOLX-associated disorder, said method comprising administering to a subject in which such treatment or prevention is desired the polypeptide of claim 1 in an amount sufficient to treat or prevent said MOLX-associated disorder in said subject.

27. The method of claim 26 wherein the disorder is selected from the group consisting of cardiomyopathy and atherosclerosis.

28. The method of claim 26 wherein the disorder is related to cell signal processing and metabolic pathway modulation.

29. The method of claim 26, wherein said subject is a human.

30. A method of treating or preventing a MOLX-associated disorder, said method comprising administering to a subject in which such treatment or prevention is desired the nucleic acid of claim 5 in an amount sufficient to treat or prevent said MOLX-associated disorder in said subject.

31. The method of claim 30 wherein the disorder is selected from the group consisting of cardiomyopathy and atherosclerosis.

32. The method of claim 30 wherein the disorder is related to cell signal processing and metabolic pathway modulation.

33. The method of claim 30, wherein said subject is a human.

34. A method of treating or preventing a MOLX-associated disorder, said method comprising administering to a subject in which such treatment or prceeition is desired the antibody of claim 15 in an amount sufficient to treat or prevent said MOLX-associated disorder in said subject.

35. The method of claim 34 wherein the disorder is diabetes.

36. The method of claim 34 wherein the disorder is related to cell signal processing and metabolic pathway modulation.

37. The method of claim 34, wherein the subject is a human.

38. A pharmaceutical composition comprising the polypeptide of claim 1 and a pharmaceutically-acceptable carrier.

39. A pharmaceutical composition comprising the nucleic acid molecule of claim 5 and a pharmaceutically-acceptable carrier.

40. A pharmaceutical composition comprising the antibody of claim 15 and a pharmaceutically-acceptable carrier.

41. A kit comprising in one or more containers, the pharmaceutical composition of claim 38.

42. A kit comprising in one or more containers, the pharmaceutical composition of claim 39.

43. A kit comprising in one or more containers, the pharmaceutical composition of claim 40.

44. A method for determining the presence of or predisposition to a disease associated with altered levels of the polypeptide of claim 1 in a first mammalian subject, the method comprising:
(a) measuring the level of expression of the polypeptide in a sample from the first mammalian subject; and
(b) comparing the amount of said polypeptide in the sample of step (a) to the amount of the polypeptide present in a control sample from a second mammalian subject known not to have, or not to be predisposed to, said disease; cl wherein an alteration in the expression level of the polypeptide in the first subject as compared to the control sample indicates the presence of or predisposition to said disease.

45. The method of claim 44 wherein the predisposition is to cancers.

46. A method for determining the presence of or predisposition to a disease associated with altered levels of the nucleic acid molecule of claim 5 in a first mammalian subject, the method comprising;
(a) measuring the amount of the nucleic acid in a sample from the first mammalian subject; and
(b) comparing the amount of said nucleic acid in the sample of step (a) to the amount of the nucleic acid present in a control sample from a second mammalian subject known not to have or not be predisposed to, the disease; cl wherein an alteration in the level of the nucleic acid in the first subject as compared to the control sample indicates the presence of or predisposition to the disease.

47. The method of claim 46 wherein the predisposition is to a cancer.

48. A method of treating a pathological state in a mammal, the method comprising administering to the mammal a polypeptide in an amount that is sufficient to alleviate the pathological state, wherein the polypeptide is a polypeptide having an amino acid sequence at least 95% identical to a polypeptide comprising an amino acid sequence of at least one of SEQ ID NOS:2, 4, 6, 8, 10, 12, 14, 16, 18, 20, 22, 24, 102, 104, 106, 108, 111, 114, 116, 118, 120, 123, and 125, or a biologically active fragment thereof.

49. A method of treating a pathological state in a mammal, the method comprising administering to the mammal the antibody of claim 15 in an amount sufficient to alleviate the pathological state.

50. A method for the screening of a candidate substance interacting with an olfactory receptor polypeptide selected from the group consisting of SEQ ID NOS:2, 4, 6, 8, 10, 12, 14, 16, 18, 20, 22, 24, 102, 104, 106, 108, 111, 114, 116, 118, 120, 123, and 125, or fragments or variants thereof, comprises the following steps:
a) providing a polypeptide selected from the group consisting of the sequences of SEQ ID NOS:2, 4, 6, 8, 10, 12, 14, 16, 18, 20, 22, 24, 102, 104, 106, 108, 111, 114, 116, 118, 120, 123, and 125, or a peptide fragment or a variant thereof;
b) obtaining a candidate substance;
c) bringing into contact said polypeptide with said candidate substance; and
d) detecting the complexes formed between said polypeptide and said candidate substance.

51. A method for the screening of ligand molecules interacting with all olfactory receptor polypeptide selected from the group consisting of SEQ ID NOS:2, 4, 6, 8, 10, 12, 14, 16, 18, 20, 22, 24, 102, 104, 106, 108, 111, 114, 116, 118, 120, 123, and 125, wherein said method comprises:
a) providing a recombinant eukaryotic host cell containing a nucleic acid encoding a polypeptide selected from the group consisting of the polypeptides comprising the amino acid sequences SEQ ID NOS:2, 4, 6, 8, 10, 12, 14, 16, 18, 20, 22, 24, 102, 104, 106, 108, 111, 114, 116, 118, 120, 123, and 125;
b) preparing membrane extracts of said recombinant eukaryotic host cell;
c) bringing into contact the membrane extracts prepared at step b) with a selected ligand molecule; and
d) detecting the production level of second messengers metabolites.

52. A method for the screening of ligand molecules interacting with an olfactory receptor polypeptide selected from the group consisting of SEQ ID NOS:2, 4, 6, 8, 10, 12, 14, 16, 18, 20, 22, 24, 102, 104, 106, 108, 111, 114, 116, 118, 120, 123, and 125, wherein said method comprises:
a) providing an adenovirus containing a nucleic acid encoding a polypeptide selected from the group consisting of polypeptides comprising the amino acid sequences SEQ ID NOS:2, 4, 6, 8, 10, 12, 14, 16, 18, 20, 22, 24, 102, 104, 106, 108, 111, 114, 116, 118, 120, 123,and 125;
b) infecting an olfactory epitheliums with said adenovirus;
c) bringing into contact the olfactory epithelium b) with a selected ligand molecule; and
d) detecting the increase of the response to said ligand molecule.
